(19) United States
(12) Patent Application Publication
(10) Pub. No.: US 2026/0100251 A1
(43) Pub. Date: Apr. 9, 2026

(54) Methods for Rule-based Genome Design

(71) Applicant: President and Fellows of Harvard College, Cambridge, MA (US)

(72) Inventors: Gleb Kuznetsov, Cambridge, MA (US); Marc J. Lajoie, Cambridge, MA (US); Matthieu M. Landon, Boston, MA (US); Michael G. Napolitano, Brookline, MA (US); Daniel Bryan Goodman, Cambridge, MA (US); Christopher J. Gregg, Melrose, MA (US); George M. Church, Brookline, MA (US); Nili Ostrov, Brookline, MA (US)

(21) Appl. No.: 18/901,470

(22) Filed: Sep. 30, 2024

Related U.S. Application Data
(63) Continuation of application No. 17/719,431, filed on Apr. 13, 2022.
Application No. 17/719,431 is a continuation of application No. 16/309,645, now Pat. No. 11,361,845.
(60) Provisional application No. 62/350,468, filed on Jun. 15, 2016.

(51) Int. Cl. G16B 35/10; A61K 38/00; C07K 14/245; C12N 15/10; C12P 21/00; G16B 20/50

(52) U.S. Cl. CPC G16B 35/10; C07K 14/245; C12N 15/1089; C12P 21/00; G16B 20/50; A61K 38/00

(57) ABSTRACT

Methods and systems for designing, testing, and validating genome designs based on rules or constraints or conditions or parameters or features and scoring are described herein. A computer-implemented method includes receiving data for a known genome and a list of alleles, identifying and removing occurrences of each allele in the known genome, determining a plurality of allele choices with which to replace occurrences in the known genome, generating a plurality of alternative gene sequences for a genome design based on the known genome, wherein each alternative gene sequence comprises a different allele choice, applying a plurality of rules or constraints or conditions or parameters or features to each alternative gene sequence by assigning a score for each rule or constraint or condition or parameter or feature in each alternative gene sequence, resulting in scores for the applied plurality of rules or constraints or conditions or parameters or features, scoring each alternative gene sequence based on a weighted combination of the scores for the plurality of rules or constraints or conditions or parameters or features, and selecting at least one alternative gene sequence as the genome design based on the scoring.

### RELATED APPLICATION DATA

This application is a continuation application which claims priority to U.S. application Ser. No. 17/719,431 and filed Apr. 13, 2022; which is a continuation application which claims priority to U.S. application Ser. No. 16/309,645, now U.S. Pat. No. 11,361,845, and filed Dec. 13, 2018; which is a National Stage Application under 35 U.S.C. 371 of co-pending PCT application PCT/US17/37596 designating the United States and filed Jun. 15, 2017; which claims the benefit of U.S. provisional application No. 62/350,468 filed on Jun. 15, 2016 each of which are hereby incorporated by reference in their entireties.

### STATEMENT OF GOVERNMENT INTERESTS

This invention was made with government support under DE-FG02-02ER63445 awarded by Department of Energy and HR0011-13-1-0002 awarded by Department of Defense. The government has certain rights in the invention.

### SEQUENCE LISTING

The instant application contains a Sequence Listing which has been submitted electronically in XML format and is hereby incorporated by reference in its entirety. The XML copy, created on Sep. 27, 2024, is named “Sequence_Listing_010498.01617_ST26” and is 2.93 MB in size.

### FIELD

Aspects described herein generally relate to genetic engineering and genetically modified cells and/or organisms. In particular, one or more aspects of the disclosure are directed to methods and computer software useful for genome design based on a predefined set of rules or conditions or parameters or features.

### BACKGROUND

Genetically modified organisms (GMOs) are being used increasingly to produce human consumables such as fuels, commodity chemicals, and therapeutics. GMOs are also used in agriculture (e.g., golden rice, Roundup Ready® crops, Frostban), bioremediation (e.g., oil spills), and healthcare (e.g., Crohn's disease and oral inflammation). Modifications in commercially implemented GMOs may often be limited to heterologous gene expression and evolution under optimizing selection. Yet synthetic genomes that differ radically from any known organism may expand potential applications.

There has been considerable interest in creating minimal (Gibson et al., 2010) and recoded (Lajoie et al., 2013a; Lajoie et al., 2013b) genomes, but genomes are not yet understood well enough to design them from scratch. While in vivo genome engineering strategies may reduce the risk of creating nonfunctional genomes (Lajoie et al., 2013a; Lajoie et al., 2013b), rational design may still be indispensable for restricting the search space to create viable genomes with a desired function. Therefore, the field of genome engineering may be in dire need of general design rules or conditions or parameters or features, methods of eliciting these rules or conditions or parameters or features, and software that may be used to generate viable and constructable genomes.

### SUMMARY

The following presents a simplified summary of various aspects described herein. This summary is not an extensive overview, and is not intended to identify key or critical elements or to delineate the scope of the claims. The following summary merely presents some concepts in a simplified form as an introductory prelude to the more detailed description provided below.

Aspects of the present disclosure provide methods, algorithms, computing platforms, and computer software for designing genomes based on satisfying a set of rules or conditions or parameters or features while minimizing disturbances to biologically relevant motifs, synthesizing the genome designs, and testing and validating the synthesized genome designs. A computing platform may generate genome designs and partition the genome designs into units that may be synthesized and/or edited, in which the genome designs satisfy user-specified constraints and maximize the probability of biological viability and constructability. Units or individual components of the redesigned genome may be tested, and design failures may be detected based on identifying components that fail testing. Rules or conditions or parameters or features for the genome design may be updated accordingly, and recommendations for subsequent iterations may be provided.

Aspects of this disclosure are directed to a method for designing genomes implemented by a computing platform. The method includes receiving, as an input at a computing platform, data for a known genome and a list of alleles to be replaced in the known genome, based on the list of alleles, identifying, by the computing platform, occurrences of each allele in the known genome, removing, by the computing platform, the occurrences of each allele from the known genome, determining, by the computing platform, a plurality of allele choices with which to replace occurrences of each allele in the known genome, generating, by the computing platform, a plurality of alternative gene sequences for a genome design based on the known genome, wherein each alternative gene sequence comprises a different allele choice from the plurality of allele choices, applying, by the computing platform, a plurality of rules or conditions or parameters or features to each alternative gene sequence by assigning a score for each rule or condition or parameter or feature in each alternative gene sequence, resulting in scores for the plurality of rules or conditions or parameters or features applied to each alternative gene sequence, scoring, by the computing platform, each alternative gene sequence based on a weighted combination of the scores for the plurality of rules or conditions or parameters or features, and selecting, by the computing platform, at least one alternative gene sequence as the genome design based on the weighted scoring.

In some embodiments, the disclosed genome design method may be implemented for any type of genome, including bacterial genomes, mycoplasma genomes, yeast genomes, human genomes, genomes for any naturally-occurring organism, or genomes for any previously evolved or engineered organism. In additional embodiments, the disclosed genome design method may be implemented for designing any genomic changes, including removing any alleles, removing sites for restriction enzymes, replacing repetitive extragenic palindromic (REP) sequences with terminators, deleting non-essential genes, inserting heterologous genes to expand function, and the like.

According to some aspects, a method for updating rules in genome design is provided. The method includes introducing one or more features of a genome design into at least one cell, testing the one or more features of the at least one cell by an assay in order to identify genome viability and evaluate the phenotype of the one or more features introduced into the at least one cell, based on the testing, determining that the one or more features introduced into the at least one cell are expected to be viable or expected to fail according to one or more predefined rules or conditions or parameters or features for the genome design, and updating the predefined rules or conditions or parameters or features for genome design based on the determination. In some embodiments, the predefined rules may be updated by leveraging statistical techniques or machine learning algorithms.

Aspects of this disclosure provide a computer-implemented method for testing and modifying genome designs. The method includes obtaining all or a portion of a known genome sequence and a genome design generated by a computing platform, determining that one or more features in the genome design fail a set of predefined rules or conditions or parameters or features, predicting modifications to the genome design to satisfy a predetermined design objective and to increase probability of viability, and testing the predicted modifications to generate an improved genome design.

Additional aspects of the disclosure provide methods for identifying sequence designs when no computationally designed solution is found to be viable or confer the desired phenotype. Degenerate DNA sequences may be tested in combinations. Viable or phenotypically correct individual sequences may be identified by screening or selection. Viable DNA sequences may be used to update or learn new computational design rules or conditions or parameters or features.

The disclosure provides an engineered organism comprising a recoded genome wherein a particular sense codon at all instances within a gene or non-coding motif in a template genome is changed to alternative codons. According to one aspect, the gene is an essential gene or a non-essential gene encoding a protein sequence. According to one aspect, an instance of a particular sense codon overlaps with a non-coding motif. According to one aspect, the non-coding motif is a ribosome binding site motif, an mRNA secondary structure, an internal ribosome pausing site motif or a promoter. According to one aspect, the protein sequence is preserved. According to one aspect, the non-coding motif is preserved. According to one aspect, the particular sense codon is a member selected from the group consisting of AGG, AGA, AGC, AGU, UUG, and UUA. According to one aspect, the engineered organism is E. coli. According to one aspect, the engineered organism is virus resistant or biocontained. According to one aspect, a cognate tRNA to the particular sense codon is eliminated from the template genome. According to one aspect, a cognate tRNA to the particular sense codon is not present in the recoded genome. According to one aspect, the particular sense codon is placed within the engineered organism and is reassigned to a non-standard amino acid. According to one aspect, the alternative codon is a synonymous codon. According to one aspect, the alternative codon is a non-synonymous codon. The present disclosure provides an engineered organism comprising a recoded genome wherein a particular sense codon at all instances within genes or non-coding motifs in a template genome are changed to alternative codons. The present disclosure provides an engineered organism comprising a recoded genome wherein a particular sense codon in a template genome is changed genome-wide to alternative codons. The present disclosure provides an engineered organism comprising a recoded genome wherein particular sense codons at all instances within an essential gene in a template genome are changed to alternative codons. The present disclosure provides an engineered organism comprising a recoded genome wherein particular sense codons at all instances within essential genes in a template genome are changed to alternative codons. The present disclosure provides an engineered organism comprising a recoded genome wherein particular sense codons in a template genome are changed genome-wide to alternative codons. The present disclosure provides an engineered organism comprising a recoded genome designed by the methods described herein. The present disclosure provides an engineered organism comprising a recoded genome wherein instances of a particular sense codon are changed to alternative codons such that the cognate tRNA to the particular sense codon can be eliminated from the engineered organism. The present disclosure provides an engineered organism comprising a recoded genome wherein instances of a particular sense codon are changed to alternative codons such that translation function of the particular sense codon can be changed. The present disclosure provides an engineered organism comprising a recoded genome wherein instances of a particular sense codon are changed to alternative codons such that translation function of the particular sense codon can be eliminated.

Further features and advantages of certain embodiments of the present disclosure will become more fully apparent in the following description of embodiments and drawings thereof, and from the claims.

### BRIEF DESCRIPTION OF THE DRAWINGS

The foregoing and other features and advantages of the present embodiments will be more fully understood from the following detailed description of illustrative embodiments taken in conjunction with the accompanying drawings in which:

FIG. 1 illustrates a block diagram of an example computing device that may be utilized to execute software in accordance with one or more example embodiments.

FIG. 2 illustrates an example block diagram of a genome design module in which various aspects of the present disclosure may be implemented in accordance with one or more example embodiments.

FIG. 3 illustrates an example flow diagram of example method steps for designing genomes in accordance with one or more example embodiments.

FIG. 4 illustrates an example graph of predicted viral resistance of recoded genomes.

FIGS. 5A-5C illustrate an example of a 57-codon E. coli genome. FIG. 5A illustrates the entire recoded genome divided into 87 segments of ˜50-kb. Codons AGA, AGG, AGC, AGU, UUA, UUG, and UAG were computationally replaced by synonymous alternatives (center). Other codons (e.g. UGC) remain unchanged. Color-coded histograms represent the abundance of the seven forbidden codons in each segment. FIG. 5B illustrates codon frequencies in non-recoded (wt; E. coli MDS42) versus recoded (rc) genome. Forbidden codons are colored. FIG. 5C illustrates the scale of DNA editing in genomes constructed by de novo synthesis. Plot area represents DNA editing as the number of modified bp compared to the parent genome. Dark gray represents percent of genome (63%) validated in vivo. Wt, wild-type.

FIG. 6 illustrates a genealogy of recoded E. coli strains, including the lineage of genome-recoded E. coli strains and their computational and biological parents. Commonly used laboratory strains are shown in green. Non-E. coli strain from which orthogonal tRNA was imported is shown in brown. Previously published recoded strains are shown in blue. Strains constructed in the current study are shown in black. The final rE. coli-57 and its bio-contained counterpart rE. coli-57C are shown in gray. (aaRS=aminoacyl-tRNA synthetase).

FIG. 7 illustrates Serine, Arginine, Leucine and Stop codon frequency is for E. coli MDS42 (dark color) and the computationally designed rE. coli-57 genome (light color, frequency labeled).

FIG. 8 illustrates an overview of the computational pipeline for recoded genome design. The software accepts as input a genome template (GenBank file) and a list of codons to be replaced. User-defined rules, both biological and technical (A-G), are then applied to generate a new recoded genome (Genbank file). Synthesis-compatible 2-4 kb sequences are generated. Rules A-G are schematized in FIGS. 9A-9G and further explained in Tables 1-2.

FIGS. 9A-9G illustrate rules or conditions or parameters or features or guidelines for computational design. FIG. 9A discloses SEQ ID NOS 2263-2265, 2264, 2266-2267, 2270, 2268-2270, respectively, in order of appearance. FIG. 9B discloses SEQ ID NOS 2271-2273, and 2272, respectively, in order of appearance. FIG. 9D discloses SEQ ID NOS 2274-2276, and 2275, respectively, in order of appearance. FIG. 9E discloses SEQ ID NOS 2277-2279, and 2278, respectively, in order of appearance.

FIGS. 10A-10C illustrate an experimental strategy for recoded genome validation. FIG. 10A illustrates a pipeline schematic comprising 1) computational design of a 57-codon genome; 2) de novo synthesis of 2 to 4-kb overlapping recoding fragments; 3) assembly of 50-kb segment in S. cerevisiae (orange) on a low copy plasmid; 4) plasmid electroporation in E. coli (wt.seg-non-recoded chromosomal segment); 5) chromosomal sequence corresponding to recoded segment (e.g., wt.seg) replaced by kanamycin cassette (Kan), such that cell viability depends solely on expression of recoded genes; 6) λ-integrase-mediated recombination of attP and attB sequences (P-episomal, B-chromosomal); 6a,b) elimination of residual vectors (see (FIG. 10C)); 7) single-copy integrated recoded segment. attL-attR sites shown in gray. FIG. 10B illustrates PCR analysis of steps 4-7. (Lanes: “L”-GeneRuler 1-kb plus ladder; “C”-control Top10; numbers 4-7 correspond to schematics in FIG. 10A). Red arrows denote PCR primers. FIG. 10C illustrates Cas9-mediated vector elimination, in which residual vector carrying recoded segment is targeted for digestion by Cas9 using attP-specific guide RNA (gRNA). In 6a) additional copies of the recoded segment carry intact attP sequence; 6b) shows Cas9 targeting of attP sequence to eliminate additional vector copies. The integrated segment is not cut since it does not contain an attP sequence. All steps were confirmed by PCR analysis. “gRNA”-guide-RNA.

FIG. 11 illustrates an example of rE. coli-57 genome construction. The genome was parsed into 87 segments, each ˜50 kb in size. All recoded segments were de novo synthesized (green). A total of 55 segments were tested in vivo thus far (blue), of which 44 were successfully validated for all gene functionality on low copy plasmids (red), and 10 segments were further successfully reduced to single copy of all recoded genes (yellow)

FIGS. 12A-12D illustrate phenotypic analysis of recoded strains. In FIG. 12A, recoded segments were episomally expressed in the absence of corresponding wild-type genes. Doubling time is shown relative to the non-recoded parent strain, FIG. 12B illustrates localization of fitness impairment in segment 21, Chromosomal genes (gray) were deleted to test for complementation by recoded genes (orange). Decrease in doubling time was observed upon deletion of rpmF-accC operon. Essential genes in FIG. 12B are framed. In FIG. 12C, fine-tuning of rpmF-accC operon promoter resulted in increased gene expression and decrease in doubling time. (Orange: Initial promoter. Green: Improved promoter). FIG. 12D illustrates RNA-Seq analysis of 208 recoded genes (blue, segments 21, 38, 44, 46, 70). (Wt gene expression shown in gray. Differentially expressed recoded genes shown in red (absolute log 2 fold-change >2, adjusted p-value <0.01). Inset: P-value distribution of recoded genes).

FIGS. 13A-13B illustrate graphs representing fitness of partially recoded strains. FIG. 13A illustrates measurements of doubling time before and after removal of the wild-type chromosomal sequence in strains carrying a recoded segment on low copy plasmid (see steps 4 and 5 in FIG. 10A). FIG. 13B illustrates measurements of doubling time before and after removal of the wild-type sequence, and after chromosomal integration (see steps 4, 5, 6 and 7 in FIG. 10A). Relative Doubling time-fold change between modified and parental strain (i.e. intact genome and no recoded segments).

FIGS. 14A-14B illustrate a transcriptional landscape of recoded segment 43, in which expression levels of all genes within segment 43 are shown. Genes were analyzed in non-recoded strain (TOP10) and after chromosomal deletion. RNA was prepared independently for the different strains, and sequenced on an Illumina MiSeq using PE150 V2 kits (Illumina). For analysis of differential expression, counts were aggregated corresponding to genes using Genomic Features (Bioconductor). Counts obtained per gene were normalized at the genome-wide level using DESeq2 package (Bioconductor) (Anders et al., 2010). FIG. 14A shows expression levels for recoded (green) and non-recoded (purple) genes. FIG. 14B shows p-value and fold changes for all recoded genes. None of the genes in segment 43 was found to be significantly differentially expressed (i.e absolute log 2 fold-change >2 and adjusted p-value <0.01).

FIGS. 15A-15B illustrate an example of troubleshooting lethal design exceptions. In FIG. 15A, recoded segment 44 (orange) did not support cell viability upon complete deletion of chromosomal sequence (Chr-Δseg44.0). The causative recoded gene (accD) was identified by successive chromosomal deletions (Chr-Δseg44.1-4. ‘X’-nonviable). Essential genes are framed. In FIG. 15B, λ-recombination was used to exchange lethal accD sequence (accD.Initial, recoded codons in orange) with an alternative recoded accD sequence (accD.Improved, alternative codons in blue). mRNA structure and RBS motif strength were calculated for both sequences. Wt shown in gray. ‘accDnuc’: the first position in each recoded codon. The resulting viable sequence (accD.Viable) carried codons from both designs. mRNA and RBS scores-ratio between predicted mRNA folding energy (kcal/moD(Markham et al., 2005) or predicted RBS strength (Salis, 2011) of recoded and non-recoded codon.

FIG. 16 illustrates an example of exploring viable alternatives for accD recoding. In order to locate the recalcitrant codon(s) in the recoded gene accD, MAGE (multiplexed automated genome engineering as is known in the art) (Wang et al., 2009) was used in a naive non-recoded strain. The N-terminal end of the gene that is the most probable loci for gene expression disruption was specifically targeted (Plotkin et al, 2011, Goodman et al., 2013, Boël et al., 2016). The first five forbidden codons of gene accD (nucleotide positions 4, 25, 52, 85, 100) were targeted by two oligonucleotides carrying degenerate bases at the recoded positions. (N represents base pairs A, T, C or G). WT represents non-recoded accD sequence (black), sequences 1-5 are viable genotypes resulting from MAGE experiment (forbidden codons shown in black), accD.Initial represents lethal recoded accD (yellow), accD.Improved represents an alternative computationally generated accD sequence. Predicted mRNA folding energy scores for each sequences are shown on the right. Predicted RBS strength scores for each codon are shown below (bars for each position are in the following order: WT (black); sequence 1-5 (gray); accD.Initial (yellow); accD.Improved (blue)). mRNA score represents the ratio between the predicted mRNA folding energy (kcal/moDof the recoded sequence and the wild-type sequence. RBS score represents the ratio between the predicted RBS strength of the recoded sequence and the wild-type sequence for each codon. RBS strength is a calculated score used as a proxy for ribosome pausing.

FIG. 17 illustrates an example of sequence alignment of the different versions of the gene accD in segment 44. WT corresponds to non-recoded sequence. accD.Initial corresponds to lethal recoded design. accD.Improved corresponds to recoded accD sequence generated by an improved algorithm. accD.Viable corresponds to the genotype of the viable clones obtained after recombincering of accD.Improved to replace accD.Initial.

FIGS. 18A-18B illustrates examples showing compatibility of 57-codon adk gene with biocontainment. In order to verify rE.coli-57 compatibility with biocontainment, seven-codon replacement for the essential gene adk was applied in two different bio-contained strains (C321.ΔA.adk_d6 and C321.ΔA.adk_d6.tyrS_d8). FIG. 18A illustrates bio-contained strains modified with 57-codon adk maintained similar fitness as their nonmodified parents. Light gray -non modified biocontainment strains (Mandell et al., 2015); Dark gray-biocontained strains with 57-codon adk. FIG. 18B illustrates escape rate of bio-contained strains with or without 57-codon adk. SC media: SDS+Chloramphenicol. SCA media: SDS+Chloramphenicol+Arabinose.

FIGS. 19A-19B illustrate an example of construction of strain C123. FIG. 19A illustrates an example workflow used to create and analyze strain C123. The design phase involved identification of 123 AGR codons in the essential genes of Escherichia coli. MAGE oligos were designed to replace all instances of these AGR codons with the synonymous CGU codon. The build phase used CoS-MAGE to convert 110 AGR codon to CGU. Multiplex allele specific colony PCR (MASC-PCR) was used to screen for desired recombinants. AGR conversions that were not observed in 96 clones screened by MASC-PSC were triaged to troubleshooting. The in vivo troubleshooting phase resolved the 13 codons that could not be readily converted to CGU. In the Study Phase, sequencing, evolution and phenotyping was performed on strain C123. FIG. 19B illustrates an example schematic of the C123 genome relative to MG1655 (Chr. 0 oriented up.). Exterior labels indicate the set groupings of AGR codons. Successful AGR to CGU conversions are indicated by radial green lines, and 13 recalcitrant codons are indicated by radial red lines.

FIGS. 20A-2B illustrate an example analysis of attempted AGR-> CGU replacements. FIG. 20A illustrates AGR recombination frequency versus normalized ORF position. AGR recombination frequency was determined 96 clones per cell population using MASC-PCR. Normalized ORF position was the residue number of the AGR codon divided by the total length of the ORF. Failed AGR to CGU conversions are indicated using vertical red lines below the x-axis. FIG. 20B illustrates doubling time of strains in the C123 lineage in LB^{L} media at 34° C. was determined in triplicate on a 96-well plate reader. Colored bars indicate which set of codons was under construction when a doubling time was determined. Recalcitrant AGR-> CGU conversions that were unsuccessful (i.e., MASC-PCR frequency <1/96) were triaged into a troubleshooting pipeline. The optimized replacement sequences for these 13 recalcitrant AGR codons were incorporated into the final strain (gray section at right, labeled with a ‘*’), and the resulting doubling times were measured.

FIGS. 21A-21D illustrate examples of failure mechanisms for four recalcitrant AGR replacements. Wild type AGR codons are indicated in bold black letters, design flaws are indicated in red letters, and optimized replacement genotypes are indicated in green letters.

FIG. 21A illustrates genes ftsI and murE overlap with each other. An AGA-> CGU mutation in fts/would introduce a non-conservative Asp3Val mutation in murE. The amino acid sequence of murE was preserved by using an AGA->CGA mutation. FIG. 21A discloses SEQ ID NOS 2280-2285, and 2284, respectively, in order of appearance. FIG. 21B illustrates gene secE overlaps with the RBS for downstream essential gene nusG. An AGG-> CGU mutation is predicted to diminish the RBS strength by 97% (47). RBS strength is preserved by using an AGG->GAG mutation. FIG. 21B discloses SEQ ID NOS 2286-2289, 2289, and 2290, respectively, in order of appearance. FIG. 21C illustrates that gene ssb has an internal RBS-like motif shortly after its start codon. An AGG-> CGU mutation would diminish the RBS strength by 94%. RBS strength is preserved by using an AGA->CGA mutation combined with additional wobble mutations indicated in green letters. FIG. 21C discloses SEQ ID NOS 2291-2294, respectively, in order of appearance. FIG. 21D illustrates that gene rnpA has a defined mRNA structure that would be changed by an AGG-> CGU mutation. The original RNA structure is preserved by using an AGG->CGG mutation. The RBS (green), start codon (blue) and AGR codon (red) are annotated with like-colored boxes on the predicted RNA secondary structures. FIG. 21D discloses SEQ ID NOS 2295-2298, respectively, in order of appearance.

FIG. 22 illustrates an example of RBS strength and mRNA structure predict synonymous mutation success. In particular, FIG. 22 illustrates a scatter plot showing predicted RBS strength (y-axis, calculated with the Salis ribosome binding site calculator (47)) versus deviations in mRNA folding (x-axis, calculated at 37° C. by UNAFold Calculator (41)). Small gray dots represent non-essential genes in E. coli MG1655 that have an AGR codon within the first 10 or last 10 codons. Large gray dots represent successful AGR-> CGU conversions in the first 10 or last 10 codons of essential genes. Orange asterisks represent unsuccessful AGR-> CGU mutations (recalcitrant codons) in essential genes. Green dots represent optimized solutions for these recalcitrant codons. The “safe replacement zone” (blue shaded region) is an empirically defined range of mRNA folding and RBS strength deviations, based on the successful AGR-> CGU replacement mutations observed in this study. Most unsuccessful AGR-> CGU mutations (Orange asterisks) cause large deviations in RBS strength or mRNA structure that are outside the “safe replacement zone.” Genes holB and ftsI are two notable exceptions because their initial CGU mutations caused amino acid changes in overlapping essential genes. Arrows show that deviations in RBS strength and/or mRNA structure are reduced for four examples of optimized replacement of recalcitrant codons (ftsA, folC, rnpA, rpsJ).

FIG. 23 illustrates an example of codon preference of 14 N-terminal AGR codons. CRAM (Crispr-Assisted MAGE) was used to explore codon preference for several AGR codons located within the first 10 codons of their CDS. Briefly, MAGE was used to diversify a population by randomizing the AGR of interest, then a CRISPR/Cas9 system as generally known in the art using guide RNA and a Cas enzyme was used to deplete the parental (unmodified) population, allowing exhaustive exploration of all 64 codons at a position of interest. Thereafter codon abundance was monitored over time by serially passaging the population of cells and sequencing using an Illumina MiSeq. The left y-axis (Codon Frequency) indicates relative abundance of a particular codon (stacked area plot). The right y-axis indicates the combined deviations in mRNA folding structure (red line) and internal RBS strength (blue line) in arbitrary units (AU) normalized to 0.5 at the initial timepoint. 0 means no deviation from wild type. The horizontal axis indicates the experimental time point in hours at which a particular reading of the population diversity was obtained. Genes bcsB and chpS are non-essential in examples of strains described herein and thus serve as controls for AGR codons that are not under essential gene pressure.

FIG. 24 illustrates an example in which RBS strength and mRNA structure predict codon preference of 14 N-terminal codon substitutions. In particular, FIG. 24 shows a scatter plot showing the results of the CRAM experiment (FIG. 23) Each panel represents a different gene. The Y-axis represents RBS strength deviation (calculated with the Salis ribosome binding site calculator (Salis, 2011)) while the X-axis shows deviations in mRNA folding energy (x-axis, calculated at 37° C. by UNAFold Calculator (Zadeh et al., 2011). Codon abundance at the intermediate time point (t=72 hrs, chosen to show maximal diversity after selection) is represented by the dot size. Green dots represent the WT codon. Blue dots represent synonymous AGR codons. Orange dots represent the remaining 58 non-synonymous codons, which may introduce non-viable amino acid substitutions. Black squares represent unsuccessful AGR-> CGU conversions observed in the genome-wide recoding effort (Table 3, FIG. 19A-19B). The “safe replacement zone” (blue shaded region) is the empirically defined range of mRNA folding and RBS strength deviations, based on the successful AGR-> CGU replacement mutations observed in this study (FIG. 21A-D). Genes bcsB and chpS are non-essential in examples of strains described and thus serve as controls for AGR codons that are not under essential gene pressure.

FIGS. 25A-25B illustrate an example in which predicting optimal replacements for AGR codons reduces the number of predicted codons that require troubleshooting. FIG. 25A illustrates empirical data from the construction of C123. 110 AGR codons were successfully recoded to CGU (green), and 13 recalcitrant AGR codons required troubleshooting (red, striped). FIG. 25B illustrates predicted recalcitrant codons for replacing all instances of the AGR codons genome-wide. The reference genome used for this analysis had insertion elements and prophages removed (Umenhoffer et al., 2010) to limit total nucleotides synthesized, leaving 3181 AGR codons to be replaced. The analysis predicts that replacing all instances of AGR with CGU would have resulted in 246 failed conversions (′Naïve Replacement′, red striped). However, implementing the rules from this work (‘Informed Replacement’) to identify the best CGN alternative reduces the predicted failure rate from 10.5% (13/123), to 2.32% (74/3181) of which only a small subset will have a direct impact on fitness due to their location in non-essential genes. Each specific synonymous CGN is identified with a unique shade of green and is labeled inside of its respective section.

FIG. 26 illustrates an example strategy for replacing each “set” of AGR codons in all of the essential genes of Escherichia coli (EcM2.1). Here the AGR codons are marked with open triangles (various colors). To start, a dual-selectable tolC cassette (double green line) is recombined into the genome using lambda red in a multiplexed recombination along with several oligos targeting nearby (<500 kb), downstream AGR loci (various colored lines). Upon selection for tolC insertion clones, correctly chosen AGR codons are also observed (filled in triangles) at a higher frequency due to strong linkage between recombination events at tolC and other nearby (<500 kb), downstream AGR loci. Next, a second recombination is carried out using the same AGR conversion oligo pool, but now paired with another oligo to disrupt the tolC ORF with a premature stop, after which the tolC counter-selection is applied, again enriching the population for AGR conversions. A third, multiplexed recombination then fixes the tolC ORF, again targeting AGR loci. After applying the tolC selection clones are assayed by MASC-PCR. Assuming most conversions in a given set had been made, the selectable marker would then be removed using a repair oligo in a singleplexed or multiplexed recombination (depending on need). The tolC counter-selection is then leveraged to both leave a scarless chromosome and free up the tolC cassette for use elsewhere in the genome.

FIGS. 27A-27C illustrate an example schematic of 3 different failures cases for recalcitrant AGR-> CGU mutations. For each case, the top row is the initial sequence, the middle row is the AGR-> CGU mutation and the third row of primary DNA sequence is the optimized solution converged on in troubleshooting. Green boxes below the DNA sequence indicates amino acid sequence in the same order (top is initial, middle results from AGR-> CGU, bottom results from troubleshot solution). FIG. 27A illustrates C-terminal overlap cases of AGR's at ends of essential genes with downstream ORF's. (i) Genes ftsI and murE overlap with each other. An AGA-> CGU mutation in fisI would introduce a non-conservative Asp3Val mutation in murE. The amino acid sequence of murE was preserved by using an AGA->CGA mutation. FIG. 27A (i) discloses SEQ ID NOS 2280-2285, and 2284, respectively, in order of appearance. (ii) Genes holB and tmk overlap with each other. An AGA-> CGU mutation in holB would introduce a non-conservative Stop214Cys mutation in tmk. The amino acid sequence of tmk was preserved by using an AGA->CGC mutation and adding 3 nucleotides. FIG. 27A (ii) discloses SEQ ID NOS 2299-2302, respectively, in order of appearance. FIG. 27B illustrates C-terminal overlap cases of AGR's at ends of essential genes with the RBS of a downstream gene. (i) Gene secE overlaps with the RBS for downstream essential gene nusG. An AGG-> CGU mutation would diminish the RBS strength by 97% (Salis et al., 2011). RBS strength is preserved by using an AGG->GAG mutation. FIG. 27B (i) discloses SEQ ID NOS 2286-2289, 2289, and 2290, respectively, in order of appearance. (ii) Gene dnaT overlaps with the RBS for downstream essential gene dnaC. An AGG-> CGU mutation would diminish the RBS strength by 77% (Salis et al., 2011). RBS strength is preserved by using an AGG->CGA mutation. FIG. 27B (ii) discloses SEQ ID NOS 2303-2305, respectively, in order of appearance. (ii) Gene folC overlaps with the RBS for downstream gene dedD, shown to be essential in the strain. An AGGAGA->CGUCGU mutation would diminish the RBS strength by 99% (Salis et al., 2011). RBS strength is preserved by using an AGG->CGGCGA mutation. FIG. 27B (iii) discloses SEQ ID NOS 2306-2311, and 2312 respectively, in order of appearance. FIG. 27C illustrates N-terminal RBS motifs causing recalcitrant AGR conversions at the beginning of essential genes. (i) Gene dnaT has an internal RBS-like motif. An AGG-> CGU mutation would increase the RBS strength 26 times (Salis, 2011). RBS strength is better preserved by using an AGA-> CGU mutation combined with additional wobble mutations. FIG. 27C (i) discloses SEQ ID NOS 2313-2316, 2316, and 2316, respectively, in order of appearance. (ii) Gene prfB has an internal RBS-like motif. This RBS motif is involved in a downstream planned frameshift in prfB (Curan, 1993). Only by removing the frameshift was AGG-> CGU mutation possible (leaving a poor RBS-like site). To maintain the frameshift, AGG->CGG mutation and additional wobble was required. In that case, local RBS strength was maintained (fourth row). .FIG. 27C (ii) discloses SEQ ID NOS 2317-2322, 2321, and 2321, respectively, in order of appearance. (iii) Gene ssb has an internal RBS-like motif. An AGG-> CGU mutation would diminish the RBS strength by 94%. RBS strength is preserved by using an AGA->CGA mutation combined with additional wobble mutations. FIG. 27C (iii) discloses SEQ ID NOS 2291-2294, respectively, in order of appearance.

FIG. 28 illustrates an example of ribosomal pausing data drawn from previous work (Li et al., 2012) for genes ssb (SEQ ID NO: 2324), dnaT (SEQ ID NO: 2325) and prfB (SEQ ID NO: 2323). Green line represents ribosome profiling data for each gene. Orange line is the average for all genes with an AGR codon within the first 30 nucleotides of the annotated start codon. Region between the two vertical red lines indicates zones of interest (centered 12 bp after the AGR codon). Interestingly, prfB and ssb show a peak after the AGR codon, where no peak is observed for dnaT. Based on predictions from the Salis calculator, replacing AGR with CGU in those 3 cases is believed to disrupt ribosomal pausing (prfB and ssb) or to introduce ribosomal pausing (dnaT).

FIG. 29 illustrates an example of mRNA folding predictions for the 4 recalcitrant AGR-> CGU mutations explained by mRNA folding variations. mRNA folding prediction of 100 nucleotides upstream and 30 nt downstream of the start codon using UNAfold (Markham et al., 2008). Both the shape of the mRNA folding and the folding energy value have to be taken into account to understand failure of the AGR-> CGU conversion. ‘AGR’ depicts the predicted, wild-type mRNA, ‘CGU’ is the mRNA folding prediction with an AGR-> CGU mutation (generally not observed) and ‘Optimized’ correspond to the mRNA folding prediction of the AGR replacement solution found after in vivo troubleshooting. Under each structure, the predicted free energy of folding of the visualized structure is listed in kcal/mol.

FIGS. 30A-30D illustrate an example of mRNA folding predictions for the gene rnpA. For folding predictions, 30 nucleotides were used upstream and 100 nucleotides downstream of the rnpA start site using UNAfold (Markham et al., 2008). FIG. 30A illustrates the wild-type rnpA sequence, with AGG (in blue box). FIG. 30B illustrates the wild-type rnpA sequence with AGG-> CGU in blue box (not observed). FIG. 30C illustrates the wild-type rnpA sequence with AGG->CGG in blue box (observed with no growth rate defect). FIG. 30D illustrates the wild-type rnpA sequence with AGG->CTG in blue box and one complementary mutation CCC->CCA to maintain the mRNA loop (in blue box) (observed, also with no growth rate defect).

FIG. 31 illustrates an example in which G15A ArgU does not affect expression and aminoacylation levels in WT and recoded E. coli strains. Northern blot Acid-Urea PAGE was performed on WT and G15A argU tRNA in wild-type E. coli (WT-WT and WT-G15A), and in the final strains C123a and b (501 and 503) at several growth conditions. Aminoacylation levels are comparable to wild-type for all conditions and combinations, suggesting no effect on charging levels despite the mutation sweeping into the population.

FIG. 32 illustrates an example of a number of reads for each codon and for each gene in the CRAM experiment at time point 24 hrs. CRAM (Crispr-Assisted MAGE) was used to explore codon preference for several N-terminal AGR codons. The left y-axis (Number of reads) indicates abundance of a particular codon. The x-axis indicates the 64 possible codons ranked from AAA to TTT in alphabetical order. Experimental time point 24 hrs is presented. Diversity was assayed by Illumina sequencing. Genes bcsB and chpS are non-essential and thus serve as controls for AGR codons that are not under essential gene pressure.

FIG. 33 illustrates an example of a number of reads for each codon and for each gene in the CRAM experiment at time point 144 hrs. CRAM (Crispr-Assisted MAGE) was used to explore codon preference for several N-terminal AGR codons. The left y-axis (Number of reads) indicates abundance of a particular codon. The x-axis indicates the 64 possible codons ranked from AAA to TTT in alphabetical order. Experimental time point 144 hrs is presented. Diversity was assayed by Illumina sequencing. Genes bcsB and chpS are non-essential and thus serve as controls for AGR codons that are not under essential gene pressure.

FIG. 34 illustrates an example of a number of predicted recalcitrant AGR codons for each AGR replacement strategy. 4 possible genomes replacing all 3222 AGRs have been designed using 4 replacement strategies. First AGRs were changed to CGU genome-wide (green bars). Second, AGR synonyms were chosen to minimize local mRNA folding deviation near the start of genes (orange bars). Third, AGR synonyms were chosen to reduce RBS strength deviation (blue bars). Finally, AGR synonyms were chosen to minimize both (purple bars). These genomes were then scored using custom software and compared. Every deviation outside of the Safe Replacement Zone is predicted to be a recalcitrant codon.

FIG. 35 illustrates an example of a representational graph of the fully recoded genome relative to MG1655. The outer ring contains the set grouping that each AGR codon (vertical line) is in. Each line contains information on troubleshooting (red if troubleshot, green if not), and relative recombination frequency (dot). Each internal ring represents the mutations accumulated during that sets creation, the active set for each ring is highlighted. The internal rings represent the troubleshooting steps during strain construction.

FIG. 36A is a schematic depicting various method steps of embodiments of the present disclosure.

FIG. 36B is a graph depicting the experimental procedure where alternative codons are introduced via MAGE at different positions in the genome. The population is then maintained at mid-logarithmic phase growth while sampling at regular intervals. Codon fractions are plotted vs time and a logarithmic decay function is fitted and the decay constant indicates fitness.

FIG. 36C compares the experimentally-measured fitness to the predicted GETK score. Each position on the x-axis corresponds to one of 95 sub-experiments testing a different genomic position. Position on the y-axis indicates fitness relative to wild-type, with more negative value indicating worse fitness and 0 indicating wild-type fitness. Inset shows fitness of measured codons grouped by good, average, or bad GETK scores. Examples with good predicted score have significantly better fitness.

FIG. 37 shows a summary of results of 62 sub-experiment testing combinations of proximal codon changes near the 5-prime ends of various genes. A library of oligos was designed with degeneracy at codon positions within the 90-mer oligo window. Sub-experiment results are presented together, but separated by codon combinations with good fitness (<7% fitness defect) or bad fitness (>13% fitness defect). A pair of good-bad fitness summaries is plotted for each of three GETK scoring metrics: change in 5-prime mRNA folding strength, change in upstream RBS motif strength, change in internal RBS motif strength. For each metric, a lower score indicates less predicted disruption of the respective motif.

FIG. 38 illustrates alternative codon trajectories for controls. Top row shows null-effect controls, where synonymous codons and early stop codons were introduced into non-essential genes LacZ and GalK at multiple positions, and showing similar effect between synonymous codons and internal stops. Bottom rows shows strong-effect controls, where synonymous codons and internal stop codons were introduced into essential genes. These show a marked difference between internal stop and synonymous codons, with a greater dynamic range of codon preference at some positions.

FIG. 39 summarizes results from testing non-synonymous and synonymous mutations observed in phylogenetically-close neighbors of E. coli in gammaproteobacteria at specific positions internal to genes (not limited to 5-prime end). These positions were prioritized according to whether internal RBS for some alternatives were predicted by GETK to be disruptive. Internal RBS score is shown to be a strong predictor of fitness of alternative allele choices.

FIG. 40 shows results from testing a mix of non-synonymous mutations predicted by conservation. These positions were prioritized according to peaks of ribosomal pausing as reported by (Li et al., 2012). Internal RBS score is shown to be a strong predictor of fitness of alternative allele choices.

### DETAILED DESCRIPTION

Embodiments of the present disclosure are based on methods, algorithms, and computer software for designing genomes based on a set of rules or constraints or conditions or parameters or features which may be generally referred to throughout as “constraints”, “a constraint,” “rules,” or “a rule” or “ruled based.” The rule-based genome design described herein includes methods and computer algorithms for implementing genome modifications while preserving known biological motifs and features in DNA and satisfying various constraints and/or rules or conditions or parameters or features for synthesis and assembly of designed genomes. As described herein, rules or conditions or parameters or features may refer to biological constraints and synthesis constraints which may be applied in synthesizing genome designs by scoring each constraint for a possible genome design. Biological motifs may include essential genes, ribosome binding site (RBS) motifs, mRNA secondary structures, internal ribosome pausing site motifs, and the like. In some embodiments, the disclosed methods for genome design may be directed to designing genetic elements, including genes, operons, genomes, and the like.

Aspects of the present disclosure include methods for empirically deriving new rules or constraints or conditions or parameters or features based on combinations of multiplex automatable genome engineering (MAGE) and targeted sequencing, along with other technologies such as CRISPR-assisted MAGE (CRAM), MAGE in combination with molecular inversion probes (MIPS), and the like. Aspects described herein may also include providing information about designed genomes based on a set of constraints and/or rules and recommending modifications that may yield phenotypic improvements in future genome design. Ultimately, the rule-based genome design methods and integrated software disclosed herein may be beneficial in the fields of genome engineering and bioproduction for improving efficiency and reducing costs of DNA construct production.

In some cases, several challenges may arise when modifying a genome, such as when choosing synonymous alleles for genome-wide allele replacement of certain alleles (which may be referred to as “forbidden alleles” or “forbidden codons” as described herein). First, to ensure biological viability, it may be important to maintain the fundamental features of a parent genome, such as GC content and regulatory elements encoded by the primary nucleotide sequence. Additionally, when forbidden alleles fall in overlapping gene regions, it may be necessary to carefully split these overlaps in a manner that avoids introducing non-synonymous mutations or disrupting regulatory features. Finally, it may be desirable for a computational design scheme to be compatible with the experimental tools being used for genome construction.

Thus, described herein is a rule-based architecture for genome recoding software, in which user-specified rules serve as constraints for finding suitable synonymous allele replacements. As an example, Tables 1 and 2 provide further examples of rules and constraints that may be implemented for genome design (e.g., for design and synthesis of a radically recoded E. coli genome). In particular, Table 1 provides examples of biological constraints or conditions or parameters or features for genome design rules, whereas Table 2 provides examples of synthesis constraints or conditions or parameters or features for genome design rules. The rule-based architecture described herein may be implemented as a computer module or software module and may be extended to general applications, as well as customized according to specific needs.

In the following description of the various embodiments, reference is made to the accompanying drawings, which form a part hereof, and in which is shown by way of illustration, various embodiments of the disclosure that may be practiced. It is to be understood that other embodiments may be utilized. A person of ordinary skill in the art after reading the following disclosure will appreciate that the various aspects described herein may be embodied as a computerized method, system, device, or apparatus utilizing one or more computer program products. Accordingly, various aspects of the computerized methods, systems, devices, and apparatuses may take the form of an embodiment consisting entirely of hardware, an embodiment consisting entirely of software, or an embodiment combining software and hardware aspects. Furthermore, various aspects of the computerized methods, systems, devices, and apparatuses may take the form of a computer program product stored by one or more non-transitory computer-readable storage media having computer-readable program code, or instructions, embodied in or on the storage media. Any suitable computer readable storage media may be utilized, including hard disks, CD-ROMs, optical storage devices, magnetic storage devices, and/or any combination thereof. In addition, various signals representing data or events as described herein may be transferred between a source and a destination in the form of electromagnetic waves traveling through signal-conducting media such as metal wires, optical fibers, and/or wireless transmission media (e.g., air and/or space). It is noted that various connections between elements are discussed in the following description. It is noted that these connections are general and, unless specified otherwise, may be direct or indirect, wired or wireless, and that the specification is not intended to be limiting in this respect.

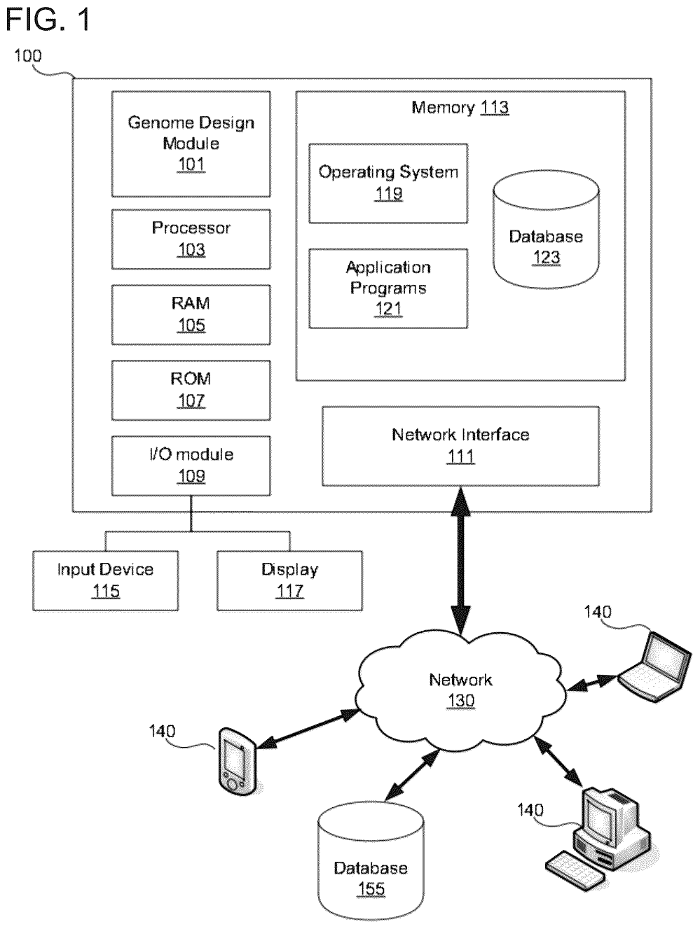

In one or more arrangements, teachings of the present disclosure may be implemented with a computing device. FIG. 1 illustrates a block diagram of a computing device 100 that may be used in accordance with aspects of the present disclosure, such as for implementing methods for genome design. The computing device 100 is a specialized computing device programmed and/or configured to perform and carry out aspects associated with rule-based genome design as described herein. The computing device 100 may have a genome design module 101 configured to perform methods and execute instructions as described herein. The genome design module 101 may be implemented with one or more specially configured processors and one or more storage units (e.g., databases, RAM, ROM, and other computer-readable media), one or more application specific integrated circuits (ASICs), and/or other hardware components. Throughout this disclosure, the genome design module 101 may refer to the software (e.g., a computer program, application, and or algorithm) and/or hardware used to receive one or more genome files or templates (e.g., one or more annotated GenBank files), receive a list of alleles to be replaced, modify a genome by applying a set of biological constraints and synthesis constraints to the genome sequences(s), generate a new genome design based on the modifications, scoring genome designs, modifying and/or creating new rules or constraints or conditions or parameters or features for genome design, and the like. Specifically, the genome design module 101 may be a part of a rule-based architecture for genome recoding software which may be further extended to other applications. The one or more specially configured processors of the genome design module 101 may operate in addition to or in conjunction with another general processor 103 of the computing device 100. In some embodiments, the genome design module 101 may be a software module executed by one or more general processors 103. Both the genome design module 101 and the general processor 103 may be capable of controlling operations of the computing device 100 and its associated components, including RAM 105, ROM 107, an input/output (I/O) module 109, a network interface 111, and memory 113.

The I/O module 109 may be configured to be connected to an input device 115, such as a microphone, keypad, keyboard, touchscreen, gesture or other sensors, and/or stylus through which a user of the computing device 100 may provide input data. The I/O module 109 may also be configured to be connected to a display device 117, such as a monitor, television, touchscreen, and the like, and may include a graphics card. The display device 117 and input device 115 are shown as separate elements from the computing device 100, however, they may be within the same structure. Using the input device 115, system administrators or users may add and/or update various aspects of the genome design module, such as rules or constraints or conditions or parameters or features, scoring, predefined thresholds, ranges, and biological and synthesis constraints related to designing a genome. The input device 115 may also be operated by users in order to design a genome by inputting a genome file and a list of alleles or sequences to be modified in the genome file by the genome design module 101.

The memory 113 may be any computer readable medium for storing computer executable instructions (e.g., software). The instructions stored within memory 113 may enable the computing device 100 to perform various functions. For example, memory 113 may store software used by the computing device 100, such as an operating system 119 and application programs 121, and may include an associated database 123.

The network interface 111 allows the computing device 100 to connect to and communicate with a network 130. The network 130 may be any type of network, including a local area network (LAN) and/or a wide area network (WAN), such as the Internet. Through the network 130, the computing device 100 may communicate with one or more computing devices 140, such as laptops, notebooks, smartphones, personal computers, servers, and the like. The computing devices 140 may include at least some of the same components as computing device 100. In some embodiments the computing device 100 may be connected to the computing devices 140 to form a “cloud” computing environment.

The network interface 111 may connect to the network 130 via communication lines, such as coaxial cable, fiber optic cable, and the like or wirelessly using a cellular backhaul or a wireless standard, such as IEEE 802.11, IEEE 802.15, IEEE 802.16, and the like. In some embodiments, the network interface may include a modem. Further, the network interface 111 may use various protocols, including TCP/IP, Ethernet, File Transfer Protocol (FTP), Hypertext Transfer Protocol (HTTP), and the like, to communicate with other computing devices 140.

According to certain aspects, the computing device 100 may interface with one or more databases 155 to access genome data (e.g., gene sequences). For example, a database 155 may be an external database that stores a collection of nucleotide sequences (e.g., DNA, RNA, cDNA, and the like) and corresponding protein translations (e.g., GenBank). In some cases, the genome design module 101 may access and/or receive a specific genome file or template from the database 155, and the genome design module 101 may utilize the file for further genome design based on a set of rules and scoring.

FIG. 1 is an example embodiment of a computing device 100. In other embodiments, the computing device 100 may include fewer or more elements. For example, the computing device 100 may use the general processor(s) 103 to perform functions of the genome design module 101, and thus, might not include a separate processor or hardware for the genome design module 101.

Although not required, various aspects described herein may be embodied as a method, data processing system, or as computer-readable medium storing computer-executable instructions. For example, a computer-readable medium storing instructions to cause a processor to perform steps of a method in accordance with aspects of the disclosed embodiments is contemplated. For example, aspects of the method steps and algorithms disclosed herein may be executed on a processor on computing device 100. Such a processor may execute computer-executable instructions stored on a computer-readable medium.

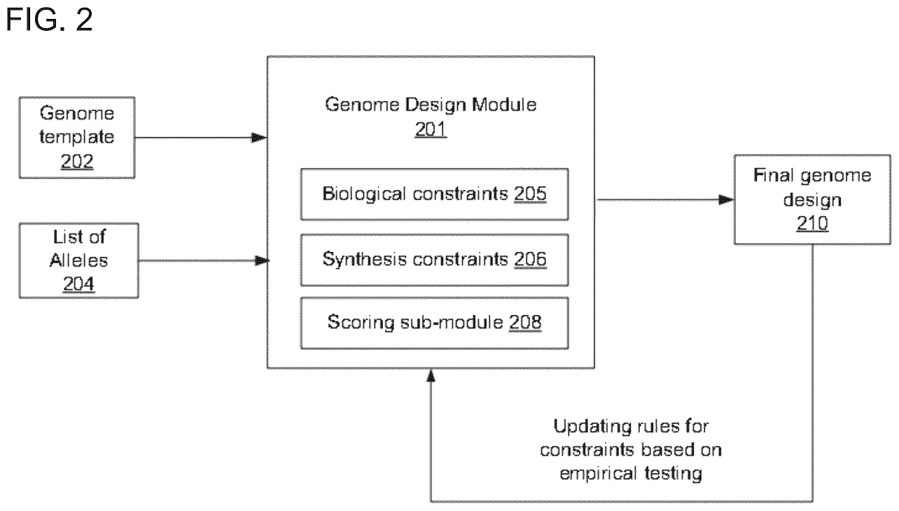

FIG. 2 illustrates an example block diagram of a genome design module in which various aspects of the present disclosure may be implemented in accordance with one or more example embodiments. In particular, FIG. 2 illustrates a genome design module 201 which may comprise a software tool that may be utilized for any genome modifications, such as a genome-wide allele replacement in a prokaryotic genome. In some embodiments, the genome design module 201 may be the same as the genome design module 101.

The genome design module 201 may utilized for a variety of purposes, including refactoring genomes such as by removing all occurrences of a particular allele throughout the genome (allowing deletion of translation factors and functional allele reassignment), rearranging operons into functionally related units, removing non-essential elements (e.g., cryptic prophages, mobile elements, non-essential genes, etc.), modifying/optimizing/introducing metabolic pathways, and the like.

As illustrated in the example in FIG. 2, the genome design module 201 may receive two inputs: a genome template file 202 and a list of alleles 204. The genome template 202 may comprise known genome sequences or a particular genome (e.g., in the form of an annotated GenBank file). In some embodiments, the genome template 202 may comprise sequences for any type of genome, including bacterial genomes, mycoplasma genomes, yeast genomes, human genomes, genomes for any naturally-occurring organism, or genomes of any previously evolved or engineered organism. As an example, an E. coli MDS42 genome template (GenBank: AP012306.1) was used as the genome template 202 as described in the Examples herein. The list of alleles 204 may comprise a list of alleles to be synonymously replaced throughout the genome. The list of alleles 204 may also include coding sequences (e.g., codons) and non-coding sequences (e.g., non-coding RNAs including tRNA and sRNA, extragenic sequence motifs that may or may not overlap with the coding sequence, repetitive extragenic palindromic (REP) sequences, or the like). In some embodiments, the list of alleles 204 may represent a list of codons, which may be referred to as “forbidden codons.” For example, the following seven codons were in the list of codons to be replaced in the E. coli example described below: AGA, AGG, AGC, AGU, UUG, UUA, and UAG.

The genome design module 201 may receive the genome template 202 and the list of alleles 204 and automatically replace all instances of alleles from the list in the genome. For example, the genome design module 201 may automatically replace, within the genome, all instances of forbidden codons from a list of codons. The genome design module 201 may also utilize a scoring sub-module 208, and the genome design module 201 may be configured to select synonymous codons that allow the resulting sequence to best adhere to biological constraints 205 and/or synthesis constraints 206. In some embodiments, the scoring sub-module 208 may be referred to as a scoring tool.

Tables 1 and 2 provide examples of biological constraints 205 and synthesis constraints 206, respectively, which may be applied in genome design, along with descriptions of rules, constraints or conditions or parameters or features, motivation, implementation, and corresponding genome annotations. The synthesis constraints 206 may include one or more experimental rules or constraints or conditions or parameters or features that may be applied for synthesizing genome designs. In some cases, the synthesis constraints 206 may be vendor and/or technology-specific rules or constraints or conditions or parameters or features that are to be satisfied during genome design. Examples of synthesis constraints 206 may include (and are not limited to) rules for removing forbidden restriction enzyme motifs, leveraging synonymous swaps to normalize high/low GC content within genes in a genome design, preserving regulatory motifs if high/low GC content is present in intergenic regions, minimizing strong secondary structures, deleting repetitive elements which may be difficult to synthesize and replacing them by terminators, leveraging synonymous swaps to diversify primary sequence if homopolymer runs are present within genes, preserving regulatory motifs if homopolymer runs are present in intergenic regions, partitioning operons to increase the likelihood of synthesizing modular genome units that contain entirety of discrete transcriptional units, etc.

The biological constraints 205 may include one or more rules or constraints or conditions or parameters or features that are applied to genome design for preserving biologically relevant motifs, in which the biological constraints 205 may be implemented as code in the genome design module 201. For example, the biological constraints 205 may include a rule for maintaining predicted secondary structure of RNA (e.g., including, but not limited to, mRNA). The genome design module 201 may compute a predicted RNA secondary structure for both an original sequence and a modified, design sequence, and the scoring sub-module 208 may provide a quantitative representation of the difference between the two. In some embodiments, the genome design module 201 may compute deviation in predicted mRNA secondary structure by comparing the predicted free energy (AG) of the original and designed sequences (e.g., a thermodynamic-based secondary structure prediction) and/or by calculating a number of nucleotides that are no longer paired with the same sister nucleotide in the designed sequence with respect to the original sequence. In some cases, a rule may be modified according to the context of a desired change. For example, for changes near a 5′ end of a gene, the genome design module 201 may compute an mRNA secondary structure spanning nucleotides-30 to +100 of a sequence and relative to the start codon of the gene.

Additionally, the biological constraints 205 may also include a rule or constraint or condition or parameter or feature for preserving ribosome binding site (RBS) motifs. A ribosome binding site may comprise a DNA sequence motif (e.g., sequence of nucleotides) found approximately ten bases upstream of a gene (e.g., upstream of a start codon). The genome design module 201 may score and rank sequence designs according to disruption to ribosome binding sites (e.g., by using the scoring sub-module 208). For example, if a RBS motif exists in overlapping genes (e.g., to support expression of a downstream, overlapping gene), it may be beneficial to only allow mutations that do not strongly impact RBS strength. In yet another example, if output design parameters conflict with preserving said RBS motif in an overlapped architecture, then coding regions may be split and an RBS motif of similar strength may be inserted to support translation of downstream genes.

In some embodiments, the genome design module 201 may implement RBS motif strength predictions by utilizing biophysical models, such as the Salis ribosome binding site calculator (Salis, 2011), or by other empirical RBS strength look-up tables. For example, the scoring sub-module 208 of the genome design module 201 may calculate a predicted expression score for the reference sequence and the designed sequence using a biophysical model (e.g., from Salis, 2001). The ratio (or log-ratio) of these scores may become a quantified expression of disruption of this rule or constraint or conditios or parameter or feature.

In yet another example, the biological constraints 205 may include a rule or constraint or condition or parameter or feature for preserving internal ribosome pausing site motifs. For example, the occurrence of ribosome binding site-like motifs (e.g., an anti-Shine-Dalgarno sequence) may correspond to translational pausing in E. coli, which may suggest that these motifs comprise a biologically important role (Li et al., 2012). Thus, the genome design module 201 may implement a design rule that leverages a biophysical model (e.g., from Salis, 2001). As described in the Examples herein, to score a proposed design change, it may be assumed that a codon might be part of an RBS by inserting a phantom ATG start codon the correct number of bases (e.g., approximately 10) downstream of the change. Based on this rule, the genome design module 201 may calculate the predicted RBS strength before and after a proposed design change, penalizing disruption of existing internal ribosome pausing sites, or introduction of strong internal ribosomal pausing sites where one did not exist before.

Additional examples of biological constraints 205 may include (and are not limited to) rules or constraints or conditions or parameters or features for ensuring that a selection of alternative alleles or codons is consistent with global distribution of allele or codon choice (both for recoding and heterologous expression), preserving known sequence motifs in a genome design (e.g., frame-shift, selenocysteine insertion sequence (SECIS) sites, recombination sites, etc.), preserving regulatory motifs such as by preserving/tuning promoter, enhancer, and/or transcription factor motifs, applying phylogenetic conservation for a genome design by choosing sequences which are closest to phylogenetically-related neighbors when considering alternatives for a genome design modification, reducing homology between redesigned regions through non-disruptive muddling, etc. In the reducing homology example, the optimal solution for performing synonymous codon swaps while preserving an overlapping regulatory motif may be to split the overlap by making a copy, which may result in adjacent regions of high homology. The homology may be broken by performing synonymous codon swaps or other changes that do not break any annotated regulatory motifs. This may be important to produce stable genomes, such as by preventing an undesired recombination that could revert the redesigned sequence.

Furthermore, the genome design module 201 may implement the rules or constraints or conditions or parameters or features of the biological constraints 205 by using the scoring sub-module 208 to score genetic sequences (e.g., genome designs) with respect to reference sequences (e.g., genome templates). In some embodiments, the scoring sub-module 208 may assign a quantitative score to every possible change to a gene or genome. This scoring may allow ranking and prioritizing designs that achieve a desired genotypic or phenotypic outcome. The scoring, ranking, and prioritization features may comprise core features of the software for the genome design module 201.

For example, for a design choice with mutually exclusive options (e.g., for choosing an allele replacement), the genome design module 201 may allow ranking of design choices. In some embodiments, the best single design choice or any number of the best single design choices may be chosen for synthesis and testing. In other embodiments, all design choices that pass a predefined score threshold may be synthesized and tested.

Additionally, the scoring sub-module 208 of the genome design module 201 may implement different types of scoring. For example, a higher score may indicate less deviation from the biological constraints 205 (e.g., a set of rules) and may thus be preferred. For example, less deviation from the constraints may indicate a higher predicted success in biological validation. In another example, a lower score may indicate less deviation from the biological constraints 205 (e.g., a set of rules), and may thus be preferred.

The genome design module 201 may further implement scoring for a genetic design as a weighted combination of scores from specific rules or constraints or conditions or parameters or features. For example, in the case where a score may be interpreted as a deviation from a biological motif value and for the genetic design of swapping alternative alleles, each choice of allele may be scored according to a combination of factors.

That is, there may be a plurality of alternative gene sequences in which each alternative gene sequence comprises a different allele choice which may be used to replace one or more forbidden alleles in a reference genome. Thus, the genome design module 201 may apply rules or constraints or conditions or parameters or features for the biological constraints 205 by assigning a score for each rule in each alternative gene sequence. In some embodiments, each allele choice may be scored according to a combination of biological constraints 205, including fold disruption of predicted mRNA secondary structure folding energy, fold disruption of predicted ribosome binding site (RBS) affinity strength, and the like.

For example, a total score for an alternative gene sequence comprising an allele choice may be computed (e.g., by the genome design module 201) using the following equation:

$$score = {w}_{1} * f ( mRNA score ) \times {w}_{2} * g ( RBS score )$$

In the above equation, w₁ and w₂ represent weights, whereas f and g represent functions of the respective quantification of the rules. Furthermore, the weights w₁ and w₂ may be determined empirically and may be updated or modified according results from synthesizing and testing genome designs. In other embodiments, the weights may be adjusted by manual specification in which a user may manually specify (e.g., enter in) each weight (e.g., as an input into the genome design module 201 and/or the computing device 100). The weights and scoring may also be applied globally or may be context-specific. For example, a first set of weights may hold true and be applied near a 5′ end of a gene, whereas a different set of weights or a different combination of rules or constraints or conditions or parameters or features may be true and may be applied in a different area of the gene (e.g., in the middle of the gene). As described in the Examples herein, it was empirically found that the following weights for codons choices in E. coli may predict a successful swap:

$$score = ( 0 \text{.65} / 1.5411 ) * m R N {A}_{ratio} \times ( 0 \text{.35} / 8.4257 ) * ( 1 + LOG ( R B {S}_{ratio} ) )$$

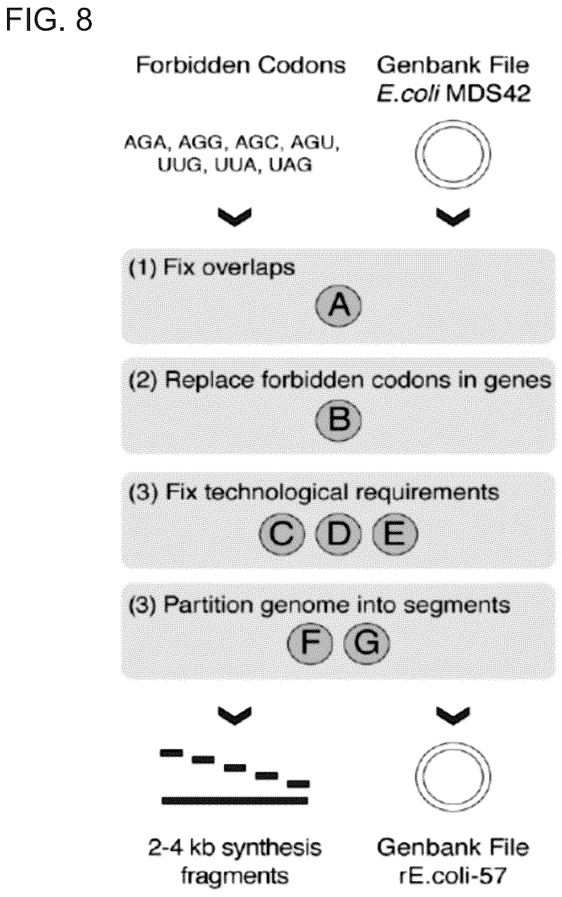

In additional embodiments, the genome design module 201 may follow an automated computational design pipeline as illustrated in FIG. 8. For example, the genome design module 201 may first implement forbidden allele replacement based on the list of alleles 204 and the genome template 202 in all instances of gene overlaps while accounting for biological constraints 205. The genome design module 201 may then apply remaining forbidden allele replacement in each gene independently while accounting for biological constraints 205. For example, for each allele that is to be replaced, there may be multiple choices for synonymous allele substitutions. A design may be minimally disruptive with respect to design rules or constraints or conditions or parameters or features that quantify deviation from the wild-type sequence (e.g. secondary structure, GC content, RBS motif strength).

However, in some embodiments, an exhaustive comparison of all possible allele or codon modifications may be computationally expensive, making iteration slow. For example, in the case of recoding E. coli, there are about 17 forbidden codons per gene and 4 possible synonymous swaps per codon, resulting in 417 possible sequences to evaluate per gene. Thus, the genome design module 201 may identify a solution that satisfies each rule or constraint or condition or parameter or feature within a threshold, rather than identifying a global minimum. To identify a satisfactory solution, the genome design module 201 may identify and represent a genome-recoding problem as a graph that is traversed using an algorithm based on depth first search. In some embodiments, the algorithm may be referred to as a graph search-based codon replacement algorithm.

For example, nodes in the graph may represent a unique alternative gene sequence. Sibling nodes in the graph may differ in the value of a specific codon. Children of a node may represent all possible changes to the next downstream codon. Each node may be assigned a score corresponding to each of the rules, including GC content, secondary structure, and codon rarity deviation. Each score may be a quantitative measure of deviation away from wild-type sequence in the respective score profile for a base pair window (e.g., a 40 base pair window or a window of any other number of base pairs) centered at a specific codon. A node may be expanded and pursued as long as all scores are below the thresholds for their respective profiles. If all nodes at a level violate the threshold, the algorithm (e.g., implemented by the genome design module 201) may backtrack to an earlier node and choose a different branch. If the algorithm is unable to find a solution for a particular gene, the threshold constraints may be modified, and a search may be restarted. In some embodiments, the graph search-based algorithm may also be applied in allele replacement for genome design.

After the graph search-based codon (or allele) selection, the genome design module 201 may apply technical rules or constraints or conditions or parameters or features considering synthesis and assembly constraints for genome design. For example, the genome design module 201 may further modify the genome template 202 using the synthesis constraints 206, in order to satisfy DNA vendor constraints, such as by removing specific restriction enzyme sites and homopolymer sequences, and balancing GC content. Finally, the genome design module 201 may partition the modified genome into segments of a predefined size (e.g., segments of any number of bases). For example, the genome design module 201 may first partition the modified genome into ˜50 kb segments and then partition each segment into 2-4 kb synthesis units or fragments.

In additional embodiments, the genome design module 201 may also allow users to provide a list of manually-specified modifications for a genome. In some embodiments, these manually-specified modifications (which may be referred to as miscellaneous design notes) may include solutions from empirical validation or special cases for which generalized rules or constraints or conditions or parameters or features have not yet been implemented. For example, in the case of recoding E. coli, the UUG codon, which encodes Leucine using tRNA^{Leu}, was chosen as one of the seven codons for replacement throughout protein coding genes. However, when the same codon (UUG) occurs as a translational start codon, it is decoded by tRNAf^{Met}, and does not need to be replaced. Thus, a miscellaneous design note was added not to replace these start codons in order to minimize perturbation of gene expression level. The miscellaneous design note may be implemented in the software in order to facilitate automated allele replacement. In another miscellaneous design note, manual substitutions were designated for AGR codons in essential genes based on previous empirical testing. In yet another miscellaneous design note, codons overlapping selenocysteine insertion sequence (SECIS) sites were manually recoded in the following genes: fdhF, fdnG, and fdoG.

The genome design module 201 may ultimately generate a plurality of alternative gene sequences (each comprising a different codon or allele choice) and select at least one alternative gene sequence as the genome design based on weighted scoring. The genome design module 201 may output a final genome design 210 which may comprise a file (e.g., a GenBank file) of the final genome design. In some cases, the genome design module 201 may identify synthesizable DNA by dividing the genome design 210 into contiguous segments, in which each segment is composed of a predetermined number of bases. For example, the genome design module 201 may also generate a list of synthesis-compatible 2-4 kilobase (kb) fragments, which may be synthesized and tested. Furthermore, one or more rules or constraints or conditions or parameters or features for the biological constraints 205 and synthesis 206 may be updated based on empirical testing resulting from the final genome design 210.

In additional embodiments, the final genome design may be based on one of: a genetic code with minor modifications from a canonical genome code, a radically redefined genetic code, a novel genetic code, or a genetic code in which codons map to non-standard amino acids (nsAAs).

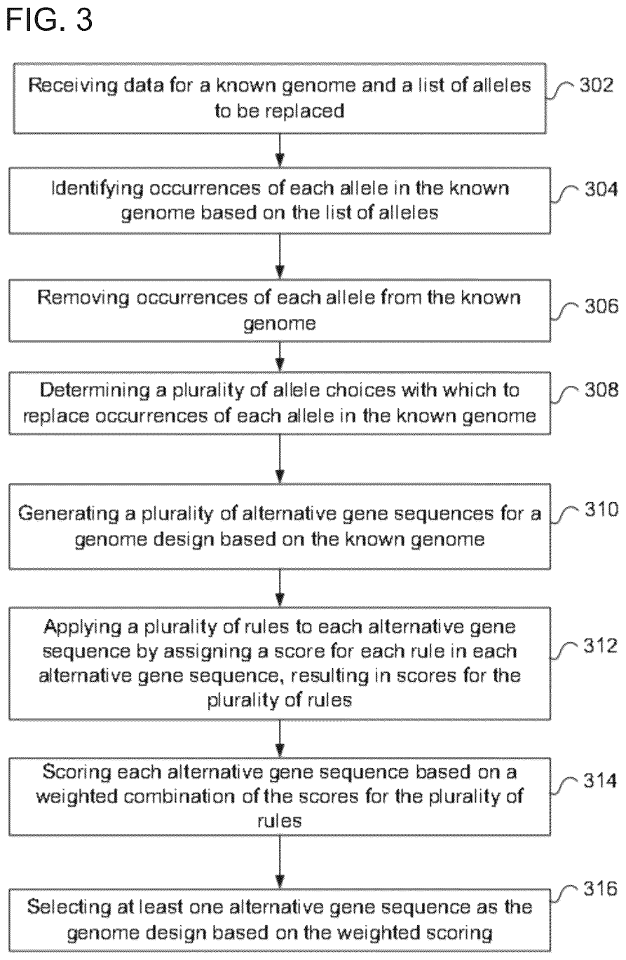

FIG. 3 illustrates a flow diagram of an example method in accordance with aspects of the present disclosure. In particular, FIG. 3 illustrates example method steps for designing genomes based on applying rules or constraints or conditions or parameters or features for biological constraints and synthesis constraints and scoring designs. The steps of FIG. 3 may be performed by a computing platform, such as by at least one of a genome design module 101, genome design module 201, scoring sub-module 208, or the like. As a result of the method of FIG. 3, a genome design may be selected and output as a final design.

The method of FIG. 3 may begin with a step 302 of a computing platform receiving data for a known genome and a list of alleles to be replaced in the known genome. For example, the genome design module 201 may receive a genome template 202 (e.g., comprising a known genome reference sequence) and a list of alelles 204 as inputs. At step 304, the computing platform may identify occurrences of each allele in the known genome based on the list of alleles. For example, the genome design module 201 may find all the alleles (e.g., forbidden codons) that are to be replaced in the genome sequence 202. At step 306, the computing platform may remove the occurrences of each allele from the known genome. For example, the genome design module 201 may apply allele replacement or removal in all occurrences in the known genome 202. In some embodiments, the genome design module 201 may apply forbidden codon replacement or removal in the known genome 202.

At step 308, the computing platform may determine a plurality of allele choices with which to replace occurrences of each allele in the known genome. For example, the genome design module 201 may identify that are there are several synonymous allele that may be utilized to replace each occurrence of each allele in the known genome 202. In alternative arrangements, steps 306 and steps 308 of the method may be combined as one step performed by the genome design module 201, in which the genome design module 201 may identify alleles to remove from the known genome and determine a plurality of allele choices with which to replace occurrences of each allele.

At step 310, the computing platform may generate a plurality of alternative gene sequences for a genome design based on the known genome. For example, the genome design module 201 may generate a plurality of alternative gene sequences, in which each alternative gene sequences includes a different allele choice from the plurality of synonymous allele choices.

At step 312, the computing platform may apply a plurality of rules or constraints or conditions or parameters or features to each alternative gene sequence by assigning a score for each rule or constraint or condition or parameter or feature in each alternative gene sequence, resulting in scores for the plurality of rules or constraints or conditions or parameters or features applied to each alternative gene sequence. For example, the genome design module 201 or the scoring sub-module 208 may utilize the one or more rules or constraints or conditions or parameters or features for the biological constraints 205 and synthesis constraints 206 to calculate sores for each rule or constraint or condition or parameter or feature with respect to each allele choice. That is, the scoring sub-module 208 calculate a score for each rule or constraint or condition or parameter or feature, including for preserving coding mRNA secondary structure, preserving ribosome binding site motifs, preserving internal ribosome pausing site motifs, and the like. Each alternative gene sequence (comprising a different allele choice) may have a score calculated for each of the rules or constraints or conditions or parameters or features.

At step 314, the computing platform may score each alternative gene sequence based on a weighted combination of the scores for the plurality of rules or constraints or conditions or parameters or features. For example, the genome design module 201 may implement scoring for each alternative gene sequence as a weighted combination of scores from the specific rules or constraints or conditions or parameters or features. At step 316, the computing platform may select at least one alternative gene sequence as the genome design based on the weighted scoring. For example, the genome design module 201 may select one or more alternative gene sequences as the final genome design 210 based on identifying which alternative gene sequences comprise a weighted score above a predefined threshold. In some cases, after selection, the genome design module 201 may output the final genome design 210 as a Genbank file which may be utilized for synthesis and testing. In some embodiments, after identifying which alternative gene sequences comprise a weighted score above a predefined threshold, the identified alternative gene sequences may be empirically tested individually or as a library (e.g., a mixture of sequences). In additional embodiments, the genome design module 201 may update one or more rules or constraints or conditions or parameters or features in the plurality of rules or constraints or conditions or parameters or features based on comparing rule predictions to empirically observed viability. For example, the final genome design 210 may be synthesized and tested for viability, and results from testing the synthesized final genome design 210 (along with results from other designs) may be used to update and derive new rules or constraints or conditions or parameters or features for future genome design.

In additional embodiments, one or more rules or constraints or conditions or parameters or features in genome design may be updated, such as by utilizing a computing platform (e.g., computing device 100 comprising the genome design module 101 or genome design module 201). First, one or more features of a genome design may be introduced into at least one cell. In some embodiments, one or more features of the genome design may be introduced into the at least one cell by using DNA cleavage to select against a wild-type genotype and/or facilitate homologous recombination. Further examples for introducing features into a cell may include using CRISPR/Cas, transcription activator-like effector nucleases (TALENs), zinc-finger nucleases (ZFNs), meganucleases, restriction endonucleases, or the like.

In other embodiments, one or more features of the genome design may be introduced into the at least one cell by using recombinases/integrases. Additional examples for introducing features into a cell may include using multiplex automated genome engineering (MAGE), lambda red-recombineering, site-specific recombinases/integrases (e.g., Cre, PhiC31, lambda integrase, Flp, etc.), recombinase-mediated cassette exchange (RMCE), or the like. In other embodiments, introducing one or more features of the genome design into the at least one cell may further include synthesizing a partial or whole genome based on the genome design. Additionally, in some embodiments, the one or more features may be tested by a growth assay using a kinetic plate reader. In other embodiments, the one or more features may be tested by an assay to test protein production. In yet additional embodiments, the one or more features may be tested by sequencing representative portions of the cell population at predetermined time points. For example, next-generation sequencing (NGS) may be used to monitor which genotypes become enriched or depleted in the population, which may be interpreted as relative fitness information.

The one or more features that have been introduced into the at least one cell may be tested by an assay in order to identify genome viability and evaluate the phenotype of the one or more features introduced into the at least one cell. In some embodiments, the one or more features may be tested on a vector (e.g., plasmid, cosmid, phagemid, bacteriophage, or artificial chromosome) or integrated into a chromosome. Based on the testing, it may be determined that the one or more features introduced into the at least one cell are expected to be viable or expected to fail according to one or more predefined rules or constraints or conditions or parameters or features for the genome design. The predefined rules or constraints or conditions or parameters or features for genome design may ultimately be updated based on the determination. In some embodiments, the one or more predefined rules or constraints or conditions or parameters or features for genome design may comprise one or more phenotypic and genotypic parameters.

In additional embodiments, the computing platform may update the predefined rules or constraints or conditions or parameters or features for genome design further based on statistical techniques and machine-learning algorithms. For example, the computing platform may update and/or automatically infer new rules or constraints or conditions or parameters or features using representation learning algorithms including, but not limited to, deep learning. Other machine learning techniques may be used for updating and learning new rules or constraints or conditions or parameters or features, including supervised or unsupervised learning, semi-supervised learning, reinforcement learning, and deep learning. These may include specific techniques, such as convolutional neural networks, random forests, hidden Markov models, autoencoders, Boltzmann machines, and the like. In another example, a user may utilize the computing platform to manually define new rules or constraints or conditions or parameters or features based on analysis.

In additional embodiments, genome designs may be generated by a computing platform (e.g., computing device 100 comprising the genome design module 101 or genome design module 201) and may be tested by the computing platform by determining one or more features in the genome design that fail a set of predefined rules or constraints or conditions or parameters or features. In some embodiments, the set of predefined rules or constraints or conditions or parameters or features may comprise one or more phenotypic and genotypic parameters. The computing platform may obtain or access a sample of a known genome sequence (e.g., a known genome sequence that the genome design is based on), the computing platform may further analyze the sample of the known genome sequence. In some embodiments, the computing may determine the one or more features in the genome design that fail a set of predefined rules or constraints or conditions or parameters or features by testing individual mutations in the genome design in parallel. In other embodiments, the computing may determine the one or more features in the genome design that fail a set of predefined rules or constraints or conditions or parameters or features by testing individual mutations in the genome design in multiplex.

The computing platform may predict modifications to the genome design that may be implemented in order to satisfy a predetermined design objective and to increase probability of viability. For example, a predetermined design objective may comprise one or more features of the natural genome that may need to be changed. A natural genome sequence may be viable, whereas a recoded genome sequence or genome design may need to be tested in order to determine if the design is still viable. After predicting the modifications, the computing platform may test the predicted modifications to generate an improved genome design. In some embodiments, the predicted modifications for the genome design may be tested as a mixture. In other embodiments, the predicted modifications for the genome design may be tested using genetic diversity and selection.

The above disclosure generally describes the present invention. All references disclosed herein are expressly incorporated by reference. A more complete understanding can be obtained by reference to the following specific examples which are provided herein for purposes of illustration only, and are not intended to limit the scope of the invention.

EXAMPLES

The following examples are given for the purpose of illustrating various embodiments of the disclosure and are not meant to limit the present disclosure in any fashion. The present examples, along with the methods described herein are presently representative of preferred embodiments, are exemplary, and are not intended as limitations on the scope of the disclosure. Changes therein and other uses which are encompassed within the spirit of the disclosure as defined by the scope of the claims will occur to those skilled in the art. Other equivalent embodiments will be apparent in view of the present disclosure, figures and accompanying claims.

### Example I

### Design, Synthesis, and Testing of a 57-Codon Genome

According to some aspects, methods are described herein for design and construction of a radically recoded Escherichia coli. Recoding, the re-purposing of genetic codons, is a powerful approach to enhance genomes with functions not commonly found in nature. The degeneracy of the canonical genetic code allows the same amino acid to be encoded by multiple synonymous codons. The near universality of a 64-codon code among natural organisms (Crick, 1963) makes codon replacement a powerful tool for genetic isolation of synthetic organisms. For example, while most organisms follow a common 64-codon template for translation of cellular proteins, deviations from this universal code found in several prokaryotic and eukaryotic genomes (Ambrogelly et al. 2007, Kano et al., 1991, Oba et al., 1991, Macino et al., 1979, Ling et al., 2015) have spurred the exploration of synthetic organisms with expanded genetic codes.

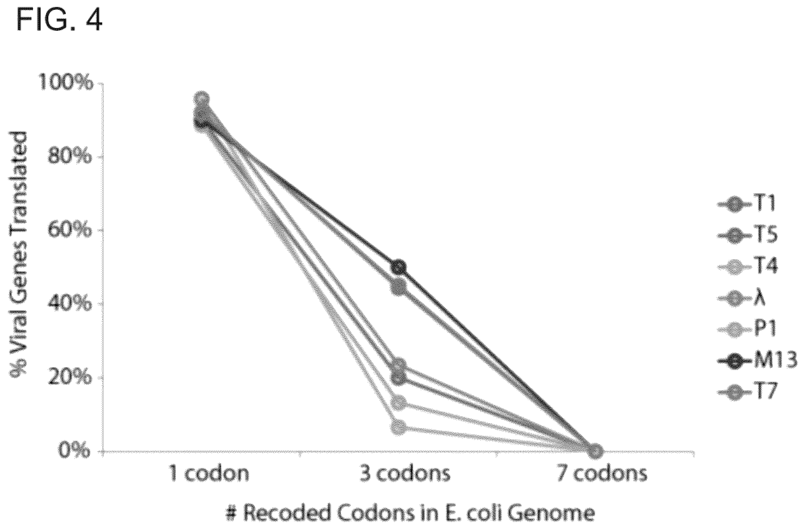

Whole-genome synonymous codon replacement provides a mechanism to construct unique organisms exhibiting genetic isolation and expanded biological functions. Once a codon is synonymously replaced genome-wide and its cognate tRNA is eliminated, the genomically recoded organism (GRO) may no longer translate the missing codon (Lajoie et al., 2013b). Therefore, genetic isolation is achieved since DNA acquired from natural viruses, plasmids and other organisms would be improperly translated, rendering the recoded strain insensitive to infection by viruses and horizontal gene transfer (FIG. 4).

For example, FIG. 4 illustrates, for a panel of coliphages, the percent of bacteriophage genes that are predicted to be properly translated in recoded E. coli strain with an increasing number of unassigned missing codons (e.g., no cognate translation). In this example, 1 codon=UAG; 3 codon=UAG, AGG, and AGA; and 7 codons=UAG, AGG, AGA, AGC, AGU, UUG, and UUA.

The gene translation percentage may be computed by the following equation:

$$Gene translation \% = \frac{\begin{matrix}Total \# of genes in given viral genome - \\ \# of viral genes containing forbidden codo ns\end{matrix}}{Total \# of genes in given viral genome}$$

Furthermore, proteins with novel chemical properties may be explored by reassigning replaced codons to incorporate non-standard amino acids (nsAAs) functioning as chemical handles for bioorthogonal reactivity, photoresponsive elements, or biophysical probes (Liu et al., 2010). Codon reassignment has also made it possible to establish metabolic dependence on nsAAs that do not naturally exist in the environment, enhancing biocontainment of GROs which may be a major consideration in environmental, industrial and medical applications (Marliere, 2009, Mandell et al., 2015, Rovner et al., 2015). In some embodiments, non-standard amino acids (nsAAs) may comprise any amino acid other than the 20 canonical protein coding amino acids. In other words, nsAAs may include any amino acid incorporated using one or more codons whose assignment differs from those of a given natural organism.

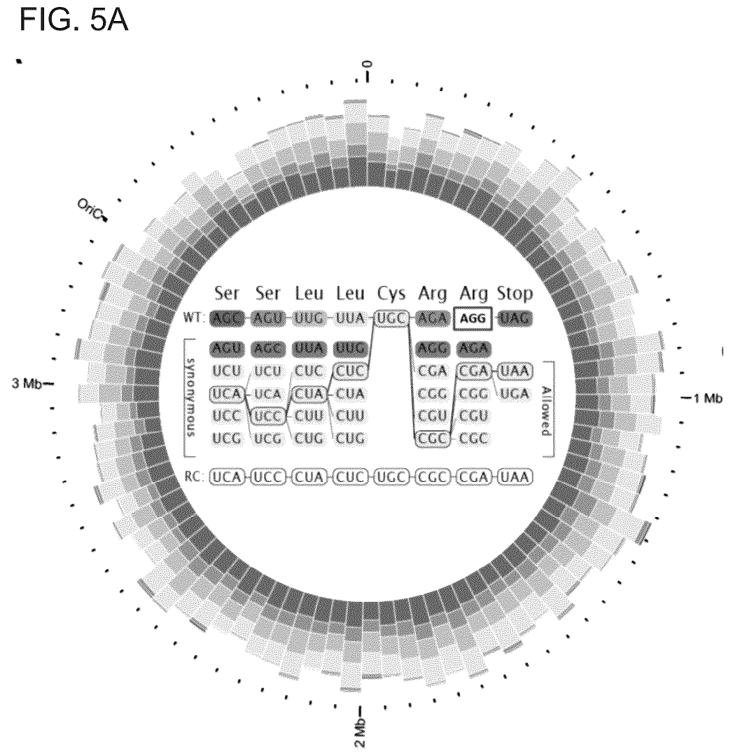

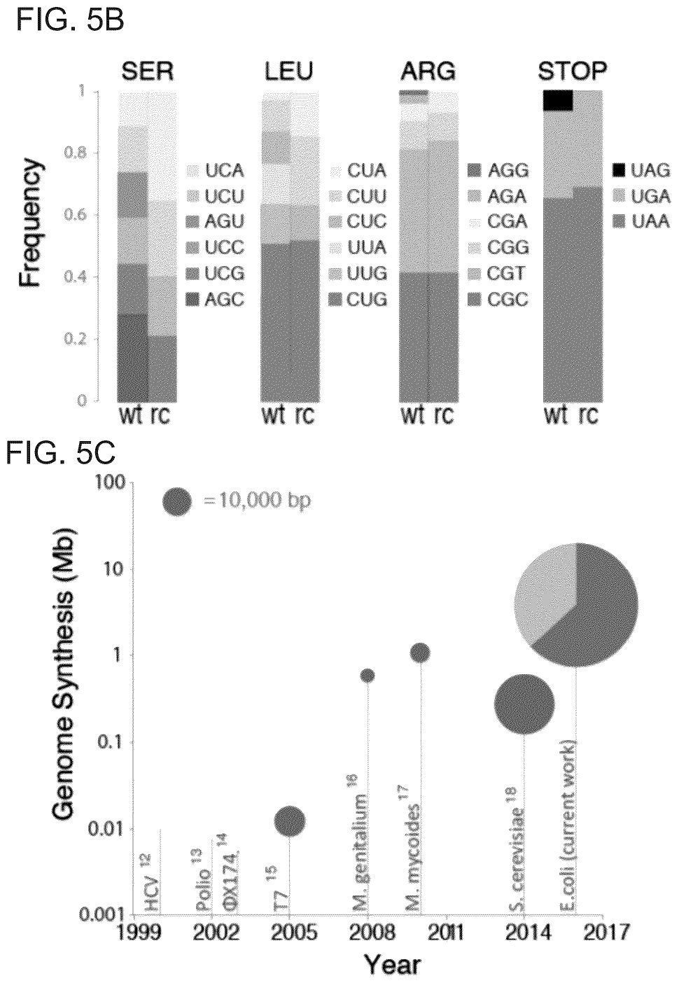

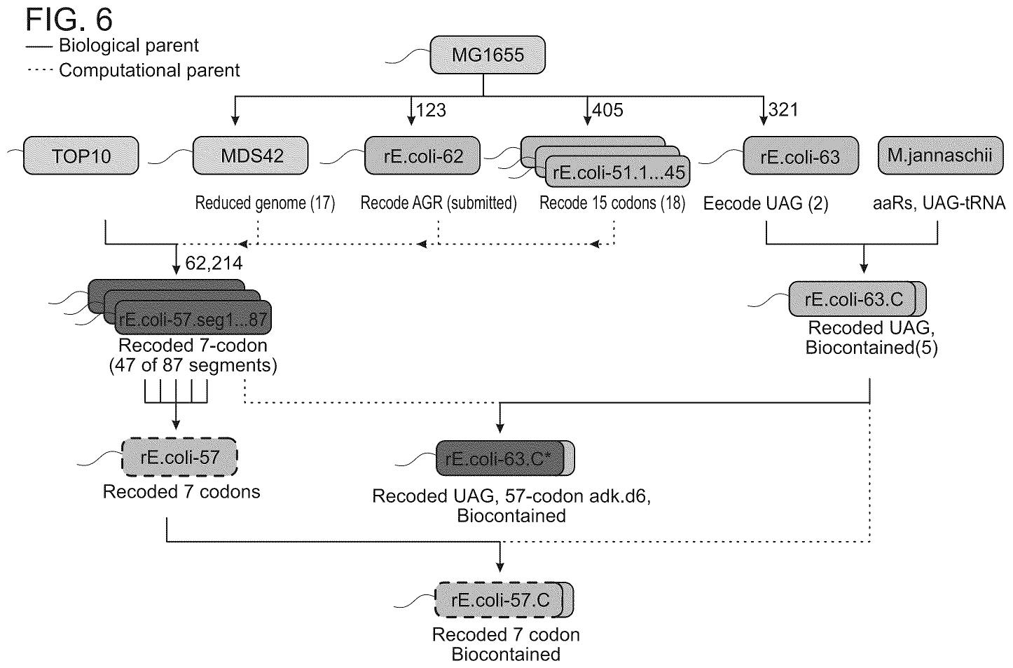

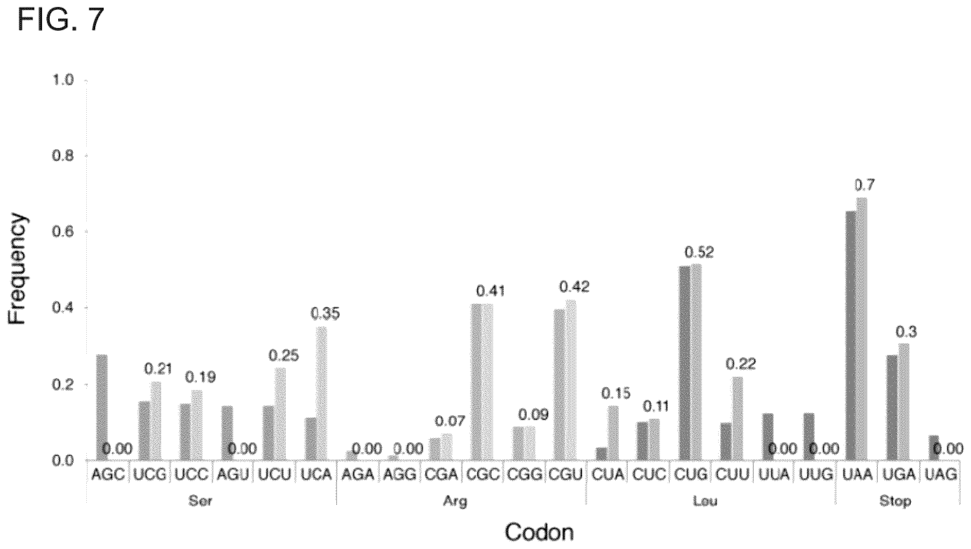

Described herein are methods for multiple codon replacements genome-wide, with the aim of producing a virus-resistant, biocontained organism relevant for industrial applications. A computational design is presented, along with experimental testing of 2.5 Mb (63%) of an E. coli genome in which all 62,214 instances of seven different codons (corresponding to 5.4% of all E. coli codons) have been synonymously replaced (FIG. 5A-5C). The new recoded genome may be referred to as rE. coli-57 as described herein and is composed of 57 of canonical 64 codons when assembled (FIG. 6). While several synthetic genomes have been previously reported (Blight et al., 2000, Cello et al., 2002, Smith et al., 2003, Chan et al., 2005, Gibson et al., 2008, Gibson et al., 2010, Annaluru et al., 2014), a functionally altered synthetic genome of this scale has not yet been explored (FIG. 5C).

In some cases, alterations of codon usage may affect gene expression and cellular fitness at multiple levels from translation initiation to protein folding (Kudla et al., 2009, Tuller et al., 2010, Plotkin et al., 2011, Goodman et al., 2013, Zhou et al., 2013, Quax et al., 2015, Boël et al., 2016). Yet, parsing the individual impact of codon choices may remain difficult, imposing a barrier to designing new genomes. The present disclosure provides prediction tools and efficient technologies to rapidly prototype synthetic genomes.

In order to address the unprecedented scale and complexity of genome engineering goals, computational tools, cost-effective de novo synthesis strategy, and a comprehensive experimental validation plan as described herein. For example, the number of modifications required to replace all instances of seven codons may be far beyond the current capabilities of single-codon editing strategies previously used for genome-wide replacement of the UAG codon (Lajoie et al., 2013b, Isaacs et al., 2011). Although it may be possible to simultaneously edit multiple alleles using MAGE (Wang et al., 2009) or Cas9 (Esvelt et al., 2013), these strategies may involve extensive screening using numerous oligos and RNA guides and may likely introduce off-target mutations (Wang et al., 2009). De novo synthesis allows for an almost unlimited number of modifications independent of biological template. Moreover, the plummeting costs of DNA synthesis are reducing financial barriers for synthesizing entire genomes.

For this example, the following three codons were chosen for replacement: the UAG stop codon and the AGA and AGG arginine codons (FIG. 6). These codons were also among the rarest codons in the genome, minimizing the number of changes required. The other codons were chosen such that their anticodon is not recognized as a tRNA identity element by endogenous aminoacyl-tRNA synthetases, so that heterologous tRNAs will not be mischarged with canonical amino acids upon incorporation of nsAAs. Lastly, to allow unambiguous reassignment, codons were chosen whose tRNA do not overlap with other synonymous codons for the same amino acid. Thus, the following seven codons (termed ‘forbidden codons’) were targeted for replacement: AGA (Arg), AGG (Arg), AGC (Ser), AGU (Ser), UUG (Leu), UUA (Lcu) and UAG (Stop) (FIG. 5A-5C, FIG. 6, FIG. 3).

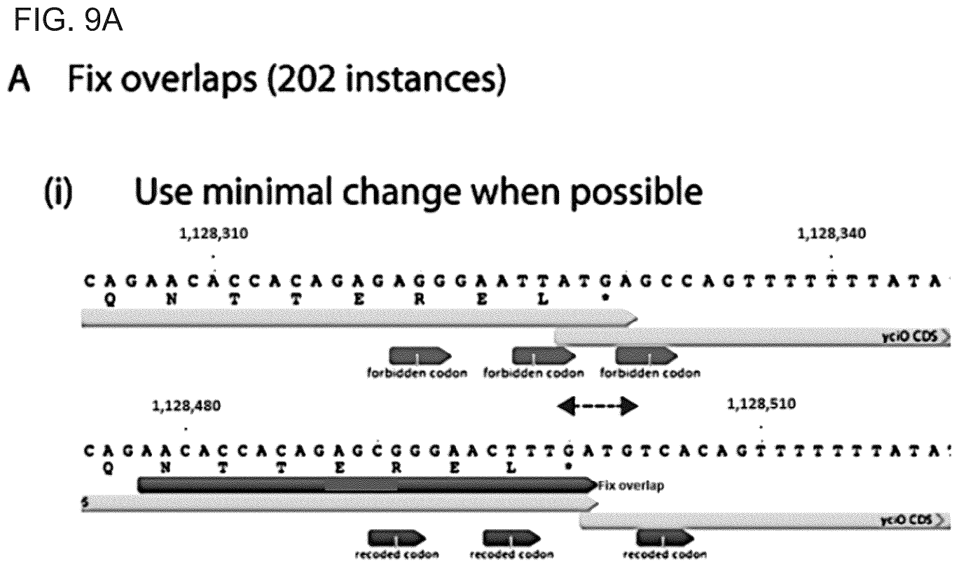

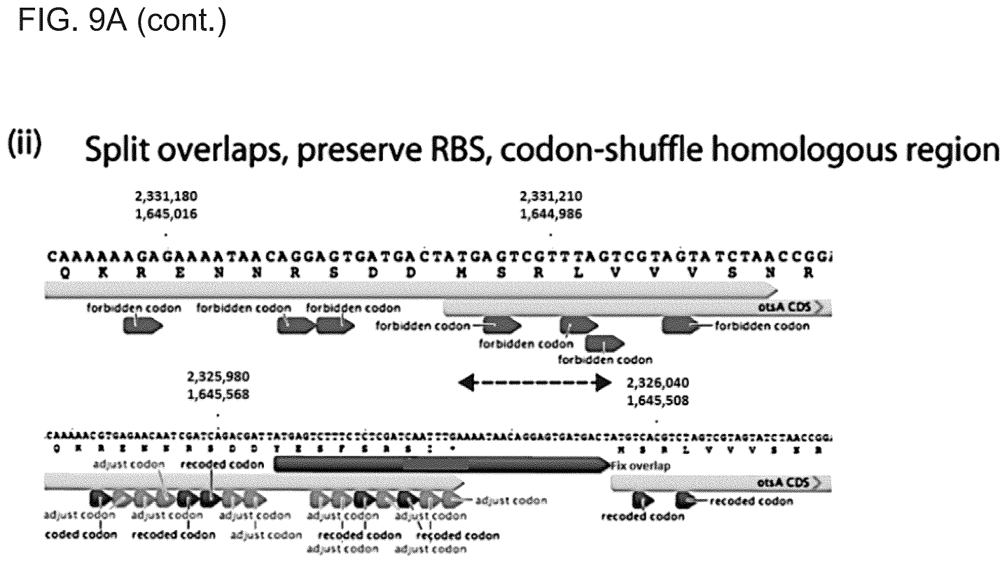

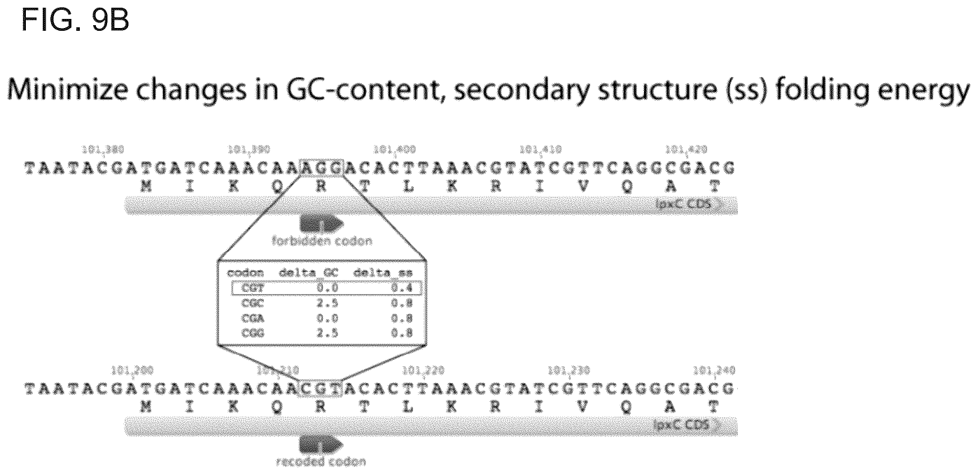

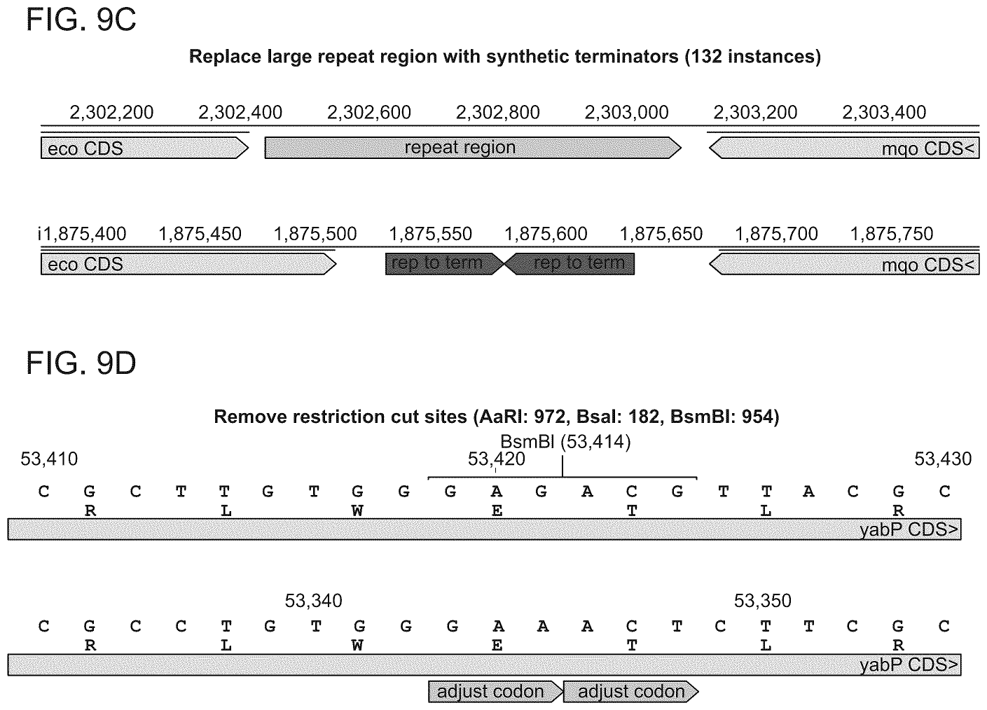

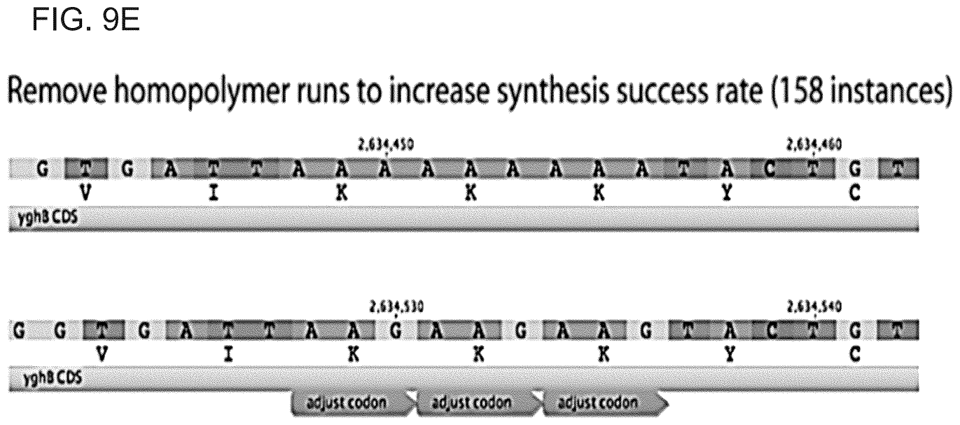

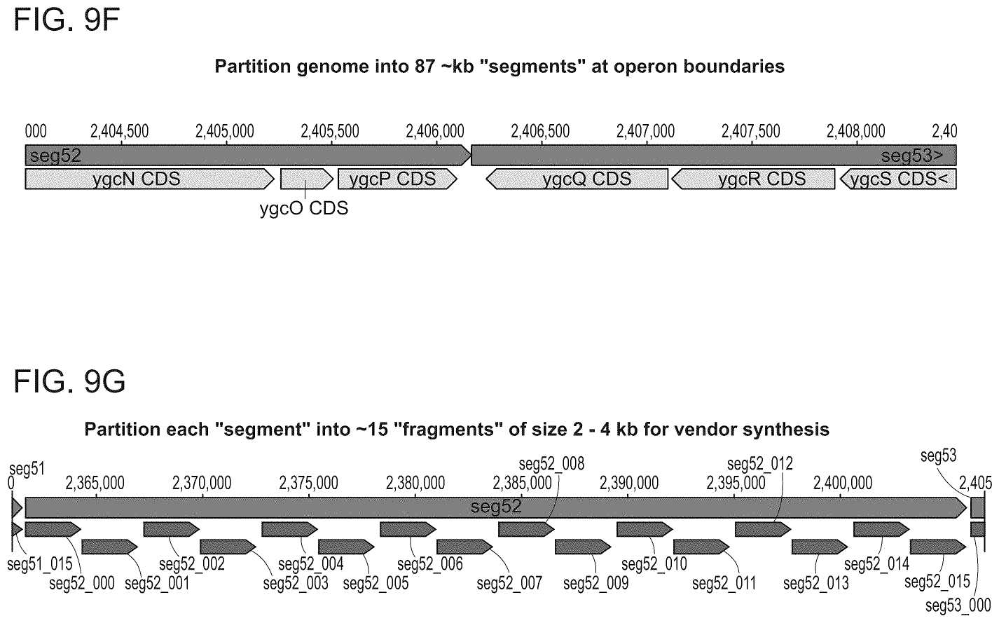

In order to minimize synthesis costs and improve genome stability, the 57-codon genome described herein is based on the reduced-genome E. coli strain MDS42 (Pósfai et al., 2006). The disclosed computational tool automates synonymous replacements for all occurrences of the target codons in all protein-coding genes while satisfying biological and technical constraints, in which examples of these constraints are illustrated in FIGS. 8-9 and Tables 1-2. In particular, amino acid sequences of all coding genes were preserved, and protein synthesis levels were maintained by separating overlapping genes carrying forbidden codons and by introducing synonymous codons to minimize potential recombination events (Chan et al., 2005, Temme et al., 2010). The relative codon usage of the remaining codons was conserved to meet translational demand (Yona et al., 2013) and to preserve characteristics of the primary nucleotide sequence, including predicted ribosome binding site (RBS) strength, mRNA secondary structure folding energy, and GC content (Lajoie et al., 2013b, Lajoie et al., 2013a). Finally, adjustments were made to avoid difficult-to-synthesize sequences from the final genome design (e.g., removing homopolymers, normalizing regions of extreme GC content and reducing repetitive sequences) (FIGS. 9A-9G).

Overall, forbidden codons were uniformly distributed throughout the genome, averaging about 17 codon changes per gene. Essential genes (Yamazaki et al., 2008), which provide a stringent test for successful codon replacement, contain about 6.3% of all forbidden codons (3,903 of 62,214 codons). Altogether, the recoded genome necessitated a total of 148,955 changes to remove all instances of forbidden codons and adjust the primary DNA sequence to accommodate design constraints.

Once designed, the recoded genome was parsed into 1,256 synthesis-compatible overlapping fragments of 2 to 4 kilobases (kb). 87 segments of about 50-kb were individually assembled and tested (FIG. 8). Segments of about 50-kb contain a manageable number of genes, averaging about 40 total genes and about 3 essential genes per segment. Additionally, it was found that 50-kb may be a convenient size for assembly in yeast and shuttling into E. coli. Importantly, based on earlier studies (Mandell, D. J. et al., Biocontainment of genetically modified organisms by synthetic protein design. Nature. 518, 55-60 (2015).; K. M. Esvelt et al., Orthogonal Cas9 proteins for RNA-guided gene regulation and editing. Nat. Methods. 10, 1116-1121 (2013)) it was estimated that each segment would on average contain only about 1 potentially lethal recoding exception.

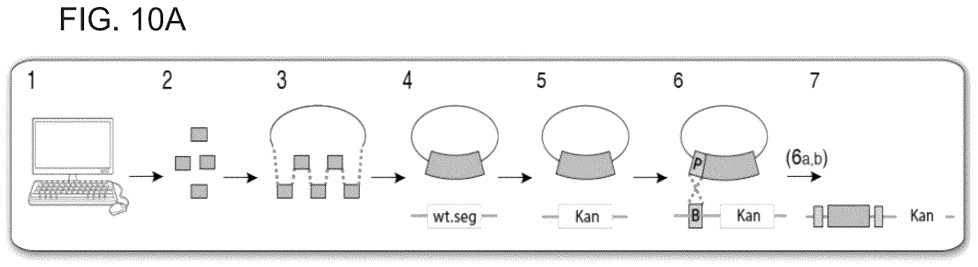

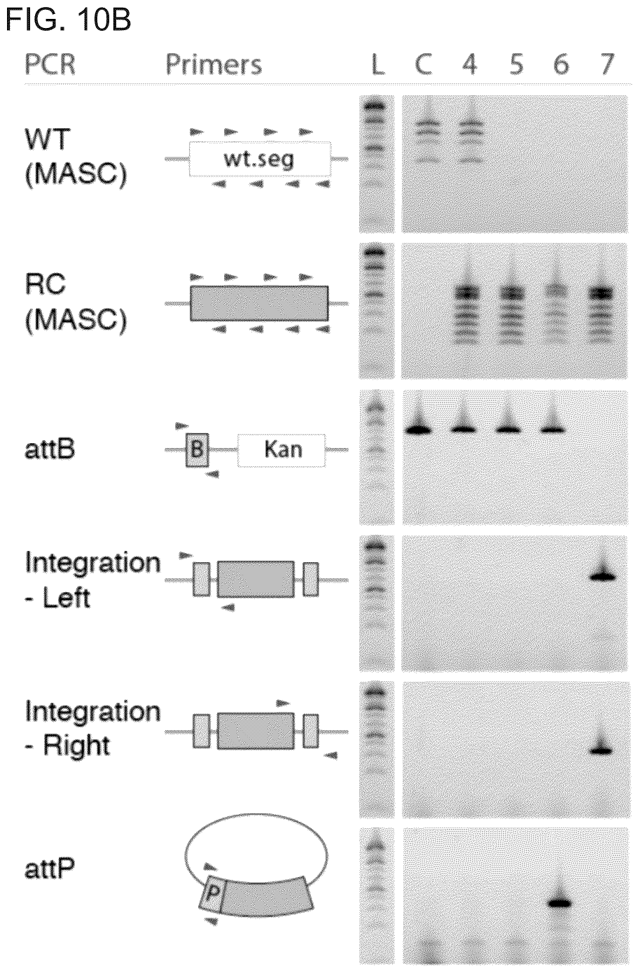

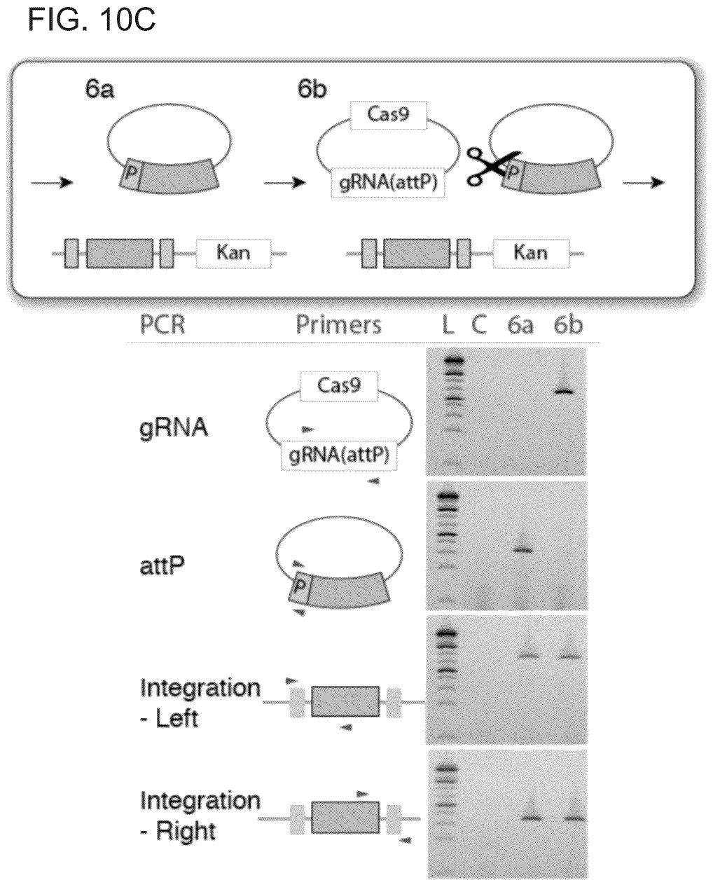

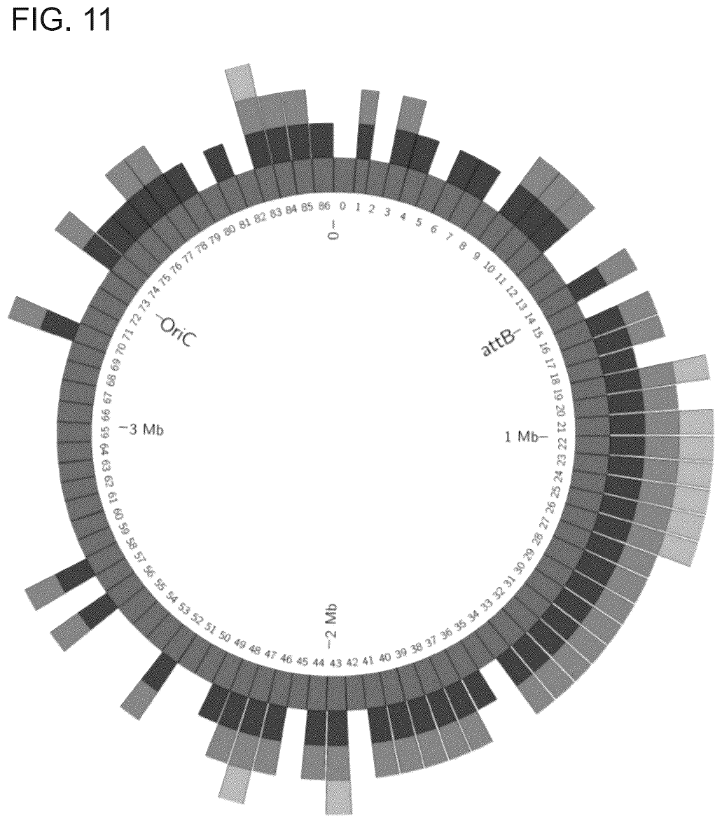

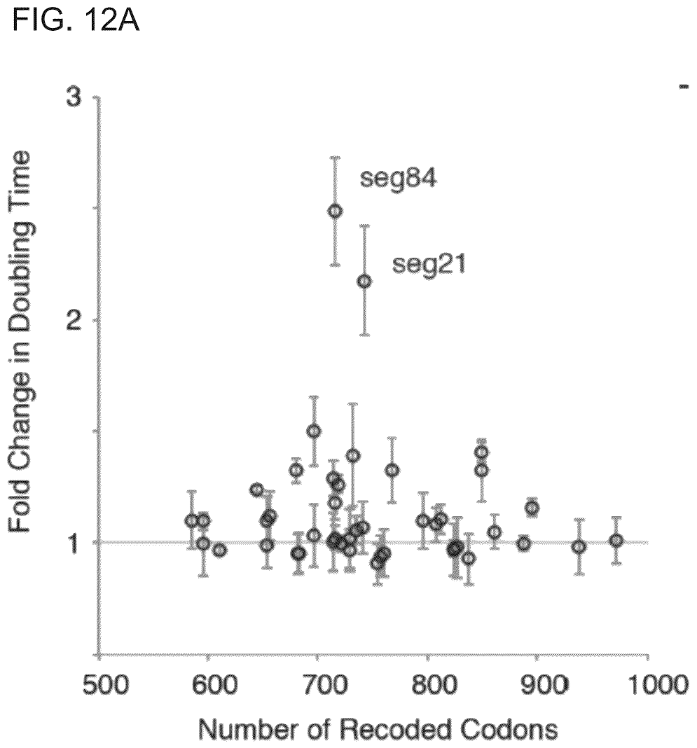

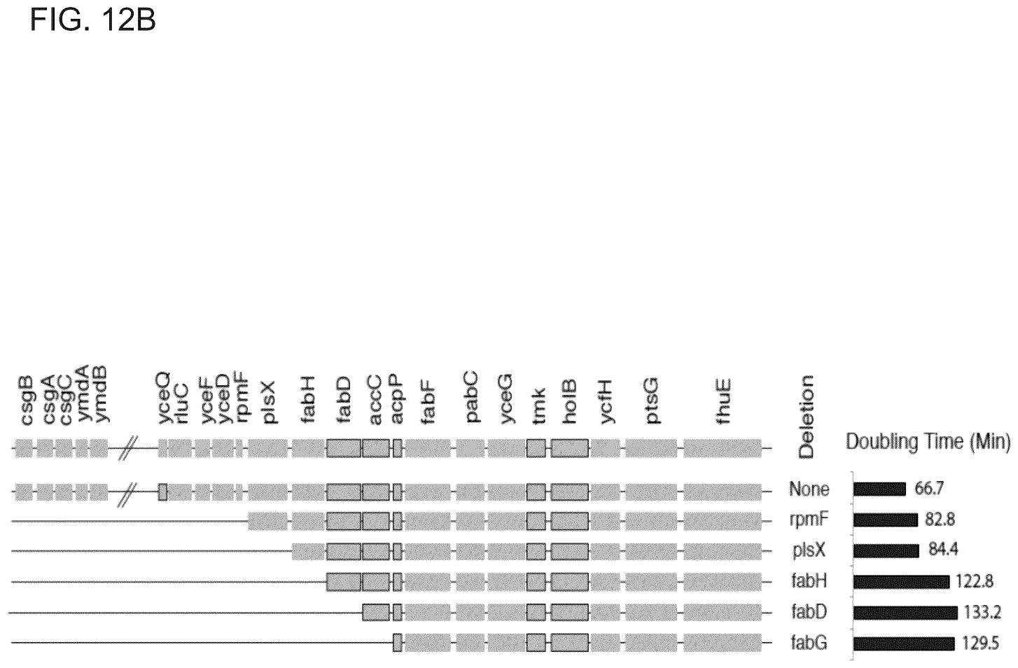

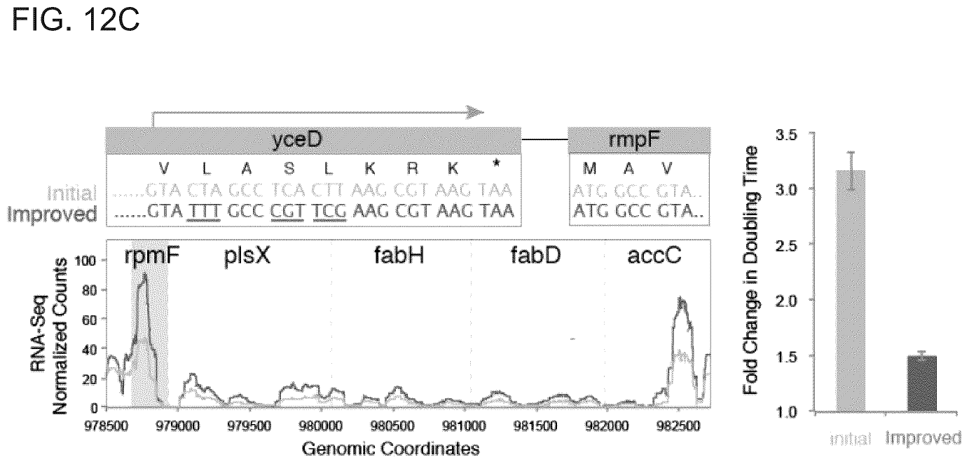

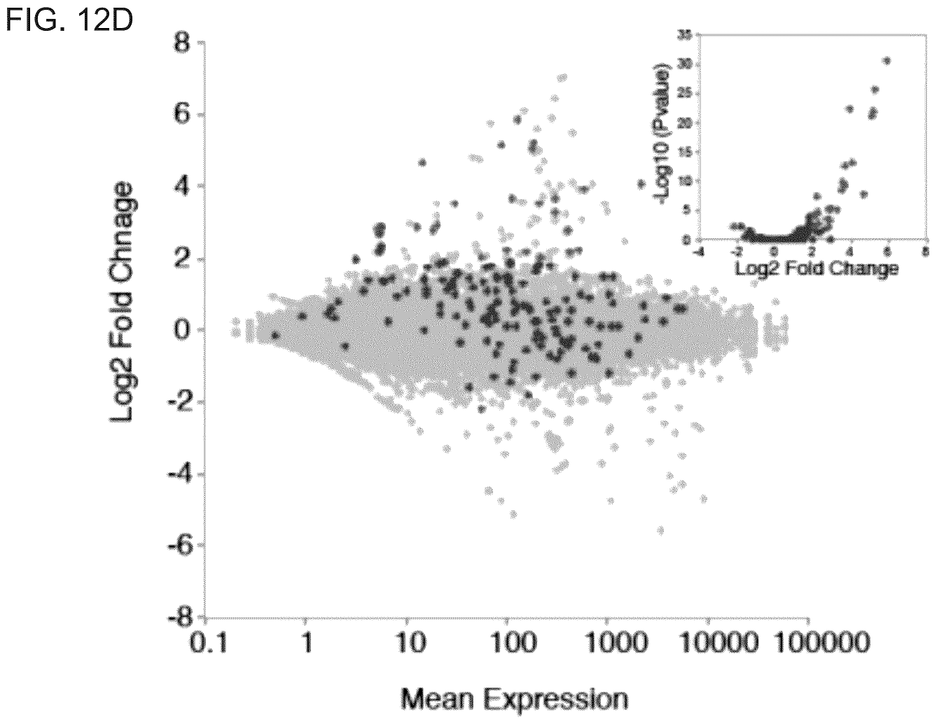

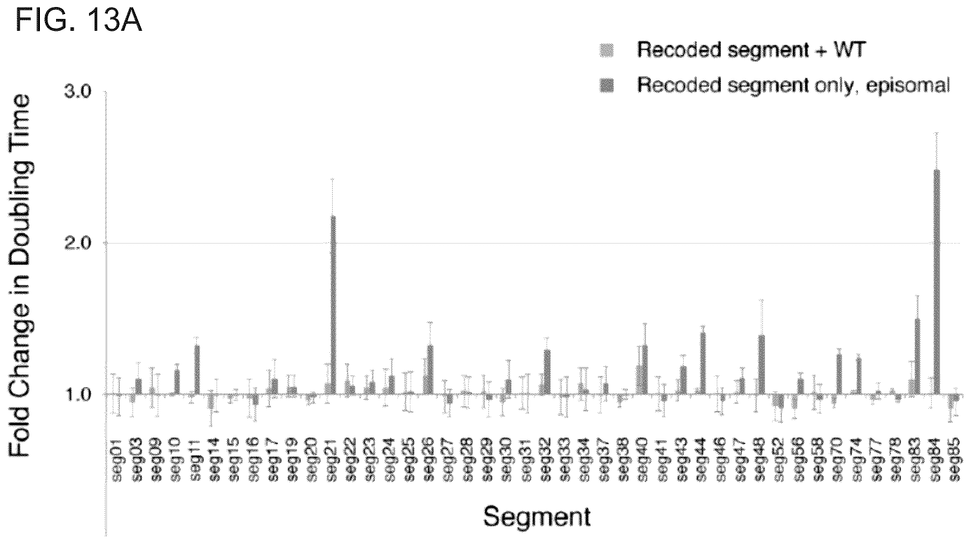

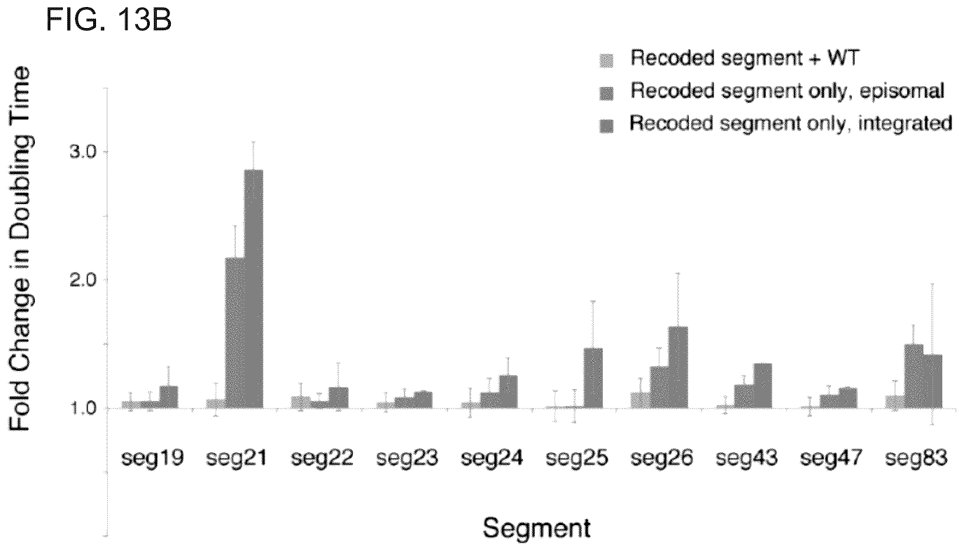

FIGS. 10A-10C outline the experimental strategy utilized in this example. In brief, each segment was assembled in S. cerevisiae and electroporated directly into E. coli on a low copy plasmid. Subsequent deletion of the corresponding chromosomal segment provides a stringent test for the function of the recoded genes because errors in essential genes would be lethal. Thus far, chromosomal deletions for 2,229 recoded genes across 55 segments have been performed, accounting for 63% of the entire genome and 53% of essential genes (FIG. 11). Additionally, all recoded genes in 44 of these 55 segments were found to complement wild-type chromosomal genes without requiring any optimization. The growth of these strains was assessed, and gene expression was analyzed via RNA-Seq (FIGS. 12A-12B). Moreover, the majority of these strains exhibited only marginal fitness impairment upon chromosomal deletion (FIG. 12A, FIGS. 13A-13B).

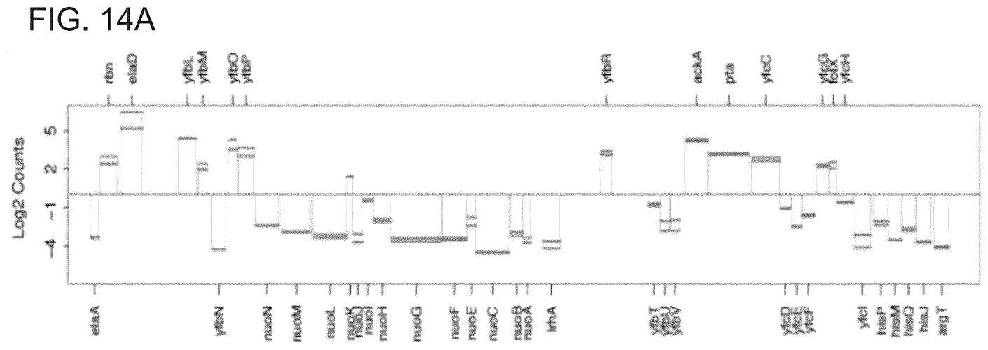

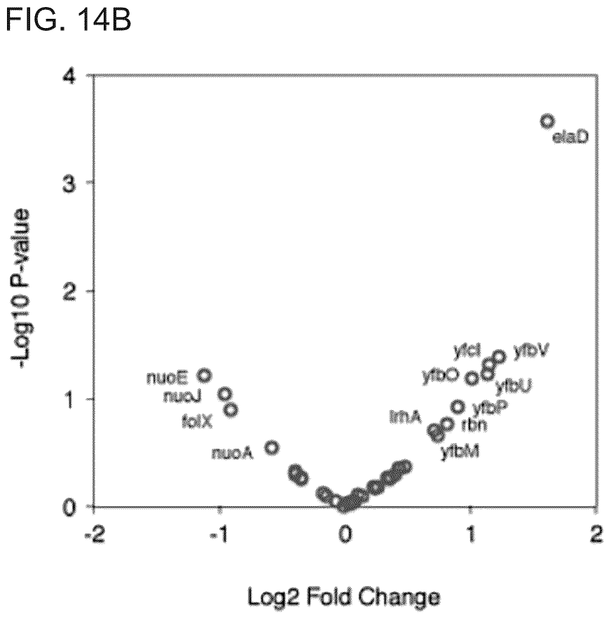

Furthermore, RNA-Seq analysis of 208 recoded genes suggests the majority show only minor change in transcription due to codon replacement (FIG. 14A-14B). Only 28 genes were found to be significantly differentially expressed (i.e., >2-fold change, p<0.01) (27 overexpressed, 1 underexpressed).

Recoded segments that failed to complement the entire wild-type segment (e.g., 11 of 55 segments) were tested by making small chromosomal deletions of the region until the causal gene(s) was localized. Overall, 13 recoded essential genes were found that failed to support cell viability due to synonymous codon replacement. In some embodiments, these may be referred to as “design exceptions.”

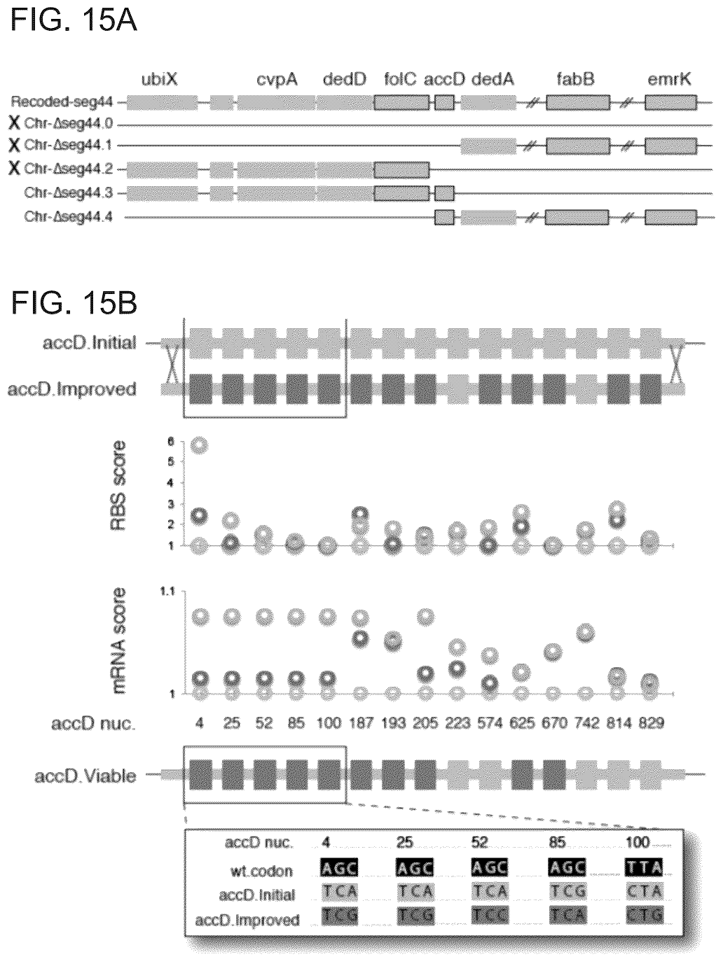

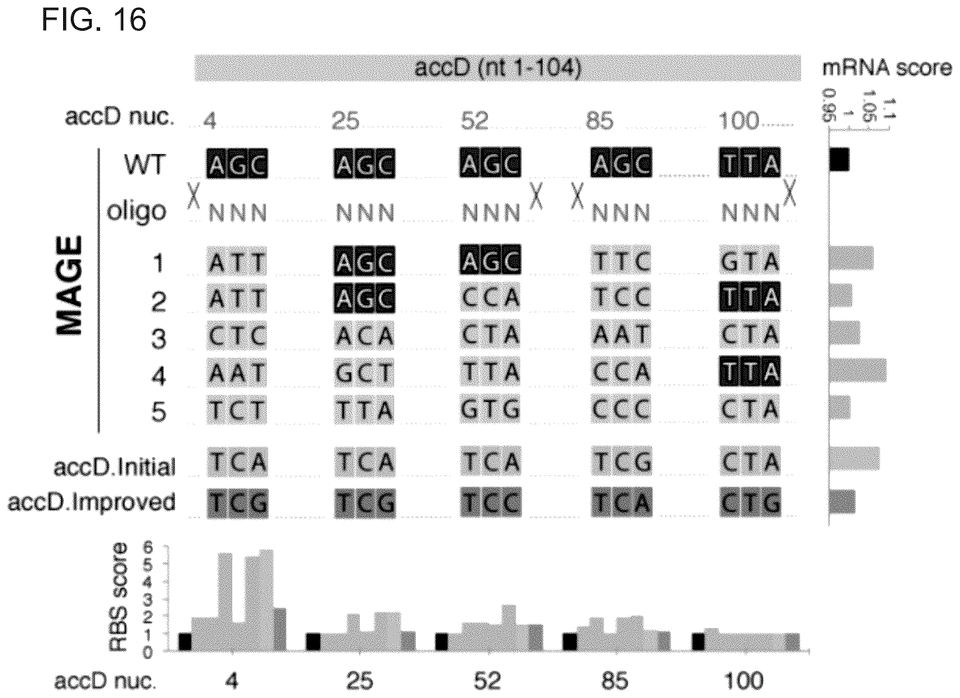

Segment 44 was selected as a test case to develop a troubleshooting pipeline for solving design exceptions (FIGS. 15A-15B). As shown for gene accD, RBS strength and mRNA folding were first analyzed to pinpoint the most probable cause of disruption in gene expression (Plotkin et al., 2011, Goodman et al., 2013, Boël et al., 2016). Then, degenerate MAGE oligos were used to rapidly prototype viable alternative codons (FIG. 16). For calculating the mRNA secondary structure score, a sliding window of 40 bp around the codon of interest was used. The algorithm was further updated to score mRNA secondary structure as a skewed interval that is-30 to +100 nucleotides relative to the codon of interest. Notably, for codons in the first 100 nucleotides, the window was centered at the start of the gene.

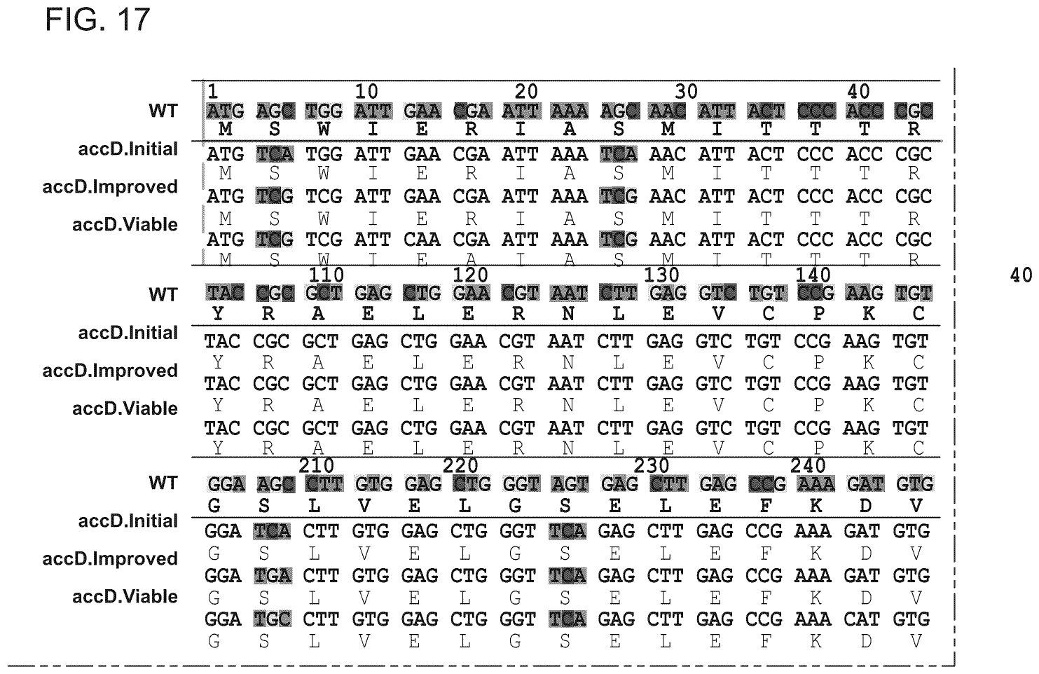

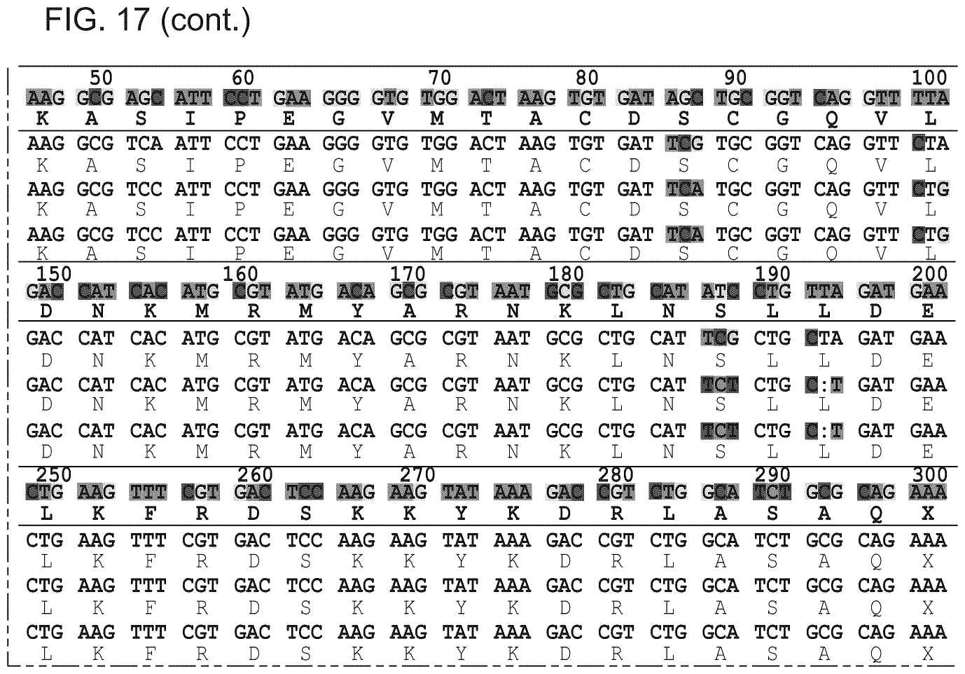

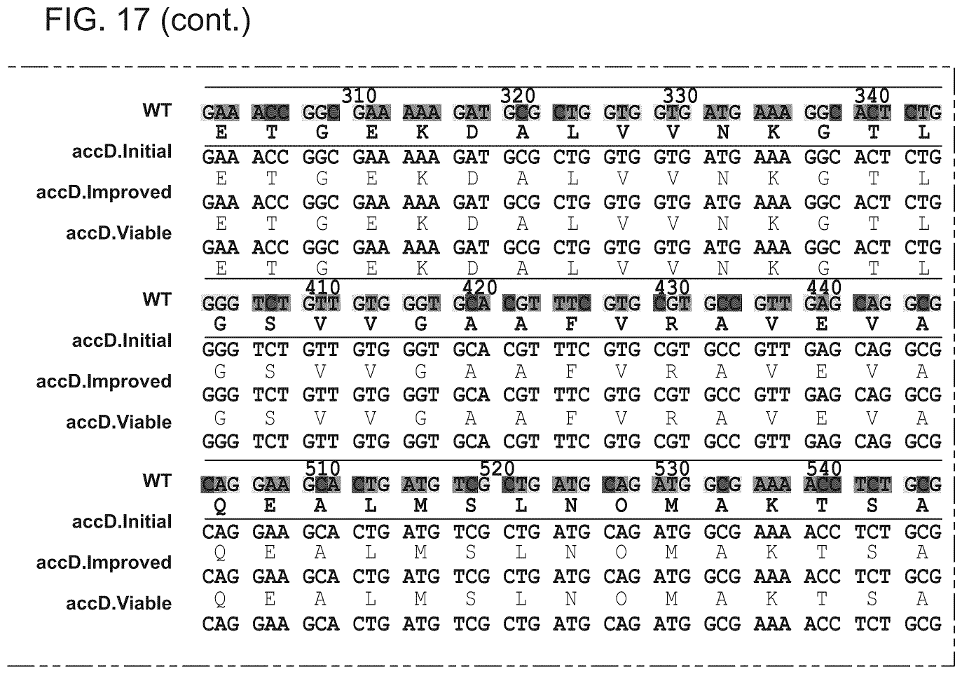

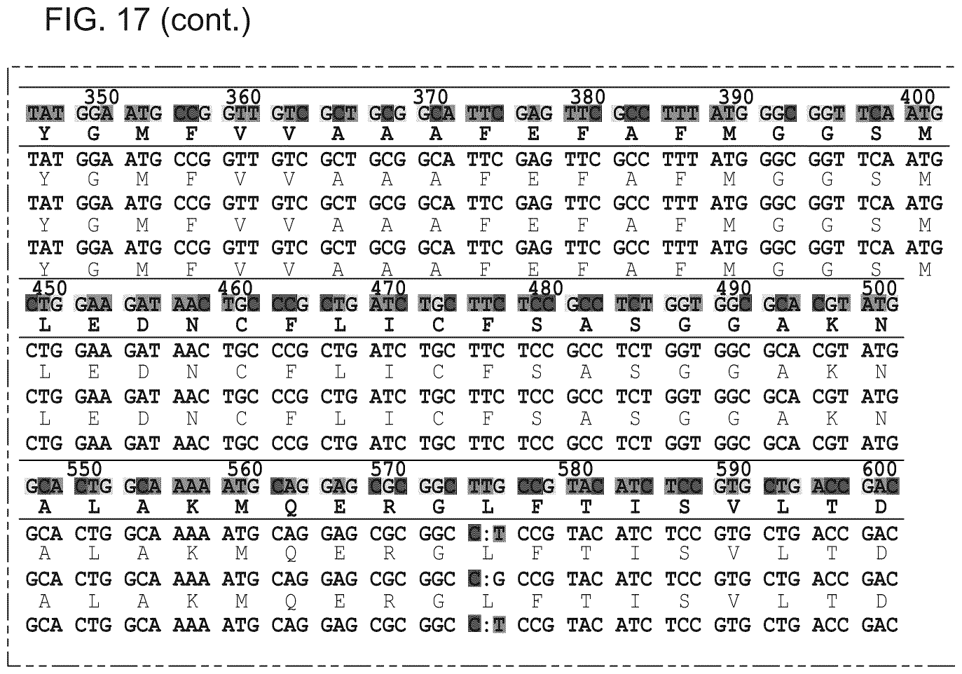

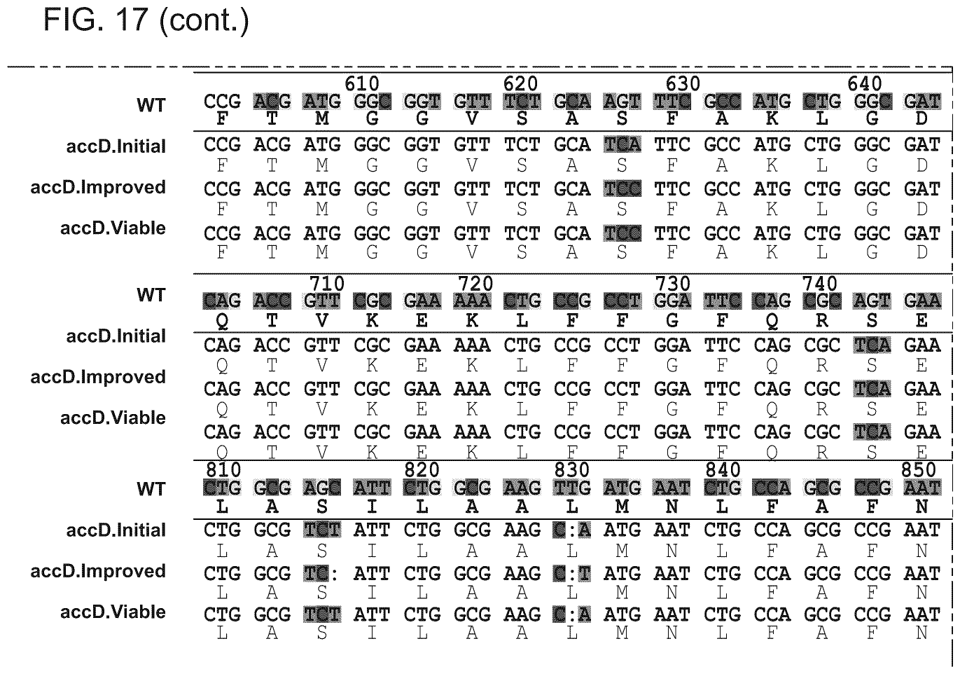

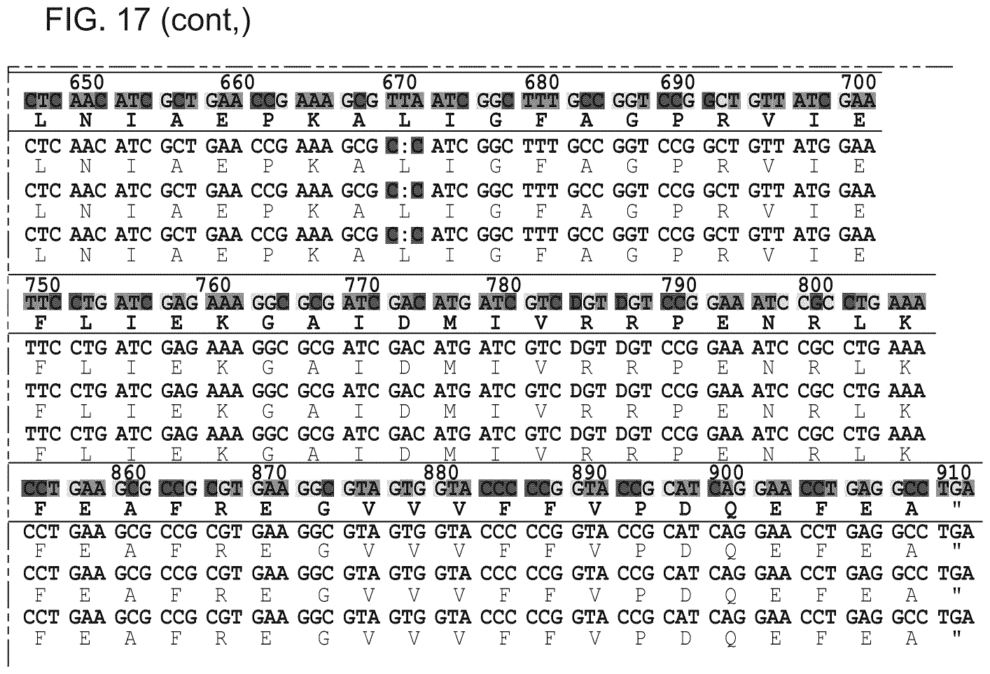

Finally, a new recoded sequence was computationally generated using more stringent mRNA and RBS scoring parameters (FIGS. 15A-15B, FIG. 17) and was introduced into the recoded segment via multiple cycles of lambda Red recombineering. Viable clones were selected by the subsequent chromosomal deletion.

In some cases, all viable clones carried a specific sequence of accD that had the N-terminal end of the improved design and the C-terminal end of the initial (lethal) design, highlighting the significance of N-terminal optimization for successful synonymous codon replacement (Kudla et al., 2009, Goodman et al., 2013). Furthermore, such recombination events, which are expected due to the high degree of homology between the two gene versions, effectively shuffle the sequences and increase the search space of viable recoded codons.

To further confirm adequate chromosomal expression, the recoded segment was integrated into the chromosome using λ-integrase. attP-specific Cas9-mediated DNA cleavage was then used to ablate all non-integrated plasmids, leaving a single integration event per genome. No fitness changes were observed upon segment integration (FIG. 13A-13B). Finally, DNA sequence analysis of all validated strains may suggest some degree of in vivo accumulation of mutations, which may be expected during strain engineering. Yet, to achieve complete genome recoding, non-lethal reversions and silent mutations may be corrected in the final strain using MAGE.

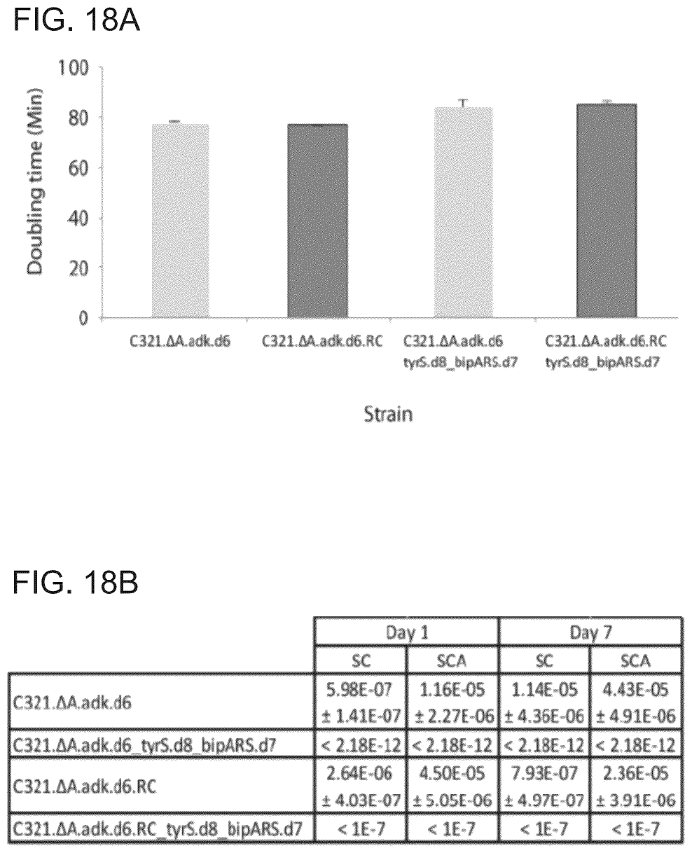

According to certain aspects, substantial modifications to both codon usage and tRNA anticodons may lead to instability of a reduced genetic code without proper selection to prevent codon reversion (Osawa et al., 1989); however, establishing functional dependence on the recoded state may both stabilize the modified genome and offer a stringent biocontainment mechanism (Marliere, 2009). As an example, a biocontained strain was developed in which all UAG codons were removed and two essential genes (adk and tyrS) were altered so that the strain required nsAAs to remain viable (Mandell et al., 2015). In order to determine whether the final rEcoli-57 strain will support a similar biocontainment mechanism, the 57-codon versions of both adk and tyrS were confirmed to be functionally active in vivo. Moreover, it was found that recoded and nsAA-dependent adk gene has the same fitness and extremely low escape rates reported for the original strain (FIG. 18A-18B).

Even after all instances of forbidden codons are removed from the genome, the genetic code may remain unchanged until the genes for five tRNAs (argU, argW, serV, leuX, leuZ) and one release factor (prfA) are removed. Once rEcoli-57 is fully recoded and these tRNAs are removed, the strain may be tested for novel properties such as resistance to viruses and horizontal gene transfer. Additionally, orthogonal aminoacyl-tRNA synthetase/tRNA pairs may be introduced to expand the genetic code by as many as 4 nsAAs.

Ultimately, the hierarchal, in vivo validation approach supported by robust design software, as described herein, may be utilized for large-scale synthetic genome construction and to radically change the genetic code. Genetically isolated and recoded genomes may expand synthetic functionality of living cells, offering a unique chassis for broad applications in biotechnology.

### DNA Synthesis

DNA was synthesized by industrial partners Gen9, SGI-DNA, Twist Biosciences, Genewiz, and IDT DNA technologies. The synthesis pipeline was developed primarily with the aim of reducing synthesis cost and turnaround time, considering constraints of synthesis error rate and QC. Gen9 synthesized the majority of DNA, providing 3,960 kb as fragments ranging in size from 1.2-4.2 kb. Additional synthesis was provided by Twist Biosciences (30 kb in fragments ranging 1 .4-2.0 kb) IDT (27 kb in fragments ranging 1 .0-1.7 kb), and Genewiz (26 kb in fragments ranging 12.4-3.0 kb). An additional 328 kb (SGI-DNA), 36 kb (Twist), and 6 kb (Gen9) were synthesized, but were not used in the final genome segment syntheses.

### PCR Amplification of Synthetic DNA

All synthetic DNA was PCR amplified and purified prior to assembly. 30 μL of PCR reaction was prepared as follows; 1 μL of diluted template DNA (1 μL synthetic template DNA (synDNA) ranging 1 to 5 ng/μL, diluted in 9 μL TE buffer), 2 μL of primer mix (10 μM each primer, mixed in 50 μL of TE buffer), 15 μL of 2×SeqAmp DNA polymerase (Clontech Laboratories, Inc.), and 15 μL of PCR grade water. PCR cycles: 95° C.-1 minute, 98° C.-10 seconds, 60° C.-15 seconds, 68° C.-2 minutes, 35 cycles. 1% agarose gel was used to analyze 1 μL of PCR product. Optimization of unsuccessful PCR was done using 2× KAPA-HiFi DNA polymerase (Kapa Biosystems). 30 μL of PCR reaction was as follows; 1 μL of diluted template DNA (as above), 2 μL of primer mix (as above), 15 μL of 2× KAPA-HiFi, and 12 μL of PCR grade water. PCR cycles: 95° C.-1 minute, 98° C.-20 seconds, 60° C.-15 seconds, 72° C.-2 minutes, for 30 or 35 cycles. PCR products were gel purified using 2% E-gel Ex (Thermo Fisher Scientific Inc.).

Segment Assembly in S. cerevisiae

For segment assembly, GeneArt High-Order Genetic Assembly System (Life Technologies) was used with modifications. The vector pYESIL was modified to include restriction sites EcoRI and BamHI used for linearization, and a S. cerevisiae uracil selective marker was added to the vector backbone (termed ‘pYESIL-URA’). Vector digestion was performed with both enzymes as follows: 5 hours at 37° C., followed by 20 minutes enzyme inactivation at 65° C. and 30 minute End Repair Module (NEB) treatment at 20° C. Linear vector was purified (Zymo DNA Clean & Concentrator) and size verified on DNA gel prior to use. Amplified synthetic fragment (400 ng of each) were mixed and purified for each assembly reaction (10-15 fragments used for each assembly), then added with 100 ng of purified linear vector pYESIL-URA. Vector/fragment DNA mix was concentrated using SAVANT DNA 120 SpeedVac concentrator (Thermo Fisher Scientific Inc.) to ˜10 μL in volume.

Transformation of MaV203 competent cells was performed according to manufacturer instructions. Cells were plated on CM glucose media without tryptophan and incubated at 30° C. for 3 days. Colony PCR was used to screen for segment assembly; yeast colony was lysed in 15 μL of 0.02 M NaOH, boiled for 5 minutes at 95° C. and kept on ice for 5 minutes, followed by dilution with 40 μL ddH2O. 1.5 μL of the mix was used as template for multiplex PCR using KAPA2G multiplex polymerase (KAPA Biosystems) and the following PCR conditions: 98° C.-5 minute, 98° C.-30 seconds, 62° C.-30 seconds, 72° C.-30 seconds, 72° C.-5 minutes (32 cycles). Only colonies showing positive PCR were used. For E. coli transformation, cells were lysed in 15 μL 0.02 M NaOH, vortexed with glass beads for 5 minutes and placed on ice. 1.5 μL of the lysis mix was added to electrocompetent TOP10 cells (Thermo Fisher Scientific), immediately electroporated (1.8 kV, 25 μFarads, 200Ω), and recovered for 1 hour at 37° C. before plating on spectinomycin selective plates.

### E. coli Methods-Strains & Culture

TOP10 electrocompetent E. coli (Thermo Fisher Scientific) were used for the entire process for all segments except segments 19,22,23,43,44,47 that were performed in BW38028 (Conway et al., 2014). EcM2.1 naïve strains were used for troubleshooting (EcM2.1 is a strain optimized for MAGE-Escherichia coli MG1655 mutS_mut dnaG_Q576A exoX_mut xonA_mut xseA_mut1255700::tolQRA Δ(ybhB-bioAB)::[2c1857 N(cro-ea59)::tetR-bla]) (Gregg et al., 2014).

Liquid culture medium consisted of the Lennox formulation of Lysogeny broth (LBL; 1% w/v bacto tryptone, 0 .5% w/v yeast extract, 0 .5% w/v sodium chloride) with appropriate selective agents: spectinomycin (95 μg/mL), chloramphenicol (50 μg/mL), kanamycin (30 μg/mL), carbenicillin (50 μg/mL), zeocin (10 μg/mL). Solid culture medium consisted of LBL autoclaved with 1 .5% w/v Bacto agar (Thermo Fisher Scientific), containing the same concentrations of antibiotics as necessary.

### Plasmid Transformation, Lambda Red Recombinations, MAGE

TOP10 and BW38028 (Conway et al., 2014) cells transformed with pYESIL-URA plasmid were the subject of all pipeline strain engineering. The average copy number for recoded segment on vector pYESIL-URA was found to be 1.8 plasmids/genome.

Knockout of the homologous chromosomal non recoded segment sequence is achieved by lambda Red recombineering specifically targeted to the genomic locus. 50 bp homology arms of the kanamycin cassette deletion are targeted to both sides of the genomic segment, which are different in sequence than the two sides of the plasmid carrying recoded segment. Therefore, the cassette specifically replaces the genomic segment.

All cells were transformed with pKD78 plasmid (Datsenko et al., 2000) to introduce the lambda Red recombineering machinery. Recombinase expression was induced for 2 hrs in Arabinose (2 μg/mDfollowed by DNA transformation, using either double-stranded PCR products or MAGE oligonucleotides. Notably, all kanamycin cassette deletions were performed with 100 ng double-stranded PCR products. Each recombination was paired with a negative control (deionized water) to monitor kanamycin selection performance. Other recombincering experiments were carried out as described previously (Wang et al., 2009), and total oligo pool was adjusted to a maximum of 5 μM. After 3 hrs of recovery at 34° C., the cells were plated in permissive media (for MAGE) or selective media (e.g. kanamycin) and incubated overnight at 34° C. The amount of cells plated was ˜103 for MAGE experiments, ˜107 for plasmid transformations and ˜108 for kanamycin cassette deletions. Resulting strains were then subjected to verification by PCR.

### Oligonucleotides, Polymerase Chain Reaction

A complete table of PCR oligonucleotides and primers can be found in Tables 3 and 4. PCR products used in recombination or for Sanger sequencing were amplified with Kapa 2G Fast polymerase according to manufacturer's standard protocols. Multiplex allele-specific PCR (mascPCR) was used for multiplexed genotyping using the KAPA2G Fast Multiplex PCR Kit, according to previous methods (Isaacs et al., 2011). Primers for mascPCR were designed using an automated software specially built for this purpose. Sanger sequencing reactions were carried out through a third party (Genewiz). mascPCR screening was performed after the pKD78 transformation, kanamycin deletion, attP-zeocin insertion and λ-Integration steps.

### Genome Integration of Recoded Segments

λ-integrase was used for integration of recoded segment plasmid into E. coli genome (Haldimann et al., 2001). attP site was added to the segment vector by lambda-red recombineering, along with zeocin resistance marker. Then, λ-integrase was heat-induced for 6 hours at 42° C., and cells were plated on spectinomycin and kanamycin plates for screening. PCR screening was performed using attP and attB specific primers (attB-seq-f: CAG GGA TGC AAA ATA GTG TTG AG (SEQ ID NO: 2326); attB-seqr:GA GAA GTC CGC GTG AGG (SEQ ID NO: 2327); attP-f: GCGCTAATGCTCTGTTACAG (SEQ ID NO: 2328); attP-r:GAAATCAAATAATGATTTTATTTTGACTGA (SEQ ID NO: 2329)) as well as allele-specific primers (Table 4) to identify clones with correct plasmid integration.

### Cas9-Induced Vector Elimination

Once integrated, a further validation step was taken to ensure no additional copies of the recoded segments remain in the cell. Before chromosomal integration, all recoded segment plasmids contain an attP site for λ-integration. Since λ-integration modifies the attP sequence upon genome integration into attB site, only non-integrated plasmids carry intact attP sequence. Residual copies of the plasmid were eliminated using attP-specific Cas9-targeting (FIG. 10C) (Esvelt et al., 2013), such that SpCas9 protein induces double stranded breaks in all episomal (non-integrated) segment plasmids. Linearized remaining plasmids are then digested, and the resulting strains are plasmid-free.

Specifically, a plasmid containing the SpCas9 protein gene was constructed as well as a tracrRNA and a guide RNA directed towards the unmodified attP sequence (Plasmid details (DS-SPcas, Addgene plasmid 48645): cloDF13 origin, carb, proC promoter, SPcas9, tracrRNA (with native promoter and terminator), J23100 promoter, 1 repeat (added to facilitate cloning in a spacer onto the same plasmid). The guide RNA sequence cloned in the spacer is: TCAGCTTTTTTATACTAAGT (SEQ ID NO: 2330). Plasmid was transformed and cells were plated 3 hrs after transformation for growth at 37° C. under selection for SpCas9 plasmid (carbenicillin) (˜10⁷ cells). Resulting cells were PCR-verified for loss of all attP sequence. Presence of the integrated vector carrying recoded segment was confirmed by mAsPCR.

### Fitness Measurements

Strain doubling time was calculated as previously described (Lajoie et al., 2013b). Briefly, cultures were grown in flat-bottom 96-well plates (150 μL LBL, 34° C., 300 r.p.m.). Kinetic growth (OD600) was monitored on a Biotek Eon Microplate reader with orbital shaking at 365 cpm at 34° C. overnight and at 5-min intervals. Doubling times were calculated by t=Δt X ln(2)/m, where Δt=5 min per time point and m is the maximum slope of In(OD600) calculated by linear regression of a sliding window of 5 contiguous time points (20 min intervals). Analysis was performed using a Matlab® script.

The average change decrease in fitness observed for all 44 segments is 15% relative to the parental non-recoded strain fitness. 75% of segments (33 segments) were observed to have <20% decrease in fitness relative to wild-type, and only 4% of segments (2 segments) were observed to have more than 50% decrease in fitness (segments 21, 84), which may be referred to as “substantial decrease.”

### Investigation of Severe Fitness Impairment

A fitness impairing recoded gene was defined when deletion of the gene resulted in a reduced doubling time relative to the parent. This suggests the recoded gene was not well expressed. Impaired genes were located by gradually deleting each chromosomal gene using lambda Red recombineering and by measuring doubling times after each deletion (FIG. 12A-12B). Once located, a fitness impairing recoded gene is addressed using a troubleshooting pipeline.

First, the gene was Sanger-sequenced with allele-specific primers which prime only on the recoded, not the wild-type sequence. Sequencing results were analyzed to decide on one of two troubleshooting routes:

1) Sequencing revealed a mutation causing fitness impairment. Specifically, these refer to mutations that are not included in the computational genome design. Those mutations were fixed using MAGE.

2) No mutations were identified in the sequence compared to computational design. The fitness impairment of the recoded gene was assumed to originate in the recoded codons.

FIG. 12A-12B (segment 21) illustrates the troubleshooting strategy. Potential deleterious codons were identified in both the fitness impairing gene (fabH) and in the promoter of the entire operon (3 recoded codons located in upstream gene yceD). MAGE was performed (Wang et al., 2009) in a naïve strain (EcM2.1 (Gregg et al., 2014)) with oligos corresponding to the original recoded scheme to find fitness impairing codons. After 3 cycles of MAGE, cells were plated on permissive media (˜103 cells). 96 clones were screened with mascPCR primers targeting the wild-type sequence. The doubling time of clones having incorporated recoded codons was measured (˜20). No significant fitness impairment was observed for codons changed in gene fabH. Thus, the original design changes in the promoter were identified as the troublesome change. MAGE was performed in a naïve strain using degenerate MAGE oligos. After 3 cycles of MAGE, cells were plated on permissive media (10³ cells). An alternative recoded design without any forbidden codons was identified.

### Biocontainment Assay

The most effective biocontainment strategy involving recoded organisms (Mandell et al., 2015) uses 3 genes that are redesigned to accommodate a non-standard-amino-acid: the tyrosyl-tRNA-synthetase (tyrS), the adenylate kinase (adk) and the biphenylalanyl-tRNA syntethase (bipARS). Confirmation that those redesigned genes are compatible with the recoding strategy is critical for assaying the biocontainment potential of the recoded strain.

The bipARS gene does not contain any of the seven forbidden codons and thus considered compatible and can be integrated into the recoded strain. The gene adk, which contains only 1 forbidden codon and 2 additional adjustment mutations, was recoded and further validated in a bio-contained strain. The gene tyrS, which contains multiple forbidden codons, was recoded successfully in the current study, but the recoded tyrS was not yet tested in the biocontainment strategy.

Strains used in this study have the following background: All strains were based on EcNR2 (Escherichia coli MG1655 AmutS::cat Δ(ybhBbioAB)::[λc1857 N (cro-ea59)::tetR-bla]). Strains C321 [strain 48999 (www.addgene.org/48999)] and C321.ΔA [strain 48998 (www.addgene.org/48998)] are available from Addgene. C321.ΔA.adk_d6 and C321.ΔA.adk.d6_tyrS.d8_bipARS.d7 are based on (Mandell et al., 2015).

Using MAGE, the 3 codon changes in adk were included in the biocontained strain C321.ΔA.adk.d6 (escape rate around 10-6) and adk.d6_tyrS.d8_bipARS.d7 (most biocontained strain with escape rate <10-12). Fitness of the resulting strains (C321.ΔA.adk.d6.rc and C321.ΔA.adk.d6.rc_tyrS.d8_bipARS.d7) was evaluated as presented above. Escape frequencies were measured as previously described (Mandell et al., 2015). Briefly, all strains were grown in permissive conditions and harvested in late exponential phase. Cells were washed twice in LB^{L} and resuspended in LBL. Viable cfu was calculated from the mean and standard error of the mean (s.e.m.) of three technical replicates of tenfold serial dilutions on permissive media. Three technical replicates were plated on non-permissive media and monitored for 7 days (˜10⁷ cells). Two different non-permissive media conditions were used: SC, LB^{L} with SDS and chloramphenicol; and SCA, LB^{L} with SDS, chloramphenicol and 0.2% arabinose.

### DNA and RNA Sequencing Methods-Genome Sequencing

Bacterial genomic DNA was purified from 1 mL overnight cultures using the Illustra Bacteria GenomicPrep Spin Kit (General Electrics), and libraries were constructed using the Nextera DNA library Prep (Illumina), or the NebNext library prep (New England Biolabs). Libraries were sequenced using a MiSeq instrument (Illumina) with PE250 V2 kits (Illumina).

### SNP Calling

Two different pipelines were used to analyze genomes. Breseq (Deatherage, 2014) which supports haploid genome analysis, was used for SNP and short indels calling for strains with only one version of the segment (i.e. recoded or non-recoded wild-type). Breseq was used with default parameters.

### RNAseq Methods

RNA was prepared from strains carrying an episomal copy of the recoded segment and deletion of the chromosomal segment. RNA was stabilized using RNAprotect (QIAGEN), and extracted with miRNeasy kit (QIAGEN). rRNA content was reduced using riboZero rRNA Removal Kit (Illumina). RNAseq libraries were constructed using the Truseq Stranded mRNA Library Kit (Illumina). Libraries were sequenced using a MiSeq instrument (Illumina) with PE150 V2 kits (Illumina).

### RNAseg Analysis

FASTQ files obtained from RNAseq experiments were mapped using BWA (Li et al., 2009a) using default parameters, and processed (indexing, sorting) using SAMTOOLS (Li et al., 2009b) to generate a bam file for each sample. Custom R scripting was used to analyze the data. The library GenomicFeatures (Bioconductor) was used to associate reads to genes, and the Bioconductor library DESeq (Anders et al., 2010) was used to perform differential expression analysis. Genes with an absolute log 2 fold change higher than 2, and adjusted p-value smaller than 0.01 were classified as differentially expressed genes. Specifically, partially recoded strains and TOP10 control were individually analyzed by RNA-Seq. The expression of each gene was then compared using DESeq2 (Anders et al., 2010) in each sample (recoded or non recoded) to the expression of the same gene in every other sample (5 independent segments) to get a representative range of gene expression across all samples. For example, expression level for gene folC in segment 44 was measured in recoded segment 44 (only recoded copy), in TOP10 (only wild-type copy) and in all other partially recoded strains (where segment 44 is not recoded, e.g. only wild-type copy of gene folC).

### Example II

### Rules for Codon Choice-Editing Rare Arginine Codons in E. coli

According to some aspects, methods are described herein for empirical validation and updating of rules or constraints or conditions or parameters or features for genome design. In particular, the rare arginine codons AGA and AGG (AGR) present a case study in codon choice, with AGRs encoding important transcriptional and translational properties distinct from the other synonymous alternatives (CGN). A strain of Escherichia coli has been created in which all 123 instances of AGR codons have been removed from all essential genes. 110 AGR codons were replaced with the synonymous CGU, whereas the remaining 13 AGRs necessitated diversification to identify viable alternatives. Successful replacement codons tended to conserve local ribosomal binding site-like motifs and local mRNA secondary structure, sometimes at the expense of amino acid identity. Based on these observations, metrics were empirically defined for a multi-dimensional ‘safe replacement zone’ (SRZ) within which alternative codons may be more likely to be viable. To further evaluate synonymous and non-synonymous alternatives to essential AGRs, a CRISPR/Cas9-based method was implemented to deplete a diversified population of a wild type allele, in which the method allowed for a comprehensive evaluation of the fitness impact of all 64 codon alternatives. Using this method, relevance of the SRZ was confirmed by tracking codon fitness over time in 14 different genes. It was found that codons that fall outside the SRZ may be rapidly depleted from a growing population.

Ultimately, the genetic code possesses inherent redundancy (Crick, 1963), with up to six different codons specifying a single amino acid. This implies that synonymous codons are equivalent (Kimura, 1977), however most prokaryotes and many eukaryotes (dos Reis et al., 2004; Newton and Wernisch, 2014) display a strong preference for certain codons over synonymous alternatives (Hershberg and Petrov, 2008; Plotkin and Kudla, 2011). While different species have evolved to prefer different codons, codon bias is largely consistent within each species (Hershberg and Petrov, 2008). However, within a given genome, codon bias differs among individual genes according to codon position, suggesting that codon choice has functional consequences. For example, rare codons are enriched at the beginning of essential genes (Chen and Inouye, 1990; Chen and Inouye, 1994), and codon usage strongly affects protein levels (Kane, 1995; Sharp and Li, 1987; Sharp et al., 1993), especially at the N-terminus (Goodman et al., 2013). This suggests that codon usage plays a poorly understood role in regulating protein expression.

Several hypotheses attempt to explain how codon usage mediates this effect, including but not limited to: facilitating ribosomal pausing early in translation to optimize protein folding (Zhou et al., 2013), adjusting mRNA secondary structure to optimize translation initiation or modulate mRNA degradation, preventing ribosome stalling by co-evolving with tRNAs levels (Plotkin and Kudla, 2011), providing a “translational ramp” for proper ribosome spacing and effective translation (Tuller et al., 2010), or providing a layer of translational regulation for independent control of each gene in an operon (Li, 2015). Additionally, codon usage may impact translational fidelity (Hooper and Berg, 2000), and the proteome may be tuned by fine control of the decoding tRNA pools (Gingold et al., 2014). Although Quax et al. provides an excellent review of how biology chooses codons, systematic and exhaustive studies of codon choice in whole genomes are lacking (Quax et al., 2015). Studies have only begun to probe the effects of codon choice in a relatively small number of genes (Goodman et al., 2013; Isaacs et al., 2011; Kudla et al., 2009; Lajoie et al., 2013a; Li et al., 2012). Furthermore, although the UAG stop codon has been completely removed from Escherichia coli (Lajoie 2013a), and the AGG codon has been ambiguously reassigned (Lee et al., 2015; Mukai et al., 2015; Zeng et al., 2014), no genomewide attempt to entirely replace a sense codon has been reported. Prior work has established there are unknown constraints to such replacement (Isaacs et al., 2011; Lajoie et al., 2013a; Lajoie et al., 2013b). Attempting to replace all essential instances of a codon in a single strain would provide valuable insight into these constraints. Additionally, while some constraints are known to exist in certain genes, no attempt has been made to explore the breakdown of synonymous codons on a genome wide scale.

As described in the Example herein, rare arginine codons AGA and AGG (comprising AGR according to IUPAC conventions) were chosen for this study because the literature suggests that they are among the most difficult codons to replace and that their similarity to ribosome binding sequences underlies important non-coding functions (Chen and Inouye, 1990, Rosenberg et al., 1993, Spanjaard et al., 1988, Spanjaard et al., 1990, Bonekamp et al., 1985. Furthermore, their sparse usage (123 instances in the essential genes of E. coli MG1655 and 4228 instances in the entire genome (Table 3) made replacing all AGR instances in essential genes a tractable goal, with essential genes serving as a stringent test set for identifying any fitness impact from codon replacement (Baba, et al., 2006). Additionally, recent work has shown the difficulty of directly mutating some AGR codons to other synonymous codons (Zeng, et al, 2014), although the authors do not explain the mechanism of failure or report successful implementation of alternative designs. All 123 instances of AGR codons were attempted to be removed from essential genes by replacing them with the synonymous CGU codon. CGU was chosen to maximally disrupt the primary nucleic acid sequence (AGR-> CGU). It was hypothesized that this strategy would maximize design flaws, thereby revealing rules for designing genomes with reassigned genetic codes. Importantly, individual codon target were not inspected a priori in order to ensure an unbiased empirical search for design flaws.

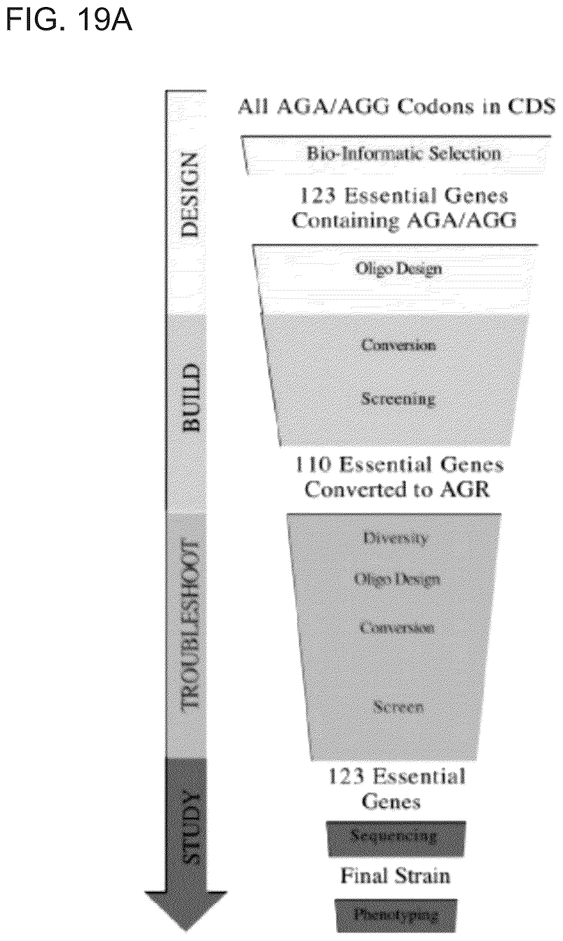

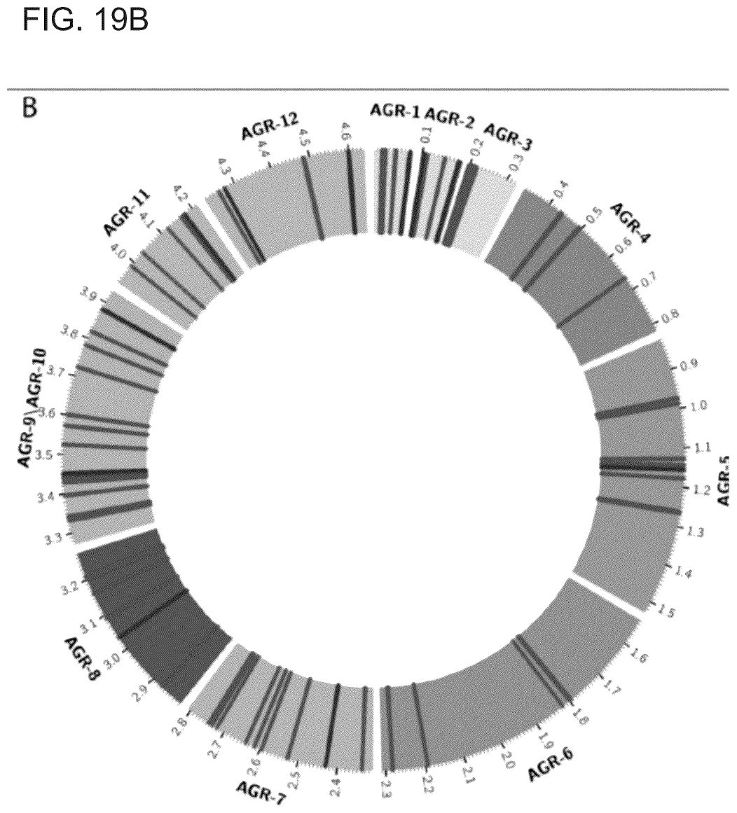

To construct this modified genome, co-selection multiplex automatable genome engineering (CoS-MAGE) was used (Carr et al., 2012, Gregg et al., 2014) to create an E. coli strain (C123) with all 123 AGR codons removed from its essential genes (FIG. 19A). CoS-MAGE leverages lambda red-mediated recombination (Yu et al., 2000, Ellis et al., 2001) and exploits the linkage between a mutation in a selectable allele (e.g. tolC) to nearby edits of interest (e.g., AGR conversions), thereby enriching for cells with those edits (Figure S1). To streamline C123 construction, E. coli strain EcM2.1 was chosen to start with, in which the strain was previously optimized for efficient lambda red-mediated genome engineering (Gregg et al., 2014, Lajoie et al., 2012). Using CoS-MAGE on EcM2.1 improves allele replacement frequency by 10-fold over MAGE in non-optimized strains but performs optimally when all edits are on the same replichore and within 500 kilobases of the selectable allele (Gregg et al., 2014). To accommodate this requirement, the genome was divided into 12 segments containing all 123 AGR codons in essential genes. A tolC cassette was moved around the genome to enable CoS-MAGE in each segment, allowing us to rapidly prototype each set of AGR-> CGU mutations across large cell populations in vivo. Of the 123 AGR codons in essential genes, 110 could be changed to CGU by this process (FIG. 1), revealing considerable flexibility of codon usage for most essential genes. Allele replacement (in this case, AGR-> CGU codon substitution) frequency varied widely across these 110 permissive codons, with no clear correlation between allele replacement frequency and normalized position of the AGR codon in a gene (FIG. 2A).

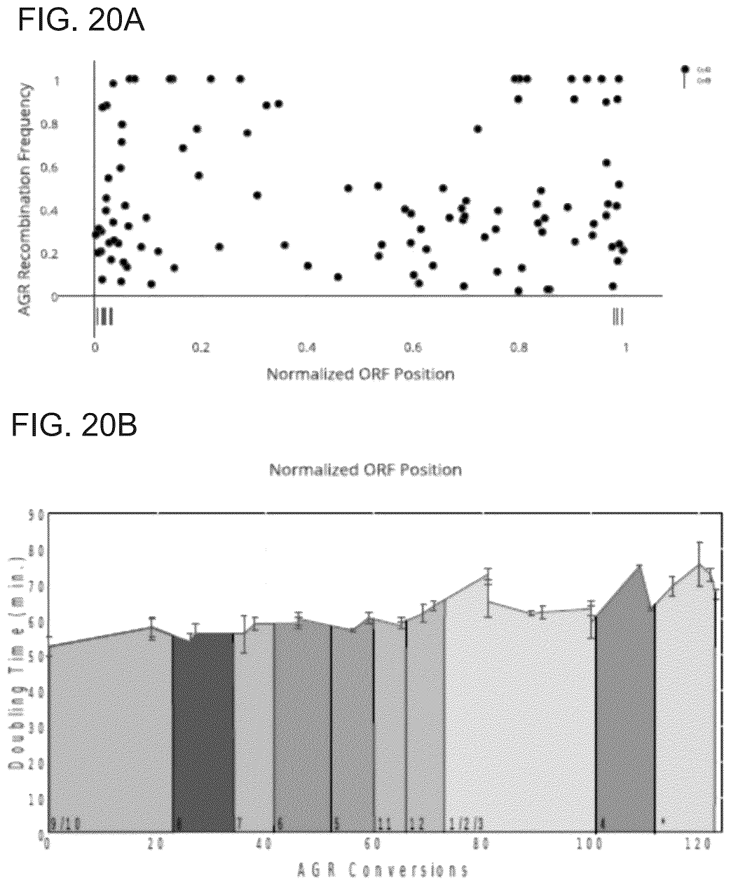

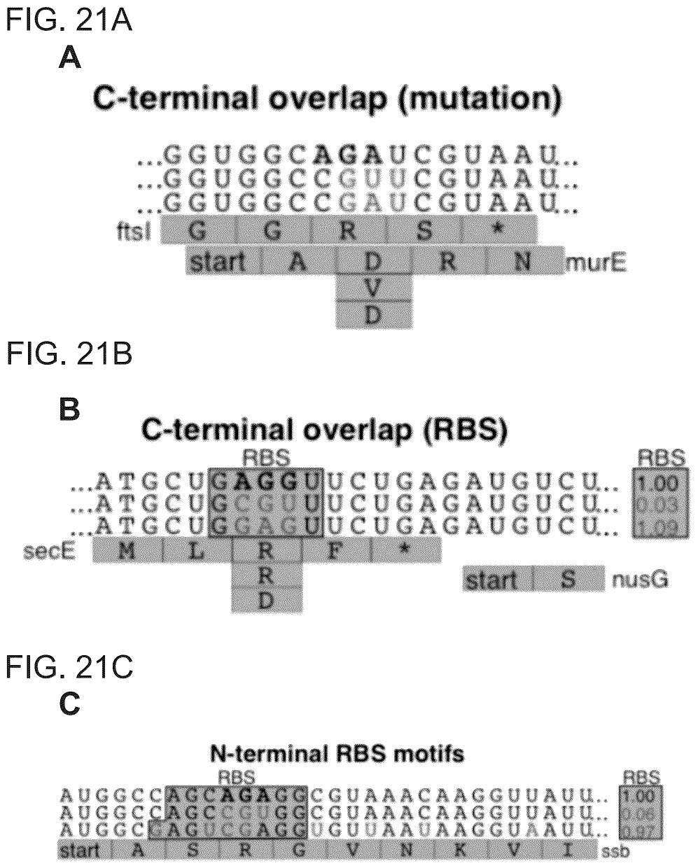

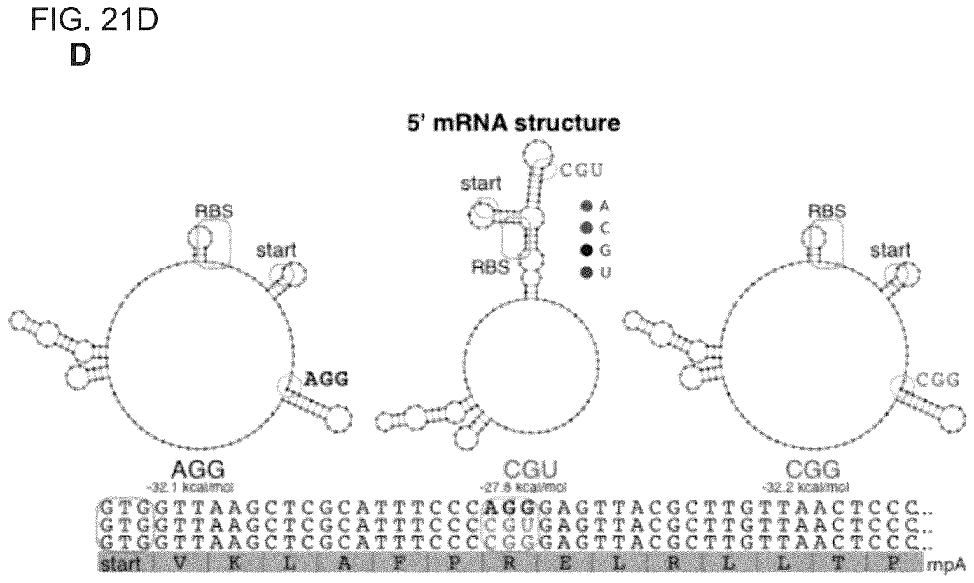

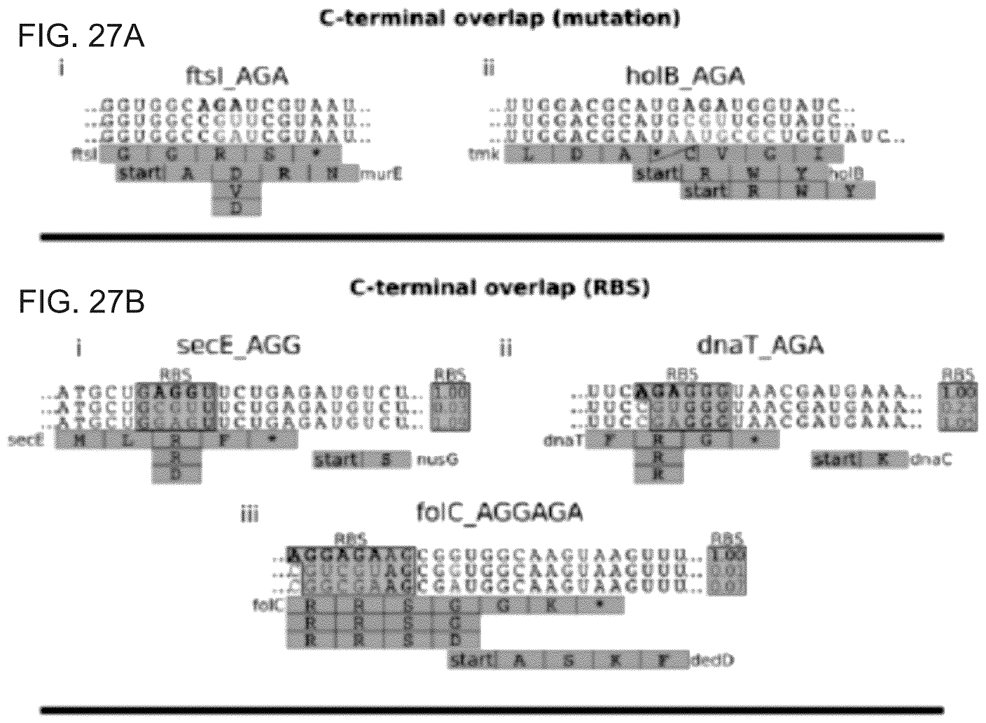

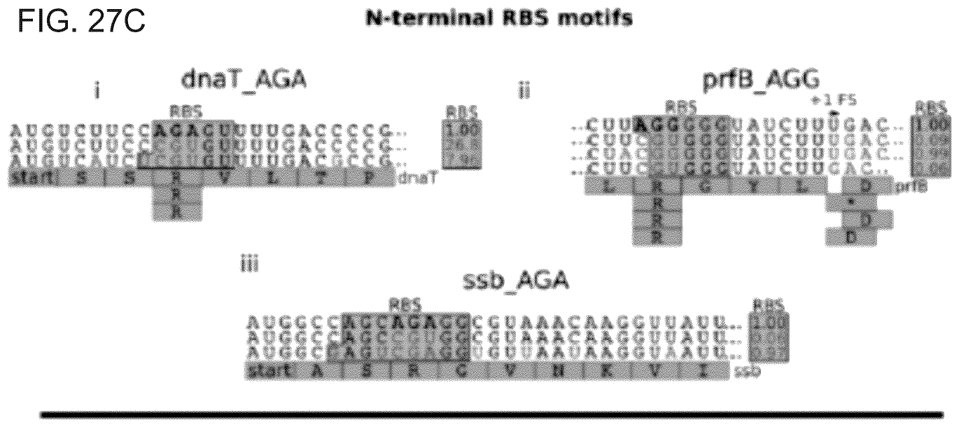

The remaining 13 AGR-> CGU mutations were not observed, suggesting a codon substitution frequency of less than the detection limit of 1% of the bacterial population. These ‘recalcitrant codons’ were assumed to be deleterious or non-recombinogenic and were triaged into a troubleshooting pipeline for further analysis (FIG. 19A-B). Interestingly, all except for one of the thirteen recalcitrant codons were co-localized near the termini of their respective genes, suggesting the importance of codon choice at these positions-seven were at most 30 nt downstream of the start codon, while five were at most 30 nucleotides (nt) upstream of the stop codon (FIG. 20A, lower panel). These failed AGR-> CGU mutations were inspected for obvious design errors. For example, ftsI_AGA1759 overlaps the second and third codons of murE, an essential gene, introducing a missense mutation (murE D3V) that may impair fitness. Replacing ftsI_AGA with CGA successfully replaced the forbidden AGA codon while conserving the primary amino acid sequence of MurE with a minimal impact on fitness (FIG. 21A). Similarly, holB_AGA4 overlaps the upstream essential gene tmk, and replacing AGA with CGU converts the tmk stop codon to Cys, adding 14 amino acids to the Cterminus of tmk. While some C-terminal extensions are well-tolerated in E. coli (Ohtake et al., 2012), extending tmk appears to be deleterious. holB_AGA was successfully with CGC by inserting three nucleotides comprising a stop codon before the holB start codon. This reduced the tmk/holB overlap, and preserved the coding sequences of both genes (FIG. 27A).

Subtler overlap errors were identified for the four remaining C-terminal failures, where it was determined that AGR-> CGU mutations disrupt RBS motifs belonging to downstream genes (secE_AGG376 for nusG, dnaT_AGA532 for dnaC, and folC_AGAAGG1249, 1252 for dedD, the latter constituting two codons). Both nusG and dnaC are essential, suggesting that replacing AGR with CGU in secE and dnaT lethally disrupts translation initiation and thus expression of the overlapping nusG and dnaC (FIG. 21B and FIG. 27B). Although dedD is annotated as non-essential (Baba, et al., 2006), it was hypothesized that replacing the AGR with CGU in folC disrupted a portion of dedD that is essential to the survival of EcM2.1 (E. coli K-12). In support of this hypothesis, the 29 nucleotides of dedD that were not deleted by Baba et al. (Baba, et al., 2006) were not deleted and did not overlap with folC, suggesting that this sequence is essential in the strains described. The unexpected failure of this conversion highlights the challenge of predicting design flaws even in well-annotated organisms. Consistent with the observation that disrupting these RBS motifs underlies the failed AGR-> CGU conversions, all three design flaws were overcome by selecting codons that conserved RBS strength, including a non-synonymous (Arg->Gly) conversion for secE.

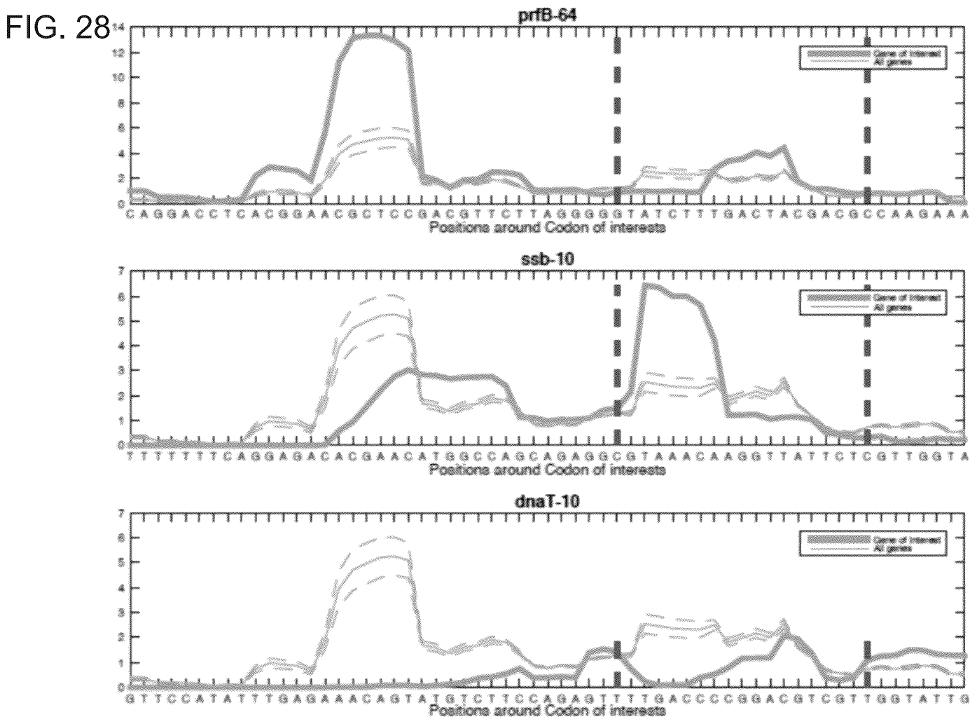

These lessons, together with previous observations that ribosomes pause during translation when they encounter ribosome binding site motifs in coding DNA sequences (Li et al., 2012), provided key insights into the N-terminal AGR-> CGU failures. As described herein, RBS-like motifs may refer to both RBS motifs (which may typically occur before a start codon) and similar motifs (which may occur in the open reading frame but do not necessarily cause translation initiation). Three of the N-terminal failures (ssb_AGA10, dnaT_AGA10 and prfB_AGG64) had RBS-like motifs either disrupted or created by CGU replacement. While prfB_AGG64 is part of the ribosomal binding site motif that triggers an essential frameshift mutation in prfB (Lajoie et al., 2013a, Craigen et al., 1985, Curran et al., 1993), pausing-motif-mediated regulation of ssb and dnaT expression has not been reported. Nevertheless, ribosomal pausing data (Li et al., 2012) showed that ribosomal occupancy peaks are present directly downstream of the AGR codons for ssb and absent for dnaT (FIG. 28); meanwhile, unsuccessful CGU mutations were predicted to weaken the RBS-like motif for prfB and ssb and strengthen the RBS-like motif for dnaT (FIG. 21C and FIG. 27C), suggesting a functional relationship between RBS occupancy and cell fitness.

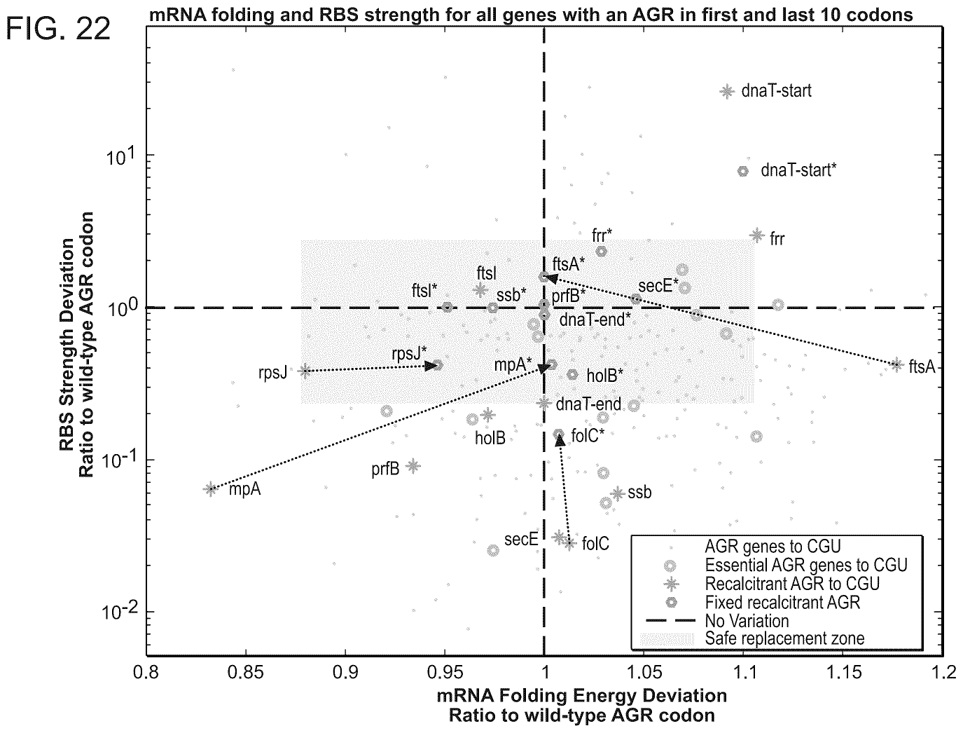

Consistent with this hypothesis, successful codon replacements from the troubleshooting pipeline conserve predicted RBS strength compared to the large predicted deviation caused by unsuccessful AGR-> CGU mutations (FIG. 22, y axis and comparison between orange asterisks and green dots). Interestingly, attempts to replace dnaT_AGA10 with either CGN or NNN failed-only by manipulating the wobble position of surrounding codons and conserving the arginine amino acid could dnaT_AGA10 be replaced (FIG. 27C). These wobble variants appear to compensate for the increased RBS strength caused by the AGA-> CGU mutation-RBS motif strength with wobble variants deviated 8-fold from the unmodified sequence, whereas RBS motif strength for AGA-> CGU alone deviated 27-fold.

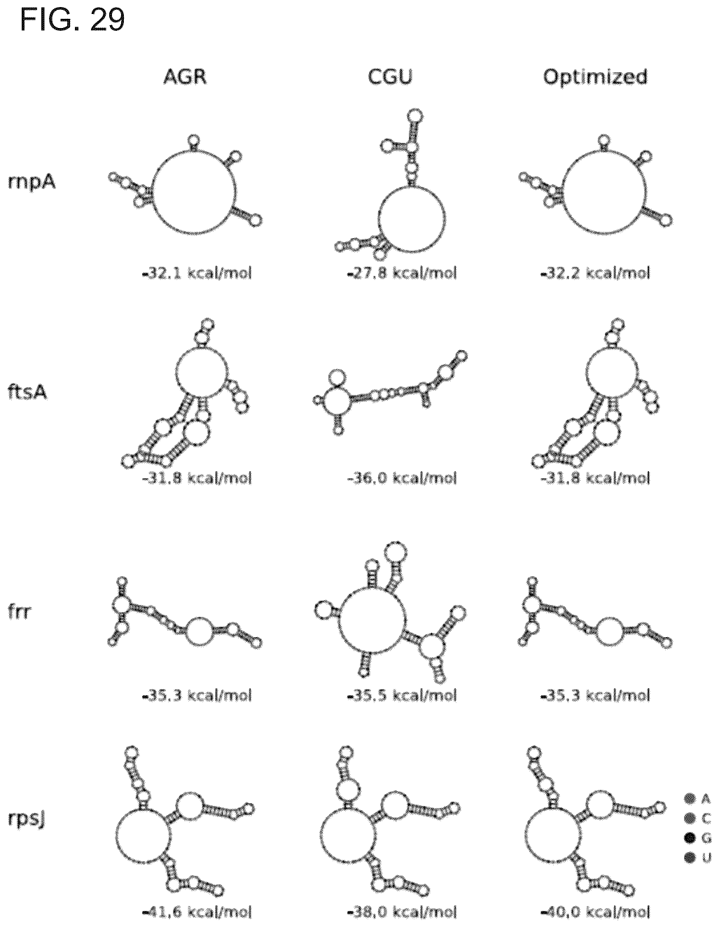

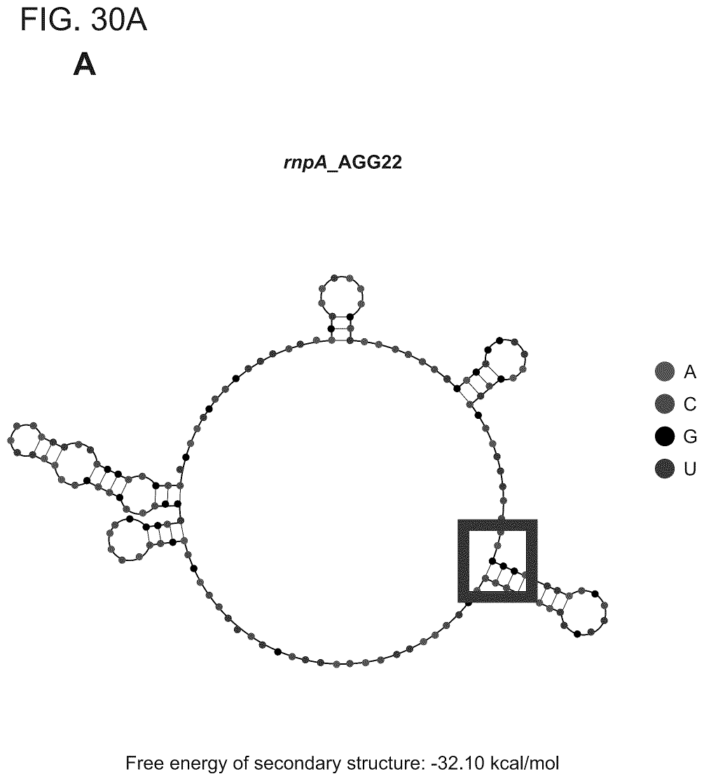

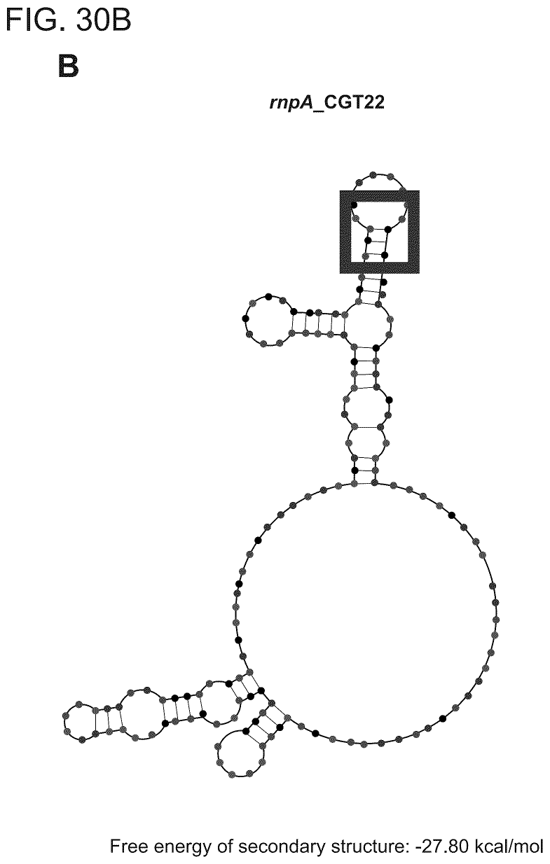

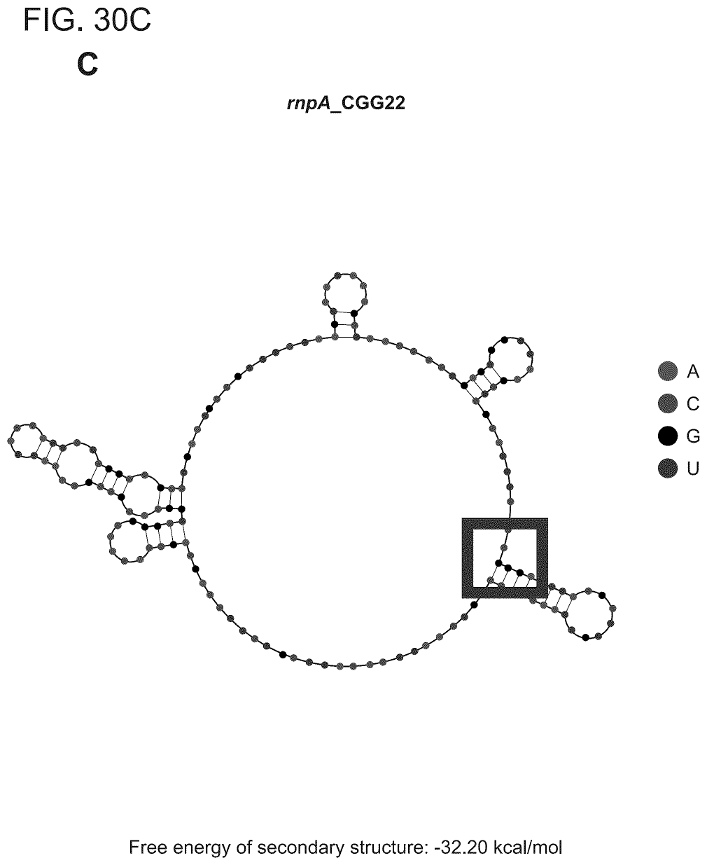

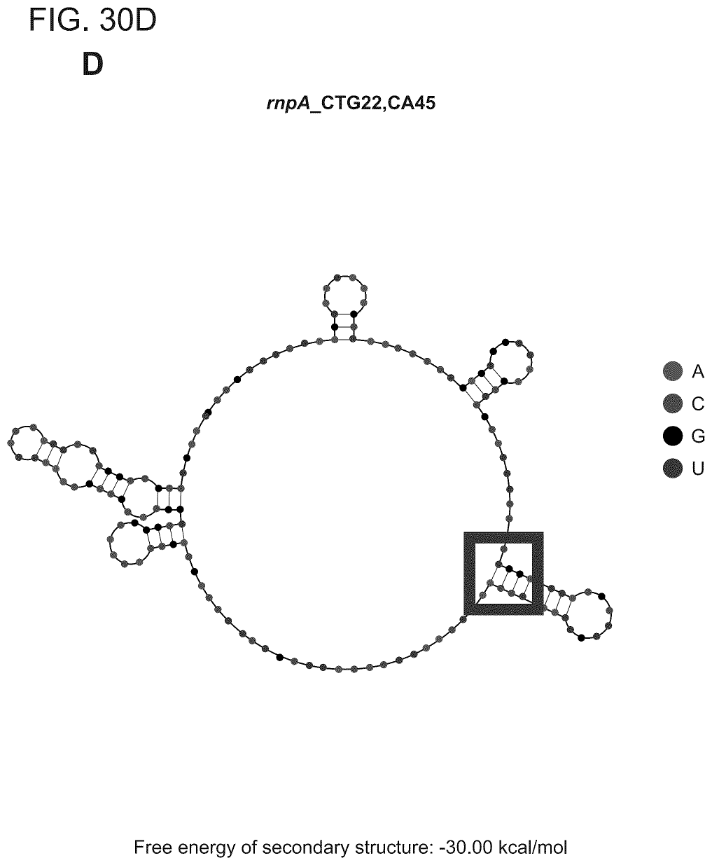

In order to better understand several remaining N-terminal failure cases that did not exhibit considerable RBS strength deviations (rnpA_AGG22, ftsA_AGA19, frr_AGA16, and rpsJ_AGA298), other potential nucleic acid determinants of protein expression were examined. Based on the observation that mRNA secondary structure near 5′ end of Open Reading Frames (ORFs) strongly impacts protein expression (Goodman et al., 2013), it was found that AGR-> CGU mutations often changed the predicted folding energy and structure of the mRNA near the start codon of target genes (FIG. 21D and FIG. 29). Successful codon replacements obtained from degenerate MAGE oligos reduced the disruption of mRNA secondary structure compared to CGU (FIG. 22, green dots). For example, rnpA has a predicted mRNA loop near its RBS and start codon that relies on base pairing between both guanines of the AGG codon to nearby cytosines (FIG. 21D, FIG. 30A). Importantly, only AGG22CGG was observed out of all attempted rnpA AGG22CGN mutations, and the fact that only CGG preserves this mRNA structure suggests that it is physiologically important (FIG. 21D, FIG. 30B-30C). In support of this, a rnpA AGG22CUG mutation (Arg->Leu) was successfully introduced only when the complementary nucleotides in the stem were changed from CC (base pairs with AGG) to CA (base pairs with CUG), thus preserving the natural RNA structure (FIG. 30D) while changing both RBS motif strength and amino-acid identity.

The analysis of all four optimized gene sequences showed reduced deviation in computational mRNA folding energy (computed with UNAFold (Markham et al., 2008)) compared to the unsuccessful CGU mutations (FIG. 22, x-axis orange asterisks and green dots). Similarly, predicted mRNA structure (computed with a different mRNA folding software: NUPACK (Zadch et al., 2011)) for these genes was strongly changed by CGU mutations and corrected in the empirically optimized solutions (FIG. 29).

Troubleshooting these 13 recalcitrant codons revealed that mutations causing large deviations from natural mRNA folding energy or RBS strength are associated with failed codon substitutions. By calculating these two metrics for all attempted AG-> CGU mutations, a safe replacement zone (SRZ) was empirically defined inside which most CGU mutations were tolerated (FIG. 22, shaded area). The SRZ is defined as the largest multi-dimensional space which contains none of the mRNA folding energy or RBS strength associated recalcitrant AGR-> CGU mutations (FIG. 22, red asterisks). It comprises deviations in mRNA folding energy of less than 10% with respect to the natural codon and deviations in RBS-like motif scores of less than a half log with respect to the natural codon, providing a quantitative guideline for codon substitution. Notably, the optimized solution used to replace the 13 recalcitrant codons always exhibited reduced deviation for at least one of these two parameters than the deviation seen with mutation to CGU. Furthermore, solutions to the 13 recalcitrant codons overlapped almost entirely with the empirically-defined SRZ. These results suggest that computational predictions of mRNA folding energy and RBS strength can be used as a first approximation to predict whether a designed mutation is likely to be lethal. By developing in silico heuristics to predict problematic alleles in turn reduces the search space required for in vivo genome engineering, making it possible to create radically altered genomes that remain viable.

Once viable replacement sequences were identified for all 13 recalcitrant codons, the successful 110 CGU conversions were combined with the 13 optimized codon substitutions to produce strain C123, which has all 123 AGR codons removed from all of its annotated essential genes. C123 was then sequenced to confirm AGR removal and analyzed using Millstone, a publicly available genome resequencing analysis pipeline (Goodman et al., 2015). Two spontaneous AAG (Lys) to AGG (Arg) mutations were observed in the essential genes pssA and cca. While attempts to revert these mutations to AAG were unsuccessful-perhaps suggesting functional compensation-they were replaced with CCG (Pro) in pssA and CAG (Gln) in cca using degenerate MAGE oligos. The resulting strain, C123a, is the first strain completely devoid of AGR codons in its annotated essential gene. This strain provides strong evidence that AGR codons can be completely removed from the E. coli genome, permitting the unambiguous reassignment of AGR translation function.

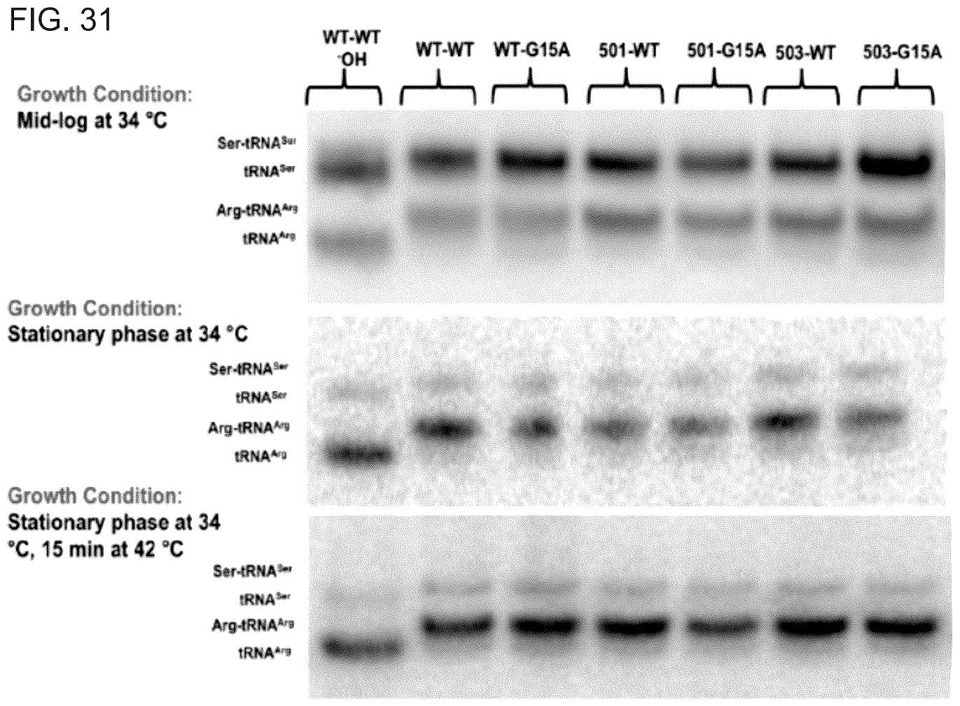

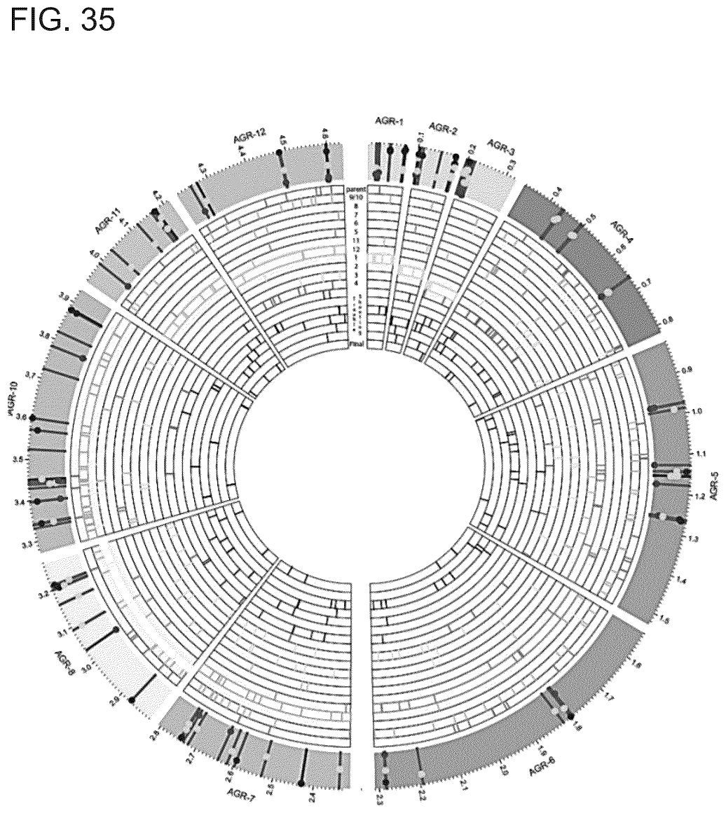

Kinetic growth analysis showed that the doubling time increased from 52.4 (+/−2 .6) minutes in EcM2.1 (0 AGR codons changed) to 67 (+/−1.5) minutes in C123a (123 AGR codons changed in essential genes) in lysogeny broth (LB) at 34° C. in a 96-well plate reader. Notably, fitness varied significantly during C123 strain construction (FIG. 20B). This may be attributed to codon deoptimization (AGR-> CGU) and compensatory spontaneous mutations to alleviate fitness defects in a mismatch repair deficient (mutS-) background. Overall the reduced fitness of C123a may be caused by on-target (AGR-> CGU) or off-target (spontaneous mutations) that occurred during strain construction. In this way, mutS inactivation is simultaneously a useful evolutionary tool and a liability. Final genome sequence analysis revealed that along with the 123 desired AGR conversions, C123a had 419 spontaneous non-synonymous mutations not found in the EcM2.1 parental strain (FIG. 35). Of particular interest was the mutation argU_G15A, located in the D arm of tRNA^{Arg} (argU), which arose during CoS-MAGE with AGR set 4. It was hypothesized that argU_G15A compensates for increased CGU demand and decreased AGR demand, but no direct fitness cost associated with reverting this mutation in C123 was observed, and argU_G15A does not impact aminoacylation efficiency in vitro or aminoacyl-tRNA pools in vivo (FIG. 31). Consistent with Mukai et al. and Baba et al. (Mukai et al., 2015, Baba, et al., 2006), argW (tRNA^{Arg}CCU; decodes AGG ArgUCU; only) was dispensable in C123a because it can be complemented by argU (tRNA^{Arg}UCU; decodes both AGG and AGA). However, argU is the only E. coli tRNA that can decode AGA and remains essential in C123a probably because it is required to translate the AGR codons for the rest of the proteome (Lajoie et al., 2013b).

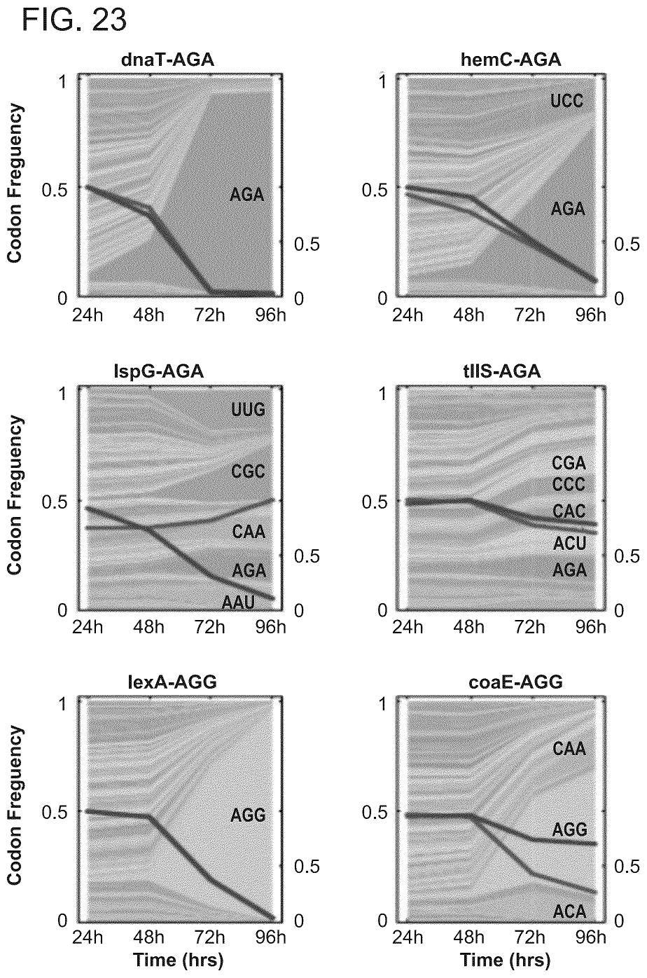

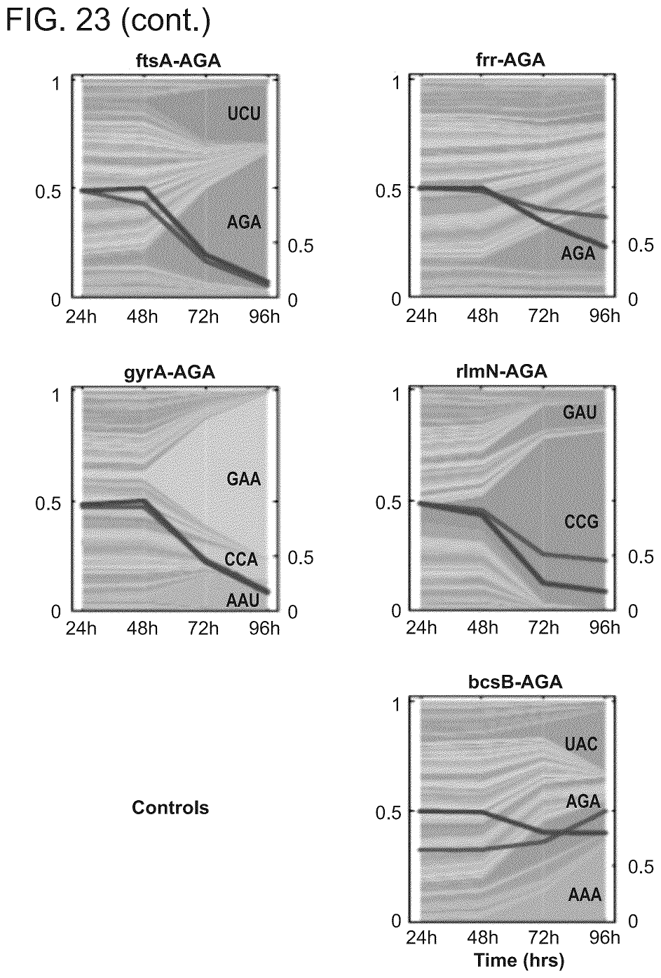

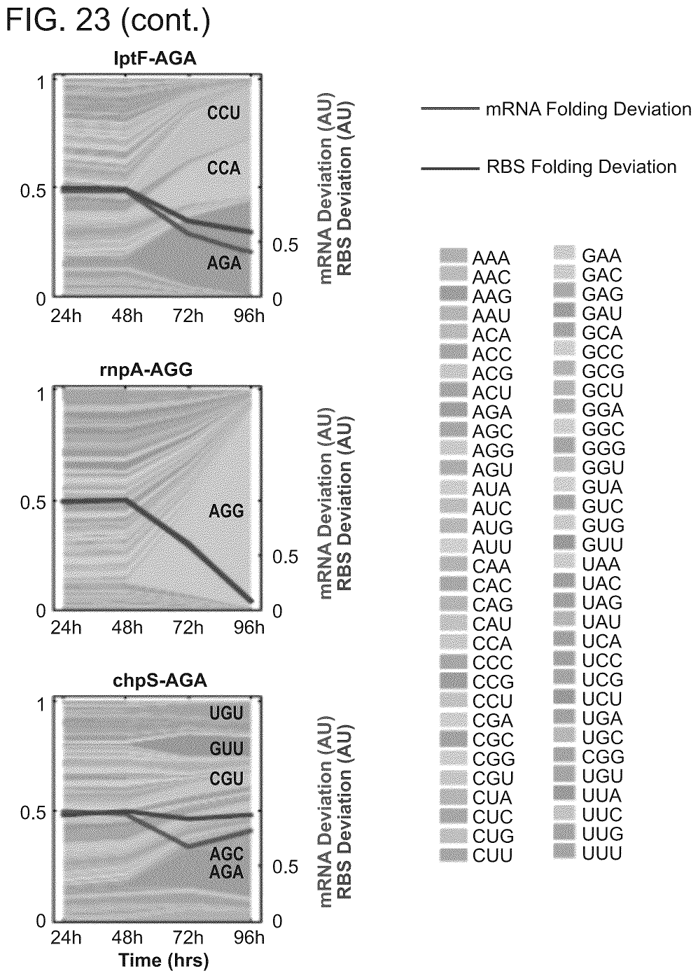

To evaluate the genetic stability of C123a after removal of all AGR codons from all the known essential genes, C123a was for passaged 78 days (640 generations) to test whether AGR codons would recur and/or whether spontaneous mutations would improve fitness. After 78 days, no additional AGR codons were detectable in a sequenced population, and doubling time of isolated clones ranged from 22% faster to 22% slower than C123a (n=60). To gain more insight into how local RBS strength and mRNA folding impact codon choice, an evolution experiment was performed to examine the competitive fitness of all 64 possible codon substitutions at each of AGR codons. While MAGE is a powerful method to explore viable genomic modifications in vivo, it was of interest to map the fitness cost associated with less-optimal codon choices, requiring codon randomization depleted of the parental genotype, which was hypothesized to be at or near the global fitness maximum. To do this, a method called CRAM (Crispr-Assisted-MAGE) was developed. First, oligos were designed that changed not only the target AGR codon to NNN, but also made several synonymous changes at least 50 nt downstream that would disrupt a 20 bp CRISPR target locus. MAGE was used to replace each AGR with NNN in parallel, and CRISPR/cas9 was used to deplete the population of cells with the parental genotype. This approach allowed exhaustive exploration of the codon space, including the original codon, but absent the preponderance of the parental genotype. Following CRAM, the population was passaged 1:100 every 24 hours for six days, and sampled prior to each passage using Illumina sequencing (FIG. 23).

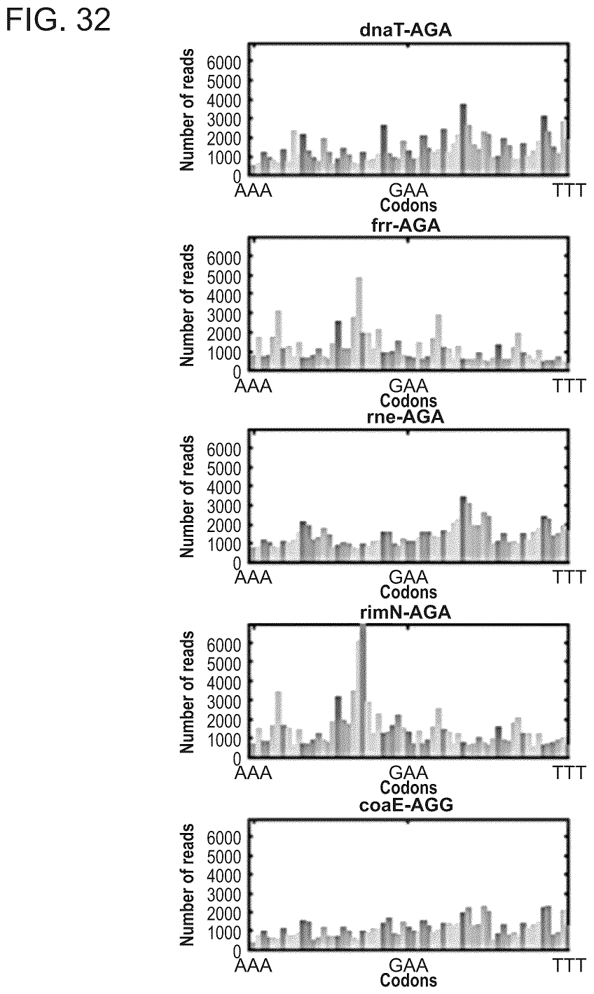

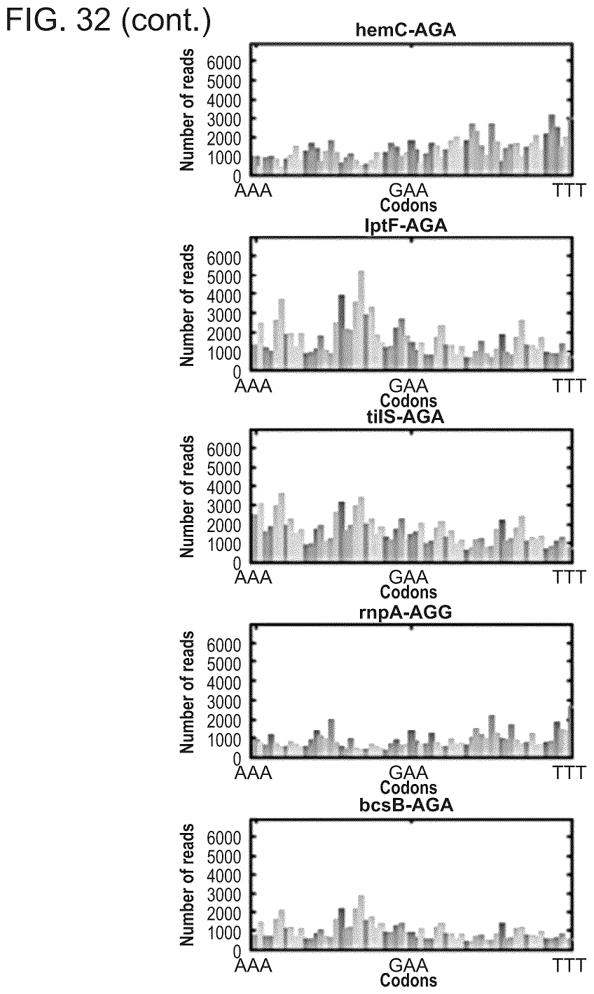

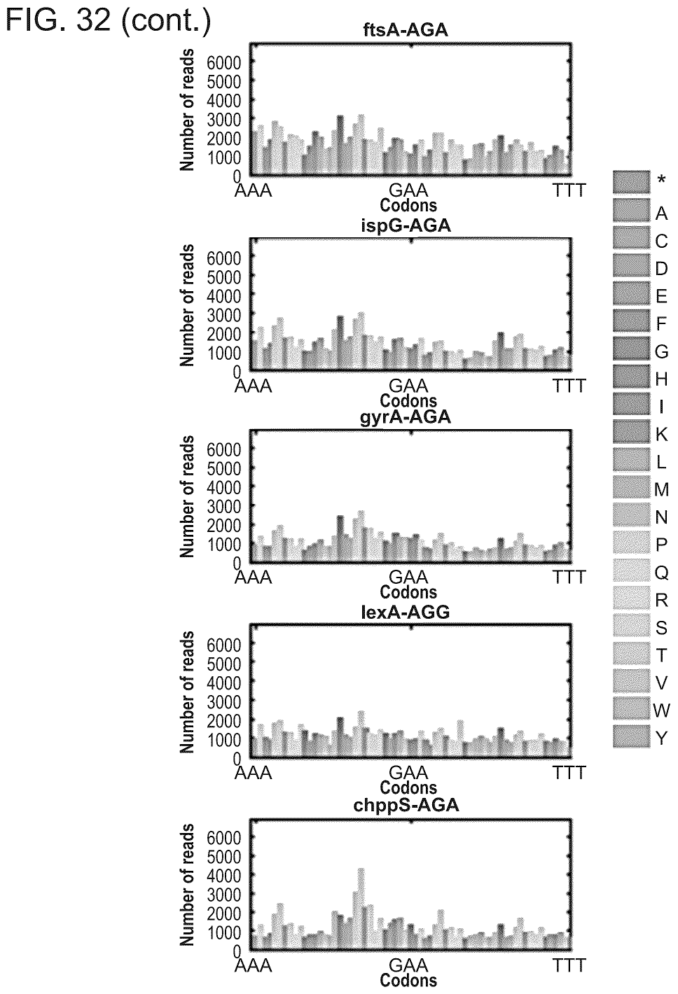

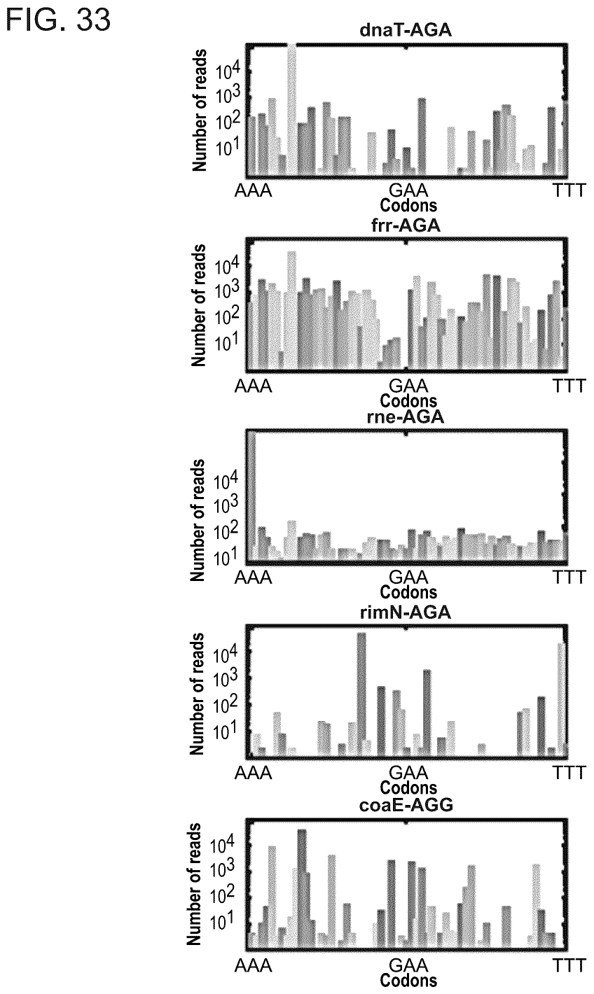

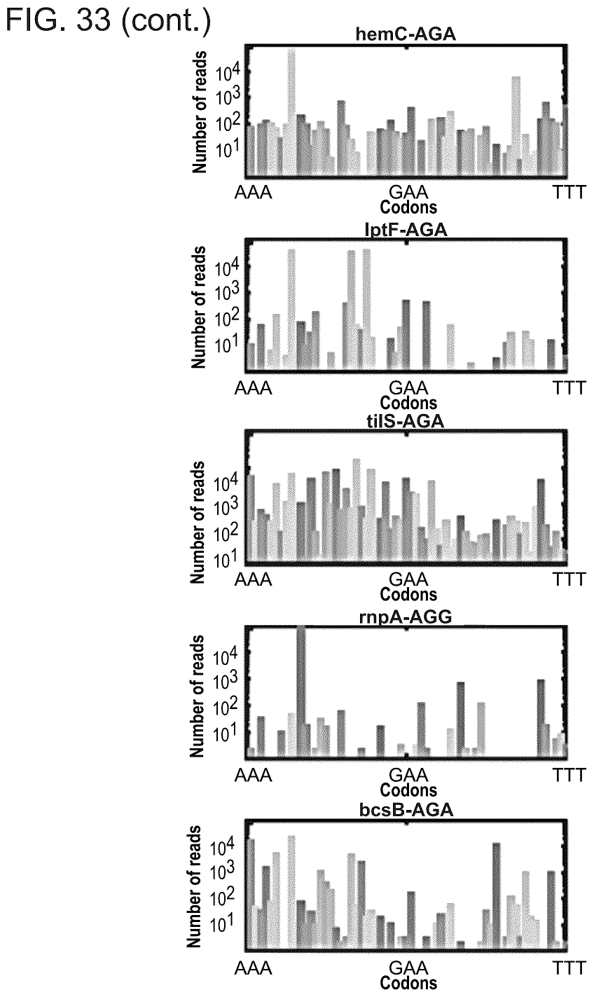

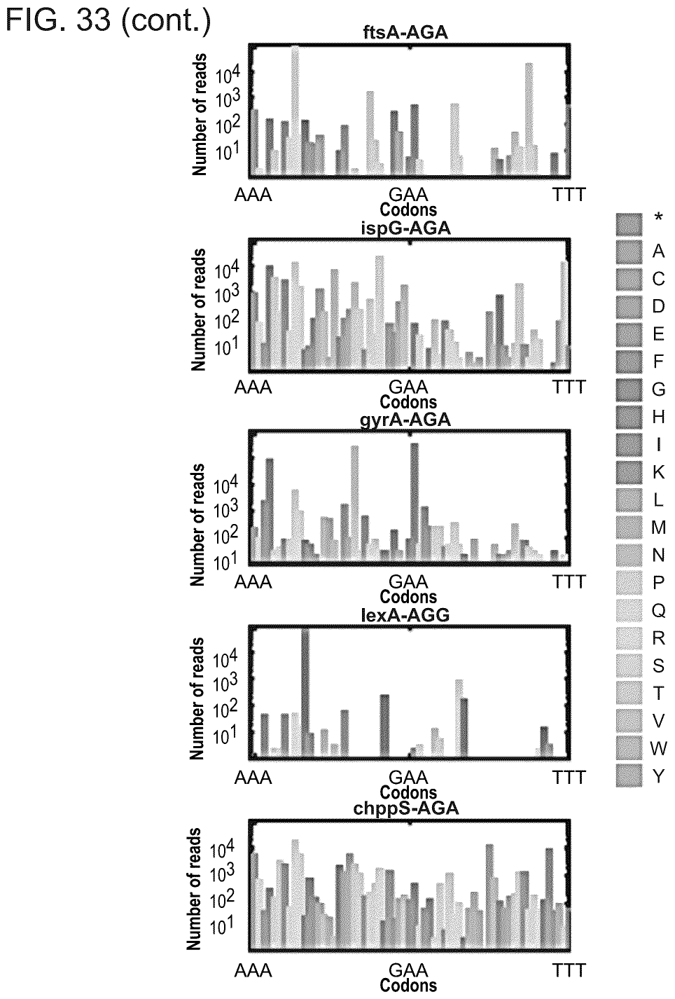

Sequencing 24 hours after CRAM showed that all codons were present (including stop codons) (FIG. 32), validating the method as a technique to generate massive diversity in a population. All sequences for further analysis were amplified by PCR with allele-specific primers containing the changed downstream sequence. Subsequent passaging of these populations revealed many gene-specific trends (FIG. 23, FIG. 33, FIG. 33). Notably, all codons that required troubleshooting (dnaT_AGA10, ftsA_AGA19, frr_AGA16, rnpA_AGG22) converged to their wild-type AGR codon, suggesting that the original codon was globally optimized. For all cases where an alternate codon replaced the original AGR, the predicted deviation in mRNA folding energy and local RBS strength (as a proxy for ribosome pausing) was computed for these alternative codons and compared these metrics to the evolution of codon distribution at this position over time. The fraction of sequences that fall within the SRZ inferred was also computed from FIG. 22. CRAM initially introduced a large diversity of mRNA folding energies and RBS strengths, but these genotypes rapidly converged toward parameters that are similar to the parental AGR values in many cases (FIG. 23, overlays). Codons that strongly disrupted predicted mRNA folding and internal RBS strength near the start of genes were disfavored after several days of growth, suggesting that these metrics can be used to predict optimal codon substitutions in silico. In contrast, non-essential control genes bcsB and chpS did not converge toward codons that conserved RNA structure or RBS strength, supporting the conclusion that the observed conservation in RNA secondary structure and RBS strength is biologically relevant for essential genes. Interestingly, tilS_AGA19 was less sensitive to this effect, suggesting that codon choice at that particular position is not under selection. Additionally, the average internal RBS strength for the ipsG populations converged towards the parental AGR values whereas mRNA folding energy averages did not, suggesting that this position in the gene may be more sensitive to RBS disruption rather than mRNA folding. Gene IptF followed the opposite trend.

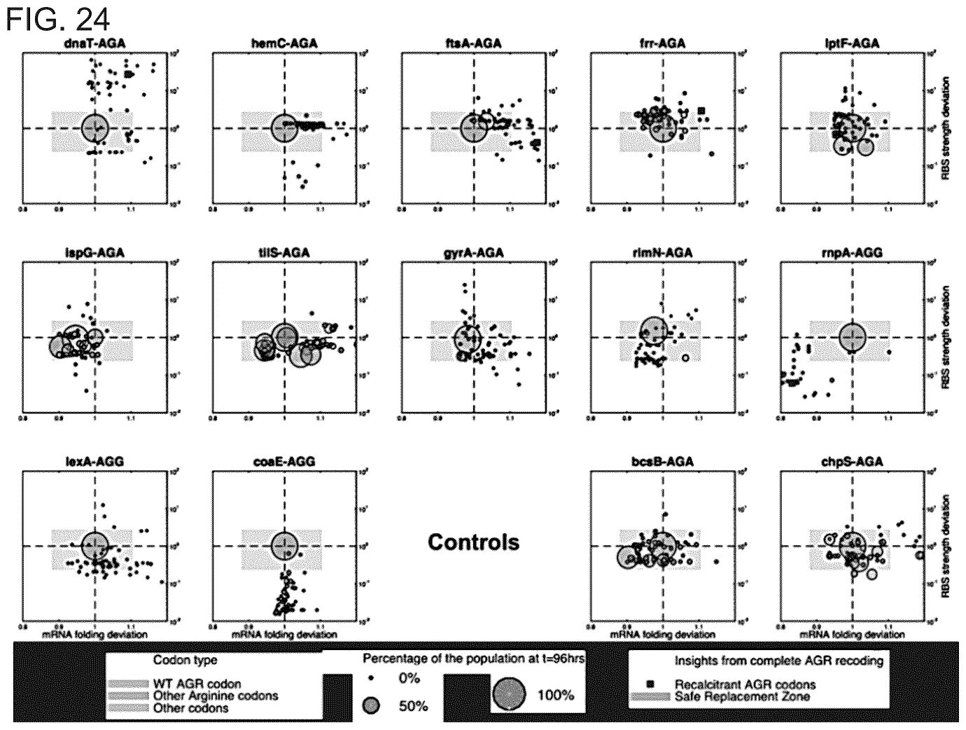

Interestingly, several genes (IptF, ipsG, tilS, gyrA and rimN) preferred codons that changed the amino acid identity from Arg to Pro, Lys, or Glu, suggesting that non-coding functions trump amino acid identity at these positions. Importantly, all successful codon substitutions in essential genes fell within the SRZ (FIG. 24), validating the heuristics based on an unbiased test of all 64 codons. Meanwhile non-essential control gene chpS exhibited less dependence on the SRZ. Based on these observations, while global codon bias may be affected by tRNA availability (Plotkin et al., 2011, Novoa et al., 2012, Ikemura, 1985), codon choice at a given position may be defined by at least 3 parameters: (1) amino acid sequence, (2) mRNA structure near the start codon and RBS (3) RBS-mediated pausing. In some cases, a subset of these parameters may not be under selection, resulting in an evolved sequence that only converges for a subset of the metrics. In other cases, all metrics may be important, but the primary nucleic acid sequence might not have the flexibility to accommodate all of them equally, resulting in codon substitutions that impair cellular fitness.

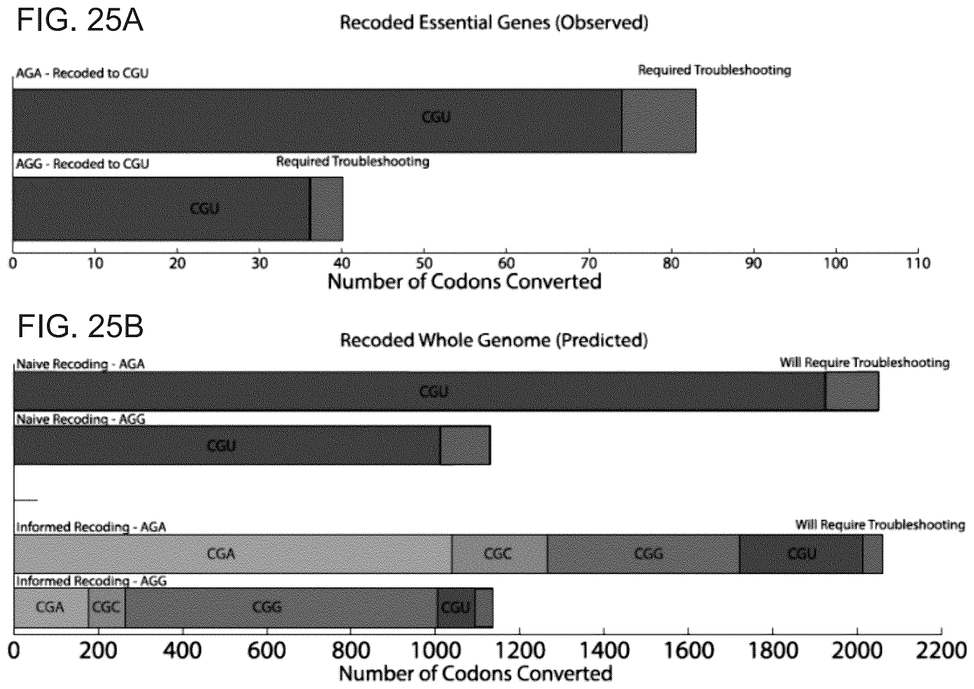

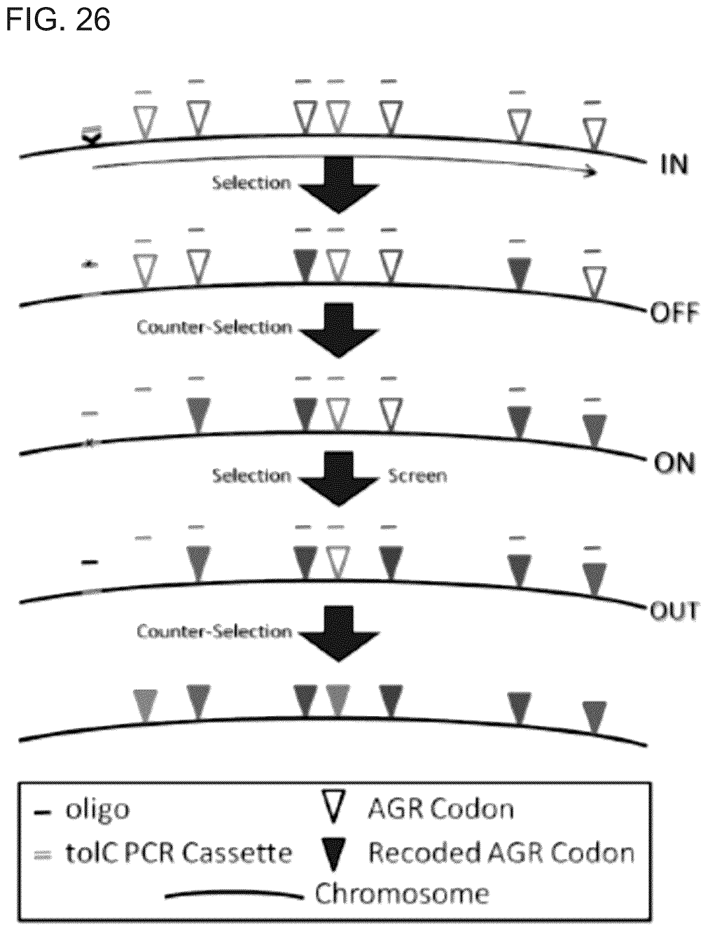

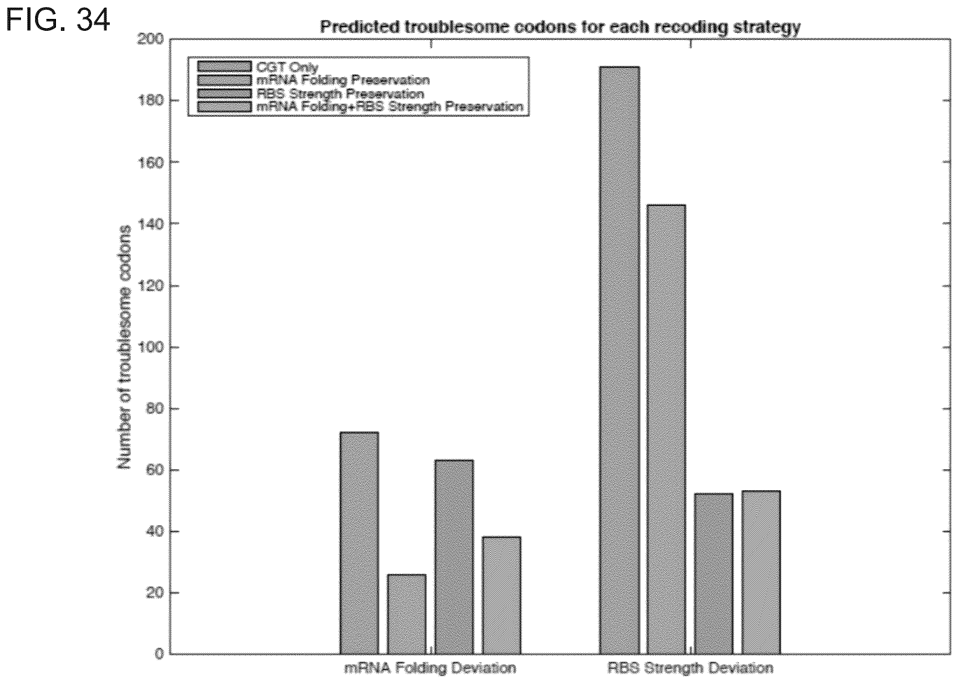

These rules were used to generate a draft genome in silico with all AGR codons replaced genome-wide, reducing by almost fourfold the number of predicted design flaws (e.g., synonymous codons with metrics outside of the SRZ) compared to the naïve replacement strategy (FIG. 25A-25B, FIG. 34). Furthermore, predicting recalcitrant codons provides hypotheses that can be rapidly tested in vivo using MAGE. Successful replacement sequences can then be implemented together in a redesigned genome. These rules are expected to increase the tractability of creating a genome completely devoid of AGR codons, which could be used for unambiguously reassigning AGR translation function.

Comprehensively removing all instances of AGR codons from E. coli essential genes revealed 13 design flaws which could be explained by a disruption in coding DNA Sequence, RBS-mediated translation initiation/pausing, or mRNA structure. While the importance of each factor has been reported, methods described herein systematically explore to what extent and at what frequency they impact genome function. Furthermore, methods described herein establish quantitative guidelines to reduce the chance of designing non-viable genomes. Although additional factors undoubtedly impact genome function, the fact that these guidelines captured all instances of failed synonymous codon replacements (FIG. 22) suggests that the disclosed genome design guidelines provide a strong first approximation of acceptable modifications to the primary sequence of viable genomes. These design rules coupled with inexpensive DNA synthesis will facilitate the construction of radically redesigned genomes exhibiting useful properties such as biocontainment, virus resistance, and expanded amino acid repertoires (Lajoie et al., 2015).

### Materials and Methods:

### Strains and Culture Methods Used:

The strains used in this work were derived from EcM2.1 (Escherichia coli MG1655 mutS_mut dnaG_Q576AexoX_mut xonA_mut xseA_mut 1255700::toIQRA Δ(ybhB-bioAB)::[λcI857 N(cro-ea59)::tetR-bla]) (Carr et al., 2012). Liquid culture medium consisted of the Lennox formulation of Lysogeny broth (LBL; 1% w/v bacto tryptone, 0 .5% w/v yeast extract, 0 .5% w/v sodium chloride) (Lennox, 1955) with appropriate selective agents: carbenicillin (50 μg/mL) and SDS (0.005% w/v). For tolC counter-selections, colicin E1 (colE1) was used at a 1:100 dilution from an in-house purification (Schwartz et al., 1971) that measured 14.4 μg protein/μL (Isaacs et al., 2011, Lajoie et al., 2013b), and vancomycin was used at 64 μg/mL. Solid culture medium consisted of LB^{L} autoclaved with 1 .5% w/v Bacto Agar (Fisher), containing the same concentrations of antibiotics as necessary. ColE1 agar plates were generated as described previously (Gregg et al., 2014). Doubling times were determined on a Biotek Eon Microplate reader with orbital shaking at 365 cpm at 34° C. overnight, and analyzed using a matlab script.

### Oligonucleotides, Polymerase Chain Reaction, and Isothermal Assembly

PCR products used in recombination or for Sanger sequencing were amplified with Kapa 2G Fast polymerase according to manufacturer's standard protocols. Multiplex allele-specific PCR (mascPCR) was used for multiplexed genotyping of AGR replacement events using the KAPA2G Fast Multiplex PCR Kit, according to previous methods (Isaacs et la., 2011, Mosberg et al., 2012). Sanger sequencing reactions were carried out through a third party (Genewiz). CRAM plasmids were assembled from plasmid backbones linearized using PCR (Yaung et al., 2014), and CRISPR/PAM sequences obtained in Gblocks from IDT, using isothermal assembly at 50° C. for 60 minutes. (Gisbon et al., 2009).

### Lambda Red Recombinations, MAGE, & CoS-MAGE

λ Red recombineering, MAGE, and CoS-MAGE were carried out as described previously (Gregg et al., 2014, Wang et al., 2009). In singleplex recombinations, the MAGE oligo was used at 1 μM, whereas the co-selection oligo was 0.2 μM and the total oligopool was 5 μM in multiplex recombinations (7-14 oligos). When double-stranded PCR products were recombined (e.g., tolC insertion), 100 ng of double-stranded PCR product was used. Since CoS-MAGE was used with tolC selection to replace target AGR codons, each recombination was paired with a control recombined with water only to monitor tolC selection performance. The standard CoS-MAGE protocol for each oligo set was to insert tolC, inactivate tolC, reactivate tolC, and delete tolC. MascPCR screening was performed at the tolC insertion, inactivation and deletion steps. All 2 Red recombinations were followed by a recovery in 3 mL LB^{L} followed by a SDS selection (tolC insertion, tolC activation) or ColE1 counter-selection (tolC inactivation, tolC deletion) that was carried out as previously described (Gregg et al., 2014).

### General AGR Replacement Strategy

AGR codons in essential genes were found by cross-referencing essential gene annotation according to two complementary resources (Baba, et al., 2006, Hashimoto et al., 2005) to find the shared set (107 coding regions), which contained 123 unique AGR codons (82 AGA, 41 AGG). optMAGE (Ellis et al., 2001, Wang et al., 2009) was used to design 90-mer oligos (targeting the lagging strand of the replication fork) that convert each AGR to CGU. The total number of AGR replacement oligos was reduced to 119 by designing oligos to encode multiple edits where possible, maintaining at least 20 bp of homology on 5′ and 3′ ends of the oligo. The oligos were then pooled based on chromosomal position into twelve MAGE oligo sets of varying complexity (minimum: 7, maximum: 14) such that a single marker (tolC) could be inserted at most 564,622 bp upstream relative to replication direction for all targets within a given set. tolC insertion sites were identified for each of the twelve pools either into intergenic regions or non-essential genes that met the distance criteria for a given pool. See Table 5 for descriptors for each of the 12 oligo pools.

### Troubleshooting Strategy

A recalcitrant AGR was defined as one that was not converted to CGU in one of at least 96 clones picked after the third step of the conversion process. The recalcitrant AGR codon was then triaged for troubleshooting (FIG. 12A) in the parental strain (EcM2.1). First, the sequence context of the codon was examined for design errors or potential issues, such as misannotation or a disrupted RBS for an overlapping gene. In most cases, corrected oligos could be easily designed and tested. If no such obvious redesign was possible, AGR was attempted to be replaced with CGN mutations. If attempting to replace AGR with CGN failed to give recombinants, compensatory, synonymous mutations were tested in a 3 amino acid window around the recalcitrant AGR. If needed, synonymous stringency was relaxed by recombining with oligos encoding AGR-to-NNN mutations. After each step in the troubleshooting workflow, 96 clones from 2 successive CoS-MAGE recombinations were screened using allele specific PCR with primers that hybridize to the wildtype genotype. Sequences that failed to yield a wild-type amplicon were Sanger sequenced to confirm conversion. Doubling time was measured of all clones in LB^{L} to pair sequencing data with fitness data, and chose the recombined clone with the shortest doubling time. Doubling time was determined by obtaining a growth curve on a Biotek plate reader (either an Eon or H1), and analyzed using web-based open source genome resequencing software. This genotype was then implemented in the complete strain at the end of strain construction using MAGE, and confirmed by MASC-PCR screening.

### MRNA Folding and RBS Strength Computation

A custom Python pipeline was used to compute mRNA folding and RBS strength value for each sequence. mRNA folding was based on the UNAFold calculator (Markham et al., 2008) and RBS strength on the Salis calculator (Salis, 2011). The parameters for mRNA folding are the temperature (37° C.) and the window used which was an average between-30: +100nt and-15:+100nt around the start site of the gene and was based on Goodman et al., 2013. The only parameter for RBS strength is the distance between RBS and promoter and between 9 and 10 nt was averaged after the codon of interest based on Li et al., 2012. Data visualization was performed through a custom Matlab code.

### Whole Genome Sequencing of Strains Lacking AGR Codons in their Essential Genes

Sheared genomic DNA was obtained by shearing 130 μL of purified genomic DNA in a Covaris E210. Whole genome library prep was carried out as previously described (Rohland et al., 2012). Briefly, 130 μL of purified genomic DNA was sheared overnight in a Covaris E210 with the following protocol: Duty cycle 10%, intensity 5, cycles/burst 200, time 780 seconds/sample. The samples were assayed for shearing on an agarose gel and if the distribution was acceptable (peak distribution ˜400 nt) the samples were size-selected by SPRI/Reverse-SPRI purification as described in (Rohland et al., 2012). The fragments were then blunted and p5/p7 adaptors were ligated, followed by fill-in and gap repair (NEB). Each sample was then qPCR quantified using SYBR green and Kapa Hifi. This was used to determine how many cycles to amplify the resulting library for barcoding using P5-sol and P7-sol primers. The resulting individual libraries were quantified by Nanodrop and pooled. The resulting library was quantified by qPCR and an Agilent Tapestation, and run on MiSeq 2×150.

Data was analyzed to confirm AGR conversions and to identify off-target mutations using Millstone, an web-based open-source genome resequencing tool.

### NNN-sequencing and CRISPR

CRISPR/Cas9 was used to deplete the wildtype parental genotype by selectively cutting chromosomes at unmodified target sites next to the desired AGR codons changes. Candidate sites were determined using the built-in target site finder in Geneious proximally close to the AGR codon being targeted. Sites were chosen if they were under 50 bp upstream of the AGR codon and could be disrupted with synonymous changes. If multiple sites fulfilled these criteria, the site with the lowest level of sequence similarity to other portions of the genome was chosen. Oligos of a length of ˜130 bp were designed for all 24 genes with an AGR codon in the first 30 nt after the translation start site. Those oligos incorporated both an NNN random codon at the AGR position as well as multiple (up to 6) synonymous changes in a CRISPR target site at least 50 nt downstream of an AGR codon. This modifies the AGR locus at the same time as disrupting the CRISPR target site, ensuring randomization of the locus after the parental genotype is deleted. Recombinations were performed in the parental strain EcM2.1 carrying the Cas9 expressing plasmid DsCas9. For each of 24 genes, five cycles of MAGE were performed with the specific mutagenesis oligo at a concentration of 1 μM. CRISPR repeat-spacer plasmids carrying guides designed to target the chosen sites, and were electroporated into each diversified pool after the last recombineering cycle. After 1 hour of recovery, both the DsCas9 and repeat-spacer plasmids were selected for, and passaged in three parallel lineages for each of the 24 AGR codons for 144 hrs. After 2 hours of selection, and at every 24 hour interval, samples were taken and the cells were diluted 1/100 in selective media.

Each randomized population was amplified using PCR primers allowing for specific amplification of strains incorporating the CRISPR-site modifications. The resulting triplicate libraries for each AGR codon were then pooled and barcoded with P5-sol and P7-sol primers, and run on a MiSeq 1×50. Data was analyzed using custom Matlab code.

For each gene and each data point, reads were aligned to the reference genome and frequencies of each codon were computed. In FIG. 23, the mRNA structure deviation (red line) and RBS strength deviation (blue line) in arbitrary units were computed based as the product of the frequencies and the corresponding deviation for each codon.

### Example III

### Genome Engineering Toolkit and Multi-locus Validation Experiment

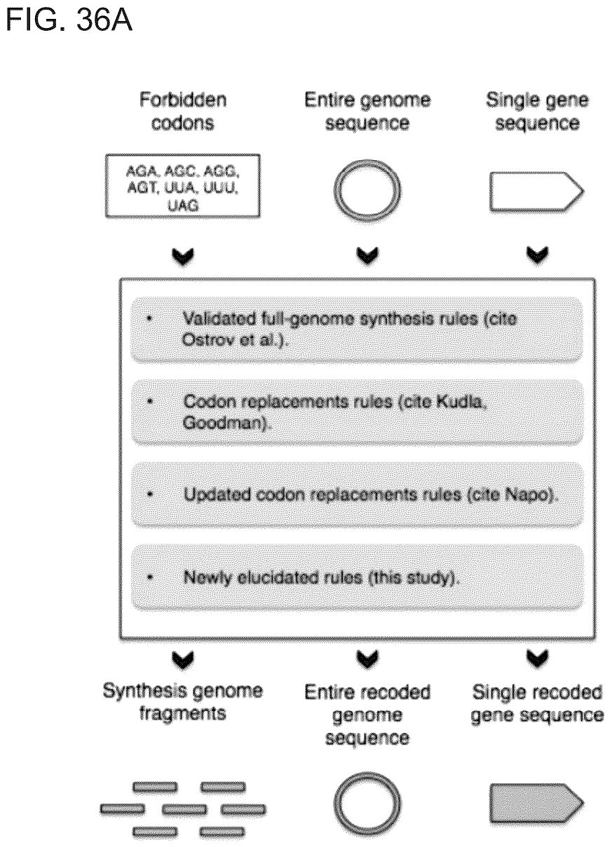

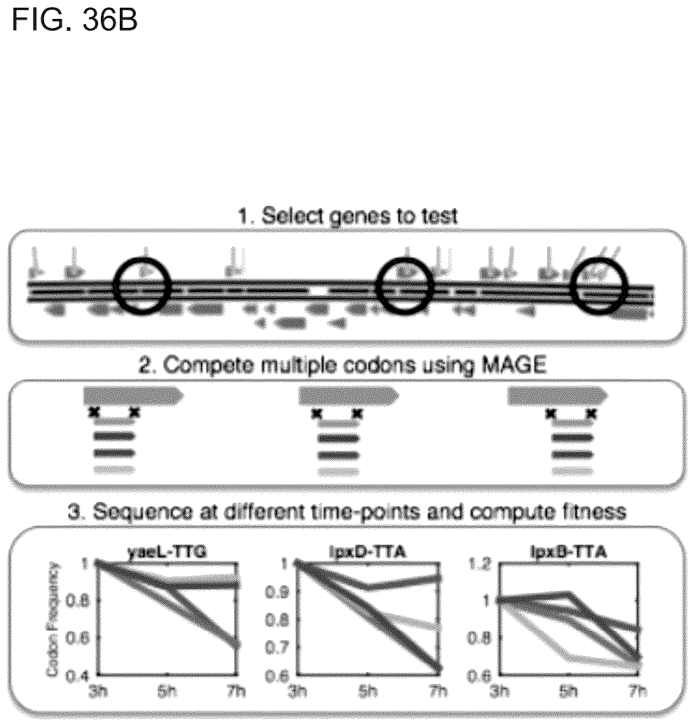

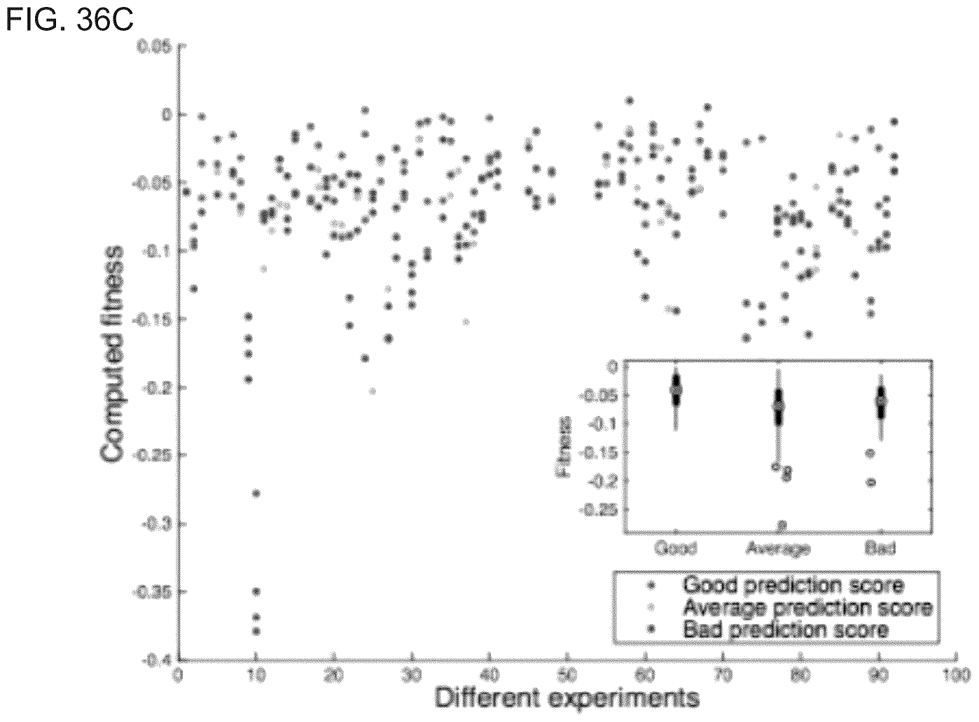

Methods described herein make use of the Genome Engineering Toolkit (GETK), a software library for reassigning codons genome-wide. GETK software supports design and synthesis of recoded genes and whole genomes (FIG. 36A). The software takes into account biophysical constraints to choose the best codon reassignment, minimizing the risk of redesigned organisms that are impaired or inviable. Using software encoding methods described herein, experiments were we carried recoding positions throughout the genome and demonstrating that the codon choices specified by the methods described herein reduce the risk of design exceptions.

To validate the design rules described herein, an experiment was carried out to test synonymous codon substitutions throughout the genome. 235 codon competition experiments were designed, and prioritized according to the predicted difficulty of codon replacement. Positions were selected where at least one of mRNA, RBS, or internal RBS were predicted by the design rules to be significantly disrupted for at least one alternative codon. The 6 forbidden sense codons as in Example 1 were considered: AGA (Arg), AGG (Arg), AGC (Ser), AGU (Ser), UUG (Leu), and UUA (Leu). Positions were prioritized where the design rule-predicted score max_{mRNA |RBS|internal_RBS} exceeded a threshold, or at least one bad recoding existed. For each sub-experiment, MAGE oligos were designed that introduce synonymous codons at the target. For some sub-experiments, MAGE oligos were designed that introduce non-synonymous mutations. Each sub-experiment was performed in a separate well and MAGE was used to electroporate the oligo set for that sub-experiment. The population was sampled at regular intervals and diluted to maintain logarithmic-phase growth. The samples were sequenced and used to quantify codon abundance, which was then used to calculate relative fitness (FIG. 36B).

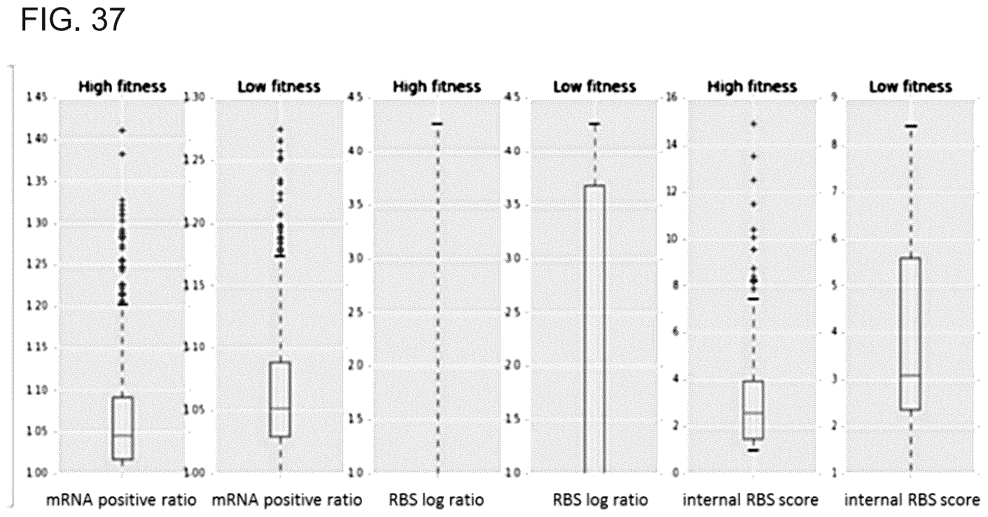

Predicted scores were compared to experimental fitness measurements (FIG. 36C). Our experiments reveal that alternative codon predictions can minimize design issues. In the case of testing single codon changes at the 5-prime ends of essential genes, codons categorized as having good scores (minimal predicted disruption of mRNA folding, ribosome binding site strength, and internal ribosome pausing sites) result in significantly less fitness impact (K-S test). Testing combinations of codon swaps within the same 90-mer oligo window showed even stronger correspondence between predicted scores and observed fitness (FIG. 37).

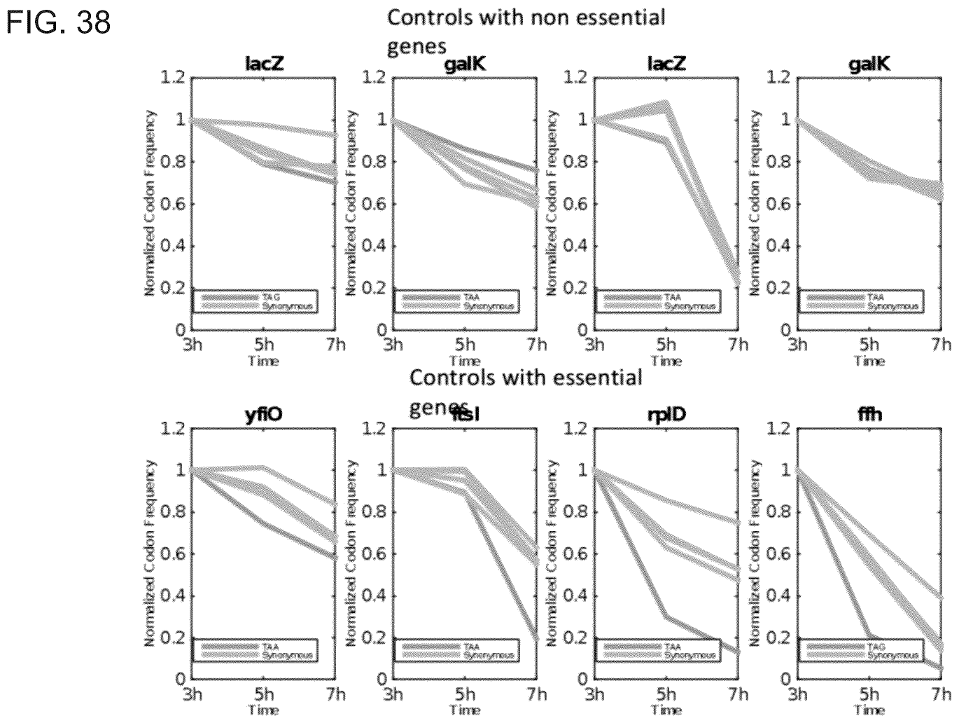

As a null-effect controls, synonymous codons and early stop codons were introduced into non-essential genes LacZ and GalK at multiple positions, showing similar effect between synonymous codons and internal stops (FIG. 38, top row). As strong-effect controls, synonymous codons and internal stop codons were introduced into essential genes. These show a marked difference between internal stop and synonymous codons, with a greater dynamic range of codon preference at some positions (FIG. 38, bottom row).

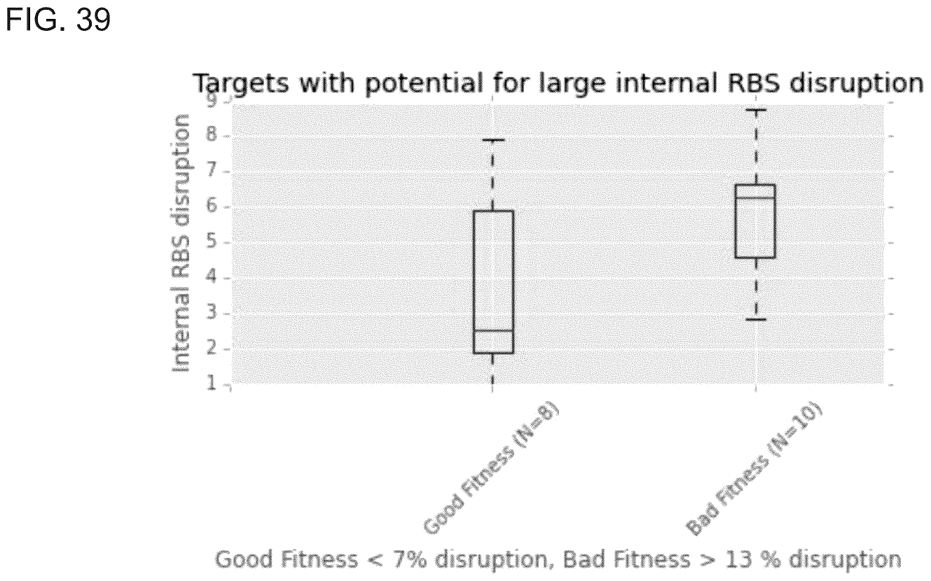

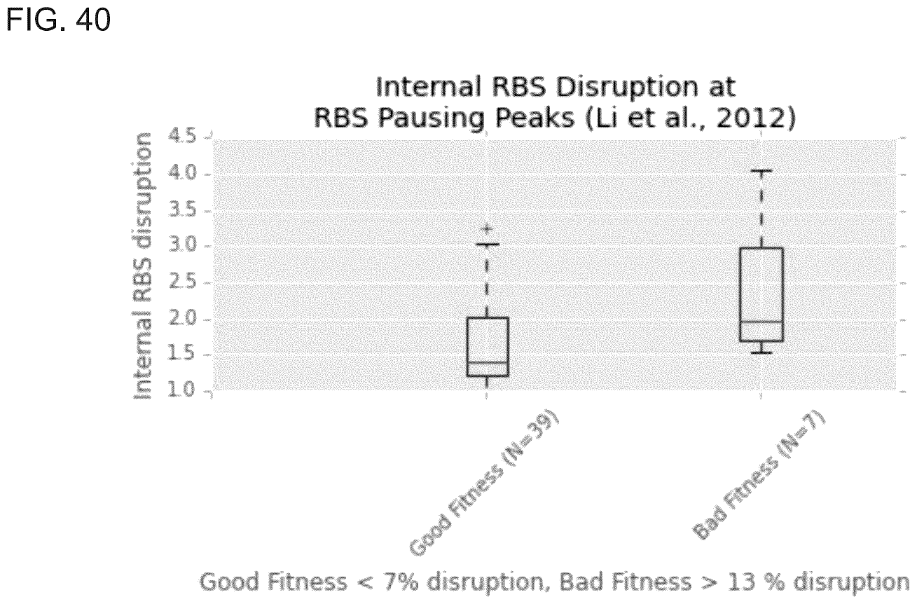

Beyond testing synonymous substitutions, non-synonymous substitutions observed in phylogenetic neighbors of E. coli (gammaproteobacteria, e.g. Salmonella enterica) that score well according to the rules described herein were tested for ability to replace codons. Preventing disruption of internal RBS motifs is an effective rule for selecting codons internal to genes, both for loci with potential high RBS disruption (FIG. 39) (Kolmogorov-Smirnov p=3.E-14) and for loci observed to have strong ribosomal pausing peaks (Li et al., 2012) (FIG. 40) (Kolmogorov-Smirnov p=7 .9E-05).

### Choosing Genomic Locus Targets

Targets for the 235-codon competition experiments were organized into three 96-well plates:

### Plate 1: Single Codon Changes in 5-Prime of Essential Genes

95 codons were chosen that occur near the 5-prime end of essential genes, (−30, +100) bases relative to the start codon. Positions were considered where the worst possible score exceeds thresholds for at least one filter (poor RBS or mRNA folding prediction), as described by the filter:

```
single_codon_any_bad_max = single_codon_agg_data_df[
  (single_codon_agg_data_df[‘max_RBS_log_ratio’] > 3.3) |
  (single_codon_agg_data_df[‘max_mRNA_positive_ratio’] > 1.1) |
  (single_codon_agg_data_df[‘max_internal_RBS_score’] > 4.1)]
```

The threshold values were chosen as follows:

RBS_log_ratio: 3.3 = 1 + math.log_e(10)
mRNA_positive_ratio: 1.1 = 10% deviation
max_internal_RBS_score: 4.1 = 3.3 + a bit more to get down to <
96-well plate

The candidate set contains targets with at least one problem in the design (i.e. the worst design is bad). At least two of these targets introduce non-synonymous mutations into overlapping genes, allowing testing the aspect of the software that balances amino acid sense against preservation of regulatory gene expression signals.

### Plate 2: Combos of Codon Changes and Adjacent Degenerate Tests

From among the single changes, those that occur adjacent to others within a 90-basepair oligonucleotide size were combined into a new set of sub-experiments that tested all combinations of adjacent oligos. There were 62 such targets.

12 sub-experiments were designed with synonymous codon swaps in non-forbidden codons adjacent to forbidden codons. Oligos were designed that bring in all synonymous codon swaps on either side of some choice forbidden codons, e.g. the region surrounding an arginine V-R-G might look like GTN-CGN-GGN in an oligo. For these, recodings were targeted which have a score that exceeds threshold values with the best synonymous codon swap, where even the best synonymous solution is bad.

### Plate 3: Testing Phylogenetic Conservation

The final 66 sub-experiments were designed to test phylogenetic conservation as a source of permitted non-synonymous substitutions. Seven strains of gammaproteobacteria were aligned and codons were identified that have non-synonymous variants relative to E. coli. Targets were tested around the 5-prime ends of essential genes as well as targets in the middle of essential genes. For conservation 5-prime targets, a subset was chosen of non-synonymous changes observed in phylogenetic conservation data for which there is a possible bad score, as described by:

```
conservation_5_prime_non_synonymous_df = conservation_5_prime_df[
  (conservation_5_prime_df[‘replacement_codon’].apply(
      lambda c: c not in FORBIDDEN_CODONS)) &
  (~conservation_5_prime_df[‘is_synonymous’])][:]
conservation_5_prime_synonymous_only_bad_df  =
conservation_5_prime_non_synonymous_df[
    (conservation_5_prime_non_synonymous_df[‘max_mRNA_positive_ratio’] >
1.1) |
    (conservation_5_prime_non_synonymous_df[‘max_RBS_log_ratio’] > 3.3) |
    (conservation_5_prime_non_synonymous_df[‘max_internal_RBS_score’] > 4.1)
][:]
conservation_5_prime_first_30nt_bad_score  =
conservation_5_prime_non_synonymous_df[
    (conservation_5_prime_non_synonymous_df[‘codon_start’] < 30) &
    ((conservation_5_prime_non_synonymous_df[‘mRNA_positive_ratio’] > 1.1) |
    (conservation_5_prime_non_synonymous_df[‘RBS_log_ratio’] > 3.3) |
    (conservation_5_prime_non_synonymous_df[‘internal_RBS_score’] > 3.3))
][:]
conservation_5_prime_targets_df = pd.concat([
  conservation_5_prime_synonymous_only_bad_df,
  conservation_5_prime_first_30nt_bad_score])
conservation_5_prime_targets_df.drop_duplicates(inplace=True)
```

These selections were competed against the corresponding single codon degenerate oligo from plate 1.

For conservation in middle of genes, the ˜3500 candidate targets in essential genes were reduced using two criteria: 1) internal RBS score with a bad potential maximum with synonymous changes and 2) locations of peaks from ribosomal pausing data (Li et al., 2012).

For internal RBS, 12 targets at 9 unique positions were chosen, for a total of 21 oligos. This filter used is:

```
conservation_middle_of_genes_df = conservation_essentals_df[
  (conservation_essentals_df[‘codon_start’] > 30) &
  (conservation_essentals_df[‘scoring_gene’] ==
      conservation_essentals_df[‘codon_gene’]) &
  (conservation_essentals_df[‘replacement_codon’].apply(
      lambda c: c not in FORBIDDEN_CODONS)) &
  (~conservation_essentals_df[‘is_synonymous’]) &
  (conservation_essentals_df[‘max_internal_RBS_score’] > 6.5) &
  (conservation_essentals_df[‘internal_RBS_score’] <
    conservation_essentals_df[‘min_internal_RBS_score’])
][:]
```

For Weissman, 14 targets at 9 unique positions, or 23 oligos were chosen.
1.0 Oligonucleotides were designed as described in (Wang et al., 2009). DNA was synthesized by industrial partners IDT DNA technologies (Coralville, IA).

### Strains & Culture

EcM2.1 naïve strains were used for the competition experiment (EcM2.1 is a strain optimized for MAGE-Escherichia coli MG1655 mutS_mut dnaG_Q576A exoX_mut xonA_mut xseA_mut 1255700:toIQRA Δ(ybhB-bioAB)::[λcI857 N(cro-ea59)::tetR-bla]).

Liquid culture medium consisted of the Lennox formulation of Lysogeny broth (LB^{L}; 1% w/v bacto tryptone, 0 .5% w/v yeast extract, 0 .5% w/v sodium chloride) with appropriate selective agents: carbenicillin (50 μg/mL). Solid culture medium consisted of LB^{L} autoclaved with 1 .5% w/v Bacto agar (Thermo Fisher Scientific Inc.), containing the same concentrations of antibiotics as necessary.

### Experiment Setup

The recombineering experiments using the EcM2.1 strain were carried out as described previously, and in the same conditions for all different competition experiment. Depending on the experiment, the total oligo pool was adjusted to a maximum of 5 μM.

After transformation of the oligos, cells were taken out at 1, 3, 5 , 7 and 24 hrs to be sequenced. Dilution were performed so as to maintain cells in constant log phase. At each timepoint, cells were plated on permissive media so as to count the number of cells present in the pools. Based on these numbers, we were able to compute the number of doublings between each timepoint.

| | # of |
|---|---|
| Timepoint | Doublings |
| 1 hr | 1 |
| 3 hr | 3 |
| 5 hr | 7 |
| 7 hr | 10 |

### Sequencing

Each population was amplified and barcoded with Illumina P5 and P7 primers, pooled, and sequenced using a MiSeq or NextSeq using a PE-150 kit. Reads were demultiplexed to the reference genome and frequencies of each codon were computed for each sub-experiment.

### Estimating Relative Allele Fitness and Scoring

For each sub-experiment, the relative frequency of each codon was calculated. Then the fractions were normalized relative to the fraction at the first timepoint. Then, for each codon, the fitness was inferred by fitting a logarithmic function to the codon fraction across all time points and taking the decay constant as a measure fitness. The mRNA structure deviation and RBS strength deviation were computed using GETK and scores were compared to empirically measured fitness.

### TABLES

TABLE 1
Genome Design Rules-Biological Constraints

| | Rule | Motivation | Implementation |
|---|---|---|---|
| A | Fix gene overlaps: | Forbidden codons may fall in the | Use synonymous codon swaps |
| | Perform minimal | overlapping region of two genes. | (Genbank annotation: adj_base_ov) |
| | synonymous codon | Sometimes it may be possible | to avoid introducing on synonymous |
| | swaps required to | to remove forbidden codons | changes in overlapping genes. |
| | properly recode | through synonymous swaps | Use computational RBS motif |
| | both overlapping | alone. In other cases, in order to | strength prediction to maintain RBS |
| | genes. | avoid introducing nonsynonymous | motif. |
| | If necessary- | mutations or disrupting regulatory | In short gene overlaps, attempt to |
| | separate by | motifs such as ribosome binding | minimize editing, for example reduce |
| | duplicating | sites (RBS), it is necessary to | 4 nucleotide overlap to 1 nucleotide |
| | overlapping regions | separate the genes first so that | (see FIG. 9A (i)) |
| | [202 instances] | codons in each gene can be | If minimal overlap fix does not |
| | | replaced independently. | preserve RBS motif, separate the |
| | | | overlap by copying the overlapping |
| | | | sequence and 15-20 base pairs |
| | | | upstream, to preserve native RBS |
| | | | (see FIG. 9A (ii)) Genbank |
| | | | annotation: fix_overlap. |
| | Reduce homology | To separate overlapping genes, | Perform synonymous codon swaps |
| | between duplicated | the sequences are duplicated, | in copied regions to reduce homology |
| | regions through | creating two tandem paralogous | while maintaining regulatory motifs. |
| | non-disruptive | regions. These two paralogs have | (Genbank annotation: adj_base_ov) |
| | shuffling of copied | the potential to recombine | |
| | region | spontaneously which could cause | |
| | | a disruptive change in either the | |
| | | upstream or downstream gene. | |
| | | This spontaneous recombination | |
| | | was prevented by shuffling the | |
| | | codons of the upstream paralog, | |
| | | thus maintaining the native | |
| | | nucleotide sequence of the N- | |
| | | terminus of the downstream gene | |
| | | and 15-20 bases upstream. This | |
| | | region has shown to be important | |
| | | for mRNA folding and translation | |
| | | initation | |
| B | Preserve 5-prime | Gene expression is affected by | Use thermodynamics-based secondary |
| | mRNA secondary | mRNA secondary structure | structure prediction to compare |
| | structure of genes | | mRNA free energy (ΔG) of wild- |
| | | | type and recorded sequence. |
| | | | Minimize ΔG change across 40-bp |
| | | | windows centered at modified codons. |
| | Preserve GC content | Related to DNA stability, mRNA | Maintain GC content when choosing |
| | | secondary structure. | among alternative codons. Minimize |
| | | | ΔGC across 40 base pair windows |
| | | | centered at modified codons. |
| | Rebalance codon | Preserve codon usage bias for | Ensure selection of alternate codons |
| | usage | remaining 57 codons in order to | is consistent with global distribution |
| | | preserve expression dynamics that | of codon choice; both for recording |
| | | are dependent on a aa-tRNA | and heterologous expression. |
| | | availability. | |

TABLE 2
Genome Design Rules-Synthesis Constraints

| | Rule | Motivation | Implementation |
|---|---|---|---|
| C | Remove repetitive (REP) | REP regions were found to be | Replace each REP sequence with |
| | sequences [132 instances] | over-enriched in DNA fragments | unique terminator sequence drawn |
| | | that failed the repetitiveness | from orthogonal set. Note that not all |
| | | metric for commercial synthesis | REPs were deleted as some were |
| | | and/or failed during synthesis. | tolerated for DNA synthesis. |
| | | Hypothesizing that these REP | Genbank annotation: |
| | | elements were used as | rep_to_term. |
| | | transcriptional terminators, it | |
| | | was tested whether they could be | |
| | | replaced with synthetic | |
| | | terminator sequences (data not | |
| | | shown). It was found that REP | |
| | | sequences could not be replaced | |
| | | with synthetic transcriptional | |
| | | terminators with no measurable | |
| | | effect. | |
| D | Remove restriction | DNA synthesis vendor constraint | Disruption of restriction enzyme |
| | sites needed for | | motifs using synonymous codon |
| | synthesis [AarI: 972 | | swaps. (Genbank annoation: |
| | instances, BsaI: 182 | | adj_base_RE) |
| | instances, BsmBI: | | Preserve functional RNA (e.g. rRNA) |
| | 954 instances] | | secondary structure when necessary. |
| | | | If outside of coding regions, |
| | | | change single nucleotides |
| | | | to avoid disrupting annotated |
| | | | regulatory motifs. (Genbank |
| | | | annotation: adj_base_RNA) |
| E | Remove homopolymer | DNA synthesis vendor constraint: | In coding sequence, synonymous |
| | runs [158 instances] | remove sequence of more than 8 | codon swaps were performed. In |
| | | consecutive A, C, T or more than | intergenic sequence, minimal |
| | | 5 consecutive G | nucleotide changes were performed |
| | | | that avoid disrupting annotated |
| | | | regulatory motifs. (Genbank |
| | | | annotation: adj_base_hp) |
| NA | Rebalance GC | DNA synthesis vendor constraint: | If coding sequence contains very |
| | content extremes | 0.30 < GC < 0.75. | high/low GC content, use synonymous |
| | | | codon swaps to normalize GC content. |
| | | | Genbank annotation: adj_base_GC) |
| | | | If intergenic sequences contains high/ |
| | | | low GC content, introduce minimal |
| | | | nucleotide changes to avoid disrupting |
| | | | annotated regulatory motifs. |
| | | | (Genbank annotation: adj_base_GC) |
| F | Partition genome into 87 | Splitting operons were avoided | Allow ±5 kb variability in segment |
| | 50-kb “segments” at | so that segments remain modular | size to find partitioning that keep |
| | operon boundaries | and can be redesigned independent | whole operons together. |
| | | of each other. | Genbank annotation: segment. |
| G | Partition each “segment” | 2-4 kb was used as the primary | Choose partitioning to minimize |
| | into ~15 | synthesis unit, as offered by | secondary structure at 50 base pair |
| | synthesiscompatible | vendors. 50 bp overlaps enable | overlaps to maximize success rate in |
| | fragments of 2-4 kb | homologous-recombination | yeast assembly. |
| | with 50 bp overlaps | based on assembly in S. cerevisae | Genbank annotation: synthesis_frag. |
| | between adjacent | | |
| | fragments | | |

TABLE 3
Primers used for PCR of kanamycin cassette for chromosomal deletion.
Forward primers disclosed as SEQ ID NOS 1-87, respectively, in order
of appearance, and reverse primers disclosed as SEQ ID NOS 88-174,
respectively, in order of appearance.

| Casette | Forward primer | Reverse primer |
|---|---|---|
| KanDeletion-seg0 | GAA AAA AAT ATC ACC AAA TAA AAA | TGC ATA TAT TCC CCA AAT CGA CAC ACG |
| | ACG CCT TAG TAA GTA TTT TTC CTG | GAT ATC AGG GCT ATC TCC TTA TTA GAA |
| | ATC CTT CAA CTC ACC AAA AGT TC | AAA CTC ATC GAG CAT CAA ATG |
| KanDeletion-seg1 | CAA TTG ACC GCA GCC GGA AAA CGG | ATA GTC AGG AAT AGT CTT ATT TAG TTT |
| | TAA AAG CAC CTT TAT ATT GTG GTG | AAG CAT ATT GAT GTC CAG TTA TTA GAA |
| | ATC CTT CAA CTC AGC AAA AGT TC | AAA CTC ATC GAG CAT CAA ATG |
| KanDeletion-seg2 | AAA TAC QCG CCA GGT GAA TTT CCC | TCA CCG GGC ATT GTG TCG TTT ATG CGC |
| | TCT GGC GCC TAG AGT ACG GGA CTG | AGC GCG TGC GCT GAC TTT TTA TTA GAA |
| | ATC CTT CAA CTC AGC AAA AGT TC | AAA CTC ATC GAG CAT CAA ATG |
| KanDeletion-seg3 | TAC ACC GAG AAA GCC GAT GGG GTG | CGT CTG AAC TGC CGC CCG GAA GTA ACG |
| | ATT TTC CAG ACT GCG GTT TAA CTG | ATG CTG GAA CTG GTG TAG TTA TTA GAA |
| | ATC CTT CAA CTC AGC AAA AGT TC | AAA CTC ATC GAG CAT CAA ATG |
| KanDeletion-seg4 | AAA TCA AAA AAT TAC CTG CTT TAT | CAC TCT TTC AAC GAG CAA TTG TAT ATT |
| | TCT GGT GAT AAA ATT CAC GAT CTG | GTT ATG TAA GCA AGT GCT TTA TTA GAA |
| | ATC CTT CAA CTC AGC AAA AGT TC | AAA CTC ATC GAG CAT CAA ATG |
| KanDeletion-seg5 | TGC GAT TTA ATG TTC TCC ATA ATG | CCT ACA GAT TCT TGC GCC ATT CGT AGG |
| | AGC AAA ATT CTG ACC GGT GTA CTG | CCG GAT AAG CGG TTC ACG TTA TTA GAA |
| | ATC CTT CAA CTC AGC AAA AGT TC | AAA CTC ATC GAG CAT CAA ATG |
| KanDeletion-seg6 | TGC GAA CGT TAC GGC GTC TGA CCT | GTG TAT GGA AAA ATC AGA AAA ACT CAG |
| | ACA TGT TCA TGC CGG ATG CGG CTG | CAA ATC CTG ATG ACT TTC TTA TTA GAA |
| | ATC CTT CAA CTC AGC AAA AGT TC | AAA CTC ATC GAG CAT CAA ATG |
| KanDeletion-seg7 | GAA AGC CGG ACG TAA CCG CAC CGA | TTG TCA CTC TAA TGA TAA TTA TTT GTT |
| | AGT GGC GGC CTG ACG TCC GGC CTG | AAA TAA TTG TTT TAT TTC TTA TTA GAA |
| | ATC CTT CAA CTC AGC AAA AGT TC | AAA CTC ATC GAG CAT CAA ATG |
| KanDeletion-seg8 | GGG AGT GCT GAA GGA GTC TGG QCG | AAA CGA TAC CAC CAA CAG GCG ATT GCC |
| | GGC AAT TGG TAT AAC CAA TGT CTG | TCA AGA AAG GCA CCT GGG TTA TTA GAA |
| | ATC CTT CAA CTC AGC AAA AGT TC | AAA CTC ATC GAG CAT CAA ATG |
| KanDeletion-seg9 | TCA TCT GCA CTT TCC GCA AAT TAT | ATC CGG TAC CCA TTG TAG GCC TGA TAA |
| | CTC GCC ATT AAC CGT TTC AGC CTG | GAT QCG TCA AGC ATC GCA TTA TTA GAA |
| | ATC CTT CAA CTC AGC AAA AGT TC | AAA CTC ATC GAG CAT CAA ATG |
| KanDeletion-seg10 | GCC TAC AAC CGC TGC CGC ATC CGG | CAG CGC CAT GCA AGT GCT GGA TAG GCT |
| | CAA TTG GTG CAC AAT GCC TGA CTG | TAA GGC GCT GTT TTA AGC TTA TTA GAA |
| | ATC CTT CAA CTC AGC AAA AGT TC | AAA CTC ATC GAG GAT CAA ATG |
| KanDeletion-seg11 | ATT TTC GCC AGA CGC CGC CGC AGG | CAG ACA CGA CTT TGT AGA AAT TGT TTT |
| | TGA CAG CGT CCG ACA GTT AAT CTG | ACA AAA ATG GCG ATG CAA TTA TTA GAA |
| | ATC CTT CAA CTC AGC AAA AGT TC | AAA CTC ATC GAG CAT CAA ATG |
| KanDeletion-seg12 | AAT CGG CTT TCG AAA GTG GGC TAT | GTG AAC GCC TTA TCC GGC CTA CAA AAT |
| | CAT CCC ACC CCG CGT CGC AGA CTG | CGC TTA AAT TCA ATA TAT TTA TTA GAA |
| | ATC CTT CAA CTC AGC AAA AGT TC | AAA CTC ATC GAG CAT CAA ATG |
| KanDeletion-seg13 | AAT TGC CTG ATG CCC TAC GCT TAT | TAA GCT AAC TTT AGT GAC ATT TAT GTT |
| | GAG GCC TAC GAG GAT GCT GCA CTG | TAA AAT GTG TGA GTT ATA TTA TTA GAA |
| | ATC CTT CAA CTC AGO AAA AGT TC | AAA CTC ATC GAG CAT CAA ATG |
| KanDeletion-seg14 | CGT CTC TTT TTA TCT TTA ATT GCC | GTT TAT GCC GGA TGC GGC GTG AAC GCC |
| | AAC CGA AAC TAA TTT CAG CCT CTG | TTA TCC GGC CTA CAA ACC TTA TTA GAA |
| | ATC CTT CAA CTC AGC AAA AGT TC | AAA CTC ATC GAG CAT CAA ATG |
| KanDeletion-seg15 | CGC TTA TCA GGC CTA CAT TTT CTC | GGC TAA ATC ATT CAC ATC ATC AAT TTC |
| | CGC AAT ATA TTG AAT TTG CGC CTG | ATC CTT ACT TTC ATT CGA TTA TTA GAA |
| | ATC CTT CAA CTC AGC AAA AGT TC | AAA CTC ATC GAG CAT CAA ATG |
| KanDeletion-seg16 | CCG TAA CAG TGT AAT AAC AAT GTG | CTA AGC CTT CGA TCT CAA AAG CAT TAT |
| | ACG CAG AGC ACA AAT TAT ATT CTC | CAG ACT GAT ACG CTA TTA TTA TTA GAA |
| | ATC CTT CAA CTC ACC AAA AGT TC | AAA CTC ATC GAG CAT CAA ATG |
| KanDeletion-seg17 | CGA TCG CTC TGA AAG CGT TCT ACG | AAA ACG GGT CAG ATC TGC CAG AGT CAG |
| | ATA ATA ATG ATA TCC TTT CAA CTG | CGT CAC CGA CCA CAA TAA TTA TTA GAA |
| | ATC CTT CAA CTC AGC AAA AGT TC | AAA CTC ATC GAG CAT CAA ATG |
| KanDeletion-seg18 | GGA CTG ATA TTC CCG CTG CTG GCG | ACT CGC CTG AGA AAA CAG GGG TAA ATT |
| | CGT AAA GCG AAT AGT AAA TAA CTG | CCC CGA ATG GCG GCG CTA TTA TTA GAA |
| | ATC CTT CAA CTC AGC AAA AGT TC | AAA CTC ATC GAG CAT CAA ATG |
| KanDeletion-seg19 | AAG ATA ACT AAA GCA CTG GGT TGA | AGA AAA ATA ACC CGA TAA TGG TAG ATC |
| | TAA ATA ACC GAA TGG CGG CAA CTG | TCC CTC TTT ATC CTG AAA TTA TTA GAA |
| | ATC CTT CAA CTC AGC AAA AGT TC | AAA CTC ATC GAG CAT CAA ATG |
| KanDeletion-seg20 | CAG TCT TAT GAA TAT CGC AAT CGG | TTT TGC AGT AAA AAA TTG TCG ACG GAG |
| | CGA ATA CCT CTG GTC GTA GAG CTG | GTG TGG AGA AAA AAC AAG TTA TTA GAA |
| | ATC CTT CAA CTC AGC AAA AGT TC | AAA CTC ATC GAG CAT CAA ATG |
| KanDeletion-seg21 | ATA TAA AAA ATA TTT CGG TGT AGT | AAA TCG TTT TGC TGC CGT ATA TAT CGC |
| | GCT TTC GTC ATG TAA AAC GTT CTG | CAT TAT TCC CAT TTC TGC TTA TTA GAA |
| | ATC CTT CAA CTC AGC AAA AGT TC | AAA CTC ATC GAG CAT CAA ATG |
| KanDeletion-seg22 | TGT CAT GTA AAC CAA ACA GAG AAT | ACG TGA TCT GTT CGG TCG CTA ATC CAT |
| | GTC TTT TCA GCG CAT TCG CAG CTG | TCG GCC CTC CTG CGG GAG TTA TTA GAA |
| | ATC CTT CAA CTC AGC AAA AGT TC | AAA CTC ATC GAG CAT CAA ATG |
| KanDeletion-seg23 | CTG ATT TAC TGA GGG TCA AAT AAA | TAC AGT GAC TTC ATA AAA ATT ATG AGA |
| | TAT ACC GGC AGG AAA AAA GCG CTG | TTT TTC ACG GTG CTG TAA TTA TTA GAA |
| | ATC CTT CAA CTC AGC AAA AGT TC | AAA CTC ATC GAG CAT CAA ATG |
| KanDeletion-seg24 | ATT TGC CGT GTG GTT ACT CGC TTT | TTT TTT CCC CCG ACA TCA TAA CGG TTC |
| | ACA TCG GTA AGG GTA GGG ATT CTG | TCG CAA ATA TTC TGA AAT TTA TTA GAA |
| | ATC CTT CAA CTC AGC AAA AGT TC | AAA CTC ATC GAG CAT CAA ATG |
| KanDeletion-seg25 | CTT GCG TAC TAG TT AACT AGT TCG | GCT GAA CTG TTA ATA CAA TTT GCG TGC |
| | ATG ATT AAT TGT CAA CAG CTC CTG | CAA TTT TTT ATC TTT TTG TTA TTA GAA |
| | ATC CTT CAA CTC AGC AAA ACT TC | AAA CTC ATC GAG CAT CAA ATG |
| KanDeletion-seg26 | ATC CTG GCA TGT TGC TGT TGA TTC | AAT CGC TGA CAG AAA CCG ATA TTG ACA |
| | TTC AAT CAG ATC TTT ATA AAT CTG | TCC TCC ACG CCC TGA AGG TTA TTA GAA |
| | ATC CTT CAA CTC AGC AAA AGT TC | AAA CTC ATC GAG CAT CAA ATG |
| KanDeletion-seg27 | CGG AAA TGA TTC AGG CGA CAG CCT | ACC ATT GCC TGC GCA ATG GTG TTT TTG |
| | GAA CGT AGC AGG GAT CCA CGT CTG | TTT TTA TCT GCT TTA TAC TTA TTA GAA |
| | ATC CTT CAA CTC AGC AAA AGT TC | AAA CTC ATC GAG CAT CAA ATC |
| KanDeletion-seg28 | GGG GCT TTT ATC GTC TTT GCT TTA | TCC AGC AAA AAT TCT TCC CGA TCG TCA |
| | CCG CCA GGG CGT CGG CCT CAA CTG | TTA CCA GCT GAC GTG ATA TTA TTA GAA |
| | ATC CTT CAA CTC AGC AAA AGT TC | AAA CTC ATC GAG CAT CAA ATG |
| KanDeletion-seg29 | TGG CAT TTC CGC GTC TGT TTA TTG | AAT CTT AAG TAG TGA TTC GTG CCG GGG |
| | TTG CCC GGC GTA TGG AGT AAA CTG | CGA TGT CTC GTT TTA CCC TTA TTA GAA |
| | ATC CTT CAA CTC AGC AAA AGT TC | AAA CTC ATC GAG CAT CAA ATG |
| KanDeletion-seg30 | CAC CTT AGA ACG CCG GAT AAA GAC | CTG GGC GGT GGC GGT GAA CGC TAT GCC |
| | TGA TAA TTG TCT TCG ACG GTC CTG | TGT GGT GTA ATT AAG TAA TTA TTA GAA |
| | ATC CTT CAA CTC AGC AAA AGT TC | AAA CTC ATC GAG CAT CAA ATG |
| KanDeletion-seg31 | TCG CAA CTT GAG CAA GCA CCA CCG | AAC AAC TCA GGC AAC ACG CAA ACC ATT |
| | CAA GGT ACG CTG GCC TCT TAA CTG | TAC TCG TCG TAT TTC AAC TTA TTA GAA |
| | ATC CTT CAA CTC AGG AAA AGT TC | AAA CTC ATC GAG CAT CAA ATG |
| KanDeletion-seg32 | ATA GTA AGT GAC TGG GGT GAA CGA | TGC CTT TGA CGA TCT ATT GCT ATA AAT |
| | ACG TAG CCG CAG CAC ATG CAA CTG | AAG TGA TCT TTT TTC TTT TTA TTA GAA |
| | ATC CTT CAA CTC AGC AAA AGT TC | AAA CTC ATC GAG CAT CAA ATC |
| KanDeletion-seg33 | ATC ATG ATT AGC AAA ACT TAA CCA | TGA ACT TAA GTC TGA GAC CTA TTT GGC |
| | TTT TAA AAT AAA TAA ACA ATT CTC | CGG TAA TCC CTC TCG AAT TTA TTA GAA |
| | ATC CTT CAA CTC AGC AAA AGT TC | AAA CTC ATC GAG CAT CAA ATG |
| KanDeletion-seg34 | TGG GTC TGT TAC AGG TTG ATG GAA | CTT TGG GGA TTG ACT TCT CTT TAG GGT |
| | GGC GGG GGG CAA AAA GAG CAA CTG | AAT TAA TAG CCG TTA ACT TTA TTA GAA |
| | ATC CTT CAA CTC AGG AAA AGT TC | AAA CTC ATC GAG CAT CAA ATG |
| KanDeletion-seg35 | ATG CAA TGA ATA AAA AGT TAT ATC | CGT ACA GCG CGC TTA CCA TAC AAA CTC |
| | ACT TTT TCT CAT AAA ACA GTC CTG | CCT TTA AAA TGG CCG ATG TTA TTA GAA |
| | ATC CTT CAA CTC ACC AAA AGT TC | AAA CTC ATC GAG CAT CAA ATG |
| KanDeletion-seg36 | GCA ATC TTC TCT TTT CTG AAT TTG | AGC AAT GCC GTG AGC ACA GGT ATC TTT |
| | CCA CCT ATC ATA GAC AGG TGC CTG | CTC TGT TGG CCG TAT TGT TTA TTA GAA |
| | ATC CTT CAA CTC AGC AAA AGT TC | AAA CTC ATC GAG CAT CAA ATG |
| KanDeletion-seg37 | TAA TAA GCT AAC CCG CAT TGA GTT | ATA ACC TCA CAT TAT CCC TGA ATT AAA |
| | AAC CAA TAA CGG ATT CCA TAC CTG | AGT GGT AAT AAT AAA ACA TTA TTA GAA |
| | ATC CTT CAA CTC AGC AAA AGT TC | AAA CTC ATC GAG CAT CAA ATG |
| KanDeletion-seg38 | ACA ATA TTT AAT ATA GTG TCT CCA | GTG AAA AGG GGT TAG ATA GTA CCA AAT |
| | CAT CCG ATA TTT CTT AAA TAA CTG | GGG AAA ATG TTA AGT AAG TTA TTA GAA |
| | ATC CTT CAA CTC AGC AAA AGT TC | AAA CTC ATC GAG CAT CAA ATG |
| KanDeletion-seg39 | GAT AAA CCA TCA GGT GAT AGT TTA | AAT CAC TTT TGC CGA GGT AAC AGC GTC |
| | CCT GAA GAA TAT AGA GAA GTA CTG | ATA ACA ACA ATT AAA GCC TTA TTA GAA |
| | ATC CTT CAA CTC AGC AAA AGT TC | AAA CTC ATC GAG CAT CAA ATG |
| KanDeletion-seg40 | CTT TTT AAA ATT CGT TCT TCC ATG | GGG TAT GGA GCT ATG GGT ATT TTC TGT |
| | CCC GGT AAC GCT CCA GAA AAC CTG | ACC CAA TGC TTT TAA CAG TTA TTA GAA |
| | ATC CTT CAA CTC AGC AAA ACT TC | AAA CTC ATC GAG CAT CAA ATG |
| KanDeletion-seg41 | AGA ACC AGA TTG ATG CAT TGA CCT | TCT CCC TTG TTT CAA TTG AAA AGT CCA |
| | TTC ATC CTA TGA AAT TAA TTG CTG | GGC TGC AAA GTC TGG GCT TTA TTA GAA |
| | ATC CTT CAA CTC AGC AAA AGT TC | AAA CTC ATC GAG CAT CAA ATC |
| KanDeletion-seg42 | TTT TTA CGG CCA CAG CCA AAC TTT | GAG GTA ATT CAG GCG TAA TCA ACA ACC |
| | ACC GTG CCC TAA TAC GAC AAA CTG | CTT GTC TAT AGT TAG TGA TTA TTA GAA |
| | ATC CTT CAA CTC AGC AAA AGT TC | AAA CTC ATC GAG CAT CAA ATG |
| KanDeletion-seg43 | ACC AAA CTG ATT AGA CAT TCT CGT | TTC AAC CGC TAT ACC TGC TAT CTT CAA |
| | TCT CCA TTT GCG TAA AAC CTG CTG | CTT CAG GAC AAT AAT GCA TTA TTA GAA |
| | ATC CTT CAA CTC AGC AAA AGT TC | AAA CTC ATC GAG CAT CAA ATG |
| KanDeletion-seg44 | TGA CGA CAA CAG TAA CAT TCA ACG | AAA ATC AGG CAT TGT ACC GAT GAT TTA |
| | TTA AAT ATG TTA ATA AGA CGT CTG | TAG TTT CAA GTT GCC ACT TTA TTA GAA |
| | ATC CTT CAA CTC AGC AAA AGT TC | AAA CTC ATC GAG CAT CAA ATG |
| KanDeletion-seg45 | TTG CAA TAC AAT TCT TAC GCC TGT | TTG CCG CCG CTG GCG GAA GCA TAA AAA |
| | AGG ATT AGT AAG AAG ACT TAT CTG | AAT GGC GCC GAT GGG CGC TTA TTA GAA |
| | ATC CTT CAA CTC AGC AAA AGT TC | AAA CTC ATC GAG CAT CAA ATG |
| KanDeletion-seg46 | CCG CTT ATC CCC ATC AAG AAG TAA | CTT GAC TTC CTT CAC TGT AGC GGC AAG |
| | TTC TTG CCG CAG TGA AAA ATG CTG | GTA CGA GCC AAT CGT GGA TTA TTA GAA |
| | ATC CTT CAA CTC ACC AAA AGT TC | AAA CTC ATC GAG CAT CAA ATG |
| KanDeletion-seg47 | TTC AGT ATA AAA GGG CAT GAT AAT | AGT CGA TAG TAA CCC GCC CTT CGG CGA |
| | TTA CAT TAA CTC CTT TTT TTC CTG | TAG CAA GCA TTT TTT CCA TTA TTA GAA |
| | ATC CTT CAA CTC AGC AAA AGT TC | AAA CTC ATC GAG CAT CAA ATG |
| KanDeletion-seg48 | GCC GCG GCA TTA TAC AGA GCG TAA | CTA TTA ACT GTA ATA TTT GAG CCC CAC |
| | CCG ATT GCA TCT ACC CCT TTT CTG | GCG CTG CCG CTC ATC ACA TTA TTA GAA |
| | ATC CTT CAA CTC AGC AAA AGT TC | AAA CTC ATC GAG CAT CAA ATG |
| KanDeletion-seg49 | CCT CCT GTA GGG TTT TTA TTA ACA | GCT GCA TCC AGA AAG TAA CAA TAG CGA |
| | ACG GGT TAT TCT AAT TAT TTT CTG | ACA GAC AAA AAG AAT ACG TTA TTA GAA |
| | ATC CTT CAA CTC AGC AAA AGT TC | AAA CTC ATC GAG CAT CAA ATG |
| KanDeletion-seg50 | CAA CCC CGT CCT GTA CGG GGT TTG | CAA ATC GCC GGA ATT TCC CGT GAT ATA |
| | TTT TTT CGA GCG CAC GTT TTG CTG | AGG GCT GAG AGC AAA TCG TTA TTA GAA |
| | ATC CTT CAA CTC AGC AAA AGT TC | AAA CTC ATC GAG CAT CAA ATG |
| KanDeletion-seg51 | TTC AGG CGT TTT TTC GCT ATC TTT | GCG GTG AAT AAT GTC GAT GAT GTC GAA |
| | GAC AAA AAA TAT CAA CTT TCT CTG | ATG ACA CGT CGA CAC GCC TTA TTA GAA |
| | ATC CTT CAA CTC AGC AAA AGT TC | AAA CTC ATC GAG CAT CAA ATG |
| KanDeletion-seg52 | TTT ATT CTT ATT AAA GAG ATT TTT | ACG GTT CTG GCC TGG GGA CTT GTA GGC |
| | AAG CTA AAG ATG AAT TTC GTC CTG | CTG ATA AGA CGC GTC AAG TTA TTA GAA |
| | ATC CTT CAA CTC ACC AAA AGT TC | AAA CTC ATC GAG CAT CAA ATG |
| KanDeletion-seg53 | TTG TAG GCC GCA CGC CAC ATC CGA | GAA CAA GAA AAA TTC CGC TTT CGT TAT |
| | CAT TCA GCG CCT GAT GCG ACG CTG | GAA CAA TAA TTT ACG TAG TTA TTA GAA |
| | ATC CTT CAA CTC ACC AAA AGT TC | AAA CTC ATC GAG CAT CAA ATG |
| KanDeletion-seg54 | AAT GGC GGC GAA AAT CAG CAT AAA | TAT CTA CCC CTC TAT TGG TGG GTT AGT |
| | ACG GGT GGT CAT GGT CGT ACC CTG | GGT TGC AAA CCT TAC GTG TTA TTA GAA |
| | ATC CTT CAA CTC ACC AAA AGT TC | AAA CTC ATC GAG CAT CAA ATG |
| KanDeletion-seg55 | ATC ACA AAC GAA ATA TGC CTG AGC | CTC GAT TCT GCT GTG GCT TTT GGG GCT |
| | AGG AGT CAG AGA CAT AAC TGG CTC | AGT GTA TCA GAA TCG CTT TTA TTA GAA |
| | ATC CTT CAA CTC AGC AAA AGT TC | AAA CTC ATC GAG CAT CAA ATG |
| KanDeletion-seg56 | TAT GGT CAC TCA TTT GAT CCA TTA | CGA TAG TCG TTA ACT GTT TTA CAC TTA |
| | TGC CTT ATT GTG CCG TGA CTA CTG | ATA AAA TAA TTT GAG GTT TTA TTA GAA |
| | ATC CTT CAA CTC AGC AAA AGT TC | AAA CTC ATC GAG CAT CAA ATG |
| KanDeletion-seg57 | CCC GCT GAC GAA GGC AAA CCC ATA | AGA GCT TCC GGC TCT GCA TGA TGA TGT |
| | GAC ATG TCG TCA GAC ATA GCG CTG | CCT TAT ATT TGG CAT TCC TTA TTA GAA |
| | ATC CTT CAA CTC ACC AAA AGT TC | AAA CTC ATC GAG CAT CAA ATG |
| KanDeletion-seg58 | ATT TAT TCC CCT CGC GTC CCG CCC | TTA CTG CAA TTG CTG CTG CTT TGT AAA |
| | GTT GTT ACT CTT GCT TGT TCA CTG | GCA CCG CGG CCT TTT TTG TTA TTA GAA |
| | ATC CTT CAA CTC AGC AAA AGT TC | AAA CTC ATC GAG CAT CAA ATG |
| KanDeletion-seg59 | GGA GAA AGC CTC GTG TAT ACT CCT | GAT TAT GGC GAG CAA GGC CAC ATA AAC |
| | CAC CCT TAT AAA AGT CCC TTT CTG | GCC AGG TTT TGG GGA TCG TTA TTA GAA |
| | ATC CTT CAA CTC AGC AAA AGT TC | AAA CTC ATC GAG CAT CAA ATG |
| KanDeletion-seg60 | AAC AAC CCG TAG CCC GGA CAA GAT | TAA AGA AAC CAG GGT GTC ATC GTC TGC |
| | GCG CCA GCA TCG CAT CCG GCA CTG | GTC GCA TGT TAA GGT CAC TTA TTA GAA |
| | ATC CTT CAA CTC AGC AAA AGT TC | AAA CTC ATC GAG CAT CAA ATG |
| KanDeletion-seg61 | ATG GCG ATG AGT GTT TCC ATT GCT | AAA CAA TGC CTC TTA AGG TTT TCT TAA |
| | GTT CTC TTT TAT ACT GTG GGC CTG | GGT TCT TCT GAA AGT GAA TTA TTA GAA |
| | ATC CTT CAA CTC AGC AAA AGT TC | AAA CTC ATC GAG CAT CAA ATC |
| KanDeletion-seg62 | CTG AAA TCG TTC TCA ATC AAC GTC | TGC TGA TGC GCA AAG TCC GTC AGC AGT |
| | ATT TGT ACA TTT TGT GCG CTT CTG | TTG CAG TGC AAT AAA GGT TTA TTA GAA |
| | ATC CTT CAA CTC AGC AAA AGT TC | AAA CTC ATC GAG CAT CAA ATG |
| KanDeletion-seg63 | TGA CGA CGC GGA GAA CCG GAA GCT | GGG TTG AGC TGG CTA GAT TAG CCA GCC |
| | AAA TAC AGA GAA GTC ATA GAA CTG | AAT CTT TTG TAT GTC TGT TTA TTA GAA |
| | ATC CTT CAA CTC AGC AAA AGT TC | AAA CTC ATC GAG CAT CAA ATG |
| KanDeletion-seg64 | TTC CAT GCT GAA AAG CCC GTT TTC | ACT GAA CGG TCC CCT CGC CCC TTT GGG |
| | AGG ATA CTC AAA TGG AAA CGC CTG | GAG AGG GTT AGG GTG AGG TTA TTA GAA |
| | ATC CTT CAA CTC AGC AAA AGT TC | AAA CTC ATC GAG CAT CAA ATG |
| KanDeletion-seg65 | CAT CCG GCG ATG CTG CCG CGT TGA | ATC TAA AAA GAT GAT CTT AAT AAA TCT |
| | ATT TTA CAT CCC GTA CGT TCC CTG | ATT AAC AAT GAG ATG GAG TTA TTA GAA |
| | ATC CTT CAA CTC AGC AAA AGT TC | AAA CTC ATC GAG CAT CAA ATG |
| KanDeletion-seg66 | TAA GTA AAG GAG TGA AAC AGT TTC | GCT ATA AAG GAA CCC GCT TTG TCA GCT |
| | ATA AGT AAA ATA TCC AGT GTG CTG | TTG TAG CCG AAC AAT AAG TTA TTA GAA |
| | ATC CTT CAA CTC AGC AAA AGT TC | AAA CTC ATC GAG CAT CAA ATG |
| KanDeletion-seg67 | TTG AAC TGC TGG CCT GGC AGA AGA | GAC TCG GCA TGT TTG GGA TTA TTA AGC |
| | AAT TTA AAG TTA AAA AAT AAC CTG | TGA CAA TTC ATA CCA TTA TTA TTA GAA |
| | ATC CTT CAA CTC AGC AAA AGT TC | AAA CTC ATC GAG CAT CAA ATG |
| KanDeletion-seg68 | CAT TCG TCA TCA ATT TGA ACA ACA | GTA ACG CTA AAG TCT CTT TTC AAA CTT |
| | CAA TAC TGA CCC ACA TTC CCG CTG | GCA TTT TTG TAA ATT TGT TTA TTA GAA |
| | ATC CTT CAA CTC AGC AAA AGT TC | AAA CTC ATC GAG CAT CAA ATG |
| KanDeletion-seg69 | ACC ACA GCA AAG GGA AAA AGT GTG | TTT TTC AAC TAT CTC TGT AAC CCT TGC |
| | GGG AAA GAG TGT GCA TGA AGC CTG | CCG TAA ATT CGT CAT AGC TTA TTA GAA |
| | ATC CTT CAA CTC AGC AAA AGT TC | AAA CTC ATC GAG CAT CAA ATG |
| KanDeletion-seg70 | ACG TGA CTG GCG AAA TCT TCG CCA | TTT ATT GTC GGC AGT GCC AGA ACT AAT |
| | GTC GGT AAC AGG TTT ACG ACA CTG | TCA TGC GCC CCG GAT GGC TTA TTA GAA |
| | ATC CTT CAA CTC AGC AAA ACT TC | AAA CTC ATC GAG CAT CAA ATG |
| KanDeletion-seg71 | AGG CGC TGA TGG CGA ACT TAG CGT | AGC ATC GTT CTC CCA TGG AGC TGA TGA |
| | AGC GTT TAT GCC GGA TGG TAT CTG | CGA TGC TGC GGT GAC GTG TTA TTA GAA |
| | ATC CTT CAA CTC ACC AAA AGT TC | AAA CTC ATC GAG CAT CAA ATG |
| KanDeletion-seg72 | CCG CTG GCG ACG CGG ATG TCG CAT | AAG CAC CTT AAT TAT CGT CGC ATT CAG |
| | CAG QGG CAG CCC GTT TAA GCG CTG | AAC AGT CTG GAT GCG ATG TTA TTA GAA |
| | ATC CTT CAA CTC AGG AAA AGT TC | AAA CTC ATC GAG CAT CAA ATG |
| KanDeletion-seg73 | CGT CTA AAC ATA ATA TCC CTT TAT | ATT CTT TGA CCG AGC TAG TTA TGG CGC |
| | GGT CCA AAG AAA GAA TTA ACG CTG | GGA GTA TTA GTT ACG CTT TTA TTA GAA |
| | ATC CTT CAA CTC ACC AAA AGT TC | AAA CTC ATC GAG CAT CAA ATG |
| KanDeletion-seg74 | AAT TAT TTG TCG TTA TGA TTT AAA | GGG TGA AAC AGT CAG TTT CCG CTA AGA |
| | TGT TTT GTT TTA CAC TCT GTC CTG | TTG CAT GCC GGA TAA GCC TTA TTA GAA |
| | ATC CTT CAA CTC AGG AAA AGT TC | AAA CTC ATC GAG CAT CAA ATG |
| KanDeletion-seg75 | CGC TAT TAC AGC AAT ATT TTT CGT | TAC ATT TCA TAG TGA TGC TCC TTA CTC |
| | GAT GAA CGT GCC GGA AAG CGA CTG | TTG AGA CAG ACA CGT TAG TTA TTA GAA |
| | ATC CTT CAA CTC AGC AAA AGT TC | AAA CTC ATC GAG CAT CAA ATG |
| KanDeletion-seg76 | ATC AGA TTC ACC CAT ATC GCC TCT | GTG AAC ATA ATA AAT CAA AAA AGA AAA |
| | TTT ATT GTG GGA TTG ACC CTG CTG | CGC CAC TAC ACG CAT TTT TTA TTA GAA |
| | ATC CTT CAA CTC AGC AAA AGT TC | AAA CTC ATC GAG CAT CAA ATG |
| KanDeletion-seg77 | GCA GGA CTT ATT CAT TTC GTG AAT | AAA TCA GGG AAG ATG AAA AAA CTT CAG |
| | TTT ATT ATT TTA TTT ATA AAC CTG | GAT GGT AAG AAA AAG AAA TTA TTA GAA |
| | ATC CTT CAA CTC AGC AAA AGT TC | AAA CTC ATC GAG CAT CAA ATG |
| KanDeletion-seg78 | ATG GTT AGT TTA TAT TTG CAG TCC | CGT ATT AGC TTT TCG CAT TAT ACG CCC |
| | GGT TTG CTT TGC ATA CCG GAT CTG | TCA ACA GAG CCT GTC TCA TTA TTA GAA |
| | ATC CTT CAA CTC AGC AAA AGT TC | AAA CTC ATC GAG CAT CAA ATG |
| KanDeletion-seg79 | ACC AGA ACC TGG CTC ATC AGT GAT | CAC TTT TAT TAA CTC AGC ATT ATT TTT |
| | TTT CTT TGT CAT AAT CAT TGC CTC | AAA CAT CAA ACC ACT TAA TTA TTA GAA |
| | ATC CTT CAA CTC ACC AAA AGT TC | AAA CTC ATC GAG CAT CAA ATG |
| KanDeletion-seg80 | CCG TAA AAG TTT CGG TGG AAT GAG | AGA AAC ACA GTT AAA AAT TGC AAA AGA |
| | ATC TTG CGA TTT TCT TAA TAA CTG | TTT TTT AGA CCT GGA GAA TTA TTA GAA |
| | ATC CTT CAA CTC AGC AAA AGT TC | AAA CTC ATC GAG CAT CAA ATG |
| KanDeletion-seg81 | AAT AAA TGC GTG AAA AAC TTT ACT | CAC CCT AAC CCT CTC CCC AGA GGG GCG |
| | TGC AAT ACA ACT TGA TAC TTC CTG | AGG GGA CCG ATT GTG CTC TTA TTA GAA |
| | ATC CTT CAA CTC AGC AAA AGT TC | AAA CTC ATC GAG CAT CAA ATG |
| KanDeletion-seg82 | CAC CCC AAT GGG GAG AGG GAG AAA | CAT TGT AAA CAT TAA ATG TTT ATC TTT |
| | ACG AGC GCA ATA TTC AAT ATC CTG | TCA TGA TAT CAA CTT GCG TTA TTA GAA |
| | ATC CTT CAA CTC AGC AAA AGT TC | AAA CTC ATC GAG CAT CAA ATG |
| KanDeletion-seg83 | TTT CTG TAA CTG AGA ACT TGA GGT | AAT CAC CGT TTG CTT AAA AAT GGA TTC |
| | TTT TTA TTA ACA CAT CAG GAT CTG | TAC CAT CGC TTT TTC AGA TTA TTA GAA |
| | ATC CTT CAA CTC AGC AAA AGT TC | AAA CTC ATC GAG CAT CAA ATG |
| KanDeletion-seg84 | AAC AGA CTG ATC GAG GTC ATT TTT | AAT AAG TTC TTC TGG CGT AAT AAC CCT |
| | GAG TGC AAA AAG TGC TGT AAC CTG | GAA CGC CGG GCT TCG GTT TTA TTA GAA |
| | ATC CTT CAA CTG AGC AAA AGT TC | AAA CTC ATC GAG CAT CAA ATG |
| KanDeletion-seg85 | GAA TAA GGT GTG TTT ATT TAT CGC | TTT TTT TAT TTC TAC TGA TAA GAA TTA |
| | GGG CAT AAA AAA ACC CTT ACT CTC | CAA GGC ACA TCA CGT TAT TTA TTA GAA |
| | ATC CTT CAA CTC AGC AAA AGT TC | AAA CTC ATC GAG CAT CAA ATG |
| KanDeletion-seg86 | GTG ATG AAG ATC ACG TCA GAA AAT | ATC CAC ACA GAG ACA TAT TGC CCG TTG |
| | TGT TAC ATT ACT ATG TTA CGC CTG | CAG TCA CAA TGA AAA GCT TTA TTA GAA |
| | ATC CTT CAA CTC AGC AAA AGT TC | AAA CTC ATC GAG CAT CAA ATG |

TABLE 4
MASC primers (SEQ ID NOS 175-2262, respectively, in order of
appearance) used for analysis of recoded segments.

| Primer | Sequence |
|---|---|
| mAsPCR-seg00.1..Recoded | CAAGCTAGACGAAGGCATGTCA |
| mAsPCR-seg00.1..Reverse | CGATATTTTCCCGTGGTTCTGAC |
| mAsPCR-seg00.1..Wild-Type | CAAGTTAGACGAAGGCATGAGT |
| mAsPCR-seg00.2..Recoded | CGACCATGGCGATCTTCAGC |
| mAsPCR-seg00.2..Reverse | TTCCAGGTATTACGCAGAAATTGTTC |
| mAsPCR-seg00.2..Wild-Type | CGACCATGGCGATTTACAGT |
| mAsPCR-seg00.3..Recoded | CTTACCGCGCAAAATTTCATCTCA |
| mAsPCR-seg00.3..Reverse | TTTTTACGCAGCACTACTTGTATATGG |
| mAsPCR-seg00.3..Wild-Type | TTAACCGCGCAAAATTTCATCAGC |
| mAsPCR-seg00.4..Recoded | CCTGTTTTCACACTACCGTTCA |
| mAsPCR-seg00.4..Reverse | TTAATTTGCATAGACCGTTTTCAGAGT |
| mAsPCR-seg00.4..Wild-Type | CCTGTTTAGCCACTACCGTAGC |
| mAsPCR-seg00.5..Recoded | CGGGAAGTGATGTTTTATCTCAACC |
| mAsPCR-seg00.5..Reverse | ACTTTCGCAGTGGCTTGTG |
| mAsPCR-seg00.5..Wild-Type | CGGGAAGTGATGTTTTATCTCAACT |
| mAsPCR-seg00.6..Recoded | TGCCGTCAGGGAGATAATTTTAG |
| mAsPCR-seg00.6..Reverse | CCCTGACCAACGCCAAAG |
| mAsPCR-seg00.6..Wild-Type | TGCCGTCAGGGAGATAATTTTGC |
| mAsPCR-seg00.7..Recoded | CACCGATGAAAAACAGCCCAAG |
| mAsPCR-seg00.7..Reverse | CGTTTTGTAGCCCGCTCTG |
| mAsPCR-seg00.7..Wild-Type | CACCGATGAAAAACAGCCCCAA |
| mAsPCR-seg00.8..Recoded | CCCGAGTGTGTATTCAGGTTCAAT |
| mAsPCR-seg00.8..Reverse | CCTGGACTTCGGTTTCACG |
| mAsPCR-seg00.8..Wild-Type | CCCGAGTGTGTATTCAGGTTCAAA |
| mAsPCR-seg01.1..Recoded | CGTCTGGAAGAGCACAAAGACT |
| mAsPCR-seg01.1..Reverse | AAAAAGTTCAAAAATTCGCTGTGGAG |
| mAsPCR-seg01.1..Wild-Type | CGTCTGGAAGAGCACAAAGACA |
| mAsPCR-seg01.2..Recoded | TGGATCTCAGATACAGAATCAGAAC |
| mAsPCR-seg01.2..Reverse | AGCCACTGATGCTGAAGGG |
| mAsPCR-seg01.2..Wild-Type | TGGATCAGCGATACAGAAAGCGAAT |
| mAsPCR-seg01.3..Recoded | GGTGCAAGCGTAACCTGTAG |
| mAsPCR-seg01.3..Reverse | GACTATTTCTACGGCACCATTCCC |
| mAsPCR-seg01.3..Wild-Type | GGTGCAAGCGTAACCTGCAA |
| mAsPCR-seg01.4..Recoded | CGACCGCGGGAAAGATAATGT |
| mAsPCR-seg01.4..Reverse | GGCTGGGTTGGCGTTTTAAA |
| mAsPCR-seg01.4..Wild-Type | CGACCGCGGGACAAATAATGA |
| mAsPCR-seg01.5..Recoded | GTGGTTGCGGGTTTGGTTAG |
| mAsPCR-seg01.5..Reverse | GCTGGTCCGAAGCCTACG |
| mAsPCR-seg01.5..Wild-Type | GTGGTTGCGGGTTTGGTCAA |
| mAsPCR-seg01.6..Recoded | CCAACCTCACGTGACAGAAATAG |
| mAsPCR-seg01.6..Reverse | GGATGACCGCAATTCTGAAAG |
| mAsPCR-seg01.6..Wild-Type | CCAACCTCACGACTCAGAAATAA |
| mAsPCR-seg01.7..Recoded | GCCCGCCAGGTTAAAAACT |
| mAsPCR-seg01.7..Reverse | CAAGAAAATTCAACATCATCGGTGTAAT |
| mAsPCR-seg01.7..Wild-Type | GCCCGCCAGGTTAAAAACA |
| mAsPCR-seg01.8..Recoded | TAGTAGTGGGATTGTAAGAACGCATC |
| mAsPCR-seg01.8..Reverse | TGGTTAAGCAAACGGAAGACATTC |
| mAsPCR-seg01.8..Wild-Type | TAGTAGTGGGATTGTAAGAACGCATA |
| mAsPCR-seg02.1..Recoded | GGAAGAACATGCCAACTTTATCTCA |
| mAsPCR-seg02.1..Reverse | CCACCGCGTTGTTCAGTTC |
| mAsPCR-seg02.1..Wild-Type | GGAAGAACATGCCAACTTTATCACT |
| mAsPCR-seg02.2..Recoded | GCAGATCTGATTGTCGCCTCA |
| mAsPCR-seg02.2..Reverse | TGTAGTTATGCTGCCCGGAAA |
| mAsPCR-seg02.2..Wild-Type | GCAGATCTGATTGTCGCCAGT |
| mAsPCR-seg02.3..Recoded | TGAAGAAGTACTTATTGAAAAATGGCTATCG |
| mAsPCR-seg02.3..Reverse | CAGCCTGACACTAGCACTGT |
| mAsPCR-seg02.3..Wild-Type | TGAAGAAGTATTGATTGAAAAATGGCTAAGT |
| mAsPCR-seg02.4..Recoded | TTTTATTCACGCGTTTATACATTTCCGAT |
| mAsPCR-seg02.4..Reverse | TGCGTACCGGTGAAGGAAAA |
| mAsPCR-seg02.4..Wild-Type | TTTTATTCACGCGTTTATATATTTCCGAG |
| mAsPCR-seg02.5..Recoded | GCAATGTATCTGCCAATTTTCCATC |
| mAsPCR-seg02.5..Reverse | CATGTCATCCGAGTCTGCGA |
| mAsPCR-seg02.5..Wild-Type | GCAATGTATCTGCCAATTTTCCATT |
| mAsPCR-seg02.6..Recoded | GGTGAGGGCAATAATCTTTACACG |
| mAsPCR-seg02.6..Reverse | TCTTGCGCGTGTGGTATATGC |
| mAsPCR-seg02.6..Wild-Type | GGTGAGGGCAATAATCTTTACACC |
| mAsPCR-seg02.7..Recoded | CAGCACGAAGATGGTCACTCA |
| mAsPCR-seg02.7..Reverse | GATACCTTCCTCAGCACCTTCC |
| mAsPCR-seg02.7..Wild-Type | CAGCACGAAGATGGTCACAGC |
| mAsPCR-seg02.8..Recoded | GCACATGGGGTTTAAACGGTAG |
| mAsPCR-seg02.8..Reverse | AAACTTCGTTAATTCGCATGGTGATAA |
| mAsPCR-seg02.8..Wild-Type | GCACATGGGGTTTAAACGGCAA |
| mAsPCR-seg03.1..Recoded | AGAGCCGAAAAGCACTGTTCG |
| mAsPCR-seg03.1..Reverse | GTTTTGGCAGCATTAGTTTCAGGA |
| mAsPCR-seg03.1..Wild-Type | GCTGCCGAATAACACTGTTCT |
| mAsPCR-seg03.2..Recoded | GGTGGTGCCTTTGTCGTTA |
| mAsPCR-seg03.2..Reverse | GGGACGATTTAAACCACAGATAAAGT |
| mAsPCR-seg03.2..Wild-Type | GGTGGTGCCTTTGTCGTTT |
| mAsPCR-seg03.3..Recoded | CAAAATCAAACAGAATATTGTGCTCTGA |
| mAsPCR-seg03.3..Reverse | CTGGCCTATATCTCTGCACTGG |
| mAsPCR-seg03.3..Wild-Type | CAAAATCAAACAGAATATTGTGCTCACT |
| mAsPCR-seg03.4..Recoded | TAGCATGCGAGAGTCTGAGTAAAGT |
| mAsPCR-seg03.4..Reverse | ATTATCCCTCAGGCTTCTGTTCG |
| mAsPCR-seg03.4..Wild-Type | CAACATGCGGCTGTCACTGTATAAA |
| mAsPCR-seg03.5..Recoded | GGTTACGCAGTTCGAGTGA |
| mAsPCR-seg03.5..Reverse | GCCTCATTTTTCCCCCGAAC |
| mAsPCR-seg03.5..Wild-Type | GGTTACGCAGTTCGAGGCT |
| mAsPCR-seg03.6..Recoded | CGACTTATCTGACGGCCCTATC |
| mAsPCR-seg03.6..Reverse | CGGATGTAGCTGATCTTTCGGTA |
| mAsPCR-seg03.6..Wild-Type | CGACTTATCTGACGGCCTTAAG |
| mAsPCR-seg03.7..Recoded | GTGGAGGATAGTCGGAATATGATG |
| mAsPCR-seg03.7..Reverse | GCCGCTAAACAGTCCTCACT |
| mAsPCR-seg03.7..Wild-Type | GTGGAGGATAGTCGGAATAGCTGC |
| mAsPCR-seg03.8..Recoded | ACGGTCATTAAAGTTCAACTGTCA |
| mAsPCR-seg03.8..Reverse | TTACCAATCGCTACGGTGTAATCA |
| mAsPCR-seg03.8..Wild-Type | ACGGTCATTAAAGTTCAACTGAGC |
| mAsPCR-seg04.1..Recoded | TTTGTGCGTCGTGAACTGAAAG |
| mAsPCR-seg04.1..Reverse | CCGTCAACTGAGCTGATTTTCATC |
| mAsPCR-seg04.1..Wild-Type | TTTGTGCGTCGTGAACACTTAA |
| mAsPCR-seg04.2..Recoded | CGTACTTCAGCATCTTTACGGATATCT |
| mAsPCR-seg04.2..Reverse | TCTTTACCACCGACTCAGCAG |
| mAsPCR-seg04.2..Wild-Type | CGTACTTCAGCATCTTTTCTGATATCG |
| mAsPCR-seg04.3..Recoded | ACATCGACTCTACCCAAGTTTCA |
| mAsPCR-seg04.3..Reverse | TCAACCTGGTCCGGTGAAC |
| mAsPCR-seg04.3..Wild-Type | ACATCGACTCTACCCAGGTCAGT |
| mAsPCR-seg04.4..Recoded | GAAGAGATCAAAGAGAAAGCGCTATC |
| mAsPCR-seg04.4..Reverse | AAGTCCCAGTGCGCGTTT |
| mAsPCR-seg04.4..Wild-Type | GAAGAGATCAAAGAGAAAGCGTTGAG |
| mAsPCR-seg04.5..Recoded | CGGCACCGCATATCAAAAATCT |
| mAsPCR-seg04.5..Reverse | ACTGGCACTACATCGTTCATCAT |
| mAsPCR-seg04.5..Wild-Type | CGGCACCGCATATCAAAAAAGC |
| mAsPCR-seg04.6..Recoded | GGCATTTACTTTATCACCGGGTTAG |
| mAsPCR-seg04.6..Reverse | CAGCTATCATCTGTGGGCGAA |
| mAsPCR-seg04.6..Wild-Type | GGCATTTACTTTATCACCGGGTCAA |
| mAsPCR-seg04.7..Recoded | GTAGTACTTTGGGATTTGAGGCAAG |
| mAsPCR-seg04.7..Reverse | TAACCTGCTCTCTTCGCGTAC |
| mAsPCR-seg04.7..Wild-Type | GCAATACTTTGGGATTGCTGGCTAA |
| mAsPCR-seg04.8..Recoded | CACCTCATGAAGTTGTCCATCTGA |
| mAsPCR-seg04.8..Reverse | GCCCGTCCGCTTTTTAACTC |
| mAsPCR-seg04.8..Wild-Type | CACCTCATGTAATTGTCCATCGCT |
| mAsPCR-seg05.1..Recoded | AAAGATCGTGCGGAAGAATGGA |
| mAsPCR-seg05.1..Reverse | CTTAAGCAGATGAAAACCATACATTTTAGTG |
| mAsPCR-seg05.1..Wild-Type | ACAAATCGTGCGGAAGAATACT |
| mAsPCR-seg05.2..Recoded | AAGACCTATAAAGCGATGGTAAAAGATCTA |
| mAsPCR-seg05.2..Reverse | GCCATATTATTTTTCCCTGCATTCAA |
| mAsPCR-seg05.2..Wild-Type | AAGACCTATAAAGCGATGGTAAAAGATTTG |
| mAsPCR-seg05.3..Recoded | GAGTTCCAGTTCGCTCAAATCGA |
| mAsPCR-seg05.3..Reverse | CCCAATGGCTGCTAACGC |
| mAsPCR-seg05.3..Wild-Type | GAGTTCCAGTTCTCTCAAATCGT |
| mAsPCR-seg05.4..Recoded | GCTCTGACTGAACCTTCACAG |
| mAsPCR-seg05.4..Reverse | CGTAGTGGGGATGCCAGATC |
| mAsPCR-seg05.4..Wild-Type | GCTCTCACTGAACCTTCACGC |
| mAsPCR-seg05.5..Recoded | CGGAAGAGGACTCACGCCTT |
| mAsPCR-seg05.5..Reverse | CATACAGCCAGACAATCGAAAAAGAA |
| mAsPCR-seg05.5..Wild-Type | CGGAAGAGGACTCACGCTTA |
| mAsPCR-seg05.6..Recoded | TCTACATGTAATACGGTTGAAACGCTA |
| mAsPCR-seg05.6..Reverse | GAGTGTTGTGTGCCGTGTTC |
| mAsPCR-seg05.6..Wild-Type | AGCACATGTAATACGGTTGAAACGTTG |
| mAsPCR-seg05.7..Recoded | ATGCTCTATCGTCTACAGCAAGTT |
| mAsPCR-seg05.7..Reverse | GGTGGGTAGATGCTGAGTGATAAA |
| mAsPCR-seg05.7..Wild-Type | ATGCTCTATCGTTTACAGCAGGTC |
| mAsPCR-seg05.8..Recoded | GGTAATTTCAGAATATGGTGGACAAAAAC |
| mAsPCR-seg05.8..Reverse | ATTCTCTTCGGTAAAAATTGAGTTCATTAAA |
| mAsPCR-seg05.8..Wild-Type | GGTAATTTCAGAATATGGTGGACAAAAAT |
| mAsPCR-seg06.1..Recoded | AGCTGATTGTTTTTAACCGTATTAAGTATAG |
| mAsPCR-seg06.1..Reverse | CTGGGGGCCGATGAAGTT |
| mAsPCR-seg06.1..Wild-Type | AACTGATTGTTTTTAACCGTATTAAGTATGC |
| mAsPCR-seg06.2..Recoded | GATTGCAGTGAGTGGCTGA |
| mAsPCR-seg06.2..Reverse | TTACCGATCTAGCAGAAGAAGCC |
| mAsPCR-seg06.2..Wild-Type | GATTGCAGTGAGTGGCGCT |
| mAsPCR-seg06.3..Recoded | CGGAAAGGGGTACTAGCACTT |
| mAsPCR-seg06.3..Reverse | GGAACGACCGCTTTTAGTGC |
| mAsPCR-seg06.3..Wild-Type | CGGAAAGGGGTATTGGCATTG |
| mAsPCR-seg06.4..Recoded | CCGTCAAAAGCTGCGATTG |
| mAsPCR-seg06.4..Reverse | TGAGCCTGGCGATCTGTTC |
| mAsPCR-seg06.4..Wild-Type | CCGTCAAAAGCTGCGATGC |
| mAsPCR-seg06.5..Recoded | CGCCGGGATATAACATGACGA |
| mAsPCR-seg06.5..Reverse | GCACTAGGTCACCAGCAAATC |
| mAsPCR-seg06.5..Wild-Type | CGGCGGGATATAACATGAGCT |
| mAsPCR-seg06.6..Recoded | CCATTGGACGTTTCACCTCA |
| mAsPCR-seg06.6..Reverse | GCGTCCCTGCTCCAGAAG |
| mAsPCR-seg06.6..Wild-Type | CCATTGGACGTTTCACCAGC |
| mAsPCR-seg06.7..Recoded | GGCGTCATTAATTTCATCCAGTGA |
| mAsPCR-seg06.7..Reverse | CTGGGGTCAGTCGGTGATC |
| mAsPCR-seg06.7..Wild-Type | GGCGTCATTAATTTCATCCAGGCT |
| mAsPCR-seg06.8..Recoded | GCGTGGTTATCAGCTAGTGTCA |
| mAsPCR-seg06.8..Reverse | GTGACTGCGGGCTTATCGA |
| mAsPCR-seg06.8..Wild-Type | GCGTGGTTATCAGTTGGTGAGC |
| mAsPCR-seg07.1..Recoded | TGAGGCTCAGTTAGTGTCGTC |
| mAsPCR-seg07.1..Reverse | TCGATGTTCCTGTCCTGCTG |
| mAsPCR-seg07.1..Wild-Type | TGAGGCTCAGTCAATGTCGTT |
| mAsPCR-seg07.2..Recoded | GCTGGCGCTTTCGGATCTA |
| mAsPCR-seg07.2..Reverse | GCAAAGCGCCACCAGAAAT |
| mAsPCR-seg07.2..Wild-Type | GCTGGCGCTTTCGGATCTG |
| mAsPCR-seg07.3..Recoded | GCCCAGGACGGTAGGATATCA |
| mAsPCR-seg07.3..Reverse | GTCTGGGCTGGCCTGATG |
| mAsPCR-seg07.3..Wild-Type | GCCCAGGACGGTAAGATATCG |
| mAsPCR-seg07.4..Recoded | GCGTGACTCCTGGTACGATC |
| mAsPCR-seg07.4..Reverse | CCCTGGCAAGTCGAAAAGC |
| mAsPCR-seg07.4..Wild-Type | GCGTGACTCCTGGTACGATT |
| mAsPCR-seg07.5..Recoded | TCAGGAAATCAATGTGCAGAATCAAC |
| mAsPCR-seg07.5..Reverse | TTTCGTTTCACAGTTCTATCATTTACGTAA |
| mAsPCR-seg07.5..Wild-Type | TCAGGAAATCAATGTGCAGAATCAAT |
| mAsPCR-seg07.6..Recoded | CGCATCAGAAAACGGCAGA |
| mAsPCR-seg07.6..Reverse | CGGGTGACTGGATCTATGTGAC |
| mAsPCR-seg07.6..Wild-Type | CGCATCAGAAAACGGCAGC |
| mAsPCR-seg07.7..Recoded | ATAATTTCTTGCGGATGATGACGAAG |
| mAsPCR-seg07.7..Reverse | CATTATTCATGTGGCAAACGGTATCA |
| mAsPCR-seg07.7..Wild-Type | ATAATTTCTTGCGGATGATGACGTAA |
| mAsPCR-seg07.8..Recoded | TGTAATGTCTCATTCTACCGATCACTC |
| mAsPCR-seg07.8..Reverse | AGAACCTGTACCACTGCCATTG |
| mAsPCR-seg07.8..Wild-Type | TGTAATGAGTCATTCTACCGATCACAG |
| mAsPCR-seg08.1..Recoded | CATGTTGTCCATCAGTTCTTTGTTTTTT |
| mAsPCR-seg08.1..Reverse | GACCGCGTAACCATCGACT |
| mAsPCR-seg08.1..Wild-Type | CATGTTGTCCATCAGTTCTTTGTTTTTG |
| mAsPCR-seg08.2..Recoded | GTCCCTTGATTTTGTTGACACGT |
| mAsPCR-seg08.2..Reverse | AAGCTGAACAAAAAAATCCCACCA |
| mAsPCR-seg08.2..Wild-Type | GTCCCTTGATTTTGTTGACACGG |
| mAsPCR-seg08.3..Recoded | AGCATTAGAAGTCGCTGGTGAAG |
| mAsPCR-seg08.3..Reverse | GTTTTTGCTCAGAACGCCATGT |
| mAsPCR-seg08.3..Wild-Type | AGCATCAATAATCGCTGGTGTAA |
| mAsPCR-seg08.4..Recoded | TCATTAGTGACGCGGGAAATG |
| mAsPCR-seg08.4..Reverse | GATGCATGAAAATCGCGAGGAG |
| mAsPCR-seg08.4..Wild-Type | TCATTAGTGACGCGGGAAATC |
| mAsPCR-seg08.5..Recoded | CCTGAGCAATTTCATCGGATGA |
| mAsPCR-seg08.5..Reverse | CGGGTATCTTACTCATATCGCTATATTCA |
| mAsPCR-seg08.5..Wild-Type | CCTGAGCAATTTCATCGCTGCT |
| mAsPCR-seg08.6..Recoded | CAGACACAGGAACACGACAATTAG |
| mAsPCR-seg08.6..Reverse | GGCGTTCTCCTCTTCTCGT |
| mAsPCR-seg08.6..Wild-Type | CAGACACAGGAACACGACAATCAA |
| mAsPCR-seg08.7..Recoded | ATACAGACGCAGCTCATGATCTAG |
| mAsPCR-seg08.7..Reverse | GTTTGTTACCGAGCGTCTGATC |
| mAsPCR-seg08.7..Wild-Type | ATACAGACGCAGCTCATGATCCAA |
| mAsPCR-seg08.8..Recoded | TCCGCGATGTCACCTCAC |
| mAsPCR-seg08.8..Reverse | CAACGCCCAGACCCAGAG |
| mAsPCR-seg08.8..Wild-Type | TCCGCGATGTCACCAGCT |
| mAsPCR-seg09.1..Recoded | GATAAGACACACGGTTAGCATATTTACAA |
| mAsPCR-seg09.1..Reverse | GCTATCTCACCAGGCCACAT |
| mAsPCR-seg09.1..Wild-Type | GATAGCTCACACGGTTAGCATATTTACAC |
| mAsPCR-seg09.2..Recoded | TATGAATATCTGGAACCGCTCGATCTA |
| mAsPCR-seg09.2..Reverse | GAAGGAATAAGTACATCATTGCGGAT |
| mAsPCR-seg09.2..Wild-Type | TATGAATATCTGGAACCGCTCGATTTG |
| mAsPCR-seg09.3..Recoded | CCAGACACCGGCAATAATCAGA |
| mAsPCR-seg09.3..Reverse | CATGATGAACACGGAAGGTAATAACG |
| mAsPCR-seg09.3..Wild-Type | CCAGACACCGGCAATAATCAGC |
| mAsPCR-seg09.4..Recoded | CGCATTAAAGCAGATAAAAAGCACCATA |
| mAsPCR-seg09.4..Reverse | ATGAAATAACCTCAGCGCTGCA |
| mAsPCR-seg09.4..Wild-Type | CGCATTAAAGCAGATAAATAACACCATC |
| mAsPCR-seg09.5..Recoded | TGTTTTTCCGTACGACTCGCT |
| mAsPCR-seg09.5..Reverse | CGCCTCAGTTCCCGTGAC |
| mAsPCR-seg09.5..Wild-Type | TGTTTTTCCGTACGACTCGCA |
| mAsPCR-seg09.6..Recoded | CGTTTCTCTGCTAATCTTTCGATGCTT |
| mAsPCR-seg09.6..Reverse | CTGCTACGCCATCCCGAAA |
| mAsPCR-seg09.6..Wild-Type | CGTTTCTCTGCTAATTTATCGATGTTA |
| mAsPCR-seg09.7..Recoded | TGTGTTTCGATATAACCGTGGGA |
| mAsPCR-seg09.7..Reverse | GGCCGAAGACTCACAAATCTTTC |
| mAsPCR-seg09.7..Wild-Type | TGTGTTTCGATATAACCGTGGCT |
| mAsPCR-seg09.8..Recoded | CTCTCAGCAGACGAGAAATCA |
| mAsPCR-seg09.8..Reverse | AGGCAAACCAGACATTCTCGT |
| mAsPCR-seg09.8..Wild-Type | CTCAGTGCAGACGAGAAAAGC |
| mAsPCR-seg10.1..Recoded | GCCAAGTACAGCGGAAAGTTTT |
| mAsPCR-seg10.1..Reverse | CAACTTATGGCGTGCTGTCG |
| mAsPCR-seg10.1..Wild-Type | GCCCAATACAGCGGAAAGTTTA |
| mAsPCR-seg10.2..Recoded | TGTAATGATGAATGACTTTTCTTTTACACCA |
| mAsPCR-seg10.2..Reverse | AATACATCCGCAATTCTCAAACCTG |
| mAsPCR-seg10.2..Wild-Type | TGTAATGATGAATGACTTTTCTTTTACACCG |
| mAsPCR-seg10.3..Recoded | GTCAGTTTATCCACGCCTGA |
| mAsPCR-seg10.3..Reverse | ACGTCTACAAGGCTTCGATACC |
| mAsPCR-seg10.3..Wild-Type | GTCAGTTTATCCACGCCGCT |
| mAsPCR-seg10.4..Recoded | TGATGCTGAACCGCATTGTAAAG |
| mAsPCR-seg10.4..Reverse | TGAAGAACAACTCGATACAGCACT |
| mAsPCR-seg10.4..Wild-Type | TGATGCTGAACCGCATTGTACAA |
| mAsPCR-seg10.5..Recoded | GAAGGTGAAAAGGTGGTTTCCTC |
| mAsPCR-seg10.5..Reverse | GGTTAGCGGATAAGTCACCTGAT |
| mAsPCR-seg10.5..Wild-Type | GAAGGTGAAAAGGTGGTTTCCAG |
| mAsPCR-seg10.6..Recoded | CACCTGATTTACCGCTTTTGGAATT |
| mAsPCR-seg10.6..Reverse | CGAGTTCTGGTTTGCGCTTATTAA |
| mAsPCR-seg10.6..Wild-Type | CACCTGATTTACCGCTTTTGGAATG |
| mAsPCR-seg10.7..Recoded | CGACCATTACCCCTTTCGGA |
| mAsPCR-seg10.7..Reverse | TGAAAATGATGCTGGAAGATGCG |
| mAsPCR-seg10.7..Wild-Type | CGACCATTACCCCTTTCGGC |
| mAsPCR-seg10.8..Recoded | ATAGAAGCTCCAGTAGATCAATCTGATGAG |
| mAsPCR-seg10.8..Reverse | CACGGGAATAACTCATCTGGCA |
| mAsPCR-seg10.8..Wild-Type | TTAACAACTCCAGCAAATCAATCTGATGAC |
| mAsPCR-seg11.1..Recoded | GGCTCATAACTACGCCATGTCA |
| mAsPCR-seg11.1..Reverse | GCCCATCAGCTCATCTTCCA |
| mAsPCR-seg11.1..Wild-Type | GGCTCATAACTACGCCATGAGT |
| mAsPCR-seg11.2..Recoded | GCGTGTATTTTGCCATGAACTCA |
| mAsPCR-seg11.2..Reverse | TGCGGTCAGGGTACAAATCAG |
| mAsPCR-seg11.2..Wild-Type | GCGTGTATTTTGCCATGAACAGC |
| mAsPCR-seg11.3..Recoded | CATATTTGATTTTAGCGATGGTTTCAGAT |
| mAsPCR-seg11.3..Reverse | GCAACACCTCAGCCTGCA |
| mAsPCR-seg11.3..Wild-Type | CATATTTGATTTTAGCGATGCTTTCAGAG |
| mAsPCR-seg11.4..Recoded | CAATAATTGACTGTGCCGGATCT |
| mAsPCR-seg11.4..Reverse | CGCTGCGCTCAATAAAAAACAG |
| mAsPCR-seg11.4..Wild-Type | CAATAATTGACTGTGCCGGATCG |
| mAsPCR-seg11.5..Recoded | CCTCGAAGACTCCGTAGCAC |
| mAsPCR-seg11.5..Reverse | ATTTCCACTGCGCGGGTAA |
| mAsPCR-seg11.5..Wild-Type | CCTCGAAGACTCCGTAGCAT |
| mAsPCR-seg11.6..Recoded | TGACAGCTCCACTTACCCTACTA |
| mAsPCR-seg11.6..Reverse | CAGACACCGTTTCCATATCCGA |
| mAsPCR-seg11.6..Wild-Type | TGACAGCTCCATTAACCCTATTG |
| mAsPCR-seg11.7..Recoded | GCTCCACGACTACTGGAAAATATTC |
| mAsPCR-seg11.7..Reverse | TCAATAGGTTAATGAATGGGGTGAGTTA |
| mAsPCR-seg11.7..Wild-Type | GCTCCACGTTTACTGGAAAATATTT |
| mAsPCR-seg11.8..Recoded | CGAAGACATAAACGAAAAGTATCAGCATAAG |
| mAsPCR-seg11.8..Reverse | TACTGACTTTATCTTCGCGGTACTG |
| mAsPCR-seg11.8..Wild-Type | CGAAGACATAAACGAATAATATCAGCATTAA |
| mAsPCR-seg12.1..Recoded | CGTAACGTTCAACCATGACTTGT |
| mAsPCR-seg12.1..Reverse | GCCATCGCCGATAAACTGAC |
| mAsPCR-seg12.1..Wild-Type | CGTAACGTTCAACCATCACCTGC |
| mAsPCR-seg12.2..Recoded | GGGTAGGGTAATACGCATCATCC |
| mAsPCR-seg12.2..Reverse | TTTGCACTTTCCACTCCGATG |
| mAsPCR-seg12.2..Wild-Type | AGGTAGGGTAATACGCATCATCA |
| mAsPCR-seg12.3..Recoded | CATAACCTATCACCAGCACCGTA |
| mAsPCR-seg12.3..Reverse | TATTTCGCGCTACTAGTGATGGTT |
| mAsPCR-seg12.3..Wild-Type | CATAACCTATCACCAGCACCGTT |
| mAsPCR-seg12.4..Recoded | CTTTAAGCGGGCCATCAATCTGA |
| mAsPCR-seg12.4..Reverse | GCTGGCCTTCTCTCCTTACG |
| mAsPCR-seg12.4..Wild-Type | CTTTTAACGGGCCATCAATCTGG |
| mAsPCR-seg12.5..Recoded | ATAATCAGGTCTGGATTCTTCTCTTTGAG |
| mAsPCR-seg12.5..Reverse | GATAACGCTCATACTGGTCACAAC |
| mAsPCR-seg12.5..Wild-Type | ATAATCAGGTCTGGATTCTTCTCTTTTAA |
| mAsPCR-seg12.6..Recoded | GACTGGTCCGGTATTTATGCCT |
| mAsPCR-seg12.6..Reverse | CCCTGTAGGTCGTCGAGAAAT |
| mAsPCR-seg12.6..Wild-Type | GACTGGTCCGGTATTTATGCCA |
| mAsPCR-seg12.7..Recoded | GCGATCAATCCAAATCTCACCT |
| mAsPCR-seg12.7..Reverse | TGACCAAGCAGGACAACAC |
| mAsPCR-seg12.7..Wild-Type | GCGATCAATCCAAATCTCACCG |
| mAsPCR-seg12.8..Recoded | CGTTTGTATAGATCTTCCGCCGAT |
| mAsPCR-seg12.8..Reverse | GAGCAAATTCTGTCACTTCTTCTAATGAA |
| mAsPCR-seg12.8..Wild-Type | CGTTTGTATAAATCTTCCGCACTG |
| mAsPCR-seg13.1..Recoded | GCTTCTTGCGGATTCATCGAT |
| mAsPCR-seg13.1..Reverse | CTCCACCTCACCGTTCTATCC |
| mAsPCR-seg13.1..Wild-Type | GCTTCTTGCGGATTCATGCTG |
| mAsPCR-seg13.2..Recoded | AAAAAAACGTCGGGCAATTCTCT |
| mAsPCR-seg13.2..Reverse | GCTACCCGCGCCTGATAAC |
| mAsPCR-seg13.2..Wild-Type | AAAAAAACGTCGGGCAATTCTCA |
| mAsPCR-seg13.3..Recoded | GGTGTGTGAAGGATTTGATGACTCT |
| mAsPCR-seg13.3..Reverse | TGTTTACAAAGCGAGGGGTGATA |
| mAsPCR-seg13.3..Wild-Type | GGTGTGTGAAGGATTTGATGACAGC |
| mAsPCR-seg13.4..Recoded | TGGAATACGTGGTCTGGTTTCTT |
| mAsPCR-seg13.4..Reverse | GGCGTCATTACCCACCAGT |
| mAsPCR-seg13.4..Wild-Type | TGGAATACGTGGTCTGGTTTTTA |
| mAsPCR-seg13.5..Recoded | GGCATTCAGGTTAGTAGAGGAC |
| mAsPCR-seg13.5..Reverse | TTAACTGGCAAAAAAAGGGTGACA |
| mAsPCR-seg13.5..Wild-Type | GGCATTCAGGTTAGTGCTGCTG |
| mAsPCR-seg13.6..Recoded | GCAGGAGTCCTCGTATGGTATC |
| mAsPCR-seg13.6..Reverse | CGTAGTCGGTTAGAACTTGCCA |
| mAsPCR-seg13.6..Wild-Type | GCAGGAGTCCTCGTATGGTAAG |
| mAsPCR-seg13.7..Recoded | TGCCGTTGTTGACCGTTCA |
| mAsPCR-seg13.7..Reverse | CCATGAAGATTTTGGTGAACTGCT |
| mAsPCR-seg13.7..Wild-Type | TGCCGTTGTTGACCGTAGT |
| mAsPCR-seg13.8..Recoded | GAATCCATTGAATTTTGATGAAAGACGT |
| mAsPCR-seg13.8..Reverse | GGCTATACCGCCTATTCTCTGG |
| mAsPCR-seg13.8..Wild-Type | GAATCCATTGAATTTACTGCTAAGACGC |
| mAsPCR-seg14.1..Recoded | CTGATGTCTAAGATTATCGCGACTCTA |
| mAsPCR-seg14.1..Reverse | TTGCGTGAAAACAAGAGAGGTG |
| mAsPCR-seg14.1..Wild-Type | CTGATGAGTAAGATTATCGCGACTTTG |
| mAsPCR-seg14.2..Recoded | CAGACGGTAAATTTATGGTAATGGTTTC |
| mAsPCR-seg14.2..Reverse | GTGACTTTGTAAGACGGGTTAGAAC |
| mAsPCR-seg14.2..Wild-Type | GCGACGGTAAATTTATGGTAATGGTCAG |
| mAsPCR-seg14.3..Recoded | GTCGAACTTATTGATCATCTTGATTCCC |
| mAsPCR-seg14.3..Reverse | GCTCTCGCAGTCGTTCAT |
| mAsPCR-seg14.3..Wild-Type | GTCGAACTTATTGATCATCTTGATAGTT |
| mAsPCR-seg14.4..Recoded | CATCTGGGATATCAAAAAGCATATCGGTTAT |
| mAsPCR-seg14.4..Reverse | CAAGACGATGGGTAATACAGGCA |
| mAsPCR-seg14.4..Wild-Type | CATCTGGGATATCAAAAAGCATATCGGTTAC |
| mAsPCR-seg14.5..Recoded | TACCAATGGCTCGTAAATGGCTA |
| mAsPCR-seg14.5..Reverse | TGCCGAGCAGTGTCTGAC |
| mAsPCR-seg14.5..Wild-Type | TACCAATGGCTCGTAAATGGTTG |
| mAsPCR-seg14.6..Recoded | AAATGTTCTTCGGCAATTATTTCGTTATTC |
| mAsPCR-seg14.6..Reverse | TGGAACATGCTGTAAATATTCTCGTC |
| mAsPCR-seg14.6..Wild-Type | AAATGTTCTTCGGCAATTATTTCGTTATTA |
| mAsPCR-seg14.7..Recoded | TCGGAGTAATCGAGGCTGA |
| mAsPCR-seg14.7..Reverse | GGTTTGGCTCTGGTCTGGTAG |
| mAsPCR-seg14.7..Wild-Type | TCGCAGTAATCGAGGCGCT |
| mAsPCR-seg14.8..Recoded | AGAGATCGAGGGCCGTTACT |
| mAsPCR-seg14.8..Reverse | CAGCCGCACACTATGAGC |
| mAsPCR-seg14.8..Wild-Type | AGAGATCTAAGGCCGTCACC |
| mAsPCR-seg15.1..Recoded | CGGTGTCGAAATGGAAGCACTC |
| mAsPCR-seg15.1..Reverse | CGATGCGCAGAGGTGACA |
| mAsPCR-seg15.1..Wild-Type | CGGTGTCGAAATGGAAGCATTA |
| mAsPCR-seg15.2..Recoded | TGTTTAGCCTCTGGACCGTAAG |
| mAsPCR-seg15.2..Reverse | CGGACTGGATGAGATTTTTACCC |
| mAsPCR-seg15.2..Wild-Type | TGTTTAGCCTCTGGACCGTAGC |
| mAsPCR-seg15.3..Recoded | CGAAAACGTCCGTGATTACTCA |
| mAsPCR-seg15.3..Reverse | GATGCCATCTTTATTGAGCTGTTCA |
| mAsPCR-seg15.3..Wild-Type | CGAAAACGTCCGTGATTACAGC |
| mAsPCR-seg15.4..Recoded | CAACCTGACGCCGCTACTT |
| mAsPCR-seg15.4..Reverse | GATTAGCATACACTTCACCTTCAGTAC |
| mAsPCR-seg15.4..Wild-Type | CAACCTGACGCCGTTGTTG |
| mAsPCR-seg15.5..Recoded | CCGTCTGAACCTTTATGCATGGA |
| mAsPCR-seg15.5..Reverse | CTGTTCCGCACTGATATCGAAAATG |
| mAsPCR-seg15.5..Wild-Type | CCGTCTGAACCTTTATGCATACT |
| mAsPCR-seg15.6..Recoded | CCATCACAAGCAGGCCAGA |
| mAsPCR-seg15.6..Reverse | CGCGGATAAAAAACTTGTTGTCG |
| mAsPCR-seg15.6..Wild-Type | CCATCACTAACAGGCCGCT |
| mAsPCR-seg15.7..Recoded | CAGCAAATATAAGACCGTTAACTGAT |
| mAsPCR-seg15.7..Reverse | CGTTTTGCTAAGGATGTCATCGTC |
| mAsPCR-seg15.7..Wild-Type | CAGCAAATATCAAACCGTTAACGCTG |
| mAsPCR-seg15.8..Recoded | CGAACTGCATGGTGACGTTAG |
| mAsPCR-seg15.8..Reverse | ATTCCAGCTCACAGTGAAATCAGA |
| mAsPCR-seg15.8..Wild-Type | CGAACTGCATGGTGACGTTAC |
| mAsPCR-seg16.1..Recoded | CGGTCACAGTCTGAATGCCT |
| mAsPCR-seg16.1..Reverse | GTGCGTCATACAGCAGATCCT |
| mAsPCR-seg16.1..Wild-Type | CGGTCACAGTCTGAATGCCG |
| mAsPCR-seg16.2..Recoded | GGTCCGCAATCTCTCTTTTTCA |
| mAsPCR-seg16.2..Reverse | CTGCCACCACGCCCATAT |
| mAsPCR-seg16.2..Wild-Type | GGTCCGCAATCTCTCTTTTAGT |
| mAsPCR-seg16.3..Recoded | GCAATAATCACGTTAGCAATGCCT |
| mAsPCR-seg16.3..Reverse | GTACAAGTAAGGATGCGACTATTTAACTG |
| mAsPCR-seg16.3..Wild-Type | GCAATAATCACGTTAGCAATGCCG |
| mAsPCR-seg16.4..Recoded | TCCGGTGGTGTACGGACAAG |
| mAsPCR-seg16.4..Reverse | ACTTTACTTCACCATCGGAGTCC |
| mAsPCR-seg16.4..Wild-Type | TCCGGTGGTGTTCTGACTAA |
| mAsPCR-seg16.5..Recoded | CTGGGAGGGGATGTTTGTTCTA |
| mAsPCR-seg16.5..Reverse | CGCAAGCAGAAGGTTACCC |
| mAsPCR-seg16.5..Wild-Type | CTGGGAGGGGATGTTTGTTTTG |
| mAsPCR-seg16.6..Recoded | GTTCGAGATGCTGGGGTCA |
| mAsPCR-seg16.6..Reverse | CGGAAAGCGTCAATCACTGA |
| mAsPCR-seg16.6..Wild-Type | GTTCGAGATGCTGGGGAGC |
| mAsPCR-seg16.7..Recoded | CTGCCATTTCTGATTGTCTTTAAAATATCA |
| mAsPCR-seg16.7..Reverse | GCCGATCAGTAGACAGCAAAATG |
| mAsPCR-seg16.7..Wild-Type | CTGCCATTTCTGATTGTCTTTAAAATAAGC |
| mAsPCR-seg16.8..Recoded | CAGGGACGGGATCAGTGA |
| mAsPCR-seg16.8..Reverse | TCTGCCGCAGAGAAAATCAATTT |
| mAsPCR-seg16.8..Wild-Type | CAGGGACGGGATCAGGCT |
| mAsPCR-seg17.1..Recoded | TGAGAGATCGACTTTATGGCATGAC |
| mAsPCR-seg17.1..Reverse | AATACCTGAAAGAAGCATGGGAATTTAC |
| mAsPCR-seg17.1..Wild-Type | GCTGAGATCGACTTTATGGCAACTG |
| mAsPCR-seg17.2..Recoded | GACAAACTCCTTACGCTGAAAG |
| mAsPCR-seg17.2..Reverse | GGTGATGATTTCTCTGCGGTTATC |
| mAsPCR-seg17.2..Wild-Type | GACAAACTCCTTACGCGCTCAA |
| mAsPCR-seg17.3..Recoded | AGAATTACCTGACCACCGTTCATT |
| mAsPCR-seg17.3..Reverse | CAAACCAGGAGCTGCACAATG |
| mAsPCR-seg17.3..Wild-Type | AGAATTACCTGACCACCGTTCATC |
| mAsPCR-seg17.4..Recoded | TATTGCACGCATTCCAGAGAAGTC |
| mAsPCR-seg17.4..Reverse | GGGTGCGCTTTCTCGATTTC |
| mAsPCR-seg17.4..Wild-Type | TATTGCACGCATTCCAGAGAAGAG |
| mAsPCR-seg17.5..Recoded | CATCTGCGCATTTACACCTTCT |
| mAsPCR-seg17.5..Reverse | GTCCGCCAAGATGAGTCAGAT |
| mAsPCR-seg17.5..Wild-Type | CATCTGCGCATTTACACCTTCA |
| mAsPCR-seg17.6..Recoded | ATACAGAGAGACAATAATAATGGTAGATTCT |
| mAsPCR-seg17.6..Reverse | GCGCCACGATTCAGAGTAATC |
| mAsPCR-seg17.6..Wild-Type | ATACAGAGAGACAATAATAATGGTAGATAGC |
| mAsPCR-seg17.7..Recoded | CCGATCGCTGTCGTTTTTACT |
| mAsPCR-seg17.7..Reverse | TTCGAGTGAAAATCTACCTATCTCTTT |
| mAsPCR-seg17.7..Wild-Type | CCGATCGCTGTCGTTTTTACC |
| mAsPCR-seg17.8..Recoded | CTGGCGGATCGTGCTTCTA |
| mAsPCR-seg17.8..Reverse | GCCATCCCCACGCTCATAT |
| mAsPCR-seg17.8..Wild-Type | CTGGCGGATCGTGCTTTTG |
| mAsPCR-seg18.1..Recoded | TCGTACCCTGGTTACCAAAAACT |
| mAsPCR-seg18.1..Reverse | CCAGGTCAACAGCCAGCT |
| mAsPCR-seg18.1..Wild-Type | TCGTACCCTGGTTACCAAAAACA |
| mAsPCR-seg18.2..Recoded | CCGCAAAAAAGTAGTTGGTTGATAGT |
| mAsPCR-seg18.2..Reverse | CCATCGGCACATCATCATAAAACG |
| mAsPCR-seg18.2..Wild-Type | CCGCAAAAAAGTAGTTGGTTGAGAGA |
| mAsPCR-seg18.3..Recoded | CTTAATGCCTATAAAGCAGCAACACTATCT |
| mAsPCR-seg18.3..Reverse | TGGGTTGAGATGCCACGTTT |
| mAsPCR-seg18.3..Wild-Type | TTAAATGCCTATAAAGCAGCAACATTAAGC |
| mAsPCR-seg18.4..Recoded | GCTGAATCTTATCCGCTGCTTCTA |
| mAsPCR-seg18.4..Reverse | GTTCAAGCTGAGCAACGTCAC |
| mAsPCR-seg18.4..Wild-Type | GCTGAATCTTATCCGCTGTTATTG |
| mAsPCR-seg18.5..Recoded | GTTTCATAGCCAACACGATCTGA |
| mAsPCR-seg18.5..Reverse | GGTGTCTACAGCGGAAGTAGG |
| mAsPCR-seg18.5..Wild-Type | GTTTCATAGCCAACACGATCGCT |
| mAsPCR-seg18.6..Recoded | CTGACGACCACACATCATATTAAGT |
| mAsPCR-seg18.6..Reverse | GCCGCCTTTTCTTTTTCCGA |
| mAsPCR-seg18.6..Wild-Type | CTGACGACCACACATCATATTAAGC |
| mAsPCR-seg18.7..Recoded | CTTGACTTCGATGCACTGATTAACT |
| mAsPCR-seg18.7..Reverse | GTCCTTCAGCATCTTCTTCCAGA |
| mAsPCR-seg18.7..Wild-Type | CTTGACTTCGATGCACTGATTAACA |
| mAsPCR-seg18.8..Recoded | CGATTAGCTCCCTGATGATATTACGA |
| mAsPCR-seg18.8..Reverse | GTAAAACCCCTGAATATTGTCATTAAGCT |
| mAsPCR-seg18.8..Wild-Type | CGATTAGCTCCCTGATGATATTAACT |
| mAsPCR-seg19.1..Recoded | GATTTTGCCAGCACCATACCAATTGA |
| mAsPCR-seg19.1..Reverse | AATTGGTTATAAGGAGAGAGTATGCGT |
| mAsPCR-seg19.1..Wild-Type | CTTTTTGCCAGCACCATACCAATACT |
| mAsPCR-seg19.2..Recoded | CGGTTCGTTTTATCTATCAGGTTCA |
| mAsPCR-seg19.2..Reverse | TATATCCGCGCCAGTCAGTTTT |
| mAsPCR-seg19.2..Wild-Type | CGGTTCGTTTTATTTAAGTGGTAGC |
| mAsPCR-seg19.3..Recoded | CGGATCTGCTATCGTGCCTT |
| mAsPCR-seg19.3..Reverse | AACAGACCAGTATCGAGATAATCCG |
| mAsPCR-seg19.3..Wild-Type | CGGATCTGCTAAGCTGCTTG |
| mAsPCR-seg19.4..Recoded | CCGACTCAGAACGTATGCATCTT |
| mAsPCR-seg19.4..Reverse | GCCACCTTCAATTCCTTCCG |
| mAsPCR-seg19.4..Wild-Type | GCGACAGTGAAAGAATGCATTTG |
| mAsPCR-seg19.5..Recoded | TGAACAAGAAACACTTCCGCTTT |
| mAsPCR-seg19.5..Reverse | AATTCACCATCGCCAATATGCAC |
| mAsPCR-seg19.5..Wild-Type | TGAACAAGAAACACTTCCGCTTA |
| mAsPCR-seg19.6..Recoded | CGATCACTTTTTGGCTCTTACTCT |
| mAsPCR-seg19.6..Reverse | GGGTATTGCGCGTAGATTTCTC |
| mAsPCR-seg19.6..Wild-Type | CGATCATTGTTTGGCAGTTACAGC |
| mAsPCR-seg19.7..Recoded | GCAAAAAGATGGCCTCGACT |
| mAsPCR-seg19.7..Reverse | GTCAGCTCCATTCCTTCTTTTTTACG |
| mAsPCR-seg19.7..Wild-Type | GCAAAAAGATGGCCTCGACA |
| mAsPCR-seg19.8..Recoded | ATGATTTCGGCCAAGAGGAGAGT |
| mAsPCR-seg19.8..Reverse | CGCCAATATCATCCGCAACATT |
| mAsPCR-seg19.8..Wild-Type | ATGATTTCGGCCAAGAGGAGAGA |
| mAsPCR-seg20.1..Recoded | GGTAACTGAATGCTCTTTTTTATGCATTAA |
| mAsPCR-seg20.1..Reverse | CTTAAACGTGAGAAACAGGACGAATC |
| mAsPCR-seg20.1..Wild-Type | GGTAACTGAATGCTCTTTTTTATGCATTAC |
| mAsPCR-seg20.2..Recoded | CGCTTTATTTTCTCTGAATCCTGGGA |
| mAsPCR-seg20.2..Reverse | GGAGGTTGGATCTTGTTTTTGTCTAC |
| mAsPCR-seg20.2..Wild-Type | CGCTTTATTTTCTCGCTATCCTGACT |
| mAsPCR-seg20.3..Recoded | CCAGCTACCGGATATGTCTTCA |
| mAsPCR-seg20.3..Reverse | GCCGATCCAACCGTTAGC |
| mAsPCR-seg20.3..Wild-Type | CCAGTTACCGGATATGAGTAGC |
| mAsPCR-seg20.4..Recoded | GAATTTTCTTGTTGTTCTTTCAGATTCA |
| mAsPCR-seg20.4..Reverse | CTATATACATCTTCAAAAACAGGCAAGGTT |
| mAsPCR-seg20.4..Wild-Type | GAATTTTCTTGTTGTTCTTTCAGATAGC |
| mAsPCR-seg20.5..Recoded | TCCCGGAGTGTTTCATCTGAT |
| mAsPCR-seg20.5..Reverse | GCAAATCATCTGCGCCTCTG |
| mAsPCR-seg20.5..Wild-Type | TCCCGTAACGTCTCATCGCTG |
| mAsPCR-seg20.6..Recoded | GACGGCGCTTTACCCAGT |
| mAsPCR-seg20.6..Reverse | GGCAAACCCGGAAAACCG |
| mAsPCR-seg20.6..Wild-Type | GACGGCGCTTTACCCAGC |
| mAsPCR-seg20.7..Recoded | GCTTCCTGACAGTACAAAAACGACTA |
| mAsPCR-seg20.7..Reverse | CCTACCAAACCCGCACTGATT |
| mAsPCR-seg20.7..Wild-Type | GCTTCCTGACAGTACAAAAAAGGCTC |
| mAsPCR-seg20.8..Recoded | CCTGAAGAGAAGATTTAGTGATGAGTAGA |
| mAsPCR-seg20.8..Reverse | CCATTTAGGGCTGATTTATTACTACACAC |
| mAsPCR-seg20.8..Wild-Type | CCTGCAAAGAAGATTTAGTGATCAACAAT |
| mAsPCR-seg21.1..Recoded | GTTATGCCGCGATCGTGAAG |
| mAsPCR-seg21.1..Reverse | ATATCACCGACTTTTCCCGTCTTAA |
| mAsPCR-seg21.1..Wild-Type | GTTATGCCGCGATCGTGTAA |
| mAsPCR-seg21.2..Recoded | CTGGCACAAAATATCTGGCAGTTTC |
| mAsPCR-seg21.2..Reverse | AAGACATTGGGATTAGCAGCAGTA |
| mAsPCR-seg21.2..Wild-Type | CTGGCACAAAATATCTGGCAGTTTT |
| mAsPCR-seg21.3..Recoded | GTCAAACCAGCCAAAAACCGA |
| mAsPCR-seg21.3..Reverse | TCTGATGCTGAACCCACTAAACTTAT |
| mAsPCR-seg21.3..Wild-Type | GTCAAACCAGCCAAAAACGCT |
| mAsPCR-seg21.4..Recoded | GTCGAGGACTACCATGAACAAGTTTC |
| mAsPCR-seg21.4..Reverse | GTTTGCATCACCGTTTGCATTTT |
| mAsPCR-seg21.4..Wild-Type | GTCGAGGACTACCATGAACAAGTTTT |
| mAsPCR-seg21.5..Recoded | CAGTGTTTCAGACGGAATGAGAG |
| mAsPCR-seg21.5..Reverse | AACTACTCTGCTCATGGTCGTC |
| mAsPCR-seg21.5..Wild-Type | CAGTGTTTCAGACGGAAGCTTAA |
| mAsPCR-seg21.6..Recoded | GTAATGCCAAATCCTTCAGACTTAAATGA |
| mAsPCR-seg21.6..Reverse | GGTATGTGTTCTTGATGGCGAAAT |
| mAsPCR-seg21.6..Wild-Type | GTAATGCCAAATCCTTCACTCTTAAAGCT |
| mAsPCR-seg21.7..Recoded | TACAAATAACCATCTCATCTGCCTGA |
| mAsPCR-seg21.7..Reverse | TTGACTCAGAAGGGTGGGTTAC |
| mAsPCR-seg21.7..Wild-Type | TACAAATAACCATCTCATCTGCCTGC |
| mAsPCR-seg21.8..Recoded | GCGATCGTAGGAGTTTGATGA |
| mAsPCR-seg21.8..Reverse | GACCGCTACAACTCAGAAAAGAC |
| mAsPCR-seg21.8..Wild-Type | GCGATCGTAACTGTTGCTGCT |
| mAsPCR-seg22.1..Recoded | CAATAATCGTAAAGGGGCAGTTTC |
| mAsPCR-seg22.1..Reverse | GCTGTAGATGCGGGGAGATATT |
| mAsPCR-seg22.1..Wild-Type | CAATAATCGTAAAGGGGCCGTCAG |
| mAsPCR-seg22.2..Recoded | CTTTCATCCATGTCATTTGCCTCA |
| mAsPCR-seg22.2..Reverse | GGTATCGTCTGGCTGTATTCGT |
| mAsPCR-seg22.2..Wild-Type | TTAAGCTCCATGTCATTTGCCAGC |
| mAsPCR-seg22.3..Recoded | TGTCTTTCACCGCCATCACA |
| mAsPCR-seg22.3..Reverse | GCACTTCCCTCGTTTGTCCA |
| mAsPCR-seg22.3..Wild-Type | TGTCTTTCACCGCCATCACT |
| mAsPCR-seg22.4..Recoded | GCTTCTGATAATACTCTTCATAAATTGAGGA |
| mAsPCR-seg22.4..Reverse | GCAGCCTTTAACTCCGATAACC |
| mAsPCR-seg22.4..Wild-Type | GCTTCTGATAATACTCTTCATAAATGCTGCT |
| mAsPCR-seg22.5..Recoded | GGGCTTATCAATGTGACCCTATCA |
| mAsPCR-seg22.5..Reverse | CGGTCATGATTTCTGCAATACCTG |
| mAsPCR-seg22.5..Wild-Type | GGGCTTATCAATGTGACCTTAAGT |
| mAsPCR-seg22.6..Recoded | CAGTTTGATCACTTCGTCATTAATAGAGAG |
| mAsPCR-seg22.6..Reverse | CGGTCTGTCACTGATTCGC |
| mAsPCR-seg22.6..Wild-Type | CAGTTTGATCACTTCGTCATTAATAGATAA |
| mAsPCR-seg22.7..Recoded | GAACCACAGAGAGAGTGAATGATGA |
| mAsPCR-seg22.7..Reverse | TGATTGACAAGGGTATTTTTTAAGCTATGAA |
| mAsPCR-seg22.7..Wild-Type | GAACCACAGATAAAGTGAAGCTACT |
| mAsPCR-seg22.8..Recoded | GGCGCTCGATCTGACACTT |
| mAsPCR-seg22.8..Reverse | TACGGACAGTGACAGCGTTG |
| mAsPCR-seg22.8..Wild-Type | GGCGCTCGATCTGACATTG |
| mAsPCR-seg23.1..Recoded | GGAACGTTTTATGCTGGAGTTTCTC |
| mAsPCR-seg23.1..Reverse | TCTGCCGGGTGATCTTGC |
| mAsPCR-seg23.1..Wild-Type | GGAACGTTTTATGCTGGAGTTTTTG |
| mAsPCR-seg23.2..Recoded | CGGTGATGACGCTATCTTCA |
| mAsPCR-seg23.2..Reverse | CCATCAAGGGTAAAGCGTGATTTATC |
| mAsPCR-seg23.2..Wild-Type | CGGTGATGACCCTAACCAGT |
| mAsPCR-seg23.3..Recoded | AAACAAAGAAAGATACAGGCTGGAATAAG |
| mAsPCR-seg23.3..Reverse | GTATCCCACTCAGCCCTAATCG |
| mAsPCR-seg23.3..Wild-Type | AAACACAAAAAGATACAGGCTGGAATTAA |
| mAsPCR-seg23.4..Recoded | TAGATGACGGTTAGTTTCAGCGAGA |
| mAsPCR-seg23.4..Reverse | TGGAAGATGCCTGGGAATATATGG |
| mAsPCR-seg23.4..Wild-Type | TAAATGACGGTTAGTTTCAGCGAGC |
| mAsPCR-seg23.5..Recoded | GAGAATGGCACCGACGAAAATT |
| mAsPCR-seg23.5..Reverse | GTCAAGGTGTTCAGGCGTTTATTT |
| mAsPCR-seg23.5..Wild-Type | GAGAATGGCACCGACGAAAATA |
| mAsPCR-seg23.6..Recoded | TGCCGCAGTTTTCATTAGGAG |
| mAsPCR-seg23.6..Reverse | CATCAAGCTCAAAATGGATAACTGG |
| mAsPCR-seg23.6..Wild-Type | TGCCGCAGTTTTCATCAACAA |
| mAsPCR-seg23.7..Recoded | CGGACAACTGAAAAGGCTGATG |
| mAsPCR-seg23.7..Reverse | ATTTTTTACATTTTCGATAAATTCATCTGCA |
| mAsPCR-seg23.7..Wild-Type | CGGACAACACTAAAGGCGCTAC |
| mAsPCR-seg23.8..Recoded | CTCTACGTGCTGATTAACCTGTTGT |
| mAsPCR-seg23.8..Reverse | GCATGGCTCCCGAAAATCAT |
| mAsPCR-seg23.8..Wild-Type | CTCTACGTGCTGATTAACCTGTTGA |
| mAsPCR-seg24.1..Recoded | TGTGAGGAGTGGTTATAGAAATAAGAAGTT |
| mAsPCR-seg24.1..Reverse | GAAAACTGTCGCCTTTAATACCAATG |
| mAsPCR-seg24.1..Wild-Type | TGGCTGGAGTGGTTATAGAAATAAGAAGTG |
| mAsPCR-seg24.2..Recoded | GATGCCATCGATGTGACCTC |
| mAsPCR-seg24.2..Reverse | TTCTTCCCAGACAGCATCCAG |
| mAsPCR-seg24.2..Wild-Type | GATGCCATCGATGTGACCAG |
| mAsPCR-seg24.3..Recoded | CGTTCCTGGTAATTGTATGAAGATTGT |
| mAsPCR-seg24.3..Reverse | AGCCCTATTTACACCGATGATTTC |
| mAsPCR-seg24.3..Wild-Type | CGTTCCTGGTAATTGTATGAAGATTGC |
| mAsPCR-seg24.4..Recoded | ACTGCTATCTTCAAATCGCTGATCT |
| mAsPCR-seg24.4..Reverse | AACAGAGTCAACAACAACAACAGAC |
| mAsPCR-seg24.4..Wild-Type | ACTGCTATCTTCAAATCGCTGATCA |
| mAsPCR-seg24.5..Recoded | GCGCCAGTTGTTTCAGGTATG |
| mAsPCR-seg24.5..Reverse | CCTATACCCGGAATATGTACATTGTGA |
| mAsPCR-seg24.5..Wild-Type | GCGCCAGTTGTTTCAGGTAGC |
| mAsPCR-seg24.6..Recoded | TCCTGTTCTGGAGGGGTCA |
| mAsPCR-seg24.6..Reverse | GGCAGGAACATGTTGATTTCGATC |
| mAsPCR-seg24.6..Wild-Type | TCCTGTTCTGGAGGGGAGT |
| mAsPCR-seg24.7..Recoded | CACGTTCAGTCATTAAAGATTCCATGT |
| mAsPCR-seg24.7..Reverse | CCATTTGCTTTTCCTCATTTAGAATCG |
| mAsPCR-seg24.7..Wild-Type | CACGTTCAGTCATTAAAGATTCCATGA |
| mAsPCR-seg24.8..Recoded | GGCACAACGTGACGGTAATCT |
| mAsPCR-seg24.8..Reverse | GCCACATACTTTATTCTCACCCAGA |
| mAsPCR-seg24.8..Wild-Type | GGCACAACGTGACGGTAATCA |
| mAsPCR-seg25.1..Recoded | CGGGGCCAATACCTCACTAC |
| mAsPCR-seg25.1..Reverse | CGGCATATTCACGTTCAACTTCA |
| mAsPCR-seg25.1..Wild-Type | CGGGGCCAATACCAGTTTGT |
| mAsPCR-seg25.2..Recoded | TCAACACCTCAGATGAAGTTATTCTTTCT |
| mAsPCR-seg25.2..Reverse | TCTATTGCCAGATTGACGAAAGC |
| mAsPCR-seg25.2..Wild-Type | TCAACACCAGTGATGAAGTTATTCTTAGC |
| mAsPCR-seg25.3..Recoded | TTACTTTAGCATATTACGAATGACATAATGT |
| mAsPCR-seg25.3..Reverse | GCACCTTCGCCAATATTCGC |
| mAsPCR-seg25.3..Wild-Type | TTACTTCAACATATTACGAATGACATAATGC |
| mAsPCR-seg25.4..Recoded | GCGGGAAGAAGATGAAGCAGTA |
| mAsPCR-seg25.4..Reverse | TTACCACCTAAATGAAGCGGAAGA |
| mAsPCR-seg25.4..Wild-Type | GCGGGAAGAAGATGAAGCAGTT |
| mAsPCR-seg25.5..Recoded | ATTTCACTTTCCCTTCTCGAAAAGC |
| mAsPCR-seg25.5..Reverse | TCTGCGTTGATGATTTTTCGTGTT |
| mAsPCR-seg25.5..Wild-Type | ATTTCACTTTCCCTTCTCGAAAAGT |
| mAsPCR-seg25.6..Recoded | TGAAAGCATTTGAAGGTCATGCGA |
| mAsPCR-seg25.6..Reverse | CCGTGCCATTGAACTGCTG |
| mAsPCR-seg25.6..Wild-Type | GCTAAGCATTTGTAAGTCATGGCT |
| mAsPCR-seg25.7..Recoded | CGCTACGACCGGGAAAAG |
| mAsPCR-seg25.7..Reverse | GAAGAAGCAGGTCTGGGTCAG |
| mAsPCR-seg25.7..Wild-Type | CGCTACGACCGGGAACAA |
| mAsPCR-seg25.8..Recoded | ATTCACTGAACTGAAAACCATCTGGATATC |
| mAsPCR-seg25.8..Reverse | GGAGAGCCCGGTATAGCC |
| mAsPCR-seg25.8..Wild-Type | ATTCACTGAACTGAAAACCATCTGGATAAG |
| mAsPCR-seg26.1..Recoded | CCTTCTCCCTGAATCGGAAATACTT |
| mAsPCR-seg26.1..Reverse | ACATTCGTTTTATTTTCTTCTTTACAGCCT |
| mAsPCR-seg26.1..Wild-Type | CTTGTTGCCTGAAAGCGAAATATTA |
| mAsPCR-seg26.2..Recoded | GAGTATGAAGATCGGGCGATTCTT |
| mAsPCR-seg26.2..Reverse | CAGCGTTTTGATCTCTTTACCTACATTC |
| mAsPCR-seg26.2..Wild-Type | GAGTATGAAGATCGGGCGATTTTA |
| mAsPCR-seg26.3..Recoded | TCCGATAAATTCCATTATGCCGGAGTA |
| mAsPCR-seg26.3..Reverse | AGTGCGTGATGAATGGATTGTTG |
| mAsPCR-seg26.3..Wild-Type | ACTGATAAATTCCATTATGCAGGTGTC |
| mAsPCR-seg26.4..Recoded | CGTATTTCGGCCATCAGTGATG |
| mAsPCR-seg26.4..Reverse | GTGGATTGACGATGACAAACC |
| mAsPCR-seg26.4..Wild-Type | CGTATTTCGGCCATCAGACTGC |
| mAsPCR-seg26.5..Recoded | GCTGACCAAATGACCAGATATGAAG |
| mAsPCR-seg26.5..Reverse | GCGCCAAACTATGCCGAAG |
| mAsPCR-seg26.5..Wild-Type | GCTGACCAAAACTCCAGATATGTAA |
| mAsPCR-seg26.6..Recoded | GAAGAGATTTATCGTGGCACCTC |
| mAsPCR-seg26.6..Reverse | CGGCGGTGATCTCAGAAATTTT |
| mAsPCR-seg26.6..Wild-Type | GAAGAGATTTATCGTGGCACCAG |
| mAsPCR-seg26.7..Recoded | CTTTTCAAATACAACGATGCTGGA |
| mAsPCR-seg26.7..Reverse | AAGTCGGGGAACTCTTCTTTTGA |
| mAsPCR-seg26.7..Wild-Type | TTGTTCAAATACAACGATGCAGGT |
| mAsPCR-seg26.8..Recoded | CTATCTCTTGAACCGGTGATCCTA |
| mAsPCR-seg26.8..Reverse | GCAGCAGTCCATAACCGAAAAG |
| mAsPCR-seg26.8..Wild-Type | CTAAGCCTTGAACCGGTGATCTTG |
| mAsPCR-seg27.1..Recoded | TTTATCCGCAAACGCATCTGTC |
| mAsPCR-seg27.1..Reverse | AAAGGTGGCAGGATGTTTACGA |
| mAsPCR-seg27.1..Wild-Type | TTTATCCGCAAACGCATCTGAG |
| mAsPCR-seg27.2..Recoded | AGAACTCACCATCTTTTATCGCAATT |
| mAsPCR-seg27.2..Reverse | CAACTCACCGAAGAACAGTACCA |
| mAsPCR-seg27.2..Wild-Type | AGAACTCACCATCTTTTATCGCAATA |
| mAsPCR-seg27.3..Recoded | CCGGATCGTCTACCTCTGCTA |
| mAsPCR-seg27.3..Reverse | GCCAATGGAAAGCTGATGTTTCA |
| mAsPCR-seg27.3..Wild-Type | CCGGATCGTTTACCTCTGTTG |
| mAsPCR-seg27.4..Recoded | GTTCACTTCTTGTTGTTTCATCATTCTCA |
| mAsPCR-seg27.4..Reverse | CTTTACCAATACCTGAGATGTAAACGG |
| mAsPCR-seg27.4..Wild-Type | GTTCATTGCTTGTTGTTTCATCATTCAGT |
| mAsPCR-seg27.5..Recoded | GATTATCTACCGCTGTATCTGGAGTATC |
| mAsPCR-seg27.5..Reverse | GATATTGATTAAGCGGCGAAGAGTC |
| mAsPCR-seg27.5..Wild-Type | GATTATCTACCGCTGTATCTGGAGTATT |
| mAsPCR-seg27.6..Recoded | TCAATCAGATGACCAGAGTACTTTGA |
| mAsPCR-seg27.6..Reverse | CGCGGGATGATCAATATGCTG |
| mAsPCR-seg27.6..Wild-Type | TCAATCAGATGACCGCTGTACTTACT |
| mAsPCR-seg27.7..Recoded | AAACAACAACGACGCAACCCTT |
| mAsPCR-seg27.7..Reverse | TTCGAAAGCAAAATCATCACGCA |
| mAsPCR-seg27.7..Wild-Type | AAACAACAACGACGCAACCTTG |
| mAsPCR-seg27.8..Recoded | AAAGTTCAAAAGAGATTATATCCCTTCTTCT |
| mAsPCR-seg27.8..Reverse | CACGCCATCCTGATCCATATGTATA |
| mAsPCR-seg27.8..Wild-Type | AAAGTTCAAAAGAGATTATATCCCTTCTTCA |
| mAsPCR-seg28.1..Recoded | GGCGGTAGGGAGTTACGAAG |
| mAsPCR-seg28.1..Reverse | TTTCATTTGCTTATGTGCTGGTCAA |
| mAsPCR-seg28.1..Wild-Type | GGCGGTAGGGAGTTACGTAA |
| mAsPCR-seg28.2..Recoded | CTTGTTACAAAGTAAGAATGGGAGTTTATGA |
| mAsPCR-seg28.2..Reverse | CGGGTTCACGGCTAAATGATAAC |
| mAsPCR-seg28.2..Wild-Type | CTTGTTACAAAGTAAGAATGGGAGTTTAACT |
| mAsPCR-seg28.3..Recoded | TTAAAATCGATAAGAAGCAAGTAACGGATC |
| mAsPCR-seg28.3..Reverse | CCAGTAGCGGGCGAATTTATG |
| mAsPCR-seg28.3..Wild-Type | TTAAAATGGATAAGAAGCAAGTAACGGATT |
| mAsPCR-seg28.4..Recoded | TGAAATTTTCATCCGTCAGTTTGAAT |
| mAsPCR-seg28.4..Reverse | CATAATGTGGTAAAGCGGTACAC |
| mAsPCR-seg28.4..Wild-Type | TGAAATTTTCATCCGTCAGTTTGAAA |
| mAsPCR-seg28.5..Recoded | ATCTGGCTGGCACAATATTACTCTT |
| mAsPCR-seg28.5..Reverse | CGACGTTATTGCCAGGTGTAGA |
| mAsPCR-seg28.5..Wild-Type | ATCTGGCTGGCACAATATTACTTTG |
| mAsPCR-seg28.6..Recoded | GCTTTCACTTTCGCTGCCACTA |
| mAsPCR-seg28.6..Reverse | CTTTATAAGCCGTGAGTACTTCTTCAA |
| mAsPCR-seg28.6..Wild-Type | GTTGTCACTTTCGCTGCCATTG |
| mAsPCR-seg28.7..Recoded | GGGTTTGCAATGGTTACTTCTGA |
| mAsPCR-seg28.7..Reverse | GTCTTTAATCATACCAATAACTCAGATGCC |
| mAsPCR-seg28.7..Wild-Type | GGGTTTGCAATGGTTACTTCACT |
| mAsPCR-seg28.8..Recoded | CGTTCATGCTTACTACGATATTCTATCA |
| mAsPCR-seg28.8..Reverse | GCTGCTGTTCTGACTCGGT |
| mAsPCR-seg28.8..Wild-Type | CGTTCATGCTTACTACGATATTTTGAGC |
| mAsPCR-seg29.1..Recoded | TGGCCATCGCTGTCTGGT |
| mAsPCR-seg29.1..Reverse | GGCAATAACCGACACAATAAGCG |
| mAsPCR-seg29.1..Wild-Type | TGGCCATCGCTGTCTGGA |
| mAsPCR-seg29.2..Recoded | GTTCTAAAGGATTTTATTGATGCACTTTCG |
| mAsPCR-seg29.2..Reverse | GAATGCCGGTGATAAGGTTAGGA |
| mAsPCR-seg29.2..Wild-Type | GTTTTAAAGGATTTTATTGATGCACTTAGT |
| mAsPCR-seg29.3..Recoded | CTACATCCACTAAATCATTACAACTCCTGA |
| mAsPCR-seg29.3..Reverse | CGCTACTGGGACGCTATGAA |
| mAsPCR-seg29.3..Wild-Type | TTACATCCATTAAATCATTACAACAGCTAG |
| mAsPCR-seg29.4..Recoded | GTGTTGCTGTCGATCCGGTA |
| mAsPCR-seg29.4..Reverse | CAAGCGGTGTCTGTGAGTTATTAATC |
| mAsPCR-seg29.4..Wild-Type | GTGTTGCTGTCGATCCGGTG |
| mAsPCR-seg29.5..Recoded | TCCTGTGAGCGCATACAGTC |
| mAsPCR-seg29.5..Reverse | AGAAGGGTATGAGTAATAAGGTGGGA |
| mAsPCR-seg29.5..Wild-Type | TCCTGTGAGCGCATACAGAG |
| mAsPCR-seg29.6..Recoded | TCACTGAGAGTTGTACGTTGTAGAGAAG |
| mAsPCR-seg29.6..Reverse | CTTGCCGCCTCCTGTTTTG |
| mAsPCR-seg29.6..Wild-Type | TCACTGAGAGTTGTACGTTGTAGAGTAA |
| mAsPCR-seg29.7..Recoded | CATAATTAGAATGCCGTGCCATG |
| mAsPCR-seg29.7..Reverse | GCCTATCCTTCCGGTGCTTT |
| mAsPCR-seg29.7..Wild-Type | CATAATCAAAATGCCGTGCCAGC |
| mAsPCR-seg29.8..Recoded | GCGGAACCCAGATAAGCAAG |
| mAsPCR-seg29.8..Reverse | CGTTTTGCCGCCGAGATC |
| mAsPCR-seg29.8..Wild-Type | GCGGAACCCAGATAAGCTAA |
| mAsPCR-seg30.1..Recoded | CAAAATAGGGAATAATCGACCACATTGA |
| mAsPCR-seg30.1..Reverse | CTTTGGTCAGTGTGGCTTGC |
| mAsPCR-seg30.1..Wild-Type | CAAAATAGGGAATAATCGACCACATACT |
| mAsPCR-seg30.2..Recoded | CAAGGGCCGCAGCTTTAAG |
| mAsPCR-seg30.2..Reverse | GGTACTGGACTAAATACCCATCCG |
| mAsPCR-seg30.2..Wild-Type | CAAGGGCCGCAGCTTTTAA |
| mAsPCR-seg30.3..Recoded | GCGATATATCCCGAAAGCCCTAG |
| mAsPCR-seg30.3..Reverse | TGCAAACCCTGAAACGGAATC |
| mAsPCR-seg30.3..Wild-Type | GCGATATATCCGCTTAACCCCAA |
| mAsPCR-seg30.4..Recoded | CCTGCAATCCTCGAAGCACTC |
| mAsPCR-seg30.4..Reverse | CCAAATACGCCGTGCATCAG |
| mAsPCR-seg30.4..Wild-Type | CCTGCAATCCTCGAAGCATTA |
| mAsPCR-seg30.5..Recoded | TTCGAGTGATGAGATTTTGCGAAATTTA |
| mAsPCR-seg30.5..Reverse | AAGTAAGCTCTGCACTTGTGGA |
| mAsPCR-seg30.5..Wild-Type | TTCGAGTGAACTGATTTTGCGAAATTTT |
| mAsPCR-seg30.6..Recoded | ATCGCCTCGGTCGTTTCT |
| mAsPCR-seg30.6..Reverse | CATCTGCACCGTCAAACAGTG |
| mAsPCR-seg30.6..Wild-Type | ATCGCCTCGGTGGTCAGC |
| mAsPCR-seg30.7..Recoded | GGCTTGATCCGAAGAAAACCT |
| mAsPCR-seg30.7..Reverse | GCCGCCTGTAGACCTTCTT |
| mAsPCR-seg30.7..Wild-Type | GGTTGGATCCGAAGAAAACCA |
| mAsPCR-seg30.8..Recoded | TCACCTGGGAGCCATTGG |
| mAsPCR-seg30.8..Reverse | GTAGCTGGTCAGGGCGTAC |
| mAsPCR-seg30.8..Wild-Type | TCACCTGACTGCCATTGC |
| mAsPCR-seg31.1..Recoded | CTATACCGATTACCCGACGCTA |
| mAsPCR-seg31.1..Reverse | CGCATCGGTTTTGGCGTT |
| mAsPCR-seg31.1..Wild-Type | CTATACCGATTACCCGACGTTG |
| mAsPCR-seg31.2..Recoded | GTCGCGGAATTTATGTACCAGTCA |
| mAsPCR-seg31.2..Reverse | GACGAAATACTTCATCAGACACCCA |
| mAsPCR-seg31.2..Wild-Type | GTCGCGGAATTTATGTACCAGAGC |
| mAsPCR-seg31.3..Recoded | GCCGCATCTTTTGGCTCA |
| mAsPCR-seg31.3..Reverse | GGGACTGGCACTTCTTCTGG |
| mAsPCR-seg31.3..Wild-Type | GCCGCATCTTTTGGCAGC |
| mAsPCR-seg31.4..Recoded | ACCAGATTGCCCTGAACTTTTCA |
| mAsPCR-seg31.4..Reverse | CCCATAGGTTCAACGACCAGAT |
| mAsPCR-seg31.4..Wild-Type | ACCAGATTGCCCTGAACTTTAGT |
| mAsPCR-seg31.5..Recoded | GAAAGGCTGGTCGTGCATA |
| mAsPCR-seg31.5..Reverse | TCTATTCGTCGCCTACTTGCC |
| mAsPCR-seg31.5..Wild-Type | GAAAGGCTGGTCGTGCATC |
| mAsPCR-seg31.6..Recoded | CGGTTGTCATTGTTGAACTCAAGT |
| mAsPCR-seg31.6..Reverse | GATGATCGAAAAATGTATCCGTGCA |
| mAsPCR-seg31.6..Wild-Type | CGGTTGTCATTGTTGAACTCGAGA |
| mAsPCR-seg31.7..Recoded | GCTGGAACACAATAAAGGTTTTTGTAACT |
| mAsPCR-seg31.7..Reverse | CGCCGTGTGAGCATTTCA |
| mAsPCR-seg31.7..Wild-Type | GCTGGAACACAATAAAGGTTTTTGTAACA |
| mAsPCR-seg31.8..Recoded | GCAATTAGCGTCCGTAGTGAA |
| mAsPCR-seg31.8..Reverse | TGTCCGTCGATGAAGATCACC |
| mAsPCR-seg31.8..Wild-Type | GCAATTAACGTCCGCAAACTG |
| mAsPCR-seg32.1..Recoded | GCTCATCTGTCCCAACGATCA |
| mAsPCR-seg32.1..Reverse | CACACTGCCAGACCGTAG |
| mAsPCR-seg32.1..Wild-Type | GCTCATCTGTCCCAAAGAAGT |
| mAsPCR-seg32.2..Recoded | TTTGCCGTCGGTAATTTCTGTTTTA |
| mAsPCR-seg32.2..Reverse | GTATTGTGATGATGCAAGTCCAGAAA |
| mAsPCR-seg32.2..Wild-Type | TTTGCCGTCGGTAATTTCTGTTTTT |
| mAsPCR-seg32.3..Recoded | AACTTAACTCTGTCTGGGTCTTTTCA |
| mAsPCR-seg32.3..Reverse | CGCGACAGAGAATTTCATGACG |
| mAsPCR-seg32.3..Wild-Type | AATTAAACAGCGTCTGGGTCTTTAGC |
| mAsPCR-seg32.4..Recoded | CCACCACCAGATGTTCAGGA |
| mAsPCR-seg32.4..Reverse | GCGCAAACTACTTCTTCAGGTAAA |
| mAsPCR-seg32.4..Wild-Type | CCACCACCAGATGTTCAGGT |
| mAsPCR-seg32.5..Recoded | AAGGACTGGCGATTGTGATGT |
| mAsPCR-seg32.5..Reverse | AGTGCTGTGATGAGAATAAGGCA |
| mAsPCR-seg32.5..Wild-Type | AAGGACTGGCGATTGTGATGA |
| mAsPCR-seg32.6..Recoded | CAGCTGGACTTCTCKTTCCT |
| mAsPCR-seg32.6..Reverse | AATCTTCTCATTACGTAGGTCTGCTT |
| mAsPCR-seg32.6..Wild-Type | CAGCTGGACTTCTCTTTGCCG |
| mAsPCR-seg32.7..Recoded | CGACCGTCGGACAACCCTT |
| mAsPCR-seg32.7..Reverse | CACAAGAGATATGCAGGACACT |
| mAsPCR-seg32.7..Wild-Type | CGACCGTCGGACAACCTTA |
| mAsPCR-seg32.8..Recoded | GGTATAAAAATCACCCAACCTAGAATACG |
| mAsPCR-seg32.8..Reverse | CTTATGATTAAGCGCCTATCATATCGC |
| mAsPCR-seg32.8..Wild-Type | GGTATAAAAATCACCCAACCCAAAATCCT |
| mAsPCR-seg33.1..Recoded | GCATCCCTATGGCGAGTGAT |
| mAsPCR-seg33.1..Reverse | AAATGGGCGAATACTACAAAGGC |
| mAsPCR-seg33.1..Wild-Type | GCATCCCTATGGCCAGACTC |
| mAsPCR-seg33.2..Recoded | CGACCCCTCCCCAAATGA |
| mAsPCR-seg33.2..Reverse | GGCTGACAGATAATCGTCGATGA |
| mAsPCR-seg33.2..Wild-Type | CGACCCCTCCCCAAAGCT |
| mAsPCR-seg33.3..Recoded | GCTGGAATCAAATAAAGCCGAAC |
| mAsPCR-seg33.3..Reverse | TTATTACCGCCCATCTCAAGGG |
| mAsPCR-seg33.3..Wild-Type | GCTGGAAAGCAATAAAGCCGAAT |
| mAsPCR-seg33.4..Recoded | GCATCGACTATGAAATCCGCTCA |
| mAsPCR-seg33.4..Reverse | GGTGGCAATGATGAAAAGCAGAATATA |
| mAsPCR-seg33.4..Wild-Type | GCATCGACTATGAAATCCGCAGC |
| mAsPCR-seg33.5..Recoded | CCATCAAGCAGACGGTTTAGT |
| mAsPCR-seg33.5..Reverse | AATGATGGCGGCAACAACTTC |
| mAsPCR-seg33.5..Wild-Type | CCATCAAGCAGCCTGTTCAAC |
| mAsPCR-seg33.6..Recoded | CTGATAGCGACACTGCTTTTCTG |
| mAsPCR-seg33.6..Reverse | TTCGGCGATGACCGGGAT |
| mAsPCR-seg33.6..Wild-Type | CTGATAGCGACACTGCTTTTCGC |
| mAsPCR-seg33.7..Recoded | AGTACCCTTGATTACTTTAACCTTTGA |
| mAsPCR-seg33.7..Reverse | GTTTCTGCTGGGTGGTATTGG |
| mAsPCR-seg33.7..Wild-Type | AGTACCCTTGATTACTTTAACCTTGCT |
| mAsPCR-seg33.8..Recoded | GTTTCATTACCGACATGCCCAAG |
| mAsPCR-seg33.8..Reverse | TGGTCGGTCAATGGAGATTATTCAT |
| mAsPCR-seg33.8..Wild-Type | GTTTCATTACCGACATGCCCTAA |
| mAsPCR-seg34.1..Recoded | TAATCAGTATTAAGTCGGCGAAGTGA |
| mAsPCR-seg34.1..Reverse | ATGGCCTGGCTATATCGTTACAC |
| mAsPCR-seg34.1..Wild-Type | TAATCAGTATTGAGACGGCGTAAACT |
| mAsPCR-seg34.2..Recoded | AGAATCTAGCCATCATCTCAAACTC |
| mAsPCR-seg34.2..Reverse | AAGTTGTCGAAAGTAGATTGCAGATG |
| mAsPCR-seg34.2..Wild-Type | AGAATTTGGCCATCATCAGCAACAG |
| mAsPCR-seg34.3..Recoded | CAATAACGGCAACCACGAAAGA |
| mAsPCR-seg34.3..Reverse | TGACCGTCACCAATAACTCGAAT |
| mAsPCR-seg34.3..Wild-Type | CAATAACGGCAACCACGAAGCT |
| mAsPCR-seg34.4..Recoded | TGTTTGATAATAATAGGCCCATTCAGCT |
| mAsPCR-seg34.4..Reverse | AATGCCACCACGCCACAG |
| mAsPCR-seg34.4..Wild-Type | TGTTTGATAATAATAGGCCCATTCAGCA |
| mAsPCR-seg34.5..Recoded | GAACCGGATAGACCCAGCGA |
| mAsPCR-seg34.5..Reverse | CGATCACCGCCAAGCTTATG |
| mAsPCR-seg34.5..Wild-Type | CTACCGGATAAACCCAGGCT |
| mAsPCR-seg34.6..Recoded | TCTTGAACAGGGTGCAATTCTCTC |
| mAsPCR-seg34.6..Reverse | TTCCACCACGAACAGCTCT |
| mAsPCR-seg34.6..Wild-Type | TCTTGAACAGGGTGCAATTTTAAG |
| mAsPCR-seg34.7..Recoded | GATAAAAGATCTCAATCAGTACTGGTTTTCT |
| mAsPCR-seg34.7..Reverse | ACTTATCAATTTTCAGCACGTCAGG |
| mAsPCR-seg34.7..Wild-Type | GATAAAAGATCTCAATCAGTACTGGTTTAGC |
| mAsPCR-seg34.8..Recoded | CAGTGCTCTACATCCAACTTTCA |
| mAsPCR-seg34.8..Reverse | GAAGACGCCACGAATATCTGATTG |
| mAsPCR-seg34.8..Wild-Type | CAGTGCTCTACATCCAACTTAGC |
| mAsPCR-seg35.1..Recoded | AGATATCAATATTATCTGGCCGATGATCCTT |
| mAsPCR-seg35.1..Reverse | CTTGCCGCGGGTTTTATGG |
| mAsPCR-seg35.1..Wild-Type | GGATATCAATATTATCTGGCCGATGATCTTA |
| mAsPCR-seg35.2..Recoded | AGAAACGCGATTACTTCTTTTGAGG |
| mAsPCR-seg35.2..Reverse | AAACAGAATTTTACGCGGATCTAAATC |
| mAsPCR-seg35.2..Wild-Type | AGAAACGCGATTACTTCTTTACTGC |
| mAsPCR-seg35.3..Recoded | GAAAGATGCTCGGCGGTTGA |
| mAsPCR-seg35.3..Reverse | CCGGCACCTTTAACCAGTTTATC |
| mAsPCR-seg35.3..Wild-Type | CTTAAATGCTCGGCGGTACT |
| mAsPCR-seg35.4..Recoded | CGAGGTCGTTTTATGCAGAGAA |
| mAsPCR-seg35.4..Reverse | TATGAACCAGGCTGTGAATATGCTAT |
| mAsPCR-seg35.4..Wild-Type | CGAGGTCGTTTTATGCAGGCTG |
| mAsPCR-seg35.5..Recoded | TGCTGGGTATGGACTACGGA |
| mAsPCR-seg35.5..Reverse | GCTACAAAAATGCCCGATCCTC |
| mAsPCR-seg35.5..Wild-Type | TGCTGGGTATGGACTACGGT |
| mAsPCR-seg35.6..Recoded | GGATTTATCAAACTCAGGAATGTATTCTGA |
| mAsPCR-seg35.6..Reverse | CAAAACTGCCGCGTACCG |
| mAsPCR-seg35.6..Wild-Type | GGATTTATCAAACTCAGGAATGTATTCGCT |
| mAsPCR-seg35.7..Recoded | GGTTTCGATTATATGGACCGCAAAC |
| mAsPCR-seg35.7..Reverse | GCGTTATGCCAAAGTGATTCCA |
| mAsPCR-seg35.7..Wild-Type | GGTTTCGATTATATGGACCGCAAAT |
| mAsPCR-seg35.8..Recoded | GCGCTCACTAAGTCCTGGT |
| mAsPCR-seg35.8..Reverse | TTTAGTGAAGATTTTACCGCGCTTAG |
| mAsPCR-seg35.8..Wild-Type | GCGCTCACTAAGTCCTGGA |
| mAsPCR-seg36.1..Recoded | CTGAATACCCTTAAAATTGCCTGGT |
| mAsPCR-seg36.1..Reverse | CGCCCACCAGATCATTTTGATATTC |
| mAsPCR-seg36.1..Wild-Type | CTGAATACCTTAAAAATTGCCTGGA |
| mAsPCR-seg36.2..Recoded | ATTTGCGGTAATCACAATCACTCA |
| mAsPCR-seg36.2..Reverse | CAGGATATTCGTCATCAGCTCGA |
| mAsPCR-seg36.2..Wild-Type | ATTTGCGGTAATCACAATCACAGT |
| mAsPCR-seg36.3..Recoded | CCAAACATGCCTTTCATTAGTTCTGA |
| mAsPCR-seg36.3..Reverse | ACAACTTAAACATCTTGGTATGGATATTGAC |
| mAsPCR-seg36.3..Wild-Type | CCAAACATGCCTTTCATTAATTCGCT |
| mAsPCR-seg36.4..Recoded | CGGAATGATGGCACTGATATGAA |
| mAsPCR-seg36.4..Reverse | GCCCCCCTATTTCTGACACC |
| mAsPCR-seg36.4..Wild-Type | CGGAATGATGGCACTGATATGAC |
| mAsPCR-seg36.5..Recoded | TAGTGATGACGCCAGAGATGAATTTCT |
| mAsPCR-seg36.5..Reverse | AGGCTGCAGTATTTTCCAAAACG |
| mAsPCR-seg36.5..Wild-Type | TAGTGATGACGCCAGAGATGAATTTCA |
| mAsPCR-seg36.6..Recoded | CCCGTCCGCTCGCTAAAC |
| mAsPCR-seg36.6..Reverse | CATCTCTTTTTCATTAAGTTTCAGTCGAAT |
| mAsPCR-seg36.6..Wild-Type | CCCGTCCGCTCGCTAAAT |
| mAsPCR-seg36.7..Recoded | TTCAGAATATTCGCTTTCTCAATATACCTCA |
| mAsPCR-seg36.7..Reverse | AATTCGAAACCTGCAGCATGG |
| mAsPCR-seg36.7..Wild-Type | TTCAGAATATTCGCTTAGCCAATATACCAGT |
| mAsPCR-seg36.8..Recoded | AACGTATTATCCATATCAGCTTTCCTCT |
| mAsPCR-seg36.8..Reverse | AGTGATGAGCGTGTCTGTAGC |
| mAsPCR-seg36.8..Wild-Type | AACGTATTATCCATATCAGTTGAGTAGC |
| mAsPCR-seg37.1..Recoded | TATCTAAAACTTTCCTCTAACGGCTATCTC |
| mAsPCR-seg37.1..Reverse | GACATCTTCGGCGGTGACT |
| mAsPCR-seg37.1..Wild-Type | TATCTAAAATTAAGCAGTAACGGCTATTTG |
| mAsPCR-seg37.2..Recoded | AACCTCCGTCACGCTATCAT |
| mAsPCR-seg37.2..Reverse | TACGCACTTTTCCGCCAGA |
| mAsPCR-seg37.2..Wild-Type | AACCTCCGTCACGCTAAGCA |
| mAsPCR-seg37.3..Recoded | GCGCATTCCTTTCCTGTTTTCA |
| mAsPCR-seg37.3..Reverse | CCAAACATTTCGGTAAACATCGGT |
| mAsPCR-seg37.3..Wild-Type | GCGCATTCCTTTCCTGTTTAGC |
| mAsPCR-seg37.4..Recoded | TAATTACCAACGCTCTTAAAACATCTGACG |
| mAsPCR-seg37.4..Reverse | GCTGTACGCGATTTATATTGGC |
| mAsPCR-seg37.4..Wild-Type | TAATTACCAACGCTCTTAAAACATCTGTCT |
| mAsPCR-seg37.5..Recoded | TGAAACACCCGCCGAAAAAC |
| mAsPCR-seg37.5..Reverse | ACCGCCCTGAGATGAATTAGTG |
| mAsPCR-seg37.5..Wild-Type | TGAAACACCCGCCGAAAAAT |
| mAsPCR-seg37.6..Recoded | GAACATAACTCTATTGCTGAGACTTTTAATC |
| mAsPCR-seg37.6..Reverse | GATTCCTAGCCCAAACATGCG |
| mAsPCR-seg37.6..Wild-Type | GAACATAACTCTATTGCTGAGACTTTTAATT |
| mAsPCR-seg37.7..Recoded | AGAGGGTTGTTTATTCTGATCACGA |
| mAsPCR-seg37.7..Reverse | CAGGCGCTCTCTCCACAG |
| mAsPCR-seg37.7..Wild-Type | AGAGGGTTGTTTATTCTGATCACGT |
| mAsPCR-seg37.8..Recoded | CGATGCTTCCTATTCGTCGTGATT |
| mAsPCR-seg37.8..Reverse | ACCACCCTGCCCTTTTTCTT |
| mAsPCR-seg37.8..Wild-Type | CGATGTTACCTATTCGTCGTGATA |
| mAsPCR-seg38.1..Recoded | CGAGCTGTAGTTGATAACCTGA |
| mAsPCR-seg38.1..Reverse | GCTTGATGAAGGCCGTCTTTC |
| mAsPCR-seg38.1..Wild-Type | CGAGCTGCAATTGATAACCGCT |
| mAsPCR-seg38.2..Recoded | CTATCAACTCTGGACGGCTCA |
| mAsPCR-seg38.2..Reverse | CGCCCGTTCTGAATGTGC |
| mAsPCR-seg38.2..Wild-Type | TTAAGTACTCTGGACGGCAGC |
| mAsPCR-seg38.3..Recoded | GCGGCTATCTGGATTATTGGCT |
| mAsPCR-seg38.3..Reverse | GTCATTTTCGCCATTACCGCTT |
| mAsPCR-seg38.3..Wild-Type | GCGGCTATCTGGATTATTGGCA |
| mAsPCR-seg38.4..Recoded | GGATACCATTCGCCTGACCTC |
| mAsPCR-seg38.4..Reverse | CGCAATCACATCCAGTTCGG |
| mAsPCR-seg38.4..Wild-Type | GGATACCATTCGCCTGACCAG |
| mAsPCR-seg38.5..Recoded | CGGCTCAAAAGGTACAGGACTT |
| mAsPCR-seg38.5..Reverse | GATTCACCACCTGTACCACAATTC |
| mAsPCR-seg38.5..Wild-Type | CGGCAGTAAAGGTACAGGTTTA |
| mAsPCR-seg38.6..Recoded | TCGGGTTTTCTGAGGTAAGTTTT |
| mAsPCR-seg38.6..Reverse | CACGTCGCCAGATTGAAGAAATT |
| mAsPCR-seg38.6..Wild-Type | TCGGGTTTTCGCTGGTCAATTTG |
| mAsPCR-seg38.7..Recoded | TCATCCCCTCAGCCATCCTT |
| mAsPCR-seg38.7..Reverse | GCCACGGTTCTGCTGATTG |
| mAsPCR-seg38.7..Wild-Type | TCATCCCCAGCGCCATCTTA |
| mAsPCR-seg38.8..Recoded | TCAATAGTTACCAGCGCGTTTGA |
| mAsPCR-seg38.8..Reverse | GCTTCGCGTGGGTGATATGTA |
| mAsPCR-seg38.8..Wild-Type | TCAATAGTTACCAGCGCGTTACT |
| mAsPCR-seg39.1..Recoded | GAGTCTTTCTTCCAGTATTCATCGAAAG |
| mAsPCR-seg39.1..Reverse | CACGAGGTCAACTTCATCTGC |
| mAsPCR-seg39.1..Wild-Type | GAGTCTTTCTTCCAGTATTCATCGAAGC |
| mAsPCR-seg39.2..Recoded | AGCCTGCCCGTTATTTCTCA |
| mAsPCR-seg39.2..Reverse | GTATGTTCCGGCCATTGTAGAATC |
| mAsPCR-seg39.2..Wild-Type | AGCCTGCCCGTTATTTCAGC |
| mAsPCR-seg39.3..Recoded | CGTTTTTATTCCCGCTCCTCA |
| mAsPCR-seg39.3..Reverse | CAATGCCAGAGCCAACGAC |
| mAsPCR-seg39.3..Wild-Type | CGTTTTTATTCCCGCAGCAGT |
| mAsPCR-seg39.4..Recoded | CAAACTATATGAAGCCAAAAACCGTCTT |
| mAsPCR-seg39.4..Reverse | CAGGGTAAACGCGGGAAGT |
| mAsPCR-seg39.4..Wild-Type | CAAATTGTATGAAGCCAAAAACCGTTTA |
| mAsPCR-seg39.5..Recoded | AAGATGTGAGTATGGGTCGTTAAAAAG |
| mAsPCR-seg39.5..Reverse | CAGCCACCTCCGATTCCT |
| mAsPCR-seg39.5..Wild-Type | CAAATGGCTGTATGGGTCGTTAAACAA |
| mAsPCR-seg39.6..Recoded | GCATCAGGGCCAGTGAAAAAAG |
| mAsPCR-seg39.6..Reverse | TGCTCGCCCTAACCGTTATAC |
| mAsPCR-seg39.6..Wild-Type | GCATCAGGGCCAGGCTAAATAA |
| mAsPCR-seg39.7..Recoded | CGGTCGTATTTTCTCTGGCTCT |
| mAsPCR-seg39.7..Reverse | TCGGTCGATTGAGTGACAGC |
| mAsPCR-seg39.7..Wild-Type | CGGTCGTATTTTCAGTGGCAGC |
| mAsPCR-seg39.8..Recoded | GTGAGAATATTAGATAGGTTGAGCAGAGAA |
| mAsPCR-seg39.8..Reverse | CGTCTTGCATCACTTCACCTTTAAG |
| mAsPCR-seg39.8..Wild-Type | GTGAGAATATTACTTAAGTTCAACAGACTT |
| mAsPCR-seg40.1..Recoded | CCAGGGCCGCTTCTTTTGA |
| mAsPCR-seg40.1..Reverse | CCACCCATTGAGTGACCTGAA |
| mAsPCR-seg40.1..Wild-Type | CCAGGGCCGCTTCTTTACT |
| mAsPCR-seg40.2..Recoded | CGGTGTACGGAATAATCAGTGA |
| mAsPCR-seg40.2..Reverse | GGTTTACTTCCTGATGACCTCACT |
| mAsPCR-seg40.2..Wild-Type | CGGTGTACGGAATAATCAGGCT |
| mAsPCR-seg40.3..Recoded | AAACTCTGCGTCACCCTTTCC |
| mAsPCR-seg40.3..Reverse | CGCATTTTCGGCTATTTCGC |
| mAsPCR-seg40.3..Wild-Type | AAACTCTGCGTCACCTTAAGT |
| mAsPCR-seg40.4..Recoded | GTTCACAGTGTCCTTGCATTATCTTTGATT |
| mAsPCR-seg40.4..Reverse | TGCGGACGATCGGTAATACC |
| mAsPCR-seg40.4..Wild-Type | GTAGTCAGTGTCCTTGCATTATCTTTGATA |
| mAsPCR-seg40.5..Recoded | CTCAGGATTCGCCCATATCTCC |
| mAsPCR-seg40.5..Reverse | ATTTCCGGCATCATCAACGC |
| mAsPCR-seg40.5..Wild-Type | CTCAGGATTCGCCCATATCAGT |
| mAsPCR-seg40.6..Recoded | CGTAATCTTCCTGCCGTGACG |
| mAsPCR-seg40.6..Reverse | ACGTTTGTGCTGGTGAAAGATAAAA |
| mAsPCR-seg40.6..Wild-Type | CGTAATCTTCCTGCCGTGAAC |
| mAsPCR-seg40.7..Recoded | GTACAGACAGAAGAGAATGGACGA |
| mAsPCR-seg40.7..Reverse | GTTTGTGGGCTGCGTGTC |
| mAsPCR-seg40.7..Wild-Type | GTACAGACAGAAGAGAATGGAGCT |
| mAsPCR-seg40.8..Recoded | GCAGGGTAAGGGTGCTTC |
| mAsPCR-seg40.8..Reverse | GCTTTAACTTTGATTTCTTTACCGTCAAC |
| mAsPCR-seg40.8..Wild-Type | GCAGGGTAAGGGTGCGAG |
| mAsPCR-seg41.1..Recoded | TGGACACTACTGCTGGCAATCT |
| mAsPCR-seg41.1..Reverse | GCACATCACGCTCAACTGAATAG |
| mAsPCR-seg41.1..Wild-Type | TGGACATTACTGCTGGCAATCA |
| mAsPCR-seg41.2..Recoded | TATCCATAGCAGGTTTTGATGGTAAGA |
| mAsPCR-seg41.2..Reverse | GTGCGACCTGTCCGGATT |
| mAsPCR-seg41.2..Wild-Type | TATCCATAACAGGTTTTGATGGTAGCT |
| mAsPCR-seg41.3..Recoded | AATCTAACTTCTCGCTGCAACTCT |
| mAsPCR-seg41.3..Reverse | GCTTCAAAACGATCCTCTTCTGAAAG |
| mAsPCR-seg41.3..Wild-Type | AATCTAACTTCTCGCTGCAACTCA |
| mAsPCR-seg41.4..Recoded | TCGTCACCAGAAGCACAATGATAAG |
| mAsPCR-seg41.4..Reverse | TTTTTTTTACCCTTCTTTACACACTTTTCA |
| mAsPCR-seg41.4..Wild-Type | TCGTCACCAGTAACACAATGATCAA |
| mAsPCR-seg41.5..Recoded | CGTCTACTGGCAGATCAGCTA |
| mAsPCR-seg41.5..Reverse | CGGACACGCTCGGCATAA |
| mAsPCR-seg41.5..Wild-Type | CGTTTGCTGGCAGATCAGTTG |
| mAsPCR-seg41.6..Recoded | ACCGCACCATTGAACTCTCA |
| mAsPCR-seg41.6..Reverse | CGATTTCTTTGAGTACTACGGACAGATA |
| mAsPCR-seg41.6..Wild-Type | ACCGCACCATTGAACTCAGT |
| mAsPCR-seg41.7..Recoded | TAGTTTCAGTTTGCCCTTTTCAGA |
| mAsPCR-seg41.7..Reverse | CTTAATCGGGTTCTTCCAGTGC |
| mAsPCR-seg41.7..Wild-Type | CAATTTCAGTTTGCCCTTTTCGCT |
| mAsPCR-seg41.8..Recoded | TTGATAGATGAGATTTCCGTTTTTGAA |
| mAsPCR-seg41.8..Reverse | AGCTCTTTTCGTCACTCCTTGA |
| mAsPCR-seg41.8..Wild-Type | TTGATGCTACTGATTTTCCGTTTTGCTT |
| mAsPCR-seg42.1..Recoded | AGACACTTCTACGGTGCAACTTT |
| mAsPCR-seg42.1..Reverse | CGAAAGAAACCCTGCCGTCT |
| mAsPCR-seg42.1..Wild-Type | AGACACTTCTACGGTGCAACTTA |
| mAsPCR-seg42.2..Recoded | CCATTGCCCATCAGCGATTG |
| mAsPCR-seg42.2..Reverse | TCTTGAACGGCATAATAGGTTAGATAAATTG |
| mAsPCR-seg42.2..Wild-Type | CCATTGCCCATCAGCGATAC |
| mAsPCR-seg42.3..Recoded | CGCAGGAAGTGGAAGTCTCA |
| mAsPCR-seg42.3..Reverse | TTCTTGACCTGGAGAAATCACGT |
| mAsPCR-seg42.3..Wild-Type | CGCAGGAAGTGGAAGTCAGT |
| mAsPCR-seg42.4..Recoded | TGTTCCGCCAGATAGAAGAATCA |
| mAsPCR-seg42.4..Reverse | GTGGTTCTGGTAGATGTATTTCGAGA |
| mAsPCR-seg42.4..Wild-Type | TGTTCCGCCAGATAGAAGAAAGC |
| mAsPCR-seg42.5..Recoded | GACATCCAGCAGTCGAGCATTAG |
| mAsPCR-seg42.5..Reverse | CCTGTATTACTCCGGCTCTGG |
| mAsPCR-seg42.5..Wild-Type | CTCATCCAGCAGTCGAGCATTAA |
| mAsPCR-seg42.6..Recoded | TACTATGCAGGGCTCGCAACTT |
| mAsPCR-seg42.6..Reverse | TCGGAATGAATTGAGATATCGCCTT |
| mAsPCR-seg42.6..Wild-Type | TACTATGCAGGGCTCGCAATTA |
| mAsPCR-seg42.7..Recoded | GCAATCCATACCAGCACATAGGA |
| mAsPCR-seg42.7..Reverse | GCGCAACTATCCCTGGGT |
| mAsPCR-seg42.7..Wild-Type | GCAATCCATACCAGCACATAACT |
| mAsPCR-seg42.8..Recoded | GAATTTAGAGTCACGTTCACCACAA |
| mAsPCR-seg42.8..Reverse | TTGCCTCACTCAATGACGATCA |
| mAsPCR-seg42.8..Wild-Type | GAATTTGCTGTCACGTTCACCACAT |
| mAsPCR-seg43.1..Recoded | GTCTACCACTTATCCAGTCTTCGC |
| mAsPCR-seg43.1..Reverse | GTTATCCGGGGCATAGCGT |
| mAsPCR-seg43.1..Wild-Type | GTTTGCCACTTATCCAGTCTTCGT |
| mAsPCR-seg43.2..Recoded | GTGAAGCAGTGGTGATAACTAGAATAGA |
| mAsPCR-seg43.2..Reverse | TTGGTCAATATGAAATAGCTTGATGGC |
| mAsPCR-seg43.2..Wild-Type | GTGAAGCAGTGGTGATAACTAAAATACT |
| mAsPCR-seg43.3..Recoded | GGATTGTGACCATCTCTGCAC |
| mAsPCR-seg43.3..Reverse | CCGTCTTTGGTTTCTGCTTTTTG |
| mAsPCR-seg43.3..Wild-Type | GGATTGTGACCATCTCTGCAT |
| mAsPCR-seg43.4..Recoded | CGGAAATATTTGATGGCAGACTGTAG |
| mAsPCR-seg43.4..Reverse | CGGTGGTATGCGTGATGGT |
| mAsPCR-seg43.4..Wild-Type | CGGAAATATTTGATGGCGCTCTGTAA |
| mAsPCR-seg43.5..Recoded | CCGCCAGGGGTAATAAATTCTGA |
| mAsPCR-seg43.5..Reverse | GCACGTCAGCATAATCTCATTATCTTC |
| mAsPCR-seg43.5..Wild-Type | CCGCCAGGGGTAATAAATTCACT |
| mAsPCR-seg43.6..Recoded | GATAATTTCTATTAATTTCGTTGGCAGAAAG |
| mAsPCR-seg43.6..Reverse | GCGCTTCATGTTTCCTGGTC |
| mAsPCR-seg43.6..Wild-Type | GATAATTTGATTAATTTCGTTGGCGCTCAA |
| mAsPCR-seg43.7..Recoded | CGGCTGACCCAGTACAAGGAG |
| mAsPCR-seg43.7..Reverse | TGGGAACGTATTTATCCGCTTGA |
| mAsPCR-seg43.7..Wild-Type | CGGCTGACCCAGTACTAACAA |
| mAsPCR-seg43.8..Recoded | CAGCAGAGTGAATAAGGATAAGGTGA |
| mAsPCR-seg43.8..Reverse | GGAGTGGGTTATATTTATGTAGTGATAGAGC |
| mAsPCR-seg43.8..Wild-Type | CAGCAGAGTGAATAAGGATAAGGACT |
| mAsPCR-seg44.1..Recoded | TATTTATGAAACGACTCATTGTAGGCATCT |
| mAsPCR-seg44.1..Reverse | ATAAGACGTTGCATTATTGTCCTGAAG |
| mAsPCR-seg44.1..Wild-Type | TATTTATGAAACGACTCATTGTAGGCATCA |
| mAsPCR-seg44.2..Recoded | GTGAAATCATTCTCGCCCAGTAG |
| mAsPCR-seg44.2..Reverse | GCTGCGTGCGTAATGACTAC |
| mAsPCR-seg44.2..Wild-Type | GTGAAATCATTCTCGCCCAGCAA |
| mAsPCR-seg44.3..Recoded | TGAGATAACCGTCATAGCACAGT |
| mAsPCR-seg44.3..Reverse | CGTTTACTTTTGCTCGTCGGTT |
| mAsPCR-seg44.3..Wild-Type | TGAGATAACCGTCATAGCACAGC |
| mAsPCR-seg44.4..Recoded | GAATAGCGTTGATGACATTGCAAG |
| mAsPCR-seg44.4..Reverse | GATCTCATTATCGACGACATCAACG |
| mAsPCR-seg44.4..Wild-Type | GAATAACGTGCTACTCATTGCCAA |
| mAsPCR-seg44.5..Recoded | GTATGCTGGTGAAGATGACGTTTC |
| mAsPCR-seg44.5..Reverse | GTCATCGCCGCCATTTTCTT |
| mAsPCR-seg44.5..Wild-Type | GTATGCTGGTGAAGATGACGTTAG |
| mAsPCR-seg44.6..Recoded | GTCTTCCTGAAGTACAACTTGGAC |
| mAsPCR-seg44.6..Reverse | CAGCAGCGCACGACCAAG |
| mAsPCR-seg44.6..Wild-Type | GTTTGCCTGAAGTACAACTTGGAT |
| mAsPCR-seg44.7..Recoded | ACCTTTATCTTCGCGCTTATGTCA |
| mAsPCR-seg44.7..Reverse | ATCCATTTAACTAAGAGGACAATGCG |
| mAsPCR-seg44.7..Wild-Type | ACCTTTATCTTCGCGTTAATGAGT |
| mAsPCR-seg44.8..Recoded | TTTCTCCGGAGTTTAAACAGTTCTTTTCA |
| mAsPCR-seg44.8..Reverse | CCATGTGAGCGCAGTTTCG |
| mAsPCR-seg44.8..Wild-Type | TTTCTCCGGAGTTTAAACAGTTCTTTAGC |
| mAsPCR-seg45.1..Recoded | GCATCAAAATCGATCGCACTATCA |
| mAsPCR-seg45.1..Reverse | CTTTTTCACGTTCGTTAGCCTGT |
| mAsPCR-seg45.1..Wild-Type | GCAAGCAAATCGATCGCATTAAGT |
| mAsPCR-seg45.2..Recoded | TGACTTCGGGCATGGTAGG |
| mAsPCR-seg45.2..Reverse | AAAATTTCGAGGTTATTAATCATGTCAGATC |
| mAsPCR-seg45.2..Wild-Type | TGACTTCGGGCATGGCAAT |
| mAsPCR-seg45.3..Recoded | GCTGTTTCGCCATGTCAATTCT |
| mAsPCR-seg45.3..Reverse | CGGATTCAGACGGATTGACGA |
| mAsPCR-seg45.3..Wild-Type | GCTGTTTCGCCATGTCAATAGC |
| mAsPCR-seg45.4..Recoded | TGAAGATCTTACCCCATCACAGTTTC |
| mAsPCR-seg45.4..Reverse | GGAACAGCCCGACACCTT |
| mAsPCR-seg45.4..Wild-Type | TGAAGATTTAACCCCAAGCCAGTTTT |
| mAsPCR-seg45.5..Recoded | CGTCGGCTGGGTAGACATTAG |
| mAsPCR-seg45.5..Reverse | TGATGTCAGGGATTTCACGCA |
| mAsPCR-seg45.5..Wild-Type | CGTCGGCTGGGTAGACATCAA |
| mAsPCR-seg45.6..Recoded | CACGACCCCCAGATAAAATATTGAAG |
| mAsPCR-seg45.6..Reverse | CCTTAAAGTCGTTGCTGTATCCG |
| mAsPCR-seg45.6..Wild-Type | CAGCTCCCCCAGATAAAATATTGCAA |
| mAsPCR-seg45.7..Recoded | TTATCAACGCGGAAGAGATTGACT |
| mAsPCR-seg45.7..Reverse | ATGACTTCAATGCCCAGTTCCT |
| mAsPCR-seg45.7..Wild-Type | TTATCAACGCGGAAGAGATTGACA |
| mAsPCR-seg45.8..Recoded | GCGCTAAAACTACAAGAAGATGAATCA |
| mAsPCR-seg45.8..Reverse | AAGGTGCTTTTTTACGCATTTTTAACA |
| mAsPCR-seg45.8..Wild-Type | GCGTTGAAACTACAAGAAGATGAAAGC |
| mAsPCR-seg46.1..Recoded | GTATTGCCTATTGTTTGTTCTAGTGTGGA |
| mAsPCR-seg46.1..Reverse | TGAAGAACTAAAATTCACCTCCGTT |
| mAsPCR-seg46.1..Wild-Type | GTATTGCCTATTGTTTGTTCTAATGTACT |
| mAsPCR-seg46.2..Recoded | AACAATCGCCGCTTTCGTAAG |
| mAsPCR-seg46.2..Reverse | ACAACGCCTGAAATGATGCATAAA |
| mAsPCR-seg46.2..Wild-Type | AACAATCGCCGCTTTCGTTAA |
| mAsPCR-seg46.3..Recoded | TACCTCAGCGACAAGAAAAAGCG |
| mAsPCR-seg46.3..Reverse | TTCGGCTTTGAGTGTCCGT |
| mAsPCR-seg46.3..Wild-Type | TACCTCAGCGACCAAAAACAAAG |
| mAsPCR-seg46.4..Recoded | GCAGAAATCAGACCGAGTGA |
| mAsPCR-seg46.4..Reverse | GTTATGGTCGCGTGAAGATTGAAG |
| mAsPCR-seg46.4..Wild-Type | GCAGAAATCAGACCGAGGCT |
| mAsPCR-seg46.5..Recoded | GTTGTTCATATTCAGTACTTTACCGACTG |
| mAsPCR-seg46.5..Reverse | CGCTGGGGCTGAAATTCATC |
| mAsPCR-seg46.5..Wild-Type | GTTGTTCATATTCAGTACTTTACCGACGC |
| mAsPCR-seg46.6..Recoded | CAACCGTAATTAACAACGCCATCT |
| mAsPCR-seg46.6..Reverse | AATCAGACGTTTATTGGTGTGTTTACG |
| mAsPCR-seg46.6..Wild-Type | CAACCGTAATTAACAACGCCATCA |
| mAsPCR-seg46.7..Recoded | CCGAACAAATCCTCGCCCTT |
| mAsPCR-seg46.7..Reverse | GAACAGACGAATGCCTTCAGAC |
| mAsPCR-seg46.7..Wild-Type | CCGAACAAATCCTCGCCTTA |
| mAsPCR-seg46.8..Recoded | CGATGTGCATTGAGTTGTGGTG |
| mAsPCR-seg46.8..Reverse | CTTTTTTTACATTGTGCTGCTGTCG |
| mAsPCR-seg46.8..Wild-Type | CGATGTGCATTGAGTTGTGGAC |
| mAsPCR-seg47.1..Recoded | TTACACCTCATGGAAAAATTGCTGATAT |
| mAsPCR-seg47.1..Reverse | AACCTCTCTTATAATTATGGGTATTCTACGG |
| mAsPCR-seg47.1..Wild-Type | CTACACCTCATGGAAAAATTGCTGATAA |
| mAsPCR-seg47.2..Recoded | GTCAAAAACCAGTGCCTCAGA |
| mAsPCR-seg47.2..Reverse | CCGCATTTTGTCCAGCATCTC |
| mAsPCR-seg47.2..Wild-Type | GTCAAAAACCAGTGCCTCGCT |
| mAsPCR-seg47.3..Recoded | TATCTTCGGTGCCAGCCATGA |
| mAsPCR-seg47.3..Reverse | CGGTCTGTCACTGCACGA |
| mAsPCR-seg47.3..Wild-Type | TATCTTCGGTGCCAGCCAACT |
| mAsPCR-seg47.4..Recoded | CAGCAGCAGTGTGATCCCTAG |
| mAsPCR-seg47.4..Reverse | CGGTAGCGCTAGGTCATTTTCT |
| mAsPCR-seg47.4..Wild-Type | CAGCAGCAGTGTGATCCCTAA |
| mAsPCR-seg47.5..Recoded | AGATTGGCGGTAATAAAATGCGAT |
| mAsPCR-seg47.5..Reverse | GGAGTCGCGGTTCTACACTG |
| mAsPCR-seg47.5..Wild-Type | AGATTGGCGGTAATAAAATGGCTG |
| mAsPCR-seg47.6..Recoded | CTGACGACGAAACCTTTGCAT |
| mAsPCR-seg47.6..Reverse | GTCGATACAGACCAGCGATAGAT |
| mAsPCR-seg47.6..Wild-Type | CTGACGACGAAACCTTTGCAA |
| mAsPCR-seg47.7..Recoded | CTGTTCCTGATTAAAACCCGGAAG |
| mAsPCR-seg47.7..Reverse | ACCAGTATCACATCGACTCAGAAC |
| mAsPCR-seg47.7..Wild-Type | CTGTTCCTGATTAAAACCCGGCAA |
| mAsPCR-seg47.8..Recoded | GGGTTCTATGGTGAATGATAAAACCCTT |
| mAsPCR-seg47.8..Reverse | CAGGACATTTGGTATTTGGCTGAA |
| mAsPCR-seg47.8..Wild-Type | GGGTTCTATGGTGAATGATAAAACCTTA |
| mAsPCR-seg48.1..Recoded | TAATCCAGTGCAGATAACCTTCAGA |
| mAsPCR-seg48.1..Reverse | AGAGCCTGCACTTCTTTCTGG |
| mAsPCR-seg48.1..Wild-Type | TAATCCAGTGCAGATAACCTTCACT |
| mAsPCR-seg48.2..Recoded | CACTGATGCTACCGGTAAAAAACTT |
| mAsPCR-seg48.2..Reverse | CGCACAGTCAACCACCATG |
| mAsPCR-seg48.2..Wild-Type | CACTGATGCTACCGGTAAAAAATTG |
| mAsPCR-seg48.3..Recoded | CGGCAGATGACTTCGGTTCA |
| mAsPCR-seg48.3..Reverse | TCTTTGATATAACGTGCGATGTTCAG |
| mAsPCR-seg48.3..Wild-Type | CGGCAGATGACTTCGGTAGC |
| mAsPCR-seg48.4..Recoded | CGTGGCGATGCGTGAACTT |
| mAsPCR-seg48.4..Reverse | CATCCAGTTCATCGGTCGTTTTTAG |
| mAsPCR-seg48.4..Wild-Type | CGTGGCGATGCGTGAATTA |
| mAsPCR-seg48.5..Recoded | CGACCGATGGATTTACGAACAAG |
| mAsPCR-seg48.5..Reverse | GTCTGTGGAACGGCATCAAA |
| mAsPCR-seg48.5..Wild-Type | CGACCGATGGATTTACGAACTAA |
| mAsPCR-seg48.6..Recoded | GAACATGCGTGACGAGCTATC |
| mAsPCR-seg48.6..Reverse | CGGCACTAGATAAACGCAGAAG |
| mAsPCR-seg48.6..Wild-Type | GAACATGCGTGACGAGTTAAG |
| mAsPCR-seg48.7..Recoded | TCAGCGTTGATCATCACACCA |
| mAsPCR-seg48.7..Reverse | GTCGGCCCGTGTGGTATG |
| mAsPCR-seg48.7..Wild-Type | TCAGCGTTGATCATCACACCG |
| mAsPCR-seg48.8..Recoded | GTGTTGATGATAGATATAGTGGACATCTG |
| mAsPCR-seg48.8..Reverse | GTTAATGAGGGATTTATGAAAACGATGC |
| mAsPCR-seg48.8..Wild-Type | GTGTTGATGATAGATATAGTGGACATCGC |
| mAsPCR-seg49.1..Recoded | CCAAATTCTGAGTGTCCCCATGA |
| mAsPCR-seg49.1..Reverse | GCGGTGTGGCTGGAAAAC |
| mAsPCR-seg49.1..Wild-Type | CCAAATTCACTGTGTCCCCAACT |
| mAsPCR-seg49.2..Recoded | CGGCGTTCTCTGGGCAATT |
| mAsPCR-seg49.2..Reverse | AAGATCATGGCGCGTTCCT |
| mAsPCR-seg49.2..Wild-Type | GGGCGTTCTCTGGGCAATA |
| mAsPCR-seg49.3..Recoded | CGCACCCAGTTCTTCGTTAAATAG |
| mAsPCR-seg49.3..Reverse | GCCTGTATGAAGCCGTTAAAGC |
| mAsPCR-seg49.3..Wild-Type | CGCACCCAGTTCTTCGTTAAACAA |
| mAsPCR-seg49.4..Recoded | CAGGGGCTTGCCCAGTCA |
| mAsPCR-seg49.4..Reverse | GTTTTGCGCCACCAGACC |
| mAsPCR-seg49.4..Wild-Type | CAGGGGCTTGCCCAGAGT |
| mAsPCR-seg49.5..Recoded | CGACAACCGCGACAACTC |
| mAsPCR-seg49.5..Reverse | GGGACCAACGCTGTTTCG |
| mAsPCR-seg49.5..Wild-Type | CGACAACCGCGACAACAG |
| mAsPCR-seg49.6..Recoded | GGTCCGTTAGCTGCTCTGA |
| mAsPCR-seg49.6..Reverse | GAGGATTAGGTGGTGAAATAAAAAGGC |
| mAsPCR-seg49.6..Wild-Type | GGTCCGTTAACTGCTCGCT |
| mAsPCR-seg49.7..Recoded | GCAGCGGTACACCCTCTTTCA |
| mAsPCR-seg49.7..Reverse | ACCCATGATAGCGCCTGTG |
| mAsPCR-seg49.7..Wild-Type | GCAGCGGTACACCTTTTGAGT |
| mAsPCR-seg49.8..Recoded | TCTGCGGTATTGGAAGTCAGATTC |
| mAsPCR-seg49.8..Reverse | GAGGCACGACGTCTTTTCT |
| mAsPCR-seg49.8..Wild-Type | TCTGCGGTATTGGAAGTCAGATTG |
| mAsPCR-seg50.1..Recoded | GTTTGGACTAATGTTCTCTGTCTCACTA |
| mAsPCR-seg50.1..Reverse | CAATCGCCGTGCATTCATCAT |
| mAsPCR-seg50.1..Wild-Type | GTTTGGATTGATGTTCTCTGTCAGTTTG |
| mAsPCR-seg50.2..Recoded | GACCATCGCCTCGTCTGA |
| mAsPCR-seg50.2..Reverse | GGAACAACAGGCGCTTATGAAA |
| mAsPCR-seg50.2..Wild-Type | GACCATCGCCTCGTCGCT |
| mAsPCR-seg50.3..Recoded | CGCTAACTATCGACCATTGTCTACTA |
| mAsPCR-seg50.3..Reverse | CTTTTTGCATTTCCGCTGATTCAAG |
| mAsPCR-seg50.3..Wild-Type | CGTTAACTATCGACCATTGTTTGTTG |
| mAsPCR-seg50.4..Recoded | ACCGATAACTATGGTGAAGACTCC |
| mAsPCR-seg50.4..Reverse | TTCCAGACTCACTCTCCGGTA |
| mAsPCR-seg50.4..Wild-Type | ACCGATAACTATGGTGAAGACAGT |
| mAsPCR-seg50.5..Recoded | CTCAGGCGTTTTCTGTTCTTTTGATGA |
| mAsPCR-seg50.5..Reverse | TGCCAGTTTTCACATTCTTCAGTT |
| mAsPCR-seg50.5..Wild-Type | CTCAGGCGTTTTCTGTTCTTTACTACT |
| mAsPCR-seg50.6..Recoded | CGAACTAATTGGCATGGACTCT |
| mAsPCR-seg50.6..Reverse | TTTCTTGTGAGTCGGCCTGAT |
| mAsPCR-seg50.6..Wild-Type | CGAATTGATTGGCATGGACAGC |
| mAsPCR-seg50.7..Recoded | CCAGCCTTTATGCAGCGTCTT |
| mAsPCR-seg50.7..Reverse | CGACGGCATCCATTACTTCC |
| mAsPCR-seg50.7..Wild-Type | CCAGCCTTTATGCAGCGTTTA |
| mAsPCR-seg50.8..Recoded | GGAAGTTTTACACCTCATATACGCTT |
| mAsPCR-seg50.8..Reverse | AGGAATGTTGGCGTGGCT |
| mAsPCR-seg50.8..Wild-Type | GGAAGTTTTACACCAGCTATACGTTG |
| mAsPCR-seg51.1..Recoded | CCCGGCTTCAGTTCGTTAG |
| mAsPCR-seg51.1..Reverse | CCCATTCATTAAGTAACTCTGCACTTG |
| mAsPCR-seg51.1..Wild-Type | CCCGGCTTCAGTTCGTTAC |
| mAsPCR-seg51.2..Recoded | GTGTAACCGTAGACCTCCTGA |
| mAsPCR-seg51.2..Reverse | GTGGGCGTGTGGTGTCTC |
| mAsPCR-seg51.2..Wild-Type | GTGTAACCGTAGACCTCCTGC |
| mAsPCR-seg51.3..Recoded | AACTGATTGGTATGGTCGCTCAA |
| mAsPCR-seg51.3..Reverse | GCTGGTAGATCTCTTCACGGT |
| mAsPCR-seg51.3..Wild-Type | AACTGATTGGTATGGTCGCTCAG |
| mAsPCR-seg51.4..Recoded | CTGCCCAACCTGTTCGGAAAG |
| mAsPCR-seg51.4..Reverse | CAAAACTAAGTACTCTATTTCGCAGCTT |
| mAsPCR-seg51.4..Wild-Type | CTGCCCAACCTGTTCACTTAA |
| mAsPCR-seg51.5..Recoded | GCATCGCATCCATCACTGA |
| mAsPCR-seg51.5..Reverse | GAAGATAAATCTATCGCGCTGCTG |
| mAsPCR-seg51.5..Wild-Type | GCATCGCATCCATCACGCT |
| mAsPCR-seg51.6..Recoded | AAGCACCATTATCGGCTGTGA |
| mAsPCR-seg51.6..Reverse | GTCGGCGAAGTCAACTCAGA |
| mAsPCR-seg51.6..Wild-Type | AAGCACCATTATCGGCTGACT |
| mAsPCR-seg51.7..Recoded | CGAGGTCAGTTTCAACCGTAAG |
| mAsPCR-seg51.7..Reverse | CGTAAAAACTCGCCGCTGAAATA |
| mAsPCR-seg51.7..Wild-Type | CGAGGTCAGTTTCAACCGTTAA |
| mAsPCR-seg51.8..Recoded | CTATTGAAAACAATGTGCCGGTGAATC |
| mAsPCR-seg51.8..Reverse | CATTCCTCAGGTGATTGTCATTTTTGA |
| mAsPCR-seg51.8..Wild-Type | CTATTGAAAACAATGTGCCGGTTGAATT |
| mAsPCR-seg52.1..Recoded | ATTACGCTTATCCCGACGCTT |
| mAsPCR-seg52.1..Reverse | AGACGTGCCTGATCTTCCTC |
| mAsPCR-seg52.1..Wild-Type | ATTACGCTTATCCCGACGTTG |
| mAsPCR-seg52.2..Recoded | CCCGCATCCAGATAGATACAAGA |
| mAsPCR-seg52.2..Reverse | GCAGGCATTTGAGTTCAGGTC |
| mAsPCR-seg52.2..Wild-Type | CCCGCATCCAGATAGATACAACT |
| mAsPCR-seg52.3..Recoded | GTTTGCAGGATTTCGCGTAG |
| mAsPCR-seg52.3..Reverse | CTCAACATACGCAACCTGGTG |
| mAsPCR-seg52.3..Wild-Type | GTTTGCAGGATTTCGCGCAA |
| mAsPCR-seg52.4..Recoded | AGAGGAAGTTGTGCAAAACGTG |
| mAsPCR-seg52.4..Reverse | AGCAAGCTACAAACGCGAAAC |
| mAsPCR-seg52.4..Wild-Type | AGAGGAAGTTGTGCAAAACGGC |
| mAsPCR-seg52.5..Recoded | GCAGACGACCAATCAGAGTTGA |
| mAsPCR-seg52.5..Reverse | CGGATGGTGCGTTTCCGTA |
| mAsPCR-seg52.5..Wild-Type | GCAGACGACCAATCAGAGTACT |
| mAsPCR-seg52.6..Recoded | CAAGGACTGTATGGTAATCACGAAG |
| mAsPCR-seg52.6..Reverse | CGTGAACATGCGATCTTATCTTATCC |
| mAsPCR-seg52.6..Wild-Type | CAAGGACTGTATGGTAATCACGCAA |
| mAsPCR-seg52.7..Recoded | ATCGCTTATTTGATACAAGTCCTGAAAG |
| mAsPCR-seg52.7..Reverse | GCGGGGCTTTCTATAAACGAT |
| mAsPCR-seg52.7..Wild-Type | ATCGCTTATTTGATACAAGTCCACTCAA |
| mAsPCR-seg52.8..Recoded | CCAGTTGCTCCGGGTTAAG |
| mAsPCR-seg52.8..Reverse | TATCGCTATCCCGTCTTTAATCCAC |
| mAsPCR-seg52.8..Wild-Type | CCAGTTGCTCCGGGTTCAA |
| mAsPCR-seg53.1..Recoded | AAAGTGAACAGATATTAATAATTTTGCGTGA |
| mAsPCR-seg53.1..Reverse | TTTCAGGTGGATTACTTTTCTCAGGT |
| mAsPCR-seg53.1..Wild-Type | ACAATGAACAGATATTAATAATTTTGCCGCT |
| mAsPCR-seg53.2..Recoded | GATTATGATCGGCTTTGATTCCTCA |
| mAsPCR-seg53.2..Reverse | AGTTAAAGTTTTTATTATGTTCCCTGCATCA |
| mAsPCR-seg53.2..Wild-Type | GATTATGATCGGCTTTGATTCCAGC |
| mAsPCR-seg53.3..Recoded | GCGTGGTAGCTAATGATCGTT |
| mAsPCR-seg53.3..Reverse | GCTCTCCCCAGTCGATATTCTC |
| mAsPCR-seg53.3..Wild-Type | GCGTGGTAGCTAATGATCGTA |
| mAsPCR-seg53.4..Recoded | GCAATGCACGCTGGATATTCTTTC |
| mAsPCR-seg53.4..Reverse | CATGTTGCACCATATCTTCCAGGA |
| mAsPCR-seg53.4..Wild-Type | GCAATGCACGCTGGATATTTTAAG |
| mAsPCR-seg53.5..Recoded | GCAAACAGTTCGATGCCCTA |
| mAsPCR-seg53.5..Reverse | AAAACAAGAACAAGAAAGGAAGGGTT |
| mAsPCR-seg53.5..Wild-Type | GCAAACAGTTCGATGCCTTG |
| mAsPCR-seg53.6..Recoded | TAAGTGAAGAGAGAAATTAGTGGACGATC |
| mAsPCR-seg53.6..Reverse | GTCGTATAAAAGGTATGAATTGTGGGTT |
| mAsPCR-seg53.6..Wild-Type | TAAGTGAAGAGAGAAATTAGTGGACGATT |
| mAsPCR-seg53.7..Recoded | GTTTCCATATGGCAGCCTATCAAT |
| mAsPCR-seg53.7..Reverse | AGTTGCCTTACGATTTTTGAGAGC |
| mAsPCR-seg53.7..Wild-Type | GTTTCCATATGGCAGCCTATCAAA |
| mAsPCR-seg53.8..Recoded | CCATCTCTGCCAGCACTTTTAG |
| mAsPCR-seg53.8..Reverse | TTCGGTTGGTATGGCGTAGG |
| mAsPCR-seg53.8..Wild-Type | CCATCTCTGCCAGCACTTTCAA |
| mAsPCR-seg54.1..Recoded | CTTCCGCCAGCGTTGCTAG |
| mAsPCR-seg54.1..Reverse | CGAGAGAAAGTGGCGCAAC |
| mAsPCR-seg54.1..Wild-Type | CTTCCGCCAGCGTTGCTAA |
| mAsPCR-seg54.2..Recoded | TTAATGATATCGGGCTACTACACTCA |
| mAsPCR-seg54.2..Reverse | GAAGAAAGCGCACCGTACC |
| mAsPCR-seg54.2..Wild-Type | TTAATGATATCGGGTTGTTGCACAGC |
| mAsPCR-seg54.3..Recoded | CGTGATAGCATGTCATCAAAACCAAG |
| mAsPCR-seg54.3..Reverse | GGTCGTCTTTGAAACCTGGAAAG |
| mAsPCR-seg54.3..Wild-Type | CGACTTAACATGTCATCAAAACCCAA |
| mAsPCR-seg54.4..Recoded | GCTATGGCGATCTCATCTGTAC |
| mAsPCR-seg54.4..Reverse | CATCCTGACGTACGACCTGAAA |
| mAsPCR-seg54.4..Wild-Type | GCTATGGCGATCAGTAGCGTAT |
| mAsPCR-seg54.5..Recoded | CGCGAAAGTCCTACTTCTTCAAATAG |
| mAsPCR-seg54.5..Reverse | ATCCACCCCTTCCTCTGTTTATAA |
| mAsPCR-seg54.5..Wild-Type | CGGCTTAATCCTACTTCTTCAAACAA |
| mAsPCR-seg54.6..Recoded | CTTATTATCGCCTCCAAAGTGTCA |
| mAsPCR-seg54.6..Reverse | CGCGTTGGTACTCTGCCA |
| mAsPCR-seg54.6..Wild-Type | TTAATTATCGCCTCCAAAGTGAGC |
| mAsPCR-seg54.7..Recoded | GGCGAACCAGACGAATCG |
| mAsPCR-seg54.7..Reverse | GGTAACGCACGGTGGTCA |
| mAsPCR-seg54.7..Wild-Type | GGCGAACCAGACGAAAGC |
| mAsPCR-seg54.8..Recoded | TGCCTGAGACATGAAGAATACTGA |
| mAsPCR-seg54.8..Reverse | TCTGCGAAAGATTGATGGTATTCC |
| mAsPCR-seg54.8..Wild-Type | TGCCTGAGACATGAAGAATACGCT |
| mAsPCR-seg55.1..Recoded | GAATATGCGCCTATGACAAATGCT |
| mAsPCR-seg55.1..Reverse | ATCACACGAGAAGTTCAGAAGCAT |
| mAsPCR-seg55.1..Wild-Type | GAATATGCGCCTATGACAAATGCG |
| mAsPCR-seg55.2..Recoded | TCCAATCGGTATCAATAATCTATCTCAATCA |
| mAsPCR-seg55.2..Reverse | AATCTCGGTTCCTATTTTAATGTTCAGAC |
| mAsPCR-seg55.2..Wild-Type | TCCAATCGGTATCAATAATTTATCTCAAAGT |
| mAsPCR-seg55.3..Recoded | GATAACGGCAATTTCTCGGAACTT |
| mAsPCR-seg55.3..Reverse | CCTTTCGCTTCACCTTCCAG |
| mAsPCR-seg55.3..Wild-Type | GATAACGGCAATTTCAGCGAATTA |
| mAsPCR-seg55.4..Recoded | TATCACCCGCAACGTCAATCA |
| mAsPCR-seg55.4..Reverse | GTGGCCGATATAACCGAGAAC |
| mAsPCR-seg55.4..Wild-Type | TATCACCCGCAACGTCAAAGC |
| mAsPCR-seg55.5..Recoded | GGCTACAACCATCACCTTTCG |
| mAsPCR-seg55.5..Reverse | CACTGAGTGAACTGAGCCTGA |
| mAsPCR-seg55.5..Wild-Type | GGCTACAACCATCACCTTAGC |
| mAsPCR-seg55.6..Recoded | AAAATACTTCCAGCCTCTATTTATGTACTT |
| mAsPCR-seg55.6..Reverse | CAATAAACCGCAGCGCAGAG |
| mAsPCR-seg55.6..Wild-Type | AAAATATTGCCAGCCTCTATTTATGTATTA |
| mAsPCR-seg55.7..Recoded | CGAAAGGAGAAACACTGATGTCA |
| mAsPCR-seg55.7..Reverse | AAGAGATCCGACGAAATGAGCAT |
| mAsPCR-seg55.7..Wild-Type | CGAAAGGAGAAACACTGATGAGC |
| mAsPCR-seg55.8..Recoded | TCCCTGGATCAATTTATCGAAGCAT |
| mAsPCR-seg55.8..Reverse | GAAATCGTTCGGGAAGGCAATC |
| mAsPCR-seg55.8..Wild-Type | AGCCTGGATCAATTTATCGAAGCAA |
| mAsPCR-seg56.1..Recoded | AACTGTATGAGCGTTATCAGCGA |
| mAsPCR-seg56.1..Reverse | CCTCACGGCTAGGTTCGC |
| mAsPCR-seg56.1..Wild-Type | AACTGTATGAGCGTTATCAGAGG |
| mAsPCR-seg56.2..Recoded | GCAGCCATTCGTGTTCTTTTGA |
| mAsPCR-seg56.2..Reverse | CGATCTGTTTATTGCCACCACTG |
| mAsPCR-seg56.2..Wild-Type | GCAGCCATTCGTGTTCTTTGCT |
| mAsPCR-seg56.3..Recoded | TCCAGTCCTAGCCAGTGTGA |
| mAsPCR-seg56.3..Reverse | GGGAGAAATCACCGCCATG |
| mAsPCR-seg56.3..Wild-Type | TCCAGTCCTAACCAGTGGCT |
| mAsPCR-seg56.4..Recoded | TGTTTACAGGCAAATTGAGGTAGTAG |
| mAsPCR-seg56.4..Reverse | CAGTTTTTGCCCTTGTTCCGT |
| mAsPCR-seg56.4..Wild-Type | TGTTTACAGGCAAATTGAGGCAATAA |
| mAsPCR-seg56.5..Recoded | TATTTTTCCATCAGATAGCGCTTAGGA |
| mAsPCR-seg56.5..Reverse | GGAAAATTATCGCCACCATGCTT |
| mAsPCR-seg56.5..Wild-Type | TATTTTTCCATCAGATAGCGCCTAACT |
| mAsPCR-seg56.6..Recoded | GGTTTCTTCACCGTCACTGA |
| mAsPCR-seg56.6..Reverse | GCATAATTCCCGTCATCAAACTTCTAG |
| mAsPCR-seg56.6..Wild-Type | GGTTTCTTCACCGTCACGCT |
| mAsPCR-seg56.7..Recoded | TTGCCGCCAAAATATTCGTATGA |
| mAsPCR-seg56.7..Reverse | GCGCTACTCGGTTCGGAA |
| mAsPCR-seg56.7..Wild-Type | TTGCCGCCAAAATATTCGTAGCT |
| mAsPCR-seg56.8..Recoded | GCTTTTCAGGCTTACTCGCTTTCC |
| mAsPCR-seg56.8..Reverse | CTGACCGTTGATATTGTTGCCT |
| mAsPCR-seg56.8..Wild-Type | GCTTTTCAGGCTTACAGTTTGAGT |
| mAsPCR-seg57.1..Recoded | AAATCGATCGAACTCGGTGTATCA |
| mAsPCR-seg57.1..Reverse | GTCTTTACGCATCAGGATCACATC |
| mAsPCR-seg57.1..Wild-Type | AAATCGATCGAACTCGGTGTAAGC |
| mAsPCR-seg57.2..Recoded | GGTTAAACTTCCTCCGCTGTCA |
| mAsPCR-seg57.2..Reverse | CGCGAACCAAACAGCGTATT |
| mAsPCR-seg57.2..Wild-Type | GGTTAAATTACCTCCGCTCAGT |
| mAsPCR-seg57.3..Recoded | CCGCACTGGTTATGGGTTTTT |
| mAsPCR-seg57.3..Reverse | GTCACGGCCATCAAGCAC |
| mAsPCR-seg57.3..Wild-Type | CCGCACTGGTTATGGGTTTTA |
| mAsPCR-seg57.4..Recoded | CTAAACAGCAAGCGAATCAGTCA |
| mAsPCR-seg57.4..Reverse | CAGAGATGTTGAAGAAGTCGAATGC |
| mAsPCR-seg57.4..Wild-Type | CTAAACAGCAAGCGAATCAGAGC |
| mAsPCR-seg57.5..Recoded | TCCAGACGGAAGATACTGAATACT |
| mAsPCR-seg57.5..Reverse | CAGAGGATTTTCGGGATGTCG |
| mAsPCR-seg57.5..Wild-Type | TCCAGACGGAAGATACTGAATAGA |
| mAsPCR-seg57.6..Recoded | TGTTAAGCTGACCAACACCATCT |
| mAsPCR-seg57.6..Reverse | GCCACCAGCGAATAGGTCA |
| mAsPCR-seg57.6..Wild-Type | TGTTAAGCTGACCAACACCATCA |
| mAsPCR-seg57.7..Recoded | CGTCGGTACTTATTGGTGCCT |
| mAsPCR-seg57.7..Reverse | GGGCTATCTTGACCGACTGAC |
| mAsPCR-seg57.7..Wild-Type | CGTCGGTATTGATTGGTGCCA |
| mAsPCR-seg57.8..Recoded | GCGAACTATCTGGATAACTTCTCCCTT |
| mAsPCR-seg57.8..Reverse | TCGACATCTTCCAGACCAATATGC |
| mAsPCR-seg57.8..Wild-Type | GCGAACTATCTGGATAACTTCAGTTTA |
| mAsPCR-seg58.1..Recoded | CCGGCTTCATCATCTTCGAAAG |
| mAsPCR-seg58.1..Reverse | CGAGAAAGTGAAGGGCGATAAAG |
| mAsPCR-seg58.1..Wild-Type | CCGGCTTCATCATCTTCGATAA |
| mAsPCR-seg58.2..Recoded | GCATTGACAAGTTTTTTAACCTGTGATAG |
| mAsPCR-seg58.2..Reverse | TTATCATGTGGCGTAAAGAAACAGG |
| mAsPCR-seg58.2..Wild-Type | GCATTGACAAGTTTTTTAACCTGACTCAA |
| mAsPCR-seg58.3..Recoded | CAACCGCTACTTCTATCTCTTCTT |
| mAsPCR-seg58.3..Reverse | CGAAGATCGTATACTTCAAGCAATGATT |
| mAsPCR-seg58.3..Wild-Type | CAACCGCTATTGCTAAGTTTGTTG |
| mAsPCR-seg58.4..Recoded | GGTATGCCTGTTCCCGTGA |
| mAsPCR-seg58.4..Reverse | TCATCGTCTATTCAACGGGCAA |
| mAsPCR-seg58.4..Wild-Type | GGTATGCCTGTTCCCGGCT |
| mAsPCR-seg58.5..Recoded | AGATTGACCCTAATAATAACCCCTCA |
| mAsPCR-seg58.5..Reverse | CTGGTACTGGATTGTATTGATCGCT |
| mAsPCR-seg58.5..Wild-Type | AGATTGACCCTAATAATAACCCCAGC |
| mAsPCR-seg58.6..Recoded | CTCTTAAATTCAAACTGGCCCTTCTT |
| mAsPCR-seg58.6..Reverse | AGTAAGTGCCGCCAGTGAG |
| mAsPCR-seg58.6..Wild-Type | GCCTTAAATTCAAACTGGCCTTGTTG |
| mAsPCR-seg58.7..Recoded | CCGCACCTGATCCCATCA |
| mAsPCR-seg58.7..Reverse | CGTCGAGCATCTCCTGTGG |
| mAsPCR-seg58.7..Wild-Type | CCGCACCTGATCCCAAGC |
| mAsPCR-seg58.8..Recoded | CAATCACAACCAAACGACTCATCA |
| mAsPCR-seg58.8..Reverse | GAACCAGTCGCCCCAGGA |
| mAsPCR-seg58.8..Wild-Type | CAATCACAACCAAACGACAGCAGT |
| mAsPCR-seg59.1..Recoded | AGCCAGTTCCGGGTCGATT |
| mAsPCR-seg59.1..Reverse | GTTAACGGCTGAAGGACATCG |
| mAsPCR-seg59.1..Wild-Type | AGCCAGTTCCGGGTCGATG |
| mAsPCR-seg59.2..Recoded | GGTACGAATCGACATATAGCCTGA |
| mAsPCR-seg59.2..Reverse | CATTTGTTGTTATTTTGCACGGTTTTTG |
| mAsPCR-seg59.2..Wild-Type | GGTACGAATCGACATATAGCCACT |
| mAsPCR-seg59.3..Recoded | ACAACTATAACTTCTGTCTTGATGGTCTT |
| mAsPCR-seg59.3..Reverse | GGTTTGCCGGACATTTTTGAGA |
| mAsPCR-seg59.3..Wild-Type | ACAACTATAACTTCTGTCTTGATGGTTTG |
| mAsPCR-seg59.4..Recoded | AACGAACGTAATACCAAACCCTCT |
| mAsPCR-seg59.4..Reverse | CGTCCAGTCTGAACGTTTGC |
| mAsPCR-seg59.4..Wild-Type | AACGAACGTAATACCAAACCCAGC |
| mAsPCR-seg59.5..Recoded | TGAGATGTATGAGTCGCCAATAGA |
| mAsPCR-seg59.5..Reverse | CCTGAAGATAAGTAAGATTTGACATAACCG |
| mAsPCR-seg59.5..Wild-Type | ACTGATGTATGAGTCGCCAATGCT |
| mAsPCR-seg59.6..Recoded | TATTCAGGCCATTCATAAGCAGAAATGA |
| mAsPCR-seg59.6..Reverse | TTCGTACACTAATTACCCTTCGCA |
| mAsPCR-seg59.6..Wild-Type | TATTCAGGCCATTCATAAGCAGAAAACT |
| mAsPCR-seg59.7..Recoded | AAGAAGAGCTTTCAAAGATTCGTTCA |
| mAsPCR-seg59.7..Reverse | CGTGATGACTGTCCGCCATA |
| mAsPCR-seg59.7..Wild-Type | AAGAAGAGTTGAGTAAGATTCGTAGC |
| mAsPCR-seg59.8..Recoded | GCAAAAATGGACTGGTACCTGAAG |
| mAsPCR-seg59.8..Reverse | TAGATTGTCGTCAGGATGCCTTC |
| mAsPCR-seg59.8..Wild-Type | GCAAAAATGGACTGCTATCTGAAA |
| mAsPCR-seg60.1..Recoded | GTTTTTACCTAGATAACCTGAAATGACTGA |
| mAsPCR-seg60.1..Reverse | GCACCGCGTGTTTCACTC |
| mAsPCR-seg60.1..Wild-Type | GTTTTTACCTAAATAACCGCTAATGACGCT |
| mAsPCR-seg60.2..Recoded | GCGCCGATTCAATACCCGAAAG |
| mAsPCR-seg60.2..Reverse | CCTACGCCAACCCGAACA |
| mAsPCR-seg60.2..Wild-Type | GCGCCGATTCAATACCACTTAA |
| mAsPCR-seg60.3..Recoded | CTTCTAAAAATAACGCCTGTTCTCATATCA |
| mAsPCR-seg60.3..Reverse | CCTCCCGGGTAAAATATTGCTT |
| mAsPCR-seg60.3..Wild-Type | TTACTAAAAATAACGCCTGTTTTAATAAGC |
| mAsPCR-seg60.4..Recoded | TAACCCATCGAAACCGCAGAAAG |
| mAsPCR-seg60.4..Reverse | ATCATTTCAGGGATTGCAGTGC |
| mAsPCR-seg60.4..Wild-Type | TAACCCATCGAAACCGCACTTAA |
| mAsPCR-seg60.5..Recoded | CACGCTATGCCAAATATTGTTCTATCA |
| mAsPCR-seg60.5..Reverse | CGTTAATGCGATTCACCGGAAC |
| mAsPCR-seg60.5..Wild-Type | CACGCTATGCCAAATATTGTTTFAAGC |
| mAsPCR-seg60.6..Recoded | GATGCGATTTTCTGGTTTACTCTTCTC |
| mAsPCR-seg60.6..Reverse | CGATGTCACCACGTTAATATGCAC |
| mAsPCR-seg60.6..Wild-Type | GATGCGATTTTCTGGTTTACTTTGTTG |
| mAsPCR-seg60.7..Recoded | GTTTACCTCTGCAACGCTATCTTC |
| mAsPCR-seg60.7..Reverse | TGTGTGAATCGGGTGTTAACAGA |
| mAsPCR-seg60.7..Wild-Type | GTTTACCTCTGCAACGCTAAGTAG |
| mAsPCR-seg60.8..Recoded | ACCACTTTCGCAGATCCTCTCT |
| mAsPCR-seg60.8..Reverse | GGTGAAAGCGCGAAGTAACAAATA |
| mAsPCR-seg60.8..Wild-Type | ACCACTTAGCCAGATCTTAAGC |
| mAsPCR-seg61.1..Recoded | CCAGCAGCAGATCCAGTGA |
| mAsPCR-seg61.1..Reverse | CTGATCTTTACCTGGTTCTGTATGCT |
| mAsPCR-seg61.1..Wild-Type | CCAGCAGCAGATCCAGACT |
| mAsPCR-seg61.2..Recoded | CGTTCCATAAGCGTTTGTTCCGA |
| mAsPCR-seg61.2..Reverse | GCACTTACGCTTGCAGGATG |
| mAsPCR-seg61.2..Wild-Type | CTTTCCATTAACGTTTGTTCGCT |
| mAsPCR-seg61.3..Recoded | GCCGCACGTTATGAAGATGAAT |
| mAsPCR-seg61.3..Reverse | CGCAAGCACCTACCGGAT |
| mAsPCR-seg61.3..Wild-Type | GCCGCACGTTATGAAGATGAAA |
| mAsPCR-seg61.4..Recoded | GGCCTTTGTTTTCCAGATTCTCA |
| mAsPCR-seg61.4..Reverse | CGCCTGCTCACCGGTATT |
| mAsPCR-seg61.4..Wild-Type | GGCCTTTGTTTTCCAGATTCTCC |
| mAsPCR-seg61.5..Recoded | TGAGGGCGACGCAATCTC |
| mAsPCR-seg61.5..Reverse | CGCACGATTATAGTTACGCTCAAT |
| mAsPCR-seg61.5..Wild-Type | TGAGGGCGACGCAATCAG |
| mAsPCR-seg61.6..Recoded | TGGCTGACGTCGGTATGC |
| mAsPCR-seg61.6..Reverse | TCGATGAGGTGAAGCAGGAC |
| mAsPCR-seg61.6..Wild-Type | TGGCTGACGTCGGTATGT |
| mAsPCR-seg61.7..Recoded | TCATCATCACCGTAGAATGAACAAG |
| mAsPCR-seg61.7..Reverse | GTCTGATTGGCGGGCAAAT |
| mAsPCR-seg61.7..Wild-Type | TCATCATCACCGTACTATGCAACAA |
| mAsPCR-seg61.8..Recoded | GAGGCCCGACTGATCATTTCA |
| mAsPCR-seg61.8..Reverse | TGGAATGACATACTCAGGTTCGC |
| mAsPCR-seg61.8..Wild-Type | GAGGCCAGACTGATCATTAGC |
| mAsPCR-seg62.1..Recoded | CATCATCTTCTCAAACACCGCAAG |
| mAsPCR-seg62.1..Reverse | AAAATTTTCGCCATGTATTACCAGGT |
| mAsPCR-seg62.1..Wild-Type | CATCATCTTCTCAAACACCGCTAA |
| mAsPCR-seg62.2..Recoded | GCTTCGCGTATTCCTGATAGTCT |
| mAsPCR-seg62.2..Reverse | CCGGAATATCGCTAAAGATCGC |
| mAsPCR-seg62.2..Wild-Type | GCTTCGCGTATTCCTGATAGTCG |
| mAsPCR-seg62.3..Recoded | CGATCTAAAAGTGGGCAAATTCTCA |
| mAsPCR-seg62.3..Reverse | GTGTGAAGAGTTCCACCATGAG |
| mAsPCR-seg62.3..Wild-Type | CGATCTAAAAGTGGGCAAATTCAGC |
| mAsPCR-seg62.4..Recoded | CAGGGTCAGTTTTACCCCTGA |
| mAsPCR-seg62.4..Reverse | CACTCCTGACTCCTTTTGACCA |
| mAsPCR-seg62.4..Wild-Type | CAGGGTCAGTTTTACCCCACT |
| mAsPCR-seg62.5..Recoded | TTTTACGAGCGCCATGTCAAAC |
| mAsPCR-seg62.5..Reverse | CGACAAAGTCCGGCAAACC |
| mAsPCR-seg62.5..Wild-Type | TTTTACGAGCGCCATGTCAAAT |
| mAsPCR-seg62.6..Recoded | CACAGCAGTAGGGATATGCGA |
| mAsPCR-seg62.6..Reverse | CGCTAAACTTGCGTGACTACA |
| mAsPCR-seg62.6..Wild-Type | CACAGCAGTAGGGATATGGCT |
| mAsPCR-seg62.7..Recoded | GAATTCCGGTAACCAGATTGACA |
| mAsPCR-seg62.7..Reverse | GAAGCCGGTCGAATTTACTACC |
| mAsPCR-seg62.7..Wild-Type | GAATTCCGGTAACCAGATTGACG |
| mAsPCR-seg62.8..Recoded | GGCCTGGTATCACTCTCCT |
| mAsPCR-seg62.8..Reverse | GCCGTTTCCAGCGCAATATT |
| mAsPCR-seg62.8..Wild-Type | GGCCTGGTAAGCCTCTCCA |
| mAsPCR-seg63.1..Recoded | CACGTCTTCAACCTGTTATTCGTC |
| mAsPCR-seg63.1..Reverse | GTATTCGCAGTACCCAGGTCAA |
| mAsPCR-seg63.1..Wild-Type | GTCGTTTACAACCTGTTATTCGTT |
| mAsPCR-seg63.2..Recoded | ATGAATATCTGAAATCTCTAGGTGCTTCA |
| mAsPCR-seg63.2..Reverse | GCTGTTTAGTGGAGTATCAATGCG |
| mAsPCR-seg63.2..Wild-Type | ATGAATATCTGAAAAGTTTAGGTGCTAGC |
| mAsPCR-seg63.3..Recoded | CATAAGCCAGTTTTGAACAATTCCAGA |
| mAsPCR-seg63.3..Reverse | TCTGAAGACCCGGCAAGAAC |
| mAsPCR-seg63.3..Wild-Type | CATTAACCAGTTTTGAACAATTCCGCT |
| mAsPCR-seg63.4..Recoded | CGCTTCCAGGGCAACAACTT |
| mAsPCR-seg63.4..Reverse | CGTTGCTCGCATATTCTGTAGG |
| mAsPCR-seg63.4..Wild-Type | CGTTACCAGGGCAACAATTG |
| mAsPCR-seg63.5..Recoded | TGCCGATTGTGCGTATCCTT |
| mAsPCR-seg63.5..Reverse | GTATTTACCAGCCCAGGAATTACC |
| mAsPCR-seg63.5..Wild-Type | TGCCGATTGTGCGTATCTTA |
| mAsPCR-seg63.6..Recoded | GCACCTTTACCACCAGCTGA |
| mAsPCR-seg63.6..Reverse | GTTGTGCCTGGTGAAACGG |
| mAsPCR-seg63.6..Wild-Type | GCACCTTTACCACCAGCACT |
| mAsPCR-seg63.7..Recoded | CCAATACCTTCTTCTGCGTACATT |
| mAsPCR-seg63.7..Reverse | TGTCAATCAGAGGGGGATTTGT |
| mAsPCR-seg63.7..Wild-Type | CCAATACCTTCTTCTGCGTACATC |
| mAsPCR-seg63.8..Recoded | ACGTGAGAATCATCATCCAGTATTAG |
| mAsPCR-seg63.8..Reverse | ACCCGTAGTATCCCCACTTATCT |
| mAsPCR-seg63.8..Wild-Type | ACGTGAGAATCATCATCCAGTATCAA |
| mAsPCR-seg64.1..Recoded | GCAGACGACCGATTGCAGA |
| mAsPCR-seg64.1..Reverse | AGCTGTGGGTAAAGCTGTCG |
| mAsPCR-seg64.1..Wild-Type | GCAGACGACCGATTGCACT |
| mAsPCR-seg64.2..Recoded | GCTCCGCTTCTGGAAAAAAACT |
| mAsPCR-seg64.2..Reverse | CGACCTTCACCACCACCAT |
| mAsPCR-seg64.2..Wild-Type | GCTCCGTTGCTGGAAAAAAACA |
| mAsPCR-seg64.3..Recoded | TAAGTGCGGAAGTTGCCAGAAG |
| mAsPCR-seg64.3..Reverse | CTATCTCTACATCCGCCAGTTCAA |
| mAsPCR-seg64.3..Wild-Type | TTAATGCGGAAGTTGCCAGTAA |
| mAsPCR-seg64.4..Recoded | ACACCGGAGACTCATCAACTAG |
| mAsPCR-seg64.4..Reverse | CGGCTGGGATGAATTTGAGTG |
| mAsPCR-seg64.4..Wild-Type | TCACCGGAGACTCATCAACCAA |
| mAsPCR-seg64.5..Recoded | GCCGCCATTTTTACCCTCTCA |
| mAsPCR-seg64.5..Reverse | ATCCGCTTGTAGTCAGTATTATTTTGC |
| mAsPCR-seg64.5..Wild-Type | GCCGCCATTTTTACCCTCACT |
| mAsPCR-seg64.6..Recoded | CTGCTATTTACCGACTCCTTCTTCTC |
| mAsPCR-seg64.6..Reverse | GGAGATAAAACCAAGCTGACCGA |
| mAsPCR-seg64.6..Wild-Type | CTGTTATTTACCGACTCCTTCTTCAG |
| mAsPCR-seg64.7..Recoded | CATCGCGATTATGCCCAGTC |
| mAsPCR-seg64.7..Reverse | CGTGACTGCCGTACCGTT |
| mAsPCR-seg64.7..Wild-Type | CATCGCGATTATGCCCAGAG |
| mAsPCR-seg64.8..Recoded | ATCAAAAACGATCTCAAGCAGCTT |
| mAsPCR-seg64.8..Reverse | TCCAGGTAAATTCCATCAGCGTTA |
| mAsPCR-seg64.8..Wild-Type | ATCAAAAACGATCTCAAGCAGTTG |
| mAsPCR-seg65.1..Recoded | GCAGGGTGTAGTCGATTGATGA |
| mAsPCR-seg65.1..Reverse | GTCTACCTGTGGCGCATCA |
| mAsPCR-seg65.1..Wild-Type | GCAGGGTGTAGTCGATACTGCT |
| mAsPCR-seg65.2..Recoded | CGCATTACACTCTGCAGCTGT |
| mAsPCR-seg65.2..Reverse | ACCTCGGCGCAATTTGTTTC |
| mAsPCR-seg65.2..Wild-Type | GCCATTACACTCTGCAGCTGA |
| mAsPCR-seg65.3..Recoded | TCATCTGAAACCTTCCGTGTGAG |
| mAsPCR-seg65.3..Reverse | TACTGATGAACCCGCCAATTAATTTT |
| mAsPCR-seg65.3..Wild-Type | TCATCGCTAACCTTCCTTGTTAA |
| mAsPCR-seg65.4..Recoded | TTTCTCGCTGGGATGCATCA |
| mAsPCR-seg65.4..Reverse | ACATCGTTATTTTCCAGCACGTTC |
| mAsPCR-seg65.4..Wild-Type | TTTCTCGCTGGGATGCAAGT |
| mAsPCR-seg65.5..Recoded | GTACATGATATCGTTTACAACCCATCA |
| mAsPCR-seg65.5..Reverse | CCACAGAAAGCGTCGACAAC |
| mAsPCR-seg65.5..Wild-Type | GTACATGATATCGTTTACAACCCAAGC |
| mAsPCR-seg65.6..Recoded | GCTTCTTCTCATCGTCACCCTT |
| mAsPCR-seg65.6..Reverse | GAATTCATAGTGTTGCGCCCAA |
| mAsPCR-seg65.6..Wild-Type | GTTATTGCTCATCGTCACCTTG |
| mAsPCR-seg65.7..Recoded | CGTGTCCATGCCGTTTCTC |
| mAsPCR-seg65.7..Reverse | AAAGTTCTGTCTCGCCATTTCAAAA |
| mAsPCR-seg65.7..Wild-Type | CGTGTCCATGCCGTTTTTG |
| mAsPCR-seg65.8..Recoded | CGGAATTGGCTTATCGATACCTTTT |
| mAsPCR-seg65.8..Reverse | GTGACCCACGGCTTCCTG |
| mAsPCR-seg65.8..Wild-Type | CGGAATTGGCTTATCGATACCTTTC |
| mAsPCR-seg66.1..Recoded | GTTCACTCCGGCTTATGTCA |
| mAsPCR-seg66.1..Reverse | GGTCGCCCATCCCTCATG |
| mAsPCR-seg66.1..Wild-Type | GGAGTCTGCGGTTGATGAGC |
| mAsPCR-seg66.2..Recoded | CAGCGAGGTAAGAATCCATTTACG |
| mAsPCR-seg66.2..Reverse | GGTGCGCTGACTATCGGT |
| mAsPCR-seg66.2..Wild-Type | CAGCGAGGTCAAAATCCATTTTCT |
| mAsPCR-seg66.3..Recoded | CGGTAAATGCGGTAAGACCTGAT |
| mAsPCR-seg66.3..Reverse | TGGTGGTTATCAGGTGGGAAATT |
| mAsPCR-seg66.3..Wild-Type | CGGTAAATGCGGTTAAACCACTG |
| mAsPCR-seg66.4..Recoded | CCCTCAGCTTCAGGAAATTCA |
| mAsPCR-seg66.4..Reverse | CGTTGGGATGATTGCGTTCC |
| mAsPCR-seg66.4..Wild-Type | CCCAGCGCTTCAGGAAATAGC |
| mAsPCR-seg66.5..Recoded | CCTGGCTGGTTACCGGTT |
| mAsPCR-seg66.5..Reverse | ACCTTAGTACCCCGCCGTA |
| mAsPCR-seg66.5..Wild-Type | CCTGGCTGGTTACCGGTA |
| mAsPCR-seg66.6..Recoded | CTCACCTTTAAACAJTTTAGAGTACCATGA |
| mAsPCR-seg66.6..Reverse | GAGTATGATGTCGAACTGGCCTTA |
| mAsPCR-seg66.6..Wild-Type | CTCACCTTTAAACATTTTGCTGTACCAACT |
| mAsPCR-seg66.7..Recoded | GTCACCATAGGCCAGGTTTGA |
| mAsPCR-seg66.7..Reverse | ATGTGCGTCTGTTCCGTGAA |
| mAsPCR-seg66.7..Wild-Type | GTCACCATAGGCCAGGTTACT |
| mAsPCR-seg66.8..Recoded | CTGATTATCGCCGGTGCCT |
| mAsPCR-seg66.8..Reverse | CAGTACCGCGGGCTTGTT |
| mAsPCR-seg66.8..Wild-Type | CTGATTATCGCCGGTGCCA |
| mAsPCR-seg67.1..Recoded | TTTTTTTAGTCGCCACGTCAGAAG |
| mAsPCR-seg67.1..Reverse | GGAACGGCATTGTCACTTACG |
| mAsPCR-seg67.1..Wild-Type | TTTTTTTAATCGCCACGTCAGTAA |
| mAsPCR-seg67.2..Recoded | TCACATTGTCAGCTTGAAAATCTCTCT |
| mAsPCR-seg67.2..Reverse | TCTGTTTTGGAGAGTGCTTTAACATC |
| mAsPCR-seg67.2..Wild-Type | AGCCATTGTCAGCTTGAAAATTTAAGC |
| mAsPCR-seg67.3..Recoded | CAATATTTTTAATCTGGGTATCAAAGAGCTA |
| mAsPCR-seg67.3..Reverse | CATCACCCCGCCAAACCA |
| mAsPCR-seg67.3..Wild-Type | CAATATTTTTAATCTGGGTATCAAAGAGTTG |
| mAsPCR-seg67.4..Recoded | GCGTGCTCATATTCTACGTCGTAATAAC |
| mAsPCR-seg67.4..Reverse | TCATCTTCTATATTAAGTAGCTGTGAAAGGA |
| mAsPCR-seg67.4..Wild-Type | GCGTGCTCATATTCTACGTAGGAATAAT |
| mAsPCR-seg67.5..Recoded | CTTCATACCGGGCTGCTACTTCTT |
| mAsPCR-seg67.5..Reverse | GATGCAGGTAGACCAAAGTACC |
| mAsPCR-seg67.5..Wild-Type | TTGCATACCGGGCTGTTATTATTG |
| mAsPCR-seg67.6..Recoded | CTATCAATAAATTCAACTGGGAAACGCTA |
| mAsPCR-seg67.6..Reverse | GCAGGAAGGGGGAAGAAG |
| mAsPCR-seg67.6..Wild-Type | CTATCAATAAATTCAACTGGGAAACGTTG |
| mAsPCR-seg67.7..Recoded | TAACTTCCTCACTCAAATAGAACGACTTAAG |
| mAsPCR-seg67.7..Reverse | TTGATTCGCAATGCATGACAGA |
| mAsPCR-seg67.7..Wild-Type | TAATTTTCTCACTCAAATTGAACGATTAAAA |
| mAsPCR-seg67.8..Recoded | CGCCGCTACCATCAGGATATTAG |
| mAsPCR-seg67.8..Reverse | GCCTCTATCACTCTGACCTTCG |
| mAsPCR-seg67.8..Wild-Type | CGCCGCTACCATCAGGATATTAC |
| mAsPCR-seg68.1..Recoded | CGCCCGCTCTTCATCTGA |
| mAsPCR-seg68.1..Reverse | ACCTGTCAAAAAATATAACGCACTAATATCA |
| mAsPCR-seg68.1..Wild-Type | CGCCCGCTCTTCATCACT |
| mAsPCR-seg68.2..Recoded | GTGAGGCCCCCTGAATTGA |
| mAsPCR-seg68.2..Reverse | CATTTCTTTGACCGATTGTTGTTCAC |
| mAsPCR-seg68.2..Wild-Type | GACTGGCCCCCTGAATACT |
| mAsPCR-seg68.3..Recoded | TCGCCACGACAATTAGGAGTAG |
| mAsPCR-seg68.3..Reverse | GTCTTCCCTGGCTGCGTT |
| mAsPCR-seg68.3..Wild-Type | TCGCCACGACAATCAACAACAA |
| mAsPCR-seg68.4..Recoded | ACCGCCGAACAGCTTTACTC |
| mAsPCR-seg68.4..Reverse | CCATATTCGGGTGCATCAGTTG |
| mAsPCR-seg68.4..Wild-Type | ACCGCCGAACAGCTTTACAG |
| mAsPCR-seg68.5..Recoded | GATAACGAGTAATTGAAGATGAATGTGCTA |
| mAsPCR-seg68.5..Reverse | TTTCTTGCCCCACAGCCA |
| mAsPCR-seg68.5..Wild-Type | GATAACGAGTAATTGAAGATGAATGTGTTG |
| mAsPCR-seg68.6..Recoded | TGATTGGGGCCATTTTTGTTCTTC |
| mAsPCR-seg68.6..Reverse | TATTCAGCCAGGCGTTAAGGTT |
| mAsPCR-seg68.6..Wild-Type | TGATTGGGGCCATTTTTGTTTTAT |
| mAsPCR-seg68.7..Recoded | GCTCCGGTTTACTCAATCAGCTTA |
| mAsPCR-seg68.7..Reverse | CGATTTGGGTTTCGTTTCGTGT |
| mAsPCR-seg68.7..Wild-Type | GCTCCGGTTTACTCAATCAGCTTC |
| mAsPCR-seg68.8..Recoded | CCAGAGTTTTAGCCTGAACCGA |
| mAsPCR-seg68.8..Reverse | GGGCAAAAAACAAAAAAGGTCAGG |
| mAsPCR-seg68.8..Wild-Type | CCAGAGTTTTAGCCTGAACACT |
| mAsPCR-seg69.1..Recoded | CGGACGTAGATGTGGGAATTTCT |
| mAsPCR-seg69.1..Reverse | GTGTAACGCTCTGTGGAAAGTC |
| mAsPCR-seg69.1..Wild-Type | CGGACGTAGATGTGGGAATTTCG |
| mAsPCR-seg69.2..Recoded | CAAAGACCGGTTTAAGATCATCTGA |
| mAsPCR-seg69.2..Reverse | ACGGCACTATCATTTTTTAACAATGAAAC |
| mAsPCR-seg69.2..Wild-Type | CAAAGACCGGTTTCAAATCATCGCT |
| mAsPCR-seg69.3..Recoded | TAAAAAATCAGACAAAGGCCGATACGT |
| mAsPCR-seg69.3..Reverse | AACCTTTACCCGTTGTGCTTTC |
| mAsPCR-seg69.3..Wild-Type | TAAAAAATCAGACATAAGCCGATACGC |
| mAsPCR-seg69.4..Recoded | CCGAAAGTGCCTGAATTGCA |
| mAsPCR-seg69.4..Reverse | CGTATAACGGTCAGGTACTTTCCA |
| mAsPCR-seg69.4..Wild-Type | CGCTTAATGCCTGAATTGCC |
| mAsPCR-seg69.5..Recoded | CTTGTTTGGAGGATACGTGTTTATTCGA |
| mAsPCR-seg69.5..Reverse | TTTAGCGCCAATCTGAATCGTTAAC |
| mAsPCR-seg69.5..Wild-Type | CTTGTTTACTGCTTACCTGTTTATTACT |
| mAsPCR-seg69.6..Recoded | TAAGGACCCGATTAAAGGCTGCTTTA |
| mAsPCR-seg69.6..Reverse | TTTTTTTCCCATCACTTCTTTCCC |
| mAsPCR-seg69.6..Wild-Type | TTAAGACCCGATTAAAGGCTGCTTTT |
| mAsPCR-seg69.7..Recoded | CCGGACTCGAGATGACCTC |
| mAsPCR-seg69.7..Reverse | GACACATCCGCCAGCATT |
| mAsPCR-seg69.7..Wild-Type | CCGGACTCGAGATGACCAG |
| mAsPCR-seg69.8..Recoded | GGGTTTACTTTCGCCTGAGA |
| mAsPCR-seg69.8..Reverse | GGTGGATCGGCTGATGGC |
| mAsPCR-seg69.8..Wild-Type | GGGTTTACTTTCGCCTGGCT |
| mAsPCR-seg70.1..Recoded | CGGACGACTATGGCTGGATC |
| mAsPCR-seg70.1..Reverse | CGCATCGGTTTATTTACACCAGTC |
| mAsPCR-seg70.1..Wild-Type | CGGACGATTGTGGCTGGATT |
| mAsPCR-seg70.2..Recoded | TGCGCCCGAATAACCGTCTA |
| mAsPCR-seg70.2..Reverse | GTCTGGAGTATTATCGTCGGCTTTA |
| mAsPCR-seg70.2..Wild-Type | TGCGCCCGAATAACAGATTG |
| mAsPCR-seg70.3..Recoded | AGCCGATATCCGGGTCTTCT |
| mAsPCR-seg70.3..Reverse | TTACTGTCAAACACTCTCTGATCTTCA |
| mAsPCR-seg70.3..Wild-Type | AGGCGATATCCGGGTCTTCA |
| mAsPCR-seg70.4..Recoded | GGAACGACACGCCCTTAGAT |
| mAsPCR-seg70.4..Reverse | AACAATGTTGGTGAGCTTGAGA |
| mAsPCR-seg70.4..Wild-Type | GGAAACTCACGCCCTTGCTG |
| mAsPCR-seg70.5..Recoded | CCTTGTTCGTGTTAATCCCAAGA |
| mAsPCR-seg70.5..Reverse | GCCAGCGTTTCGTACCATG |
| mAsPCR-seg70.5..Wild-Type | CCTTGTTCGTGTTAATCCCAGCT |
| mAsPCR-seg70.6..Recoded | AAGAACTCAACGCGCTACTTC |
| mAsPCR-seg70.6..Reverse | GCTTTTATGGGGGCCGAGA |
| mAsPCR-seg70.6..Wild-Type | AAGAACTCAACGCGCTATTGT |
| mAsPCR-seg70.7..Recoded | ACTGGAGCTTATCAGTGTTAATTCCATAC |
| mAsPCR-seg70.7..Reverse | TTCTGAATGTTTAAATGTTGCCTATGGT |
| mAsPCR-seg70.7..Wild-Type | ACTGGAGCTTATCAGTGTTAATTCTATAT |
| mAsPCR-seg70.8..Recoded | CCAATAAAAAGCACTGCATGATCAATAAG |
| mAsPCR-seg70.8..Reverse | CGAGGCTATCAGGTTGTGCT |
| mAsPCR-seg70.8..Wild-Type | CCAATAAAAAGCACTGCATGATCAATTAA |
| mAsPCR-seg71.1..Recoded | GCTGGGTAAATGGGCTGATCTT |
| mAsPCR-seg71.1..Reverse | GATGGTCTTTTAGTGCGGCAAC |
| mAsPCR-seg71.1..Wild-Type | GCTGGGTAAATGGGCTGATTTA |
| mAsPCR-seg71.2..Recoded | AAATGAGCTAAAAGAACATAACAAACAACTT |
| mAsPCR-seg71.2..Reverse | GGGGAGGGGAAATTGATAACTTGTA |
| mAsPCR-seg71.2..Wild-Type | AAATGAGTTGAAAGAACATAACAAACAATTG |
| mAsPCR-seg71.3..Recoded | GCGACCATCTTTCTCTTCCGTATTA |
| mAsPCR-seg71.3..Reverse | TGCTCAACCATGCTCTAGGTG |
| mAsPCR-seg71.3..Wild-Type | GCGACCATCTTTCTCTTCCGTATTC |
| mAsPCR-seg71.4..Recoded | GCGTGGTTTATGGGCATGCTA |
| mAsPCR-seg71.4..Reverse | CCGGTTCTGGAATGTGTTGTAC |
| mAsPCR-seg71.4..Wild-Type | GCGTGGTTTATGGGCATGTTG |
| mAsPCR-seg71.5..Recoded | GACGGAATTATGGTTGAAATCTGGTC |
| mAsPCR-seg71.5..Reverse | CGACGACATCTGGGATTGCT |
| mAsPCR-seg71.5..Wild-Type | GACGGAATTATGGTTGAAATCTGGAG |
| mAsPCR-seg71.6..Recoded | GTCCAAAAGCCTCAATTCTTTCA |
| mAsPCR-seg71.6..Reverse | GCAATCTTATCAATCACCCGAAGTC |
| mAsPCR-seg71.6..Wild-Type | GTCCAAAAGCCAGCATTTTGAGC |
| mAsPCR-seg71.7..Recoded | GATGATTGCCTTCTACGCCCTT |
| mAsPCR-seg71.7..Reverse | CGACGGGAAGATAAACATGCC |
| mAsPCR-seg71.7..Wild-Type | GATGATTGCCTTCTACGCCTTA |
| mAsPCR-seg71.8..Recoded | CGGAATCGGCAGAATAAAAAGAATT |
| mAsPCR-seg71.8..Reverse | GCCTGCTTACCTCATATAAAACGC |
| mAsPCR-seg71.8..Wild-Type | CGGAATCGGCAGAATAAACAAAATA |
| mAsPCR-seg72.1..Recoded | TACATCGCCGCCCCTTTTG |
| mAsPCR-seg72.1..Reverse | CGGTATCTACGCTAACCAGTCC |
| mAsPCR-seg72.1..Wild-Type | TACATCGCCGCCCCTTTAC |
| mAsPCR-seg72.2..Recoded | TGAAATCTGCGGAGTTAAGTCGAATA |
| mAsPCR-seg72.2..Reverse | TCACCGCCAGACAAGCAC |
| mAsPCR-seg72.2..Wild-Type | TGAAATCTGCGGAGTTAAGTCGAATT |
| mAsPCR-seg72.3..Recoded | AATCCCCTCCAGCGACGA |
| mAsPCR-seg72.3..Reverse | TGAGGTTTAPCACGACTCTCTGTG |
| mAsPCR-seg72.3..Wild-Type | AATCCCCTCCAGCGAGCT |
| mAsPCR-seg72.4..Recoded | CTACTCCtTTTAAAGGATTAATCATGAAGCTA |
| mAsPCR-seg72.4..Reverse | GCCAGTGCCTTTTCTTCTTCG |
| mAsPCR-seg72.4..Wild-Type | CTACTCGTTTAAAGGATTAATCATGAAGTTG |
| mAsPCR-seg72.5..Recoded | ATTTCCATCTCCGCACCAGA |
| mAsPCR-seg72.5..Reverse | TGCGCGTACAGATTGGCT |
| mAsPCR-seg72.5..Wild-Type | ATTTCCATCTCCGCACCGCT |
| mAsPCR-seg72.6..Recoded | AAGCACGTCAGGGTTCACTT |
| mAsPCR-seg72.6..Reverse | GCCTGTTCAATTTCCTGCCA |
| mAsPCR-seg72.6..Wild-Type | AAGCACGTCAGGGTAGTTTG |
| mAsPCR-seg72.7..Recoded | GGTTTTTCCGGTCGCGAATC |
| mAsPCR-seg72.7..Reverse | GTCCAGCGCCCAGGTATC |
| mAsPCR-seg72.7..Wild-Type | GGTTTTTCCGGTCGCGAAAG |
| mAsPCR-seg72.8..Recoded | ATTACCGAAGATTACCAGGAAATGT |
| mAsPCR-seg72.8..Reverse | GCAGTTATCGTACCAGGGCTTA |
| mAsPCR-seg72.8..Wild-Type | ATTACCGAAGATTACCAGGAAATGA |
| mAsPCR-seg73.1..Recoded | ACAATCAGGTACTTATCTTATTCTATTCTCA |
| mAsPCR-seg73.1..Reverse | GCAGGTTGACGCCATATACC |
| mAsPCR-seg73.1..Wild-Type | ACAAAGTGGTACTTATTTAATTTTATTCAGC |
| mAsPCR-seg73.2..Recoded | ATCAGAGAGACAATAATGCCACCTAG |
| mAsPCR-seg73.2..Reverse | CCGGGTGCAATTGGTTATGTT |
| mAsPCR-seg73.2..Wild-Type | ATCAGAGAGACAATAATGCCACCCAA |
| mAsPCR-seg73.3..Recoded | ATACGTACCTGCGGATGACC |
| mAsPCR-seg73.3..Reverse | CATTGCCATATCACCCTCCGA |
| mAsPCR-seg73.3..Wild-Type | ATACGTACCTGCGGATGACT |
| mAsPCR-seg73.4..Recoded | CAGCTACTGGTGGTGATAGCAT |
| mAsPCR-seg73.4..Reverse | CGAGAATGTACGCAGGTCCA |
| mAsPCR-seg73.4..Wild-Type | CAGTTACTGGTGGTGATAGCAA |
| mAsPCR-seg73.5..Recoded | CATATAGCGCTTCCAGGGATGA |
| mAsPCR-seg73.5..Reverse | GCCCGCGCGTTTGAATAT |
| mAsPCR-seg73.5..Wild-Type | CATATAACGCTTCCAGACTGCT |
| mAsPCR-seg73.6..Recoded | TCAAACAACAAACCGCAGAATCC |
| mAsPCR-seg73.6..Reverse | GCGAGTATAGATGCCACTAAGC |
| mAsPCR-seg73.6..Wild-Type | TCAAACAACAAACCGCAGAAAGT |
| mAsPCR-seg73.7..Recoded | ATCTGACCGATGACAATGCCT |
| mAsPCR-seg73.7..Reverse | CCATCGGTTGTTTTCAGAAGCAT |
| mAsPCR-seg73.7..Wild-Type | ATCTGACCGATGACAATGCCA |
| mAsPCR-seg73.8..Recoded | CACGTTAATTTTTAGAAGATCGCGAATAAG |
| mAsPCR-seg73.8..Reverse | AGATTGCGATGCTTAATGGTTGC |
| mAsPCR-seg73.8..Wild-Type | CACGTTAATTTTCAAAAGATCGCGAATCAA |
| mAsPCR-seg74.1..Recoded | CTTGGACGAGGAAAGGCTTGA |
| mAsPCR-seg74.1..Reverse | TTCGGCATGTGGGAAAGTCA |
| mAsPCR-seg74.1..Wild-Type | CTTGGACGAGGAAAGGCTTAG |
| mAsPCR-seg74.2..Recoded | GACATCATCACCGTCGATTCT |
| mAsPCR-seg74.2..Reverse | GGTGCCATGTGAGCGATAGT |
| mAsPCR-seg74.2..Wild-Type | GACATCATCACCGTCGATAGC |
| mAsPCR-seg74.3..Recoded | CTAACCCGGACGATGACTCA |
| mAsPCR-seg74.3..Reverse | AAACTCCAGCCCTTTCGAC |
| mAsPCR-seg74.3..Wild-Type | CTAACCCGGACGATGACAGC |
| mAsPCR-seg74.4..Recoded | CAGGAGCCAAAGATATAACCCAGT |
| mAsPCR-seg74.4..Reverse | GTCTTCGTGGTTATACTTCTGCTAATAATTT |
| mAsPCR-seg74.4..Wild-Type | CAGGAGCCAAAGATATAACCCAGG |
| mAsPCR-seg74.5..Recoded | CTGAACTACTTTTCCTGATATGTCGCTT |
| mAsPCR-seg74.5..Reverse | ACAAAAACCAGCGCCATCAG |
| mAsPCR-seg74.5..Wild-Type | TTGAACTACTTTTCCTGATATGTCGTTG |
| mAsPCR-seg74.6..Recoded | CGTGGCTGTTTTTCCTTGTATC |
| mAsPCR-seg74.6..Reverse | GGTGTCGCGAGTGAGATAGAG |
| mAsPCR-seg74.6..Wild-Type | CGTGGCTGTTTTTCCTCGTCAG |
| mAsPCR-seg74.7..Recoded | ACCGTTCTGAATACATCAAGCAAC |
| mAsPCR-seg74.7..Reverse | TTTGGGTAGTTATCGAAGTGGCA |
| mAsPCR-seg74.7..Wild-Type | ACCGTTCTGAATACATCAAGCAAT |
| mAsPCR-seg74.8..Recoded | GCCAGAGTGCAAGTGGTG |
| mAsPCR-seg74.8..Reverse | ATCCACTGCCAGACCTCATTTT |
| mAsPCR-seg74.8..Wild-Type | GCCAGAGTGCAAGTGGGC |
| mAsPCR-seg75.1..Recoded | GTCGATTAGTTCCATAAATCGCTGAAG |
| mAsPCR-seg75.1..Reverse | GGATACCAACAACATTCAGTACGC |
| mAsPCR-seg75.1..Wild-Type | GTCGATTAATTCCATAAATCGCTGCAA |
| mAsPCR-seg75.2..Recoded | GCTTGCAGATGAAATTGAAAATATCTATTCT |
| mAsPCR-seg75.2..Reverse | AACAAATGGTTCTATGAGAAAGAGGTAAA |
| mAsPCR-seg75.2..Wild-Type | GTTGGCAGATGAAATTGAAAATATCTATAGC |
| mAsPCR-seg75.3..Recoded | TTCCAGACAGGTAAGGGTAGAGAAT |
| mAsPCR-seg75.3..Reverse | CGCTTCTTTCTCCCGACCA |
| mAsPCR-seg75.3..Wild-Type | TTCCAGACAGGTTAAGGTAGAGAAA |
| mAsPCR-seg75.4..Recoded | CACTTTTGCTACCAGACCTGA |
| mAsPCR-seg75.4..Reverse | CCGATTCAGGCAATGTGATTTGT |
| mAsPCR-seg75.4..Wild-Type | CACTTTTGCTACCAGACCGCT |
| mAsPCR-seg75.5..Recoded | GGGCAAGTATCTACAGCACTCA |
| mAsPCR-seg75.5..Reverse | GCAATAATTAGTAGCTGCCAAATGGA |
| mAsPCR-seg75.5..Wild-Type | GGGCAAGTATTTACAGCACAGT |
| mAsPCR-seg75.6..Recoded | GCCCAGGAACACCTCGAAC |
| mAsPCR-seg75.6..Reverse | GTTGCCGGATCGACAATGTC |
| mAsPCR-seg75.6..Wild-Type | GCCCAGGAACACCTCGAAA |
| mAsPCR-seg75.7..Recoded | TTTTCACGTGGTTCACTACAAC-TTC |
| mAsPCR-seg75.7..Reverse | ACAAAAAAGGTCTGGGTAAAAGCG |
| mAsPCR-seg75.7..Wild-Type | TTTAGCCGTGGTTCATTGCAATTGT |
| mAsPCR-seg75.8..Recoded | AGCTTTGAGGTATCCATTCGTGA |
| mAsPCR-seg75.8..Reverse | TATGGATGTTGATAAGCCAGGCAAA |
| mAsPCR-seg75.8..Wild-Type | AGCTTTGAGGTATCCATTCGACT |
| mAsPCR-seg76.1..Recoded | CCAGTTTACTTTTAATGGTGATGGTTCA |
| mAsPCR-seg76.1..Reverse | TTTCCGCATCCATTCCTTCAGA |
| mAsPCR-seg76.1..Wild-Type | CCAGTTTACTTTTAATGGTGATGGTAGT |
| mAsPCR-seg76.2..Recoded | CTTGTCCACGCCTTGTTTCTTTAG |
| mAsPCR-seg76.2..Reverse | AAATCCGCCTTTTATTATGGTTCAGG |
| mAsPCR-seg76.2..Wild-Type | CTTGTCCACGCCTTGTTTCTTCAA |
| mAsPCR-seg76.3..Recoded | CAGATCCTCAACTCGCTGATTAACT |
| mAsPCR-seg76.3..Reverse | AGACGGTCGACCAGATTTCG |
| mAsPCR-seg76.3..Wild-Type | CAGATCCTCAACTCGCTGATTAACA |
| mAsPCR-seg76.4..Recoded | CGAGCAGCATGAAGATCTTAAATCA |
| mAsPCR-seg76.4..Reverse | TGATTTTCTGGAAGTGGTGTTTCAG |
| mAsPCR-seg76.4..Wild-Type | CGAGCAGCATGAAGATTTAAAAAGT |
| mAsPCR-seg76.5..Recoded | GATGTTCCGTTGTGATGTGGGA |
| mAsPCR-seg76.5..Reverse | CGCACACTTACACCCTGAAATATC |
| mAsPCR-seg76.5..Wild-Type | GATCTTCCGTTGTGATGTGACT |
| mAsPCR-seg76.6..Recoded | CCTGGCCAAACAAAGTCCTCT |
| mAsPCR-seg76.6..Reverse | ATTCATTCATTTATTCCTTTATCCAGTCGTT |
| mAsPCR-seg76.6..Wild-Type | CCTGGCCAAACAAAGTCCTCA |
| mAsPCR-seg76.7..Recoded | CGAAATCTTTGGCGACGAAACT |
| mAsPCR-seg76.7..Reverse | GTATGGAGCCAACGAAGAATAAAAATTT |
| mAsPCR-seg76.7..Wild-Type | CGAAATCTTTGGCGACGAGACG |
| mAsPCR-seg76.8..Recoded | GCGACGGCGGAAAATTCA |
| mAsPCR-seg76.8..Reverse | TCGACAGACAACCGATCACTTT |
| mAsPCR-seg76.8..Wild-Type | GCGACGGCGGAAAATAGC |
| mAsPCR-seg77.1..Recoded | GTTATCACCAAGAAACAGACCTGA |
| mAsPCR-seg77.1..Reverse | CGGAGAAAGTCAACGCGTTT |
| mAsPCR-seg77.1..Wild-Type | GTTATCACCAAGAAACAGACCGCT |
| mAsPCR-seg77.2..Recoded | AAAAGCGTCGAAAAGTGGTTGG |
| mAsPCR-seg77.2..Reverse | GCAGCCCTATACCATCACC |
| mAsPCR-seg77.2..Wild-Type | AAAAGCGTCGAAAAGTGGTTAC |
| mAsPCR-seg77.3..Recoded | CCGACAATACTGGAGATGAATATGTCT |
| mAsPCR-seg77.3..Reverse | CCACACATCCAGGCCCATAAT |
| mAsPCR-seg77.3..Wild-Type | CCGACAATACTGGAGATGAATATGAGC |
| mAsPCR-seg77.4..Recoded | GGTTCGGCACTATTCCTGTTTCTA |
| mAsPCR-seg77.4..Reverse | CGTGAGCGCCTGAAACAC |
| mAsPCR-seg77.4..Wild-Type | GGTTCGGCACTATTCCTGTTTTTG |
| mAsPCR-seg77.5..Recoded | CTTCACATCCTGAGTATCCTTACCG |
| mAsPCR-seg77.5..Reverse | GCTTTTCTCACTGGCGGGTA |
| mAsPCR-seg77.5..Wild-Type | CTTCACATCCTGAGTATCTTTACCA |
| mAsPCR-seg77.6..Recoded | ACCCACACCGAAGAAAATGAGTAG |
| mAsPCR-seg77.6..Reverse | GCGAATGATCTAACAAACATGCATCAT |
| mAsPCR-seg77.6..Wild-Type | ACCCACACCGAAGAAAATCAACAA |
| mAsPCR-seg77.7..Recoded | CAAAATCAGCAGGAAAAAACCTTTATCGATC |
| mAsPCR-seg77.7..Reverse | CCCTTGCTCATATAGATAATTTACTGCATC |
| mAsPCR-seg77.7..Wild-Type | CAAAATCAGCAGGAAAAAACCTTTATCGATT |
| mAsPCR-seg77.8..Recoded | GTAGAATCACCATCTAATCCACTCCTT |
| mAsPCR-seg77.8..Reverse | GACCGTTCAGATATTTCGTGCAT |
| mAsPCR-seg77.8..Wild-Type | GTAGAAAGCCCAAGTAATCCATTGTTA |
| mAsPCR-seg78.1..Recoded | CAGTAGGTTCACGAAGAAGTCATTT |
| mAsPCR-seg78.1..Reverse | GTGCCTGGTTCAAACTGACG |
| mAsPCR-seg78.1..Wild-Type | CAGGAAGTTCACGAAGAAGTCATTG |
| mAsPCR-seg78.2..Recoded | TCATCGGGATCATGATTTTCAGTGA |
| mAsPCR-seg78.2..Reverse | GCACCACCTCACATACGGT |
| mAsPCR-seg78.2..Wild-Type | TCATCGGGATCATGATTTTCAGGCT |
| mAsPCR-seg78.3..Recoded | CCTGAGTCGCGTCCATAATTTTAAG |
| mAsPCR-seg78.3..Reverse | CGCATCTCATGTAACGTTGTGG |
| mAsPCR-seg78.3..Wild-Type | CCTGAGTCGCGTCCATAATTTTTAA |
| mAsPCR-seg78.4..Recoded | GCTTCGGTATGACGCGTTG |
| mAsPCR-seg78.4..Reverse | CTGCTACTCTCTCGCTGGAAA |
| mAsPCR-seg78.4..Wild-Type | GCTTCGGTATGACGCGTGC |
| mAsPCR-seg78.5..Recoded | CATGATGATGACGCTGAAAGGAC |
| mAsPCR-seg78.5..Reverse | CACCTGTGAGAATTTCTGAAGCTC |
| mAsPCR-seg78.5..Wild-Type | CATGATGATGACGCTGAAAGGTT |
| mAsPCR-seg78.6..Recoded | AAGACGTACCACTTTTTCGGCAAG |
| mAsPCR-seg78.6..Reverse | CAATCATCGCACCTTTCCTTACC |
| mAsPCR-seg78.6..Wild-Type | CAAACGTACCACTTTTTCGGCTAA |
| mAsPCR-seg78.7..Recoded | AGTCAGGAGTATTTAGCCTTGGAC |
| mAsPCR-seg78.7..Reverse | CGAGATTCCCCCAGTAGCG |
| mAsPCR-seg78.7..Wild-Type | AGTCAGGAGTATTTAGCCTTGGAG |
| mAsPCR-seg78.8..Recoded | TAATCCATCCCAGACTGAAGGACATTTAG |
| mAsPCR-seg78.8..Reverse | CTGGTGAAGTTTGTTTCCGATCTC |
| mAsPCR-seg78.8..Wild-Type | TAATCCATCCCGCTCTGTAAGACATTTAA |
| mAsPCR-seg79.1..Recoded | AGCGAACATGGAGCTGTCA |
| mAsPCR-seg79.1..Reverse | GAGTCGGGTGCACATCCC |
| mAsPCR-seg79.1..Wild-Type | AGCGAACATGGAGCTGAGC |
| mAsPCR-seg79.2..Recoded | GCCAGAATCCTTCAACGTACTTC |
| mAsPCR-seg79.2..Reverse | TCAGGATCTGCTGACGTTCC |
| mAsPCR-seg79.2..Wild-Type | GCCAGAATCCTTCAACGTATTGT |
| mAsPCR-seg79.3..Recoded | GCGCAGATGGTTTGCACAAG |
| mAsPCR-seg79.3..Reverse | CCCGTGAATCAGCCGCTAT |
| mAsPCR-seg79.3..Wild-Type | GCGCAGATGGTTTGCACTAA |
| mAsPCR-seg79.4..Recoded | CATCGCCCATTCGGTTTTGG |
| mAsPCR-seg79.4..Reverse | TTGACTCCGCAAGTTTGTATTCAAA |
| mAsPCR-seg79.4..Wild-Type | CATCGCCCATTCGGTTTTGC |
| mAsPCR-seg79.5..Recoded | TATTTTTATCGCCGTTGATGCCTCA |
| mAsPCR-seg79.5..Reverse | CCTCTTTCGCCATAACTTGTGC |
| mAsPCR-seg79.5..Wild-Type | TATTTTTATCGCCGTTGATGCCACT |
| mAsPCR-seg79.6..Recoded | GATACCGGCTTTGTCAGAAACTG |
| mAsPCR-seg79.6..Reverse | GCACAGAGTTATCCACAATCATCAAT |
| mAsPCR-seg79.6..Wild-Type | GATACCGGCTTTGTCAGAAACAC |
| mAsPCR-seg79.7..Recoded | CTCATTAACCGCGACCCAAAG |
| mAsPCR-seg79.7..Reverse | TCAAGGAAAAGACTACGTTAGAATATAAGAA |
| mAsPCR-seg79.7..Wild-Type | CTCATTAACCGCGACCCACAA |
| mAsPCR-seg79.8..Recoded | TTTCCCCGGCACTTATGGAACTT |
| mAsPCR-seg79.8..Reverse | TCTTCAATGGCGTCGCGAA |
| mAsPCR-seg79.8..Wild-Type | TTTCCCCGGCATTAATGGAATTA |
| mAsPCR-seg80.1..Recoded | CTTTATCCATCACGCGAAACTTCTT |
| mAsPCR-seg80.1..Reverse | GCCGACCACATTCATGCC |
| mAsPCR-seg80.1..Wild-Type | CTTTATCCATCACGCGAAATTGTTG |
| mAsPCR-seg80.2..Recoded | GAGTTTATTCGCGGCATGTCA |
| mAsPCR-seg80.2..Reverse | GCGTCATTTTCCTGGTCAGC |
| mAsPCR-seg80.2..Wild-Type | GAGTTTATTCGCGGCATGAGT |
| mAsPCR-seg80.3..Recoded | TAGCGTTTTGGCCTCGGAA |
| mAsPCR-seg80.3..Reverse | CAACAAAAATGGGTCACTCAGGATC |
| mAsPCR-seg80.3..Wild-Type | TAGCGTTTTGGCCTCACTG |
| mAsPCR-seg80.4..Recoded | ACATCTTTAACCTTTCACTCCTCCA |
| mAsPCR-seg80.4..Reverse | CGTAATTTTCGCGTATCTGGGT |
| mAsPCR-seg80.4..Wild-Type | ACATCTTTAACCTTTCACACCACCT |
| mAsPCR-seg80.5..Recoded | ACTTGTTAAAGCCCTTCAGGACTGA |
| mAsPCR-seg80.5..Reverse | CTGGGATATTTCTGGTCCTGGTG |
| mAsPCR-seg80.5..Wild-Type | ACTTGTTAAAGCCCTTCAGGACACT |
| mAsPCR-seg80.6..Recoded | ACATCTCCCGCGACGTAC |
| mAsPCR-seg80.6..Reverse | GACGGGTTGGCGGAAAGTA |
| mAsPCR-seg80.6..Wild-Type | ACATCTCCCGCGACGTAT |
| mAsPCR-seg80.7..Recoded | TACAGGTATGCGTTTAAACCCAGTTAAAC |
| mAsPCR-seg80.7..Reverse | CTCAAAGTGGGGGTTAAGAATGTC |
| mAsPCR-seg80.7..Wild-Type | TACAGGTATGCGTTTAAACCCAGTTAAAT |
| mAsPCR-seg80.8..Recoded | AGAAGCAGTACAGGTTTGGTGATA |
| mAsPCR-seg80.8..Reverse | GCCCCTGCCTCAAAAATGG |
| mAsPCR-seg80.8..Wild-Type | AGTAACAGTACAGGTTTGGTGATT |
| mAsPCR-seg81.1..Recoded | CATCTGAATAAAGCGCACTGGTC |
| mAsPCR-seg81.1..Reverse | CGTGCGACCAGTGCAAAG |
| mAsPCR-seg81.1..Wild-Type | CATCTGAATAAAGCGCACTGGAG |
| mAsPCR-seg81.2..Recoded | TGACCACCCACAAAACCTCA |
| mAsPCR-seg81.2..Reverse | GGAATTATACTCCCCAACAGATGAATT |
| mAsPCR-seg81.2..Wild-Type | TGACCACCCACAAAACCAGT |
| mAsPCR-seg81.3..Recoded | GTCACATCACCATCACATACAAAGAAG |
| mAsPCR-seg81.3..Reverse | TTTTCCATGATGGCGAAGTTGAAAT |
| mAsPCR-seg81.3..Wild-Type | GTCACAAGTCCATCACATACAAAGAAA |
| mAsPCR-seg81.4..Recoded | GATCGTGCAAAAGGTTCTGTCT |
| mAsPCR-seg81.4..Reverse | GCGACACCAAGCCAGAAC |
| mAsPCR-seg81.4..Wild-Type | GATCGTGCAAAAGGTTCTGAGC |
| mAsPCR-seg81.5..Recoded | TACTATCTGTGGCAAAACGATTACTCA |
| mAsPCR-seg81.5..Reverse | TCGCCATATTAATCGACTCAACCA |
| mAsPCR-seg81.5..Wild-Type | TACTATCTGTGGCAAAACGATTACAGC |
| mAsPCR-seg81.6..Recoded | GCGAGAATCTCTGCGTGCAC |
| mAsPCR-seg81.6..Reverse | GTTTTTTTGAATAGGGTATGCAGATGGA |
| mAsPCR-seg81.6..Wild-Type | GCGAGAATCTCTGCGTGCAT |
| mAsPCR-seg81.7..Recoded | CAGTAAGCGCAATAACAATACGTGAA |
| mAsPCR-seg81.7..Reverse | TGTAATTTTCCCTCTTCAGCACGA |
| mAsPCR-seg81.7..Wild-Type | CAGTTAACGCAATAACAATCCTGCTC |
| mAsPCR-seg81.8..Recoded | CACCGAAGCCTTCAAAAAAGCAT |
| mAsPCR-seg81.8..Reverse | CAACACCCATTGCCATCGT |
| mAsPCR-seg81.8..Wild-Type | CACCGAAGCCTTCAAAAAAGCAA |
| mAsPCR-seg82.1..Recoded | GGGCGATATCTTCATACAGTTTTACT |
| mAsPCR-seg82.1..Reverse | CTGGTGTTCGGCATGTCTGA |
| mAsPCR-seg82.1..Wild-Type | GGGCGATATCTTCATACAGTTTCACC |
| mAsPCR-seg82.2..Recoded | CTCTTGATAGCGTGTTGGGTATGA |
| mAsPCR-seg82.2..Reverse | CTGGCGGTGGTTCTCTCC |
| mAsPCR-seg82.2..Wild-Type | CTCTTGATAGCGTGTTGGGTAGCT |
| mAsPCR-seg82.3..Recoded | GGCGCAGAACACCATCTCA |
| mAsPCR-seg82.3..Reverse | CATTTTGTTGACGCAGAGCCA |
| mAsPCR-seg82.3..Wild-Type | GGCGCAGAACACCATCAGT |
| mAsPCR-seg82.4..Recoded | TGTGTATCTGACTCGGTTTACCAAATAAT |
| mAsPCR-seg82.4..Reverse | CGTCATATCATACGCCTGCATTC |
| mAsPCR-seg82.4..Wild-Type | TGTGTAAGTGACAGCGTTTATCAAATTAT |
| mAsPCR-seg82.5..Recoded | GCTTTTTCCCGATCGCCTAG |
| mAsPCR-seg82.5..Reverse | ATTCCTTCATAACCGGGTAAGCAA |
| mAsPCR-seg82.5..Wild-Type | GCTTTTTCCCGATCGCCCAA |
| mAsPCR-seg82.6..Recoded | CAATACCCGGTATCCACTCGTC |
| mAsPCR-seg82.6..Reverse | GTTACCTTTCGCCAGCATGATC |
| mAsPCR-seg82.6..Wild-Type | CAATACCCGGTATCCACTCGTT |
| mAsPCR-seg82.7..Recoded | CCGAGAACAGTACCGCAGA |
| mAsPCR-seg82.7..Reverse | CCCCGGAATCTTCATACAGCA |
| mAsPCR-seg82.7..Wild-Type | CCGAGAACAGTACCGCACT |
| mAsPCR-seg82.8..Recoded | CCAGCCATCAGATTCCGTACG |
| mAsPCR-seg82.8..Reverse | GCACACCACCACTTCTCC |
| mAsPCR-seg82.8..Wild-Type | CCAGCCATCAGATTCCGTTCT |
| mAsPCR-seg83.1..Recoded | CTGTAAAGAGTTTGAGAAATACACCTTCT |
| mAsPCR-seg83.1..Reverse | TTGCTACCATCGCCGGATC |
| mAsPCR-seg83.1..Wild-Type | CTGTAAAGAGTTTGAGAAATACACCTTCA |
| mAsPCR-seg83.2..Recoded | TCAGGAATATCTGAGATTTTGTTGTTTGA |
| mAsPCR-seg83.2..Reverse | CGTACCAGTGACATACCGATAACT |
| mAsPCR-seg83.2..Wild-Type | TCAGGAATATCACTGATTTTGTTGTTGCT |
| mAsPCR-seg83.3..Recoded | CCTGAAAATTGTTCTTTGCCTGA |
| mAsPCR-seg83.3..Reverse | ATGGAACTGCGCGACCTG |
| mAsPCR-seg83.3..Wild-Type | CCGCTAAATTGTTCTTTGCCACT |
| mAsPCR-seg83.4..Recoded | CAGTTACCGCCCAGAGTGA |
| mAsPCR-seg83.4..Reverse | CAGGGCAAAGTAGAATCATCGAAAG |
| mAsPCR-seg83.4..Wild-Type | CAGTTACCGCCCAGAGACT |
| mAsPCR-seg83.5..Recoded | ACGTCAGGATCTCGACCGT |
| mAsPCR-seg83.5..Reverse | CGCGAGGTGTCATCCATAAC |
| mAsPCR-seg83.5..Wild-Type | ACGTCAGGATCTCGACAGA |
| mAsPCR-seg83.6..Recoded | CGCAATATCGGTTATCGCGTAC |
| mAsPCR-seg83.6..Reverse | CCTGGGGAGTCAATCACATCA |
| mAsPCR-seg83.6..Wild-Type | CGCAATATCGGTTATCGCGTAT |
| mAsPCR-seg83.7..Recoded | TATTGGCGATCCTGATTATGCGTTTTC |
| mAsPCR-seg83.7..Reverse | CAGTGTAATTCGAGCCATTCTGC |
| mAsPCR-seg83.7..Wild-Type | TATTGGCGATCCTGATTATGCGTTTAG |
| mAsPCR-seg83.8..Recoded | GGCATACGAACTTGCAGAGA |
| mAsPCR-seg83.8..Reverse | GCTTTTTCAGGCTCTAACGGA |
| mAsPCR-seg83.8..Wild-Type | GGCATACGAACTTGCAGACT |
| mAsPCR-seg84.1..Recoded | GTTGACGGACGCACATAGTAT |
| mAsPCR-seg84.1..Reverse | AACTGGTCTTCACTCGTCGTC |
| mAsPCR-seg84.1..Wild-Type | GTTGACGGACGCACATAGTAG |
| mAsPCR-seg84.2..Recoded | CGTACTTAAAGGTTGTTCAGATTCTTCT |
| mAsPCR-seg84.2..Reverse | CGCAGAGTAAAACGGTAAGCC |
| mAsPCR-seg84.2..Wild-Type | CGTATTGAAAGGTTGTAGCGATAGTAGC |
| mAsPCR-seg84.3..Recoded | AGTACAACAAATCTCAGTCCATCACTC |
| mAsPCR-seg84.3..Reverse | ACAACTTTCAGACCGACCTCTAC |
| mAsPCR-seg84.3..Wild-Type | AGTACAACAAAAGTCAGTCCATCACTT |
| mAsPCR-seg84.4..Recoded | GGTGGTGATCAAGCCCTCA |
| mAsPCR-seg84.4..Reverse | CATCTTTCCCCCAGGCGAA |
| mAsPCR-seg84.4..Wild-Type | GGTGGTGATCAAGCCCAGC |
| mAsPCR-seg84.5..Recoded | CATCCATCCCTCCGTTCTCA |
| mAsPCR-seg84.5..Reverse | CTCTACGGCCTTTAGTCAGTCTATG |
| mAsPCR-seg84.5..Wild-Type | CATCCATCCCTCCGTTCAGC |
| mAsPCR-seg84.6..Recoded | GATGCCACACGCCAGTTT |
| mAsPCR-seg84.6..Reverse | GATAAAGATCGGCGGCATTACG |
| mAsPCR-seg84.6..Wild-Type | GATGCCACACGCCAGTTC |
| mAsPCR-seg84.7..Recoded | TGGAGTTCAAATTTACCCCGTTTAAG |
| mAsPCR-seg84.7..Reverse | ACGAAGAAATACCCATAACAATAAATGAAT |
| mAsPCR-seg84.7..Wild-Type | TGGAGTTCAAATTTACCCCGTTTTAA |
| mAsPCR-seg84.8..Recoded | CTGAATCTGACGGCGGAACTA |
| mAsPCR-seg84.8..Reverse | ACGGGTAAAGATGGGGTTTATCAT |
| mAsPCR-seg84.8..Wild-Type | CTGAATCTGACGGCGGAATTG |
| mAsPCR-seg85.1..Recoded | CTTTCTCGATCAGGTCTATCAAGTTTC |
| mAsPCR-seg85.1..Reverse | TCAATCAGGCGGATGATCTCG |
| mAsPCR-seg85.1..Wild-Type | CTTTCTCGATCAGGTCTATCAGGTCAG |
| mAsPCR-seg85.2..Recoded | GAAATGCCGGTGGTCTTGG |
| mAsPCR-seg85.2..Reverse | GGCGTCATCACCTTGATCGA |
| mAsPCR-seg85.2..Wild-Type | CTAATGCCGGTGGTCTTGC |
| mAsPCR-seg85.3..Recoded | CCTCGAAATCCCGTGACAACTC |
| mAsPCR-seg85.3..Reverse | TTTTTTAATGAATTTGCTGGTTGAAAAATC |
| mAsPCR-seg85.3..Wild-Type | CCAGTAAATCCCGTGACAACAG |
| mAsPCR-seg85.4..Recoded | CAATCTCGCCATTGTGACCT |
| mAsPCR-seg85.4..Reverse | GAAACAGAAAGTGATCGTCAAACATCT |
| mAsPCR-seg85.4..Wild-Type | CAATCTCGCCATTGTGACGC |
| mAsPCR-seg85.5..Recoded | TGTACTACCATATATTAATGAACAGCGTCTT |
| mAsPCR-seg85.5..Reverse | GCAAGAAAATGGCGGAAGAATT |
| mAsPCR-seg85.5..Wild-Type | TGTATTACCATATATTAATGAACAGCGTTTA |
| mAsPCR-seg85.6..Recoded | CTACCTGCCAATTCATCATCATCA |
| mAsPCR-seg85.6..Reverse | ATACAGATGAATCGTACGCGTTTAG |
| mAsPCR-seg85.6..Wild-Type | CTACCTGCCAATAGTTCAAGTAGT |
| mAsPCR-seg85.7..Recoded | CCACGACGATGCAGGAAG |
| mAsPCR-seg85.7..Reverse | GCTAAGATAATTATACTCAACGGATTCACC |
| mAsPCR-seg85.7..Wild-Type | CCACGACGATGCAGGCAC |
| mAsPCR-seg85.8..Recoded | GCCCGACACCTGAATCTACTAG |
| mAsPCR-seg85.8..Reverse | GCTGTTTATTGCCATTGTTATTGCG |
| mAsPCR-seg85.8..Wild-Type | GCCCGACACCGCTATCTACTAA |
| mAsPCR-seg86.1..Recoded | GTATACCCATCATCTGCTGGAATCT |
| mAsPCR-seg86.1..Reverse | GCCCACTTTATCCCAATCCG |
| mAsPCR-seg86.1..Wild-Type | GTATACCCATCATCTGCTGGAAAGC |
| mAsPCR-seg86.2..Recoded | GCATTGTTCATGTTATCTGCTGAAAG |
| mAsPCR-seg86.2..Reverse | GGTAAATCCGTACTTATCATCACCGT |
| mAsPCR-seg86.2..Wild-Type | GCATTGTTCATGTTATCTGCGCTTAA |
| mAsPCR-seg86.3..Recoded | TCACAAACAGAACGTGGATCTTCT |
| mAsPCR-seg86.3..Reverse | CGGGAGGGGGCATCATTTAA |
| mAsPCR-seg86.3..Wild-Type | TCACAAACAGAACGTGGATCTTCA |
| mAsPCR-seg86.4..Recoded | CGTCGATTCTCAGGCACAATCA |
| mAsPCR-seg86.4..Reverse | GCTGGACTGGCTTTGGATAAAATT |
| mAsPCR-seg86.4..Wild-Type | CGTCGATTCTCAGGCACAAAGT |
| mAsPCR-seg86.5..Recoded | TGATGGACGTGAAAGTGGGTTC |
| mAsPCR-seg86.5..Reverse | AGCACCGCCTGTAGTTTCG |
| mAsPCR-seg86.5..Wild-Type | TGATGGACGTGAAAGTGGGTAG |
| mAsPCR-seg86.6..Recoded | CTTCAGAGATTCGTTCCTGACCT |
| mAsPCR-seg86.6..Reverse | GGCTGGAACAAAACCGTCTG |
| mAsPCR-seg86.6..Wild-Type | CTTCACAGATTCGTTCCTGACCG |
| mAsPCR-seg86.7..Recoded | GGATAAACCGACGCTTATGTCA |
| mAsPCR-seg86.7..Reverse | TGGTAGGCATTCTTAAGCAGGTC |
| mAsPCR-seg86.7..Wild-Type | GGATAAACCGACGTTGATGAGC |
| mAsPCR-seg86.8..Recoded | CAGAAAGATCGCCGGTACCT |
| mAsPCR-seg86.8..Reverse | CGTGGTATTGGTGTGGTGAAAG |
| mAsPCR-seg86.8..Wild-Type | CAGAAAGATCGCCGGTACCG |

TABLE 5
Summary of AGR codons changed by location in
the genome, and failure rates by pool.

| | # AGR | # | # | % |
|---|---|---|---|---|
| AGR pool | codon | Successful | Failed | Success |
| AGR. 1 | 11 | 10 | 1 | 91 |
| AGR. 2 | 12 | 10 | 2 | 83 |
| AGR. 3 | 10 | 10 | 0 | 100 |
| AGR. 4 | 7 | 7 | 0 | 100 |
| AGR. 5 | 14 | 13 | 1 | 93 |
| AGR. 6 | 8 | 8 | 0 | 100 |
| AGR. 7 | 13 | 11 | 2 | 85 |
| AGR. 8 | 9 | 8 | 1 | 89 |
| AGR. 9 | 10 | 9 | 1 | 90 |
| AGR. 10 | 13 | 12 | 1 | 92 |
| AGR. 11 | 7 | 6 | 1 | 86 |
| AGR. 12 | 9 | 6 | 3 | 67 |
| Total | 123 | 110 | 13 | 89 |

### REFERENCES

The specification identifies the references by author with the complete citations provided below. The disclosure of each reference cited is hereby incorporated by reference in its entirety.
1. Gibson, D. G., Glass, J. I., Lartigue, C., Noskov, V. N., Chuang, R. Y., Algire, M. A., Benders, G. A., Montague, M. G., Ma, L., Moodie, M. M., et al. (2010). Creation of a bacterial cell controlled by a chemically synthesized genome. Science 329, 52-56.
2. Lajoie, M. J., Kosuri, S., Mosberg, J. A., Gregg, C. J., Zhang, D., and Church, G. M. (2013a). Probing the limits of genetic recoding in essential genes. Science 342, 361-363.
3. Lajoie, M. J., Rovner, A. J., Goodman, D. B., Aerni, H. R., Haimovich, A. D., Kuznetsov, G., Mercer, J. A., Wang, H. H., Carr, P. A., Mosberg, J. A., et al. (2013b). Genomically recoded organisms expand biological functions. Science 342, 357-360.
4. Crick, F. H. (1963). On the genetic code. Science 139, 461-464.
5. Liu, C. C., Schultz, P. G. Adding new chemistries to the genetic code. Annu. Rev. Biochem. 79, 413-444 (2010).
6. P. Marliere, The farther, the safer: a manifesto for securely navigating synthetic species away from the old living world. Syst. Synth. Biol. 3 , 77-84 (2009).
7. Mandell, D. J. et al., Biocontainment of genetically modified organisms by synthetic protein design. Nature. 518, 55-60 (2015).
8. Rovner, A. J. et al., Recoded organisms engineered to depend on synthetic amino acids. Nature. 518, 89-93 (2015).
9. A. Ambrogelly, S. Palioura, D. Soll, Natural expansion of the genetic code. Nat. Chem. Biol. 3 , 29-35 (2007).
10. A. Kano, Y. Andachi, T. Ohama, S. Osawa, Novel anticodon composition of transfer RNAs in Micrococcus luteus, a bacterium with a high genomic G+C content. Correlation with codon usage. J. Mol. Biol. 221, 387-401 (1991).
11. T. Oba, Y. Andachi, A. Muto, S. Osawa, CGG: an unassigned or nonsense codon in Mycoplasma capricolum. Proc. Natl. Acad. Sci. U.S.A 88, 921-925 (1991).
12. G. Macino, G. Coruzzi, F. G. Nobrega, M. Li, A. Tzagoloff, Use of the UGA terminator as a tryptophan codon in yeast mitochondria. Proc. Natl. Acad. Sci. U.S.A 76, 3784-3785 (1979).
13. J. Ling, P. O'Donoghue, D. Soll, Genetic code flexibility in microorganisms: novel mechanisms and impact on physiology. Nat. Rev. Microbiol. 13, 707-721 (2015).
14. K. J. Blight, A. A. Kolykhalov, C. M. Rice, Efficient initiation of HCV RNA replication in cell culture. Science. 290, 1972-1974 (2000).
15. J. Cello, A. V. Paul, E. Wimmer, Chemical synthesis of poliovirus cDNA: generation of infectious virus in the absence of natural template. Science. 297, 1016-1018 (2002).
16. H. O. Smith, C. A. Hutchison, C. Pfannkoch, J. C. Venter, Generating a synthetic genome by whole genome assembly: φX174 bacteriophage from synthetic oligonucleotides. Proceedings of the National Academy of Sciences. 100, 15440-15445 (2003).
17. L. Y. Chan, S. Kosuri, D. Endy, Refactoring bacteriophage T7. Mol. Syst. Biol. 1 , 2005.0018 (2005).
18. D. G. Gibson et al., Complete chemical synthesis, assembly, and cloning of a Mycoplasma genitalium genome. Science. 319, 1215-1220 (2008).
19. N. Annaluru et al., Total synthesis of a functional designer eukaryotic chromosome. Science. 344, 55-58 (2014).
20. G. Kudla, A. W. Murray, D. Tollervey, J. B. Plotkin, Coding-sequence determinants of gene expression in Escherichia coli. Science. 324, 255-258 (2009).
21. T. Tuller, Y. Y. Waldman, M. Kupiec, E. Ruppin, Translation efficiency is determined by both codon bias and folding energy. Proc. Natl. Acad. Sci. U.S.A. 107, 3645-3650 (2010).
22. J. B. Plotkin, G. Kudla, Synonymous but not the same: the causes and consequences of codon bias. Nat. Rev. Genet. 12, 32-42 (2011).
23. D. B. Goodman, G. M. Church, S. Kosuri, Causes and effects of N-terminal codon bias in bacterial genes. Science. 342, 475-479 (2013).
24. M. Zhou et al., Non-optimal codon usage affects expression, structure and function of clock protein FRQ. Nature. 495, 111-115 (2013).
25. T. E. F. Quax, N. J. Claassens, D. Soll, J. van der Oost, Codon Bias as a Means to Fine-Tune Gene Expression. Mol. Cell. 59, 149-161 (2015).
26. G. Boël et al., Codon influence on protein expression in E. coli correlates with mRNA levels. Nature. 529, 358-363 (2016).
27. F. J. Isaacs et al., Precise manipulation of chromosomes in vivo enables genome-wide codon replacement. Science. 333, 348-353 (2011).
28. H. H. Wang et al., Programming cells by multiplex genome engineering and accelerated evolution. Nature. 460, 894-898 (2009).
29. K. M. Esvelt et al., Orthogonal Cas9 proteins for RNA-guided gene regulation and editing. Nat. Methods. 10, 1116-1121 (2013).
30. G. Pósfai et al., Emergent properties of reduced-genome Escherichia coli. Science. 312, 1044-1046 (2006).
31. K. Temme, D. Zhao, C. A. Voigt, Refactoring the nitrogen fixation gene cluster from Klebsiella oxytoca. Proc. Natl. Acad. Sci. U.S.A 109, 7085-7090 (2012).
32. A. H. Yona et al., tRNA genes rapidly change in evolution to meet novel translational demands. Elife. 2, e01339 (2013).
33. Y. Yamazaki, H. Niki, J.-I. Kato, in Microbial Gene Essentiality: Protocols and Bioinformatics, A. L. Osterman, S. Y. Gerdes, Eds. (Humana Press, Totowa, NJ, 2008), vol. 416 of Methods in Molecular Biology™, pp. 385-389.
34. S. Anders, W. Huber, Differential expression analysis for sequence count data. Genome Biol. 11, R106 (2010).
35. S. Osawa, T. H. Jukes, Codon reassignment (codon capture) in evolution. J. Mol. Evol. 28, 271-278 (1989).
36. H. M. Salis, The ribosome binding site calculator. Methods Enzymol. 498, 19-42 (2011).
37. T. Conway et al., Unprecedented high-resolution view of bacterial operon architecture revealed by RNA sequencing. MBio. 5, e01442-14 (2014).
38. C. J. Gregg et al., Rational optimization of tolC as a powerful dual selectable marker for genome engineering. Nucleic Acids Res. 42, 4779-4790 (2014).
39. K. A. Datsenko, B. L. Wanner, One-step inactivation of chromosomal genes in Escherichia coli K-12 using PCR products. Proc. Natl. Acad. Sci. U.S.A 97, 6640-6645 (2000).
40. A. Haldimann, B. L. Wanner, Conditional-replication, integration, excision, and retrieval plasmid-host systems for gene structure-function studies of bacteria. J. Bacteriol. 183, 6384-6393 (2001).
41. D. E. Deatherage, J. E. Barrick, Identification of mutations in laboratory-evolved microbes from next-generation sequencing data using breseq. Methods Mol. Biol. 1151, 165-188 (2014).
42. H. Li, R. Durbin, Fast and accurate short read alignment with Burrows-Wheeler transform. Bioinformatics. 25, 1754-1760 (2009a).
43. H. Li et al., The Sequence Alignment/Map format and SAMtools. Bioinformatics. 25, 2078-2079 (2009b).
44. S. Anders, W. Huber, Differential expression analysis for sequence count data. Genome Biol. 11, R106 (2010).
45. Carr P A, et al. (2012) Enhanced multiplex genome engineering through co-operative oligonucleotide co-selection. Nucleic Acids Res 40 (17): e132
46. Lennox E S (1955) Transduction of linked genetic characters of the host by bacteriophage P1. Virology 1 (2): 190-206.
47. Schwartz S A & Helinski D R (1971) Purification and characterization of colicin E1. The Journal of biological chemistry 246 (20): 6318-6327.
48. Mosberg J A, Gregg C J, Lajoie M J, Wang H H, & Church G M (2012) Improving Lambda Red Genome Engineering in Escherichia coli via Rational Removal of Endogenous Nucleases. PLoS One 7 (9): e44638.
49. Yaung S J, Esvelt K M, & Church G M (2014) CRISPR/Cas9-mediated phage resistance is not impeded by the DNA modifications of phage T4. PLoS One 9 (6): e98811.
50. Gibson D G, et al. (2009) Enzymatic assembly of DNA molecules up to several hundred kilobases. Nat Methods 6 (5): 343-345.
51. Baba T, et al. (2006) Construction of Escherichia coli K-12 in-frame, single-gene knockout mutants: the Keio collection. Mol Syst Biol 2:2006 0008.
52. Hashimoto M, et al. (2005) Cell size and nucleoid organization of engineered Escherichia coli cells with a reduced genome. Mol Microbiol 55 (1): 137-149.
53. Ellis H M, Yu D, DiTizio T, & Court D L (2001) High efficiency mutagenesis, repair, and engineering of chromosomal DNA using single-stranded oligonucleotides. Proc Natl Acad Sci USA 98 (12): 6742-6746.
54. Markham N R & Zuker M (2008) UNAFold: software for nucleic acid folding and hybridization. Methods in molecular biology 453:3-31.
55. Rohland N & Reich D (2012) Cost-effective, high-throughput DNA sequencing libraries for multiplexed target capture. Genome research 22 (5): 939-946.
56. Zadeh J N, et al. (2011) NUPACK: Analysis and design of nucleic acid systems. J Comput Chem 32 (1): 170-173.
57. Li G W, Oh E, & Weissman JS (2012) The anti-Shine-Dalgarno sequence drives translational pausing and codon choice in bacteria. Nature 484 (7395): 538-541.
58. Chen G F & Inouye M (1990) Suppression of the negative effect of minor arginine codons on gene expression; preferential usage of minor codons within the first 25 codons of the Escherichia coli genes. Nucleic Acids Res 18 (6): 1465-1473.
59. Rosenberg A H, Goldman E, Dunn J J, Studier F W, & Zubay G (1993) Effects of consecutive AGG codons on translation in Escherichia coli, demonstrated with a versatile codon test system. J Bacteriol 175 (3): 716-722.
60. Spanjaard R A & van Duin J (1988) Translation of the sequence AGG-AGG yields 50% ribosomal frameshift. Proc Natl Acad Sci USA 85 (21): 7967-7971.
61. Spanjaard R A, Chen K, Walker J R, & van Duin J (1990) Frameshift suppression at tandem AGA and AGG codons by cloned tRNA genes: assigning a codon to argU tRNA and T4 tRNA (Arg). Nucleic Acids Res 18 (17): 5031-5036.
62. Bonekamp F, Andersen H D, Christensen T, & Jensen K F (1985) Codon-defined ribosomal pausing in Escherichia coli detected by using the pyrE attenuator to probe the coupling between transcription and translation. Nucleic Acids Res 13 (11): 4113-4123.
63. Zeng Y, Wang W, & Liu W R (2014) Towards reassigning the rare AGG codon in Escherichia coli. Chembiochem: a European journal of chemical biology 15(12):1750-1754.
64. Yu D, et al. (2000) An efficient recombination system for chromosome engineering in Escherichia coli. Proc Natl Acad Sci USA 97 (11): 5978-5983.
65. Lajoie M J, Gregg C J, Mosberg J A, Washington G C, & Church G M (2012) Manipulating replisome dynamics to enhance lambda Red-mediated multiplex genome engineering. Nucleic Acids Res 40 (22): e170.
66. Curran J F (1993) Analysis of effects of tRNA: message stability on frameshift frequency at the Escherichia coli RF2 programmed frameshift site. Nucleic Acids Res 21 (8): 1837-1843.
67. Ohtake K, et al. (2012) Efficient decoding of the UAG triplet as a full-fledged sense codon enhances the growth of a prfA-deficient strain of Escherichia coli. | 194 (10): 2606-2613.
68. Craigen W J, Cook R G, Tate W P, & Caskey C T (1985) Bacterial peptide chain release factors: conserved primary structure and possible frameshift regulation of release factor 2. Proc Natl Acad Sci USA 82 (11): 3616-3620.
69. Goodman D, Kuznetsov, G., Lajoie, M., Ahern, B., (2015) Millstone, a web based genome engineering and analysis software.
70. Novoa E M & Ribas de Pouplana L (2012) Speeding with control: codon usage, tRNAs, and ribosomes. Trends in genetics: TIG 28 (11): 574-581.
71. Novoa E M, Pavon-Eternod M, Pan T, & Ribas de Pouplana L (2012) A role for tRNA modifications in genome structure and codon usage. Cell 149 (1): 202-213.
72. Ikemura T (1985) Codon usage and tRNA content in unicellular and multicellular organisms. Mol Biol Evol2 (1): 13-34.
73. Lajoie M J, Soll D, & Church G M (2015) Overcoming challenges in engineering the genetic code. J Mol Biol.
74. N. R. Markham, M. Zuker, DINAMelt web server for nucleic acid melting prediction. Nucleic Acids Res. 33, W577-81 (2005).

1.-26. (canceled)

27. An engineered organism comprising a recoded genome, wherein the organism is E. coli, wherein the recoded genome comprises at least one trinucleotide sequence corresponding to a particular codon at all or substantially all instances in a corresponding template genome that is replaced with a trinucleotide sequence corresponding to an alternative codon, wherein the particular codon is UAG, and wherein the prfB gene comprises a mutation relative to the corresponding template genome.

28. The engineered organism of claim 27, wherein the mutation is in a trinucleotide sequence corresponding to a forbidden sense codon.

29. The engineered organism of claim 28, wherein the forbidden sense codon is selected from the group consisting of: AGA, AGG, AGC, AGU, UUA, and UUG.

30. The engineered organism of claim 29, wherein the forbidden codon is AGG or AGC.

31. The engineered organism of claim 27, wherein the mutation comprises a frameshift mutation.

32. The engineered organism of claim 27, wherein the mutation is in the following prfB gene template sequence: 5′-CTTAGGGGGTATCTTTGAC-3′.

33. The engineered organism of claim 32, wherein the mutation comprises a mutation in the trinucleotide sequence corresponding to at least one of XXX¹, XXX², and XXX³ in the following prfB gene template sequence: 5′-XXXXXX²GGGTATCTTXXX³C-3′.

34. The engineered organism of claim 33, wherein the mutation comprises a wobble mutation in the trinucleotide sequence corresponding to XXX¹.

35. The engineered organism of claim 33, wherein the mutation comprises a frameshift mutation in the trinucleotide sequence corresponding to XXX³.

36. The engineered organism of claim 27, wherein the mutation comprises removing the nucleotide corresponding to X in the following prfB gene template sequence: 5′-CTTAGGGGGTATCTTXGAC-3′.

37. The engineered organism of claim 27, wherein the nucleotide corresponding to X in the following prfB gene template sequence is mutated in the prfB gene: 5′-CTXAGGGGGTATCTTTGAC-3′.

38. The engineered organism of claim 27, wherein the nucleotide corresponding to X2 in the following prfB gene template sequence is an adenine: 5′-CTTAGGGGGTATCTTX¹X²-3′.

39. The engineered organism of claim 27, wherein the mutation in prfB results in a mutated codon that is not reassigned to the non-standard amino acid.

40. The engineered organism of claim 27, wherein the mutation in prfB comprises a first mutation in a trinucleotide sequence corresponding to a forbidden sense codon and a second mutation, wherein the second mutation results in a mutated codon that is not reassigned to the non-standard amino acid.

41. The engineered organism of claim 40, wherein the forbidden sense codon is AGG or AGC.

42. The engineered organism of claim 40, wherein the second mutation is in the following prfB gene template sequence: 5′-CTTAGGGGGTATCTTTGAC-3′.

43. The engineered organism of claim 42, wherein the second mutation comprises a mutation in the trinucleotide sequence corresponding to XXX¹ or XXX² in the following prfB gene template sequence: 5′-XXX¹AGGGGGTATCTTXXX²C-3′.

44. The engineered organism of claim 27, wherein the mutation comprises a mutation in a trinucleotide sequence corresponding to a forbidden sense codon, and wherein the nucleotide corresponding to X2 in the following prfB gene template sequence is an adenine: 5′-CTTAGGGGGTATCTTX¹X²-3′.

45. The engineered organism of claim 44, wherein the forbidden sense codon is AGG or AGC.

46. The engineered organism of claim 27, wherein the at least one trinucleotide sequence corresponding to the particular codon is reassigned to a non-standard amino acid.

47. The engineered organism of claim 27, wherein ribosome binding site (RBS) strength is modulated by the mutation.

48. The engineered organism of claim 27, wherein a gene encoding release factor 1 (RF1) is removed from the recoded genome.

49. The engineered organism of claim 27, wherein the expression or function of release factor 1 (RF1) is impaired.

50. The engineered organism of claim 27, further comprising an orthogonal aminoacyl-tRNA synthetase and/or tRNA.

51. The engineered organism of claim 27, wherein the engineered organism is viable.

52. A method comprising culturing the engineered organism of claim 27 in growth media that comprises at least one non-standard amino acid.

53. A polypeptide comprising a non-standard amino acid, wherein the polypeptide is made using the engineered organism of claim 27.
